(19) United States
(12) Patent Application Publication
(10) Pub. No.: US 2020/0185100 A1
(43) Pub. Date: Jun. 11, 2020

(54) SYSTEMS AND METHODS FOR HEALTH TRACKING AND MANAGEMENT

(71) Applicant: ZYUS Life Sciences US Ltd., Saskatoon (CA)

(72) Inventor: Cedric Francois, Prospect, KY (US)

(21) Appl. No.: 16/596,827

(22) Filed: Oct. 9, 2019

Related U.S. Application Data
(63) Continuation of application No. 15/373,802, filed on Dec. 9, 2016, now abandoned.
Application No. 15/373,802 is a continuation of application No. PCT/US2015/034875, filed on Jun. 9, 2015.
(60) Provisional application No. 62/009,748, filed on Jun. 9, 2014.

(51) Int. Cl. G16H 50/20; G16H 10/60; G06N 5/02; G06Q 10/10; G06Q 10/06; G06Q 50/22; G16B 20/00

(52) U.S. Cl. CPC G16H 50/20; G16H 10/60; G06N 5/02; G16B 20/00; G06Q 10/0631; G06Q 50/22; G06Q 10/10

(57) ABSTRACT

In some aspects, the present disclosure provides systems and computer program products for use in health tracking and management.

This application is a continuation of U.S. Non-Provisional application Ser. No. 15/373,802, entitled “Systems and Methods for Health Tracking and Management,” filed Dec. 9, 2016, which is a continuation of PCT/US15/34875, entitled “Systems and Methods for Health Tracking and Management,” filed Jun. 9, 2015, which claims priority to U.S. Provisional Application No. 62/009,748, entitled “Systems and Methods for Health Tracking and Management,” filed Jun. 9, 2014, the disclosures of which are incorporated by reference herein.

### BACKGROUND

Interactions between patients and physicians have traditionally been episodic. Patients typically seek treatment from a physician for newly arising medical conditions and may return to the same physician for follow-up or be referred to a different physician who may then assume responsibility for treating the patient for that condition. Patients may visit more than one physician for the same complaint or consult different specialists for different complaints. Many patients suffer from several chronic diseases, require multiple medications, and are under the care of multiple physicians, who may practice at different health care institutions and may not even be aware that they each care for a particular patient. Health data pertaining to a particular patient or patients is often dispersed among diverse systems, is organized in diverse manners, and may employ a variety of different formats. On the level of the individual physician and patient, a physician often does not have ready access to data that has been obtained by other physicians who care for the patient currently or previously cared for the patient, particularly in the case of patients that have multiple physicians and/or in the case of data obtained by physicians working in different health care institutions or for different health care organizations. In addition, physicians may have an incomplete picture of the patient's health due to factors such as patient forgetfulness or lack of time to discuss the relevant questions during patient appointments. Between follow-up appointments, patients are often largely on their own to deal with questions and issues relating to their health that may arise from day to day, as is often the case with chronic diseases. The fragmented and intermittent nature of health care is particularly problematic for the increasing number of patients with chronic diseases.

### SUMMARY

In some aspects, the present invention encompasses the recognition that there is a need for systems and methods that provide health data that is structured so as to facilitate its use by physicians, patients, researchers, and/or other individuals or entities concerned with health care and human biology. There is a need for systems and methods that obtain and organize health data from diverse sources and integrate it in a structured format that allows it to be leveraged more effectively for improved health care and medical research. There is a need for systems and methods that organize health data and make it available in a manner that facilitates its use in treating patients or in understanding conditions and outcomes in accordance with the way physicians and researchers think about medicine and human biology as a result of their training and experience. There is a need for systems and methods that organize health data and make it available in a manner that facilitates patients' use of the data to become more active and informed participants in their own health care. There is also a need for systems and methods that assist physicians in treating their patients and that assist patients in understanding and engaging in their own health care. There is a need for systems and methods that accomplish such aims in ways that do not significantly interfere with typical workflows of physicians and other health care providers or associated personnel, do not require significant expenditure of time on the part of health care providers, yet have the flexibility to accommodate different treatment approaches and the needs, preferences, resources, and environments of individual patients and health care providers.

In some aspects, the present invention leverages the ever-growing number of devices of diverse kinds that permit physiological data to be obtained from patients. Aspects described herein encompass the recognition that, while these devices permit the collection of vast amounts of health data, the potential value of such data for improving patient care and conducting medical research currently remains to a large extent unrealized. In some aspects, the invention provides methods that make health data obtained by diverse devices accessible to physicians through a single user interface without requiring physicians to master the various interfaces and diverse modes of data presentation employed by multiple different device manufacturers and that permit a physician who manages a particular condition in a patient to rapidly access the particular data relevant to the condition of interest at a particular time from among a large amount of data relevant to the patient. In some aspects, the invention provides methods that make data obtained by diverse devices available through a single user interface to the patient to whom the data pertains. In some embodiments, such methods may obtain multiple types of data collected by diverse devices and present the data to the patient through a single user interface, thus obviating the need for the patient to check multiple apps or visit multiple different websites to obtain the data. In some embodiments, the system may thereby serve as a personal quantification platform for patients. In some embodiments the system organizes multiple types of data according to its relevance to particular conditions that a patient has and presents the data to the patient in a condition-centric manner. For example, physiological data relevant to a particular condition may be collected by several different devices, obtained by the system, integrated, and presented together as a cohesive module.

In some aspects, the invention provides systems and methods that integrate health data obtained from a patient in the course of his or her daily life with health data obtained from other sources, such as the data from the patient's electronic medical records maintained by one or more health care institutions or data from payers to which claims for reimbursement for the provision of health care services have been submitted.

In some aspects, the invention provides computer-implemented systems and methods for providing patients with physician-approved health tracking and management plans for use outside of medical facilities. In some embodiments, a health tracking and management plan includes a health tracking module that a patient may use between appointments with a health care provider to assess the significance of his or her symptoms and obtain management recommendations that have been approved for that particular patient by the health care provider. In some embodiments, a health tracking and management plan includes a health tracking module that a patient may use after discharge from hospital to assess the significance of his or her symptoms and obtain management recommendations that have been approved for that particular patient by a health care provider responsible for the patient's care during the hospitalization.

In some aspects, the invention provides computer-implemented systems and methods for monitoring patients' health status while they are outside of medical facilities. In some embodiments, such monitoring may permit detection of an adverse change in a patient's health status so that one or more appropriate actions can be taken. Data obtained through monitoring may be analyzed to derive information of various kinds. Data and/or information derived from the data may be stored on a computer-readable medium and made available to health care providers, patients, or both, in various formats such as in the form of a dashboard.

In some aspects, the invention provides computer-implemented systems and methods for managing the treatment of chronic conditions during the short term after a patient has been discharged from hospital. In some aspects, systems and methods described herein may reduce the likelihood that a patient will be admitted to hospital within 30 days of being discharged. In some aspects, systems and methods described herein may reduce the likelihood that a patient who has been admitted to hospital for a particular condition and discharged will be admitted to hospital for treatment of the same condition within 30 days of discharge. In some aspects, systems and methods described herein may reduce hospital readmissions for, and/or patient mortality from, various conditions in the 30 day period after patient discharge.

In some embodiments, patients may use a health tracking module to assess their condition, in accordance with their physicians' instructions, by participating in evaluations of symptoms conducted via a computer or communication device that comprises, accesses, or is controlled by a computer program product of the invention. The patient may be prompted to participate in these evaluations periodically. The patient may also initiate these evaluations voluntarily. In some embodiments the evaluations consist of one or more questions regarding symptoms of a condition and, in some embodiments, associated concurrent conditions present in that patient, as well as, in some embodiments, requests for relevant physiological data. Typically at least some of the questions are in a yes/no or multiple choice format. Underlying algorithms determine which questions and requests for data are presented to the patient, based upon factors such as importance and the patient's characteristics (e.g., a subset of the total set of potential questions/requests may be presented at any given time). Based upon the responses to these evaluations, the patient may be provided with recommendations in the form of instructions for managing the condition. In some embodiments, algorithms linking symptom data and physiological data to particular patient instructions are reviewed and approved by a health care provider, health care facility, or health care organization that uses the system.

In some embodiments the invention provides at least some of the following features for health care providers: (1) Ability to view a patient list along with biographical information, data regarding compliance, condition, and patient photo (if available); (2) Ability to view and prescribe a T-plan by a “drag-and-drop” functionality; (3) Ability to view algorithms linking symptom data and physiological data to particular patient instructions; (4) Ability to change/confirm patient profile characteristics for characteristics that are significant in the context of the underlying condition; (5) Ability to customize treatment plan details by adding questions or requests for data to a health tracker, monitoring for particular physiological data, and/or adding educational modules; (6) Ability to create templates of the health care provider's preferred treatment plan (medication and follow-up protocol) for particular conditions or use pre-existing templates provided by the system and provide them to patients in the form of a schedule for access and use by the patient on a computer; (7) Ability to view T-plans that have been prescribed to the patient by other health care providers who also use a system of the present invention, including, in some embodiments, health care providers who use different electronic medical record systems and/or work for different or unaffiliated health care institutions or health care organizations; (8) Ability to view information regarding procedures that have been performed on the patient and medications that have been prescribed to the patient by other health care providers including, in some embodiments, health care providers who use different electronic medical record systems and/or work for different or unaffiliated health care institutions or health care organizations.

In some embodiments, the invention provides at least some of the following features for patients: (1) View consolidated data on compliance, conditions, and hospitalizations, as well as physiological data and educational modules; (2) Receive periodic updates/information on conditions; (3) Prompting to perform periodic evaluation of conditions; (4) Option to perform evaluation of conditions at any time; (5) Receive schedule of upcoming procedures, appointments with health care providers; and (6) Receive medication reminders. In some embodiments, caregivers of the patient may have permission to access the program on the patient's behalf. Caregivers may be allowed to view at least some of the patient information, may receive instructions in addition to or instead of the patient, or both.

In some aspects, systems and methods for automatically configuring a health tracking and management plan for a patient with a condition, based only on the condition and a set of patient characteristics, are described. In some embodiments a health tracking and management plan is embodied as a computer program product comprising one or more data collection modules that obtain health data comprising symptom data, physiological data, behavioral data, environmental data, or any combination thereof, determine a course of action to manage the condition based at least in part on the health data, and provide directions to the patient to implement the course of action. In some embodiments a health tracking and management plan provides a schedule of health care events as determined appropriate for managing the condition. The schedule may be stored on a computer-readable medium, provided in electronic format to the patient for viewing on a computer and may automatically remind the patient of actions he or she needs to take, such as making or keeping appointments with health care providers according to the schedule.

In some embodiments, a system of the present invention is implemented at least in part through a combination of an application for use by physicians and an application for use by patients. In some embodiments the system is implemented at least in part through a combination of a web application for use by physicians and an application for a mobile device for use by patients.

In some aspects, the present invention provides computer-implemented systems and methods for collecting data and developing clinically useful information regarding patient symptoms, physiological data, and/or behavior in the context of a variety of conditions.

In some aspects, described herein is a computer program product comprising a computer-readable medium having computer-executable instructions stored thereon for performing a method for configuring a health tracking and management plan (T-plan) for a condition, the method comprising: (a) receiving information that identifies a condition; (b) receiving information that identifies a set of patient characteristics; (c) selecting one or more data collection modules based on the condition identified by step (a) and the patient characteristics identified by step (b); and (d) storing information that identifies the set of data collection modules selected in step (c) in association with an identifier for the T-plan, and an identifier of the condition, thereby configuring a T-plan for the condition. In some embodiments the patient characteristics are a selected from a predetermined set of patient characteristics that are deemed significant in the context of the condition.

In some aspects, described herein is a computer program product comprising a set of data collection modules selected and configured as a health tracking and management plan according to the method performed by a computer program product set forth above, wherein the health tracking and management plan has been assigned to a patient. In some embodiments, the health tracking and management plan has been prescribed to a patient by a health care provider of the patient.

In some aspects, described herein is a computer program product comprising a computer-readable medium having stored thereon computer-executable instructions for performing a method comprising: providing a user interface on a computer display that permits a user to select or configure a computer program product as set forth above and assign it to a patient. In some embodiments, the data collection modules, themselves or through one or more associated modules, determine directions for management of the condition by a patient or caregiver based on data received by the data collection modules and provide them to a patient or caregiver, and wherein the user interface permits the user to (i) view a representation of a process by which directions for management of the condition by a patient or caregiver are determined based on a given set of data, optionally wherein the process is represented by displaying the various combinations of data that result in particular directions, (ii) remove a feature that provides directions for management of the condition by a patient or caregiver, (iii) customize one or more elements of the directions, or (iv) any combination of (i)-(iii). In some embodiments, the user interface permits a health care provider to (i) view a patient list, (ii) select a patient from a patient list, (iii) initiate assignment of a T-plan for a condition to a patient, optionally by dragging an icon representing a T-plan to a photo of the patient or an icon representing the patient, (iv) after initiating assignment of a T-plan for a condition to a patient, select a set of patient characteristics to be used in selecting modules for the T-plan from a predetermined set of patient characteristics deemed significant in the context of the condition, (iv) after initiating assignment of a T-plan for a condition to a patient, select one or more patient characteristics to be used in selecting modules for the T-plan from a universal set of patient characteristics, (v) generate, review, modify, or confirm a patient profile for the patient, wherein the patient profile indicates one or more patient characteristics that apply to the patient and is stored on a computer-readable medium in association with an identifier of the patient; (vi) view, on a patient-specific and condition-specific basis, data collected by data collection modules of T-plans assigned to patients of the health care provider, and, optionally, analyses performed on said data, (vii) configure and store a health care event module that specifies health care events for managing the condition and timeframes therefor; (viii) select, from a set of preconfigured health care event modules, a health care event module that specifies health care events for managing the condition and timeframes therefor; (ix) view a representation of a T-plan prior to or after assignment of the T-plan to a patient; (x) initiate enrollment of a patient in a system that provides the patient with an interface through which the patient can interact, through an application on a mobile device or through an electronic communication network, with modules assigned to the patient as part of a T-plan, optionally, wherein initiating enrollment of a patient requires only the entry of the patient's email address; (xi) view a photo of the patient or an icon representing a patient together with icons representing T-plans assigned to the patient and associated conditions, (xii) select an icon representing a T-plan of interest and view condition-specific data collected by data collection modules of the T-plan, and, optionally, analyses performed on said data, (xiii) prescribe a connected monitoring device for obtaining physiological data from the patient, or (xiv) any combination of (i)-(xiii).

In some aspects, described herein is a computer program product comprising computer-executable instructions stored on a computer-readable medium for performing a method comprising: conducting an interactive health evaluation regarding a condition and presenting directions for management of the condition to the patient, wherein the directions are determined based on data obtained through the interactive health evaluation, wherein the interactive health evaluation (i) is conducted automatically upon detection of physiological data indicative of a deterioration or potential deterioration or notable change in the patient's health and/or (ii) is conducted automatically on a recurring basis, optionally wherein the interactive health evaluation is, in addition to (i) and/or (ii), also conducted at any time in response to a request received from a patient who suffers from the condition. In some embodiments the interactive health evaluation is, in addition to (i) and/or (ii), also conducted at any time in response to a request received from a patient who suffers from the condition. In some embodiments, the directions are determined based on data obtained by one or more connected monitoring devices and data obtained through the interactive health evaluation. In some embodiments, the directions are determined using an algorithm that applies a set of rules that map from (1) a condition and a subset of patient characteristics selected from a predetermined set of patient characteristics that are deemed material to management of the condition to (2) a subset of a set of directions relevant to managing that condition.

In some aspects, described herein is a computer program product comprising a computer-readable medium having stored thereon: (a) a library of modules comprising one or more data collection modules each comprising computer-executable instructions for performing a method comprising: obtaining symptom data, physiological data, behavioral data, environmental data, health care event data, other health or health-related data, or a combination thereof regarding or relating to a patient, wherein at least some of the data collection modules are configured to be made accessible to a patient through an application on a mobile device or through an electronic communication network; (b) a configuration module comprising computer-executable instructions that configure a T-plan for a condition by (i) selecting a set of one or more data collection modules that are relevant to the condition based on input that identifies a condition and a subset of patient characteristics selected from a set of patient characteristics deemed significant in the context of the condition; (ii) associating identifiers of the set of one or more data collection modules with an identifier of a patient based on input that identifies the patient; and (iii) associating an identifier of the T-plan and an identifier of the condition with the set of data collection modules, optionally wherein the configuration module adds, removes, or customizes one or more of the data collection modules based on input that specifies such addition, removal, or customization; (c) a data storage module that receives data obtained by data collection modules assigned to patients and stores the data identified according to its type in one or more databases in association with an identifier of the patient to whom the data pertains, and, optionally, an identifier of the source of the data; and (d) a data extraction module that extracts data pertaining to a particular patient from said one or more databases according to its type in response to a request and provides it to one or more other modules of the computer program product. In some embodiments, the modules further comprise (e) an analysis module that performs diverse types of analyses on data obtained by the data collection modules; and (f) a display module that generates one or more screens that display data collected by the data collection modules of a T-plan and analyses performed thereon by the data analysis module. In some embodiments, the library of modules comprises, for each of a plurality of conditions, one or more health tracking modules that obtain symptom data and/or physiological data of one or more types that are relevant to a condition and one or more monitoring modules that obtain physiological data of one or more types that are relevant to a condition. In some embodiments the library of modules further comprises, for each of a plurality of conditions, one or more monitoring modules that obtain data pertaining to behaviors that are relevant to a condition, one or more environment tracking modules that obtain data pertaining to environmental factors that are relevant to a condition, and/or one or more educational modules that provide educational material relevant to a condition and obtain behavioral data concerning use of the educational module by a patient. In some embodiments, the library of modules further comprises one or more health care event modules that specify health care events for managing a condition and a timeframe therefor, optionally wherein a health care event module itself or through one or more associated modules, is capable of generating a schedule of health care events for use by a patient or caregiver through an application on a mobile device or through an electronic communication network. In some embodiments, at least one of the data collection modules specifies at least one type of data element to be collected and a timeframe for receiving data elements of that type, wherein the timeframe specifies a frequency and a time window, and wherein the data collection modules, themselves or through one or more associated scheduling/notification modules, perform a method that comprises: (i) receiving and/or monitoring receipt of data relating to a patient; and (ii) performing one or more scheduling or notification functions based on said receiving and/or monitoring and the specified frequency and time window. In some embodiments, one or more of the scheduling and notification functions is selected from (i) determining when the next data element of that type is due, (ii) determining whether a data element is current (not expired), (iii) determining whether a data request or notification should be issued; (iv) determining when a data request or notification should be issued, and (v) issuing a data request or notification. In some embodiments, at least some of the data collection modules, themselves or together with one or more associated modules, collectively comprise computer-executable instructions for: (1) obtaining data comprising symptom data, physiological data, behavioral data, and/or environmental data of one or more types that are relevant to the condition identified in step (a); and (2) determining directions for management of the condition by a patient or caregiver based on the data and providing the directions to a patient or caregiver; (3) determining a compliance score based on the data, (4) determining a health score based on the data, (5) determining whether the patient has experienced a significant deterioration in health within a selected time period or is at risk of experiencing a significant adverse change in health within a selected time period based on the data, (6) determining whether a change in the type of data to be collected and/or in the timing of data collection is warranted based on the data; (7) determining whether a notification to a caregiver or health care provider should be issued based on the data; (8) determining, based on the location of a patient's mobile device, that the patient likely experienced a hospitalization event, and, optionally, (A) adjusting one or more modules of a T-plan assigned to the patient to a heightened sensitivity mode or post-discharge use mode, further optionally switching the module out of heightened sensitivity mode or post-discharge use mode after a predetermined time period; and/or (B) notifying a health care provider or caregiver of the patient of the likely hospitalization event, or (9) any combination of (1)-(8). In some embodiments, a likely hospitalization event is considered to be a hospitalization event if a patient's device is determined to be located at a hospital for at least 12 hours.

In some aspects, described herein is a computer program product comprising a computer-readable medium having computer-executable instructions stored thereon, the computer-executable instructions for performing a method comprising: (a) receiving from a user information that identifies a patient and a condition; (b) presenting to the user a user interface configured to permit the user to select a subset of patient characteristics that apply to the patient from a predetermined set of patient characteristics that are deemed significant in the context of the condition; (c) receiving from the user information selecting a subset of the set of patient characteristics; (d) selecting a set of one or more data collection modules based on the condition and the patient characteristics selected by the user, wherein at least one of the data collection modules obtains symptom data, physiological data, or both, regarding the patient; (e) providing access to at least some of the data collection modules selected in step (d) to the patient or a caregiver through an application on a mobile device or through an electronic communication network; (f) receiving data entered by the patient or a caregiver or collected by a connected monitoring device over a course of time; and (g) storing at least some of the data received in association with an identifier of the patient. In some embodiments, the method further comprises determining directions for management of the condition by a patient or caregiver based on the data; and providing the directions to the patient or a caregiver through the application on a mobile device or through the electronic communication network.

In some aspects, described herein is a computer program product comprising a computer-readable medium having computer-executable instructions stored thereon for performing a method comprising: (a) retrieving a set of data that is relevant to a particular condition in a particular patient from a database comprising symptom data, physiological data, or both, obtained through an application on a mobile device or through an electronic communication network from each of multiple patients while such patients are not inpatients; and (b) displaying at least some of the retrieved data or analyses of at least some of the retrieved data to a health care provider of the patient, optionally wherein the health care provider is the individual who assigned the data collection module to the patient. In some embodiments, the data pertaining to any particular patient was obtained by a data collection module assigned to such patient by a health care provider of the patient. In some embodiments, the database comprises data of multiple types relevant to multiple conditions

In some aspects, described herein is a computer program product for health tracking and management comprising a computer-readable medium having computer-executable instructions stored thereon for performing (i) a first method comprising providing a user interface on a computer display that permits a health care provider to (a) select or configure a health tracking and management plan (T-plan) comprising one or more data collection modules and assign it to a patient, wherein the T-plan pertains to a condition and comprises a health tracking module that obtains symptom data and/or physiological data relevant to the condition or a monitoring module that obtains physiological data relevant to the condition; and (b) view, on a patient-specific and condition-specific basis, data collected by data collection modules of T-plans assigned to patients of the health care provider, and, optionally, analyses performed on said data; (ii) a second method comprising (a) receiving, through the user interface of the first method, information that identifies a patient and information that identifies a T-plan for a condition or is sufficient for configuration of a T-plan for the condition for that patient; and (b) storing information that identifies the one or more data collection modules in association with an identifier for the T-plan, an identifier of the condition, and an identifier of the patient, thereby configuring a T-plan for the condition and assigning it to the patient; (iii) a third method comprising providing, in an application on a mobile device or through an electronic communication network, a user interface that permits a patient or caregiver to interact with a T-plan assigned to the patient according to the second method; and (iv) a fourth method comprising obtaining symptom data, physiological data, behavioral data, health care event data, or a combination thereof regarding a patient, as specified by a T-plan assigned to the patient according to the second method, wherein at least some of the data are obtained from the patient through an application on a mobile device or through an electronic communication network; and storing the data in association with an identifier of the patient. In some embodiments, at least one of the data collection modules interactively obtains data from the patient through an application on a mobile device or through an electronic communication network or obtains data collected by a connected monitoring device. In some embodiments, the computer-executable instructions further perform (v) a fifth method comprising determining directions for management of a condition by a patient or caregiver based on data obtained interactively from the patient or caregiver through an application on a mobile device or through an electronic communication network by one or more data collection modules of a T-plan assigned to the patient and providing the directions to the patient or a caregiver through the application or electronic communication network.

In some aspects, described herein is a computer program product comprising a computer-readable medium having computer-executable instructions stored thereon for performing a method comprising: (a) selecting, based on input from a health care provider, a set of data elements of one or more types that are relevant to a condition and timeframes that specify at least a frequency for obtaining data elements of each type; (b) storing information that identifies the data element types and timeframes in association with an identifier of the patient and an identifier of the condition; (c) performing one or more processes that obtain such data elements over a course of time; and (d) storing the data elements so obtained and, optionally, a relevant time, in a patient record. In some embodiments, step (a) further comprises selecting, based on input from a health care provider, a set of health care events of one or more types that are relevant to a condition and timeframes that specify at least a frequency for performing health care events of each type, step (b) further comprises storing information that identifies the health care event types and timeframes in association with an identifier of the patient and an identifier of the condition, step (c) further comprises performing one or more processes seek evidence of the occurrence of such health care events over a course of time, and step (d) further comprises storing information indicating the occurrence of those health care events of which evidence is detected. In some embodiments, step (c) causes the patient to be presented periodically, through an application on a mobile device or through an electronic communication network, with requests for data elements of one or more types relevant to the condition, wherein the requests for data elements of a particular type are timed based on the timeframe specified for obtaining data elements of that type, wherein optionally at least some of the requests are in the form of an interactive health evaluation, further optionally wherein the patient may participate in an interactive health evaluation at any time. In some embodiments, the interactive health evaluation comprises determining directions for management of the condition by a patient based on data submitted in response to the requests, and the method further comprises presenting the directions to the patient. In some embodiments, the directions may be determined using an algorithm that applies a set of rules that map from (1) a condition and a subset of patient characteristics selected from a predetermined set of patient characteristics that are deemed material to the management of the condition to (2) a subset of a set of directions relevant to managing that condition, and the patient record comprises information that identifies the particular subset of patient characteristics that the patient actually has among those patient characteristics that are deemed material to the management of the condition.

In some aspects, described herein is a computer program product comprising a computer-readable medium having a health tracking module comprising computer-executable instructions stored thereon for performing a method comprising: (a) obtaining, from a patient with a condition, health data of one or more types that are relevant to the condition; (b) determining directions for management of the condition by a patient or caregiver based on the health data; and (c) presenting the directions to the patient via a computer or electronic communication network, wherein the health tracking module is configured to be assignable to a patient or has been assigned to a patient. In some embodiments, the directions are customizable. In some embodiments, types of health data to be obtained, the method by which the directions are determined, or both, are determined based on the condition and a set of patient characteristics, wherein the set of patient characteristics is a subset of patient characteristics selected from a predetermined set of patient characteristics that are deemed significant in the context of the condition. In some embodiments, step (a) comprises (i) conducting an interactive health evaluation with the patient or caregiver, optionally wherein the interactive health evaluation comprises questions presented by an on-screen image and/or a recording of the patient's health care provider; (ii) obtaining data collected by a connected monitoring device; or (iii) both (i) and (ii), optionally wherein the questions asked, the frequency with which questions are asked, or both, are determined at least in part based on physiological data collected by a connected monitoring device. In some embodiments, the method comprises automatically conducting the interactive health evaluation on a recurring basis and/or automatically conducting the interactive health evaluation upon detection of physiological data indicative of a deterioration or potential deterioration or notable change in the patient's health, and conducting the interactive health evaluation at any time in response to a request, optionally wherein the frequency or time at which the interactive health evaluation is conducted automatically is determined at least in part by the patient, is determined at least in part by a prescriber, or is automatically adjusted based on health data regarding or relating to the patient that was previously obtained by the computer program product. In some embodiments, the computer program product of any of originally filed claims 100-112A, wherein the method comprises determining whether the patient is at sufficient risk of experiencing a significant adverse change in health to warrant an action and, if so, providing directions to take such action to the patient or caregiver. In some embodiments, the method further comprises (d) storing at least some of the health data, in a patient record; and (e) presenting at least some of the health data, or analyses of at least some of the health data, on a display of a computer used by health care provider who manages the condition in the patient, optionally wherein at least some of the data is presented in a dashboard format, comprises a compliance score, comprises health-related event data, or any combination of the foregoing. In some aspects, described herein is a mobile device comprising the computer program product or a user interface thereto.

In some aspects, described herein is a computer-implemented method comprising: (a) receiving information that identifies a condition and a set of patient characteristics; (b) configuring a health tracking and management plan (T-plan) based on the condition and the patient characteristics, wherein the T-plan identifies (i) the condition and (ii) a set of data elements to be obtained and timeframes for their collection, wherein the data elements are relevant to the management of condition; and (c) storing the T-plan on a computer readable medium. In some embodiments, the method further comprises receiving information that identifies a patient and storing information that identifies the patient in association with information that identifies the T-plan.

In some aspects, described herein is a computer-implemented method of providing a patient with health care support comprising: (a) receiving information identifying a patient; (b) receiving an electronic prescription for a health tracking and management plan (T-plan) from an authorized prescriber; and (c) making the T-plan available to the patient. In some embodiments, the T-plan is at least in part designed for post-discharge use, optionally wherein the prescriber is an inpatient health care provider. In some embodiments, information identifying the patient is received while the patient is not hospitalized for the condition, and the T-plan is at least in part designed for chronic care use. In some embodiments, information identifying the patient is received while the patient is hospitalized for the condition, and the T-plan is designed at least in part for post-discharge use, optionally wherein the prescriber is an inpatient health care provider. In some embodiments, the T-plan information identifying the patient is received while the patient is not hospitalized for the condition, and the T-plan is at least in part designed for chronic care use.

In some aspects, described herein is a computer-readable medium having stored thereon data obtained by a computer program product described herein, the data regarding one or more patients, wherein the data regarding a particular patient are stored in association with an identifier of the patient. In some embodiments, the data further comprise health care event data regarding one or more of the patients. In some embodiments, at least some of the health care event data are obtained from a source other than the patient to whom it pertains, optionally wherein the source comprises a claim for reimbursement for health care services or an electronic medical record pertaining to the patient. In some aspects, described herein is a database, tangibly embodied on a computer-readable medium, comprising data obtained by a computer program product described herein, the data regarding a plurality of patients, wherein the database comprises data obtained from at least one thousand different patients.

### BRIEF DESCRIPTION OF THE DRAWINGS

FIG. 1 is a schematic diagram of a health tracking and management system according to certain embodiments.

FIG. 2 is a schematic diagram of a high level design of a health tracking and management system according to certain embodiments.

FIGS. 3A-3J are images showing various portions of a patient interface according to certain embodiments.

FIGS. 4A-4D show a process in the form of a decision tree for conducting an interactive health evaluation for congestive heart failure in accordance with certain embodiments.

FIG. 5 is a schematic diagram depicting an architecture which can be used in obtaining health data collected by a connected monitoring device according to certain embodiments.

FIGS. 6A-6E are images showing various portions of a physician interface according to certain embodiments.

FIG. 7 shows a process for automated patient check-in for a health care appointment in accordance with certain embodiments.

FIG. 8 is a schematic diagram of a patient data integration system according to certain embodiments.

### GLOSSARY

Certain terms used in the present application are collected here for convenience. General or specific features of the description of terms in this glossary may be applied in or to any aspect, embodiment, context, description, or claim in which such term is used.

“Computer-implemented method” means a method or process that is at least in part performed by a computer. The computer may perform the method in response to input from a user.

“Condition-centric” as it pertains to health data means that the relationship of health data to a condition is the guiding principle used to determine which health data to obtain, analyze, store, and/or present to a user, and/or to determine how the health data are presented to a user.

The term “knowledge base” refers to a collection of information that a system may use for performing one or more processes or methods. The term “knowledge base” is not intended to imply any particular structure. The information in a knowledge base may be embodied as a set of rules, as entries in a table, or in any other suitable way. Knowledge in a knowledge base may embody the knowledge of practitioners in medicine or a particular health care discipline.

The term “monitoring device” refers to a device that can be used for detecting or measuring one or more physiological variables, environmental variables, or behavioral variables relevant to a patient. Many monitoring devices of interest herein are devices that can be used by a patient in his or her daily life, i.e., while the patient is not under care in a health care facility. Such monitoring devices may be referred to as “personal monitoring devices”. Unless otherwise indicated a monitoring device may be a personal monitoring device. Monitoring devices include: body weight scale, pulse oximeter, blood pressure monitor, thermometer, activity tracking device, heart rate monitor, blood glucose measuring device (glucometer), stethoscope, medication dispensers that in some way monitor medication usage, and any other device capable of obtaining physiological data, environmental data, or behavioral data relating to a patient in his or her daily life.

The term “connected monitoring device” refers to a monitoring device that is or can be connected to a communication network or to a computer that is or can be connected to a communication network such that data collected by the device can be obtained by a system of the present invention without a user entering the data manually. A computer, e.g., a mobile device, equipped with or in communication with one or more appropriate sensors may serve as a connected monitoring device. Connected monitoring devices also include implanted, indwelling, or swallowed devices that are capable of wirelessly transmitting physiological data to a computer. Connected monitoring devices may be located in (as an integral part) or on of a mobile device or mobile device case. A connected monitoring device is typically a personal monitoring device. One of ordinary skill in the art will appreciate that a large and growing number of connected monitoring devices are available, any of which may be used in various embodiments of the present invention to obtain physiological data.

The term “activity tracking device” refers to a monitoring device for monitoring and tracking fitness-related physiological parameters such as movement (e.g., distance walked or run, steps climbed), calories used, heart rate, sleep-related physiological parameters such as sleep duration, sleep depth, and any of a variety of others. An activity tracking device may use a three-dimensional accelerometer to sense user movement and measure steps taken, which it may use, sometimes together with user data, to calculate metrics such as distance walked, calories burned, floors climbed, and activity duration and intensity. Often an activity tracking device is a wearable electronic device that is or can be synchronized, in many cases wirelessly, to a computer or mobile device such as a smartphone. An activity tracking device may in some embodiments monitor activity in a room or within a home by means of heat-sensing (e.g., infrared), light-sensing, or other devices that detect movement or heat without necessarily being worn by or connected to the patient. Such devices may, for example, determine whether a patient has deviated from his or her normal level of activity within a home, failed to get out of bed, etc.

The term “condition” refers to a circumstances or set of circumstances which can be used to characterize the state of an individual's health. Thus, a “condition” would include a health problem or abnormality or any state of abnormal or normal health or function for which an individual seeks or obtains care from a health care provider. A “condition” would also include the state of good health which an individual might wish to maintain, but for which he or she would not necessarily seek or obtain care from a health care provider. “Condition” encompasses diseases, disorders, illnesses, injuries, disabilities, syndromes, symptoms, and health-related complaints. “Disease” refers to a health problem or abnormality or state of abnormal health or function and is used interchangeably herein with “disorder” or “illness”. A condition may be a complication of a disease or may be a particular symptom or group of symptoms. A “complication” of a condition may be any unfavorable evolution, manifestation, or consequence of the condition or of a therapy for the condition. For example, a disorder can become significantly more severe, become widespread throughout the body instead of localized or affect additional organ systems. Such occurrences may be recognized as distinct complications. In certain embodiments a condition is a diagnostic entity that has been assigned a code in the International Statistical Classification of Diseases and Related Health Problems, 10th Revision, 2007 (“ICD-10”, World Health Organization) or in the International Statistical Classification of Diseases and Related Health Problems, 10th Revision, Clinical Modification, 2011 (“ICD-10-CM”), developed by the US National Center for Health Statistics (NCHS), or any predecessor or update of either. In some embodiments, a condition is a diagnostic entity discussed in Goldman's Cecil Textbook of Medicine, Saunders, 24^{th} ed. (2012) or Lango, D., et al., Harrison's Principles of Internal Medicine, McGraw Hill, 18^{th} ed. (2011).

“Health care discipline” refers to an area of health care practice. Broadly, disciplines may be classified as medical (in which the main diagnostic and therapeutic activities are not major surgery) or surgical (in which surgery is a significant or main part of the diagnostic and/or therapeutic activities), by age range of patients (e.g., pediatrics), body system (where symptoms and diseases typically treated arise from or mainly affect a particular organ system or physiological system), as mainly diagnostic or mainly therapeutic, or based on techniques used (e.g., radiology), or by site of care (e.g., hospital, emergency room). A discipline may be a specialty or subspecialty in which certification is offered by a member board of the American Board of Medical Specialties (http://www.abms.org), such as the American Board of Internal Medicine (http://www.abim.org/) or by the American Board of Physician Specialties (ABPS). Specialties and subspecialties include, e.g., allergy and immunology; cardiovascular disease (cardiology); dermatology; endocrinology, diabetes & metabolism; family medicine; gastroenterology; hematology; hospital medicine; infectious disease; internal medicine; nephrology; neurology; obstetrics & gynecology; oncology (e.g., medical oncology); ophthalmology; otolaryngology; pediatrics; pulmonary disease (pulmonology); psychiatry; rheumatology; and urology. In some embodiments a discipline may be long term care, rehabilitation, or physical therapy. A specialty may embrace multiple specialties and/or subspecialties. For example, internal medicine encompasses multiple subspecialties. In some embodiments, multiple subspecialties or specialties may be aggregated into a single discipline. It will be understood that various specialties and subspecialties may overlap in terms of the conditions treated. Those of ordinary skill in the art are aware which conditions are commonly treated by health care providers practicing in a particular specialty or subspecialty and would understood that many conditions can be appropriately treated by providers in different health care disciplines.

A “hospitalist” is a physician whose professional focus is hospital medicine, i.e., the health care discipline concerned with the medical care of hospitalized patients such as those hospitalized for an acute illness or an exacerbation of a chronic illness. A hospitalist may be certified by the American Board of Hospital Medicine or may have earned an Internal Medicine Certification with a Focused Practice in Hospital Medicine by the American Board of Internal Medicine.

“Health care institution”, also referred to as a “health care facility” refers to an institution that provides health care to multiple persons on a systematic basis, such as a hospital, health clinic, health center, surgery center, skilled nursing facility, or physicians' practice. A health care institution may provide inpatient health care (care in which a patient is “admitted” to the institution), outpatient health care (care in which the patient is not admitted), or both. For purposes of the present disclosure a patient is considered “admitted” to a health care facility and thus an “inpatient” if the patient is considered admitted by the institution according to its records and/or is under care in a health care institution and the patient's medical record indicates that the patient is expected to remain (or has in fact remained) in the health care institution for at least 24 hours, at least two midnights, or is legally considered an “inpatient” according to applicable law or regulation. A patient is considered to be “readmitted” if the patient is admitted within 30 days of being discharged. For purposes of the present disclosure a patient who is “admitted” to hospital is considered to be “hospitalized”.

“Health care organization” refers to an organization that comprises two or more health care institutions that are commonly owned, controlled, or operated or have agreed to interact with each other and in some way operate together in caring for patients or present themselves to the public as a single entity or network. The health care institutions may be of any one or more types, e.g., two or more hospitals, two or more physician practices, one or more hospitals and one or more physician practices.

“Health care provider” refers to health care practitioners within the fields of medicine (deemed to include surgery), nursing, dentistry, and allied health professions. A health care provider may be a practitioner in any health care discipline. Health care providers include physicians, advanced clinical practice nurses (nurses with post-graduate education in nursing such as a master's degree or doctorate such as nurse practitioners and clinical nurse specialists), registered nurses, nurses, physician assistants, physical therapists, nutritionists, psychologists, and dentists. A health care provider may be licensed and/or certified in a particular health care profession, specialty, or subspecialty. For purposes of description, the present disclosure often uses the term “physician”, but unless otherwise indicated, embodiments are disclosed in which such description may refer to a health care provider of any type. In certain embodiments of any aspect of the present disclosure, the term health care provider refers to a physician.

A “T-plan” is a specification comprising information that identifies one or more types of health data elements to be collected and, for at least some of the types of data elements, also identifies a timeframe.

“Assign”, “assigning” and like terms, when used herein in the context of assigning a T-plan to an individual, means to authorize the use of the T-plan for an individual. A T-plan may be assigned providing acceptable confirmation to the system that the T-plan is to be used for the patient as an active T-plan, in that the system is to collect data relating to the patient as specified by the T-plan and is to make the various patient-directed features specified by the T-plan available to the patient for use. Such confirmation may be provided by an individual who authenticates himself or herself appropriately to the system, such as by providing appropriate log-in credentials, and is appropriately authorized to provide such confirmation. Where the authorization is provided by a health care provider of a patient or an individual acting under direction of a health care provider who is authorized to do so, assigning a T-plan may be referred to as “prescribing” the T-plan to the patient. Typically a health care provider who prescribes a T-plan to a patient or authorizes prescription of the T-plan to a patient is at least in part responsible for managing the care of the patient at the time the T-plan is prescribed. In general, assigning a T-plan to a patient associates an identifier of a patient with information sufficient to identify the T-plan and information sufficient to identify the individual who authorized the assignment of the T-plan to the patient. Information associating an identifier of a patient with information sufficient to identify a T-plan may be stored on a computer-readable medium, e.g., in a database.

As used herein, a T-plan is “configured to be assignable” if it comprises or is associated with appropriate computer-executable instructions so that an authorized user of the system can provide information that identifies a patient and the T-plan and confirmation that causes the system, following receipt of the confirmation, to use the T-plan for the patient as an active T-plan, in that the system collects data relating to the patient as specified by the T-plan and makes the various patient-directed features specified by the T-plan available to the patient for use. Typically the confirmation will also cause a record of the assignment to be stored, e.g., by causing information that identifies the T-plan to be stored in association with an identifier of the patient and an identifier of the individual who provided the authorization.

The term “based on” is used to mean that a thing is determined at least in part by whatever it is described as being “based on”. If something is determined entirely by a thing, it will be described as being “based solely on” that thing. Where the term “based on” is used herein, embodiments are provided in which the thing is determined in part by whatever it is described as being based on and embodiments in which the thing is based solely on that thing. To “determine” something means to produce or generate, compute, establish, or specify that thing. For example, something may be “determined” by selecting it from a group of alternatives or options, by analyzing data (e.g., by applying an algorithm to the data) to generate a result.

The terms “notify” or “notifying” refers to providing information by a system to a user to inform the user of some circumstance or occurrence. A “notification” is the message or communication that provides the information, e.g., by displaying it on a screen. A notification may, for example, inform the user of the occurrence of an event, inform the user that a scheduled event failed to occur within a specified timeframe, or remind the user of an upcoming event or action that the user should take. A notification may comprise written or spoken information and may or may not request a response or acknowledgement. A notification may or may not be provided within the normal user interface of an application of the present invention. In some embodiments when a notification is issued, the user's device emits an intermittent or continuous sound, vibration, or other stimulus. A sound may be a verbal message, which may inform the patient of the notification and may, in some embodiments, inform the patient of at least some of the content or prompt the patient to check his or her notifications.

“Primary care provider” (sometimes abbreviated PCP) refers to a health care provider who provides initial care for patients with an undiagnosed health condition or health concern and typically provides continuing care of patients with varied medical conditions not generally limited to specific organ systems or diagnoses. A PCP may make referrals to specialists who provide health care for particular conditions within their specialty. A PCP is often a physician but may be an advanced clinical practice nurse, registered nurse, or other health professional. A PCP may have undertaken postgraduate training in a primary care program, such as family medicine (also called family practice or general practice), pediatrics, or internal medicine.

“Associated personnel” generally refers to assistive personnel and support workers who are part of health care teams and/or work with health care providers but do not directly provide patient care, although they may interact with patients in various ways. They may, for example, perform scheduling, care coordination, address patient questions that do not require direct interaction of the patient with a health care provider, and/or perform clerical functions.

“Health data” is used herein in a broad sense to refer to any data or information about or relating to an individual or group of individuals that is descriptive of, informative about, affects, or potentially affects the health of the individual or group of individuals in more than merely a tangential or insignificant way, as well as health care event data (as described below). Health data encompasses demographic data, medical history data, symptom data, physiological data, genotypic data, omics data, health-related behavioral data, health-related physical environment data, health-related social environment data, health care event data, and any type of data that may be of use to a health care provider in diagnosing or managing a condition. “Health-related”, as it pertains to data, e.g., physical environment data, social environment data, or behavioral data, means that data of the particular type, or the circumstances that the data reflects, may reasonably be expected to have an effect on health. Health data typically does not refer to data that is specifically concerned with the financial aspects of health care provision. In this regard it should be understood that the health data in claims, such as codes specifying particular procedures or medications or identifying particular health care providers or health care facilities, are distinct from the data specifically concerned with the financial aspects of health care provision, such as the cost of particular procedures, eligibility criteria, co-payments, deductibles, and the like. Any such financial information or any other type of information may be expressly excluded from the scope of health data. Health data may comprise metadata. “Metadata” refers to data that provides information about data of interest. Metadata may include any information that may be necessary or helpful in interpreting, understanding, or making use of the data of interest, e.g., information regarding the type, structure or format of the data, information identifying or describing the data content, e.g., identifying the particular parameter being measured, identifying a particular procedure or instrument that yielded the data, or whatever other information is appropriate in the context. Where metadata are associated with a particular data element, the data element may be referred to as being “tagged” with the metadata.

“Health care event” refers to those acts and occurrences that take place as part of a patient's health care. Health care events may be classified as “physician events” and “patient events”. “Physician events” encompass any procedures or other activities or services (collectively “procedures”) that may be performed on or directed or delivered to a patient by a HCP as part of providing health care to a patient, e.g., with the object of improving health, treating disease or injury, or making a diagnosis. Procedures include diagnostic procedures (e.g., medical tests), therapeutic procedures (including surgical procedures), and patient encounters with a health care provider (e.g., initial patient visit, follow-up visits). Physician events may include, for example, procedures performed at least in part by a clinical laboratory, imaging center, or other entity that provides health care services. Physician events may include any physician service, physical or occupational therapy service, radiologic procedure, clinical laboratory tests, other medical diagnostic procedures (e.g., pathology, molecular diagnostic tests, imaging), etc. A physician event may be classified as a diagnostic procedure or a therapeutic procedure but the term encompasses procedures performed for disease prevention, diagnosis and/or management (e.g., treatment, monitoring). It will be appreciated that many procedures that may be performed for diagnostic procedures to determine whether a patient has a disease or which disease a patient has may be performed after diagnosis for purposes of monitoring the patient (e.g., assessing the condition of the patient, response to treatment, progression or resolution of the disease, etc.). Typically a procedure takes place or is performed in a health care facility but may take place or be performed anywhere that a HCP provides health care. A physician event may have (i) a code in the ICD-9-PCS and/or ICD-10-PCS, (ii) a code in the Healthcare Common Procedural Coding System (HCPCS), e.g., a Common Procedural Terminology (CPT)® code set, (iii) a Logical Observation Identifiers Names and Codes (LOINC) code, or (iv) any of the foregoing. In certain embodiments, a procedure has an ICD-9-PCS and/or ICD-10-PCS code if performed on a hospital inpatient and a HCPCS code, e.g., a CPT code, if performed on a patient who is not a hospital inpatient. “Patient events” encompass any actions that may be performed by a patient or caregiver as part of managing the patient's health, particularly those actions that may be undertaken upon the recommendation of a health care provider or according to a prescription of a health care provider. Patient events include, for example, self-administration of medications or administration of medications by a caregiver, performing physical activity in accordance with a health care provider's recommendation or prescription, and making and attending appointments with health care providers. Certain events, such as a patient's visit to a health care provider, may be both a physician event and a patient event. Certain types of procedures may be a physician event or a patient event depending on the context in which they are performed. For example, a blood glucose test may be a physician event (a diagnostic procedure) if performed by a health care provider when a patient visits his or her physician and a patient event (body monitoring) if performed by the patient at home. Similarly, an insulin injection may be a physician event (a treatment procedure) if performed by a health care provider when a patient visits his or her physician and a patient event (self-administration of medication) if performed by the patient at home.

“Health care event data” refers to health care event occurrence data and health care event result data. “Health care event occurrence” data refers to data indicating whether or that a health care event occurred and data pertaining to the occurrence of a health care event, such as the date and time the event occurred, the location at which the event occurred, the health care provider who ordered or performed the event, the health care facility at which the event occurred, and other data relevant to the occurrence of the health care event. “Health care event result data” refers to data generated from or in the course of a health care event that may be used in or relevant to the health care of the patient, e.g., data that a health care provider may find useful in providing health care to a patient, e.g., in establishing or ruling out a diagnosis, determining a prognosis, selecting a treatment, evaluating the efficacy, side effects, or risk/benefit ratio of a treatment, advising a patient or caregiver in regard to the patient's health, or the like. Health care event result data may comprise primary data, processed data, information derived from analysis of the data by a machine or human being. A health care event that generates health care event data or is of a type that would generate health care event data may be referred to as an “underlying health care event” with respect to such health care event data.

“Symptom data” refers to data pertaining to symptoms. In general, the term “symptom” refers to those features of a condition that are noticed by a patient or would ordinarily be noticeable by a patient. Symptoms encompass any of the myriad departures from normal function or feeling that a patient with a condition may experience. Symptoms are often features about which a patient may tell a physician when seeking health care and/or about which a physician may question a patient when evaluating a patient for purposes of diagnosis, prognosis, and/or treatment. For example, a patient with COPD may complain about shortness of breath (dyspnea), cough, and sputum production. A physician suspecting that a patient may have COPD or deciding whether a patient with COPD would benefit from a change in medication may ask a patient about the presence or severity of these symptoms. In general, symptoms are features that are or can be perceived or observed without the use of devices or other equipment. Many symptoms are subjective, i.e., they relate to the way the patient feels and may not be directly measurable, although their presence or severity may correlate with one or more measurable parameters. For example, shortness of breath is a feeling experienced by a patient and is not directly measurable but may correlate with an abnormally high respiratory rate. Symptom data may include any information about a symptom, such as data indicating the presence, absence, intensity, or frequency of a symptom, or a change over time in any of these.

“Physiological data” encompass qualitative and quantitative measurements of any indicator of a biological state, function, structure, process, response, or condition in a patient. Such indicators include any of the numerous variables (parameters) that are commonly measured in medicine to evaluate a patient for purposes such as diagnosis, prognosis, and/or treatment. Typically, indicators of interest herein are those whose values (which may be quantitative or qualitative) reflect, characterize, or are related to the function or structure of organs and organ systems and/or whose values reflect, characterize, or are related to the presence or severity of conditions. Examples include indicators of pulmonary function, cardiovascular function, kidney function, liver function, immunological function, digestive function, metabolic function, hematologic function. Physiological data are typically obtained through use of a monitoring device or other measuring or testing equipment, although certain physiological data such as heart rate or respiratory rate may be measured or estimated, e.g., with use of a clock or conventional watch.

Physiological data elements encompass individual values, such as heart rate, oxygen saturation, or body weight, collections of related values such as the data that make up an electrocardiogram (EKG), and values derived by processing or analyzing data that is directly measured from a patient. Multiple items of data of one or more types may be analyzed to obtain useful information. For example, heart rate and time spent sleeping may be combined to produce an average heart rate during sleep. Analyses of these types may be used to derive indicators useful for evaluating a patient's condition, may be reported to health care providers, or otherwise used by the computer program product. Processing or analysis may be performed by a monitoring device, health tracking module, or both, depending, e.g., on the particular data and monitoring device. In some embodiments a computer, e.g., a mobile device, is capable of obtaining an electrocardiogram (EKG) or serving as an electronic stethoscope.

Physiological data also encompass the presence, amount (e.g., concentration), or activity of any of a variety of endogenous or exogenous substances in biological samples obtained from a patient, wherein the presence, level (e.g., concentration), or activity of the substance(s) is informative about the state of one or more physiological functions, organs, organ systems or other body structures, or conditions. Biological samples include body fluids such as blood, exudate, nasal secretions, pus, saliva, sputum, sweat, tears, urine; feces; skin; exhaled air, or any other biological material that can be obtained from a patient. The substances may be small molecules (e.g., glucose), proteins (e.g., total protein, or specific proteins of interest), metals or ions (e.g., sodium, bicarbonate, potassium, phosphate, magnesium, calcium), nucleic acids, hormones, metabolites, nutrients, or antibodies. In some embodiments, the substance is detected or measured using a test substrate such as a dipstick or test strip, which may contain one or more reagents for detection of the substance, such as by a colorimetric readout. Physiological data may include any measurement of a “biomarker”, which term refers to a characteristic or parameter that can be objectively measured and evaluated as an indicator of normal biological processes, pathogenic processes (e.g., the presence, prognosis, status, or degree of severity of a condition), or responses to a therapeutic intervention. In certain embodiments, physiological data may be acquired by a camera (e.g., in a mobile device), which may be used to take a picture of a body part, wound, or lesion, or of a physical substrate such as a test strip, dipstick, filter, or other substrate that provides means for detecting or measuring the presence of a substance in a biological sample obtained from a subject. The image may then be analyzed by an application that runs on the mobile device or may be transmitted over a communication network to a remote computer for analysis to obtain an indication of the state of the body part, wound, or lesion, or the presence or level of the substance. In some embodiments, it is contemplated to detect antibodies against pathogens (e.g., viruses, bacteria, fungi) or pathogen components (e.g., pathogen-derived DNA, RNA, or protein) in a biological sample and/or to detect the presence of pathogens or pathogen components in a biological sample from a patient for purposes such as diagnosing the presence of an infection or determining the cause of an infection.

“Behavioral data” refers to any data pertaining to the way an individual acts, particularly those ways that may affect or reflect the individual's health. Behavioral data encompasses data pertaining to the individual's diet, physical activity, sleep, smoking, use of drugs with addictive potential, and other health-related behaviors and habits that may affect or reflect an individual's health. Behavioral data may also encompass data pertaining to the way an individual interacts with a system of the invention or components thereof, such as time spent accessing or otherwise engaging with educational materials made available to the patient. Behavioral data may also encompass data pertaining to a patient's compliance with a treatment plan.

“Genotypic data” refers to any data pertaining to the genetic makeup of a cell, population of cells, or an individual, usually with reference to a specific characteristic under consideration, specific gene or genes of interest, or specific nucleotide position(s) in a gene or genes of interest. Genotypic data may indicate which nucleotide is present at a particular position in a chromosome or chromosomes, which allele(s) are present, copy number of a genomic region. Genotypic data may indicate the presence or absence of a mutation or particular genetic variant the presence of which is associated with an increased or decreased risk of disease as compared with the risk in the absence of the mutation or variant and/or is known or believed to contribute to causing a disease.

“Environmental data” refers to data concerning a patient's indoor or outdoor surroundings. “Indoor environment” refers to inside the patient's home and/or workplace or such other place as the patient spends a significant amount of time (e.g., at least 10 hours per week). “Outdoor environment” refers to the environment outside of buildings. Aspects of the outdoor environment include outdoor temperature, levels of pollen or pollutants or others substances in the air.

“Population-based data” refers to data about groups of individuals, e.g., groups of patients having a particular condition or living in a particular geographic region. Geographic data refers to data about a geographic region, which may include data about environmental conditions in the region or people in the region. A geographic region may be a city, county, zip code area, state, or any other region as defined or used by the system. Population-based data and geographic data may include epidemiologic data, such as data about the prevalence of particular infectious diseases in the geographic region where a patient lives.

“Omics data” encompasses data pertaining to any field of biology ending in the suffix “omics”. Such fields include genomics (including epigenomics), lipidomics, metabolomics (including metabonomics), proteomics, and transcriptomics, and the term “omics data” thus encompasses genomic data, lipidomic data, metabolomic data, proteomic data, secretomic data, and transcriptomic data. “Genomic data” refers to data pertaining to the genome, which term refers to the DNA of a cell, population of cells, tissue, organ, or organism and should be understood to include epigenomic data, which refers to the set of epigenetic modifications on the genetic material and DNA-associated proteins of a cell or population of cells. “Lipidomic data” refers to data pertaining to the lipidome, which term refers to the entire complement of cellular lipids, including the modifications made to a particular set of lipids, produced by a cell, population of cells, tissue, organ, or organism. “Metabolomic data” refers to data pertaining to the metabolome, which term refers to the collection of all metabolites in a cell, population of cells, tissue, organ, or organism, which are the end products of cellular processes. Metabolomics should be understood to include metabonomics, which extends metabolic profiling to include information about perturbations of metabolism caused by environmental factors (including diet and toxic substances), disease processes, and the involvement of extragenomic influences, such as gut microflora. “Proteomic data” refers to data pertaining to the proteome, which term refers to the entire set of proteins expressed by a genome, such as the genome of a cell, population of cells, tissue, organ, or organism. Proteomic data may indicate the abundance, post-translational modification state, activity, interactions, or other characteristics of proteins. Secretomic data refers to data pertaining to the secretome, which is the totality of secreted organic molecules and inorganic elements by a cell, population of cells, tissue, organ, or organism. “Transcriptomic data” refers to data pertaining to the transcriptome, which is the set of all RNA molecules, including mRNA, rRNA, tRNA, and other non-coding RNA produced in or present in a cell, population of cells, tissue, organ, or organism. For purposes hereof, genomic data, lipidomic data, metabolomic data, proteomic data, secretomic data, and transcriptomic data, shall be understood to encompass any data obtained through the large-scale analysis of DNA, lipids, metabolites, proteins, secreted substances, or RNA in a subject or in a sample obtained from a subject. Omics data may be obtained through methods that are adapted to the obtaining of large-scale biologic data, such as microarray analysis, ChIP-Chip, ChIP-Seq, RNA-Seq, mass spectrometry, NMR spectrometry, and the like. Omics data need not concern the entire genome, lipidome, metabolome, proteome, secretome, or transcriptome so long as it concerns a substantial fraction of the relevant type of biomolecule, e.g., at least 5%, 10%, 20%, 50%, 75%, 90%, or more of those molecules present at a detectable level in a sample and/or applies a technique suitable for the large-scale analysis of such molecules. Furthermore, omics data may be analyzed to extract particular data of interest, such as data pertaining to genes, proteins, metabolites, secreted substances, or the like, which are known or suspected biomarkers. It will be understood that genotypic data and omics data are often obtained by analysis of a cell or population of cells in a sample obtained from a subject.

A “treatment plan” refers to a predetermined set of procedures, treatments, and other diagnostic and therapeutic interventions that a health care provider recommends or prescribes to the patient or performs for purposes of managing one or more conditions in the patient. The procedures, treatments, and other diagnostic and therapeutic interventions of a treatment plan are typically to be performed according to a predefined schedule, which is part of the treatment plan.

As used herein, a “timeframe” (which term is used interchangeably herein with “timing”) refers to a specified time period in which something (e.g., obtaining a data element, performing a health care event) occurs or is planned to take place or the spacing of occurrences or planned occurrences of something in time. A timeframe may be specified in any of a wide variety of ways, as the invention is not limited in this respect. The particular way a timeframe for data elements of a particular type is specified may be selected as appropriate for data elements of that type. A timeframe may comprise or consist of a specified time or times when something occurs or is planned to take place. A timeframe may be absolute or relative. As an example of a relative timeframe, a second thing may occur or be planned to take place within a specified time period after the occurrence of a first thing, which may itself have a timeframe. In the case of something that occurs or is planned to take place more than once but at irregular intervals, a timeframe may specify the intervals between successive occurrences or planned occurrences of the thing. A timeframe may specify whether or that something is planned to take place once or more than once. In the case of something that occurs or is planned to take place more than once, a timeframe may specify whether or that the thing is planned to take place at regular intervals (meaning that individual occurrences of the thing are equally spaced or planned to be equally spaced in time) or irregular intervals (meaning that the time period between successive occurrences or planned occurrences of the thing differs). In the case of something that occurs or is planned to take place at regular intervals, a timeframe may specify a frequency that indicates how often that thing occurs or is planned to occur over a given time period and/or indicates the time interval between consecutive occurrences or planned occurrences of that particular thing. For example, a timeframe for taking a medication may be specified by a frequency, e.g., “daily” or “twice daily”. In the case of something that occurs or is planned to take place at irregular intervals or changing intervals, a timeframe may specify the time interval(s) between successive occurrences or planned occurrences of that thing. A timeframe may specify one or more timeframes that change over time. For example, in some embodiments, a timeframe may specify particular time intervals between certain occurrences or planned occurrences of the thing and may specify a frequency for other occurrence or planned occurrences. In some embodiments, a timeframe may specify a first frequency at which occurrences are planned to take place during a first time period and a second frequency at which occurrences are planned to take place during a second time period. In some embodiments, a timeframe may specify one or more things which, if detected, trigger the occurrence of one or more other things. The timeframe for the one or more other things may comprise information that identifies one or more things the occurrence of which serves as a trigger for occurrence of the other things. In some embodiments, it is assumed that the thing is to occur whenever a need for the particular thing arises or whenever the thing is requested. This may be the case, for example, if no particular frequency, time interval, or trigger for a thing is specified, or if a need for the thing may arise at unpredictable times. For example, if a data element of a particular type may be needed to evaluate the status of a patient's health, the data element may be obtained during the course of the evaluation, optionally in addition to according to any specified timeframe.

A timeframe may specify one or more time windows in addition to, or instead of, a frequency. A time window may specify a particular period of time prior to an occurrence or planned occurrence of something. A time window may have any of a variety of meanings associated with it and may have any of a variety of uses. For example, a time window may indicate a time period during which a data element or data elements of a given type remain valid (acceptable for use) for one or more purposes after being obtained. A data element that no longer remains valid, typically because too much time has passed since its acquisition, may be referred to as “expired” or “invalid”. A time window may indicate a time period within which something should preferably occur relative to a specific absolute time or relative to a particular time specified or implied by a frequency or time interval. A timeframe may be approximate, in that something may still be considered to have taken place according to the timeframe if it takes place within a specified time window before or after a specific absolute time or before or after a particular time specified or implied by a frequency or time interval. A time window may be used to trigger the occurrence of one or more actions, such as issuance of a notification if a particular thing has not occurred within the specified time window relative to a planned occurrence of that thing. A notification may inform a patient, caregiver, or health care provider of the non-occurrence of the thing and may, in some embodiments, provide directions relating to the non-occurrence of the thing, such as directions to reschedule a missed appointment or directions not to take a missed medication dose. A time window may alternately or additionally be used to trigger the issuance of a notification prior to a planned occurrence of a thing. The notification may, for example, comprise a reminder of the upcoming planned occurrence or may comprise a reminder of something else that needs to occur first in order for the planned occurrence to take place.

A “time” may be any indicator of when or approximately when something (e.g., an event, a measurement, obtaining data) took place or is to take place within the continued progress of existence that individuals (e.g., physicians, patients) experience. Times for future events that are to occur on a recurring basis may often be expressed as frequencies (e.g., every 3 months) or time intervals (e.g., 3 months apart). A time may specify a particular date and may further include a specific time as determined by a clock (e.g., a particular timepoint within a 24 hour day) on a specific date. Where a date is understood, a time may simply specify the particular timepoint on that date. A “timestamp” should be understood as information that specifies both a particular date and a time on that date (which may be specified to within a particular unit such as an hour, minute, second, or fraction thereof).

A “data element” refers to an individual unit of data. A data element may be considered indivisible or may consist of one or more data items. A data element may, for example, be something as simple as an individual measurement of a physiological variable such as body weight or a patient's response to a yes/no question about the presence or severity of a symptom, or may be more complex, such as an X-ray image, a pathology report, or the like. A “data element type” or “type of data element” refers to a particular kind or category of data element. A data element type may have a specific name, e.g., “body weight”, “cough severity change”, “6 minute walk distance”, or “chest X-ray” and a definition. Data element types may be named and defined in any suitable way. A data element may be tagged with metadata that indicates its type or may be stored in a location or obtained from a data source that indicates its type or may be analyzed to determine its type.

“Electronic health record” (EHR) generally refers a collection of health data about an individual patient that is in digital format and is stored in any of a variety of computer-readable media and typically organized in a systematic manner.

“Electronic medical record” (EMR) is an EHR that is controlled by one or more health care organizations and in which most or essentially all the health data is acquired or entered by one or more health care providers or under control of a health care institution or organization. Typically an EMR contains information that describes the systematic documentation of a single patient's medical history and care across time within one particular health care provider's, health care institution's, or health care organization's jurisdiction. The electronic medical record includes a variety of types of entries made over time by health care professionals, recording observations and administration of drugs and therapies, orders for the administration of drugs and therapies, test results, images, reports, etc.

“Personal health record” (PHR) refers to an EHR that an individual patient controls and typically primarily contains health data that is entered by or under the control of the patient.

“Managing” or “management” in the context of managing a condition, generally refers to activities undertaken cure, alleviate symptoms of, slow or stop progression of, reverse, reduce risk of future morbidity or mortality due to, or otherwise control a condition in a patient, usually following a diagnosis. Management typically includes evaluating the status of, tracking, and monitoring the condition as well as providing, prescribing, or administering treatment. A health care provider's activities for managing a condition may include performing procedures, prescribing therapeutic interventions such as medications, and/or making recommendations concerning behaviors (e.g., diet, physical activity, smoking) or other matters that may affect a patient's health. A patient's activities for managing a condition may include complying with physician recommendations and taking those actions necessary in order for the physician to carry out his or her activities for managing the condition, such as taking prescribed medications as directed, making and keeping appointments with health care providers, and engaging in behaviors consistent with physician recommendations.

“Medications” refer to pharmaceutical agents (drugs), which may be defined as any substance or product comprising such that is used or intended for use in the diagnosis, treatment, or prevention of disease or to otherwise enhance physical or mental well-being. A medication may be any substance, product, or combination product listed in the official formulary or pharmacopeia of a nation, e.g., the United States Pharmacopeia (USP) or the National Formulary (NF) (both published by The United States Pharmacopeial Convention, Rockville, Md.) or listed in The Anatomical Therapeutic Chemical (ATC) classification system (WHO Collaborating Centre for Drug Statistics Methodology (WHOCC) (Oslo, Norway) or having an assigned code in the US National Drug Code (NDC) numbering system (http://www.fda.gov/Drugs/InformationOnDrugs/ucm142438.htm) or an entry in the National Drug Data File Plus (First DataBank). Drugs may be available or provided in various dosage forms, such as tablets, pills, capsules, caplets, inhalers, injections (e.g., intravenous, intramuscular, subcutaneous), creams, liquids, ointments, gels, suppositories, etc. A medication may be a drug that is to be taken or used on a regular basis, e.g., one or more times per day, every other day, etc., or one that a patient may take on an as-needed (PRN) basis (typically within certain limits). A medication may be a prescription medication (available to patients only if prescribed through an appropriate prescription process by an authorized health care provider) or may be available generally without such a prescription (sometimes referred to as an “over-the-counter” medication).

A “patient” is an individual who seeks or receives health care from a health care provider. A patient may be a patient of one or more health care providers. For example, a patient may have a primary care physician and one or more specialist physicians each of whom manage particular condition(s) within that physician's specialty.

The term “caregiver” refers to anyone who provides physical assistance with health care needs or activities of daily living to a patient outside a health care facility, typically on a reasonably frequent or regular basis. A caregiver may be a patient's family member, friend, or someone for whom caregiving is an occupation, job, or vocation. The term “remote caregiver” refers to anyone who is involved with or concerned about the patient's health but who typically does not provide the patient with physical assistance with health care needs or activities of daily living on a frequent basis. A remote caregiver may provide emotional support, e.g., through phone calls or other communication means, financial support, visits, or other forms of support. Remote caregivers may be, for example, children, siblings, or other family members living in different cities from the patient.

The term “interaction” in regard to a user and a system refers to an exchange of inputs and outputs between the user and the system. In general, an input from a user is related to and responsive to an output from the system, or an output from the system is related to and responsive to an output from a system or component. A user-system interaction may resemble a conversation between two human beings, in which the system presents a question to the user through a suitable user interface and the user responds to the question through the same interface. A user may be, e.g., a patient, health care provider, or caregiver.

“Encounter” as it refers to a patient and a health care provider, refers to interactions in which a patient communicates with the health care provider in real time or close to real time. Encounters may be in person, e.g., when a patient visits a physician to receive health care, by phone, by videoconference, or using any suitable communication channel that allows real-time interactive communication between the patient and the health care provider.

The term “patient record” refers to a collection of information regarding or relating to a patient, stored in association with an identifier of the patient. The information may be stored in one or more databases or data stores, which may be physically located in different places. It is considered to constitute a patient record as long as it is stored in a way that allows the system to determine which patient the information pertains to or that the information belongs to a particular patient. The information in a patient record may include any type of health data described herein, patient characteristics, patient's name, contact information (e.g., email address, phone number(s)), demographic information, patient's health care provider(s), patient's caregivers, family members, and any other information obtained or generated by a system of the invention regarding or relating to the patient. It should be noted that a patient record may be de-identified in that it lacks personally identifiable information. Thus a patient identifier need not personally identify the patient but may simply serve to identify particular items of information as pertaining to the same patient.

A “patient characteristic” refers to any attribute, quality, trait, feature, or characteristic that a patient may have. Typically, patient characteristics are characteristics that an ordinary skilled medical practitioner would consider relevant to a patient's health or its management. Examples of patient characteristics may include demographic characteristics such as age and gender; conditions; contraindications to medications; current or past behaviors that may affect the patient's health or risk of developing certain conditions or ability to adhere to treatment recommendations (e.g., smoking, substance abuse), and generally any characteristic that may be identified in the course of taking a medical history or performing a physical examination. Patient characteristics are typically personal characteristics but may include features of a patient's physical environment, living circumstances, and/or social support systems, particularly as relevant to the individual's health. For example, patient characteristics may include the availability of caregivers.

The term “payer” refers to an entity or individual that at least in part pays for health care services and/or health-care related products such as medications and medical devices. Such payment may be referred to as “reimbursement”. A request or submission seeking reimbursement from a payer may be referred to as a “reimbursement claim” or simply a “claim”. A payer may be a public (government) entity or a non-government operated entity. A payer may be a US government payer such as Medicare, Medicaid, Children's Health Insurance Program (CHIP) or Program of All-Inclusive Care for the Elderly (PACE); an insurance company such as Aetna, BlueCross BlueShield insurance companies and organizations, Humana, HealthNet, UnitedHealthcare, WellPoint or any other company that sells health insurance; or an employer that at least in part covers employee health care-associated costs through self-insurance itself or through a captive insurance company. In some embodiments a payer is a government payer operating under a “single-payer” system, i.e., a system in which the government pays for health care costs for all citizens (or residents) for at least a minimum set of health care services. Unless otherwise indicated, “payer” generally refers to an entity rather than the patient or another individual person who pays on behalf of the patient.

A “set” as used herein, is a collection of one or more things, which may be referred to as “elements” of the set. A “subset” of a set A is a collection consisting of any one or more elements of A, i.e., a subset of A does not include any elements that are not also in A. A subset of a set may include all the elements of the set. The elements of a set may be related to each other in some way, e.g., they may all be of a particular type, or they may all pertain to a particular patient.

The term “user” refers to an individual or entity that uses a system, apparatus, or product (e.g., a computer program product or database). An individual user may for example, be a health care provider, associated personnel, a patient, a patient's parent or legal guardian or a representative authorized by the patient to provide or access health data regarding the patient, a patient's caregiver, a researcher, an authorized employee or agent of an entity such as a health care institution, health care organization, payer, or pharmaceutical company, or anyone else who makes use of a system, apparatus, or product functionality or service. Users in any of the afore-mentioned categories may sometimes be referred to as “end users”. The term “user” also includes individuals who create, maintain, develop, update, administer, provide end user support, and/or otherwise support a system, apparatus, or product. Such users may be referred to as “support users”. Users may be authorized to use and/or modify a system, apparatus, and/or product to any extent compatible with their role(s). A user will typically have a user account or the right to use a user account. The user account allows a user to authenticate to a system, apparatus or product and be granted access to at least part of it or to one or more services provided by it. A user account is associated with a collection of data pertaining to the user and/or user account. User account data may comprise, for example, personal identifiers such as a user's name, password, biometric identifier (e.g., fingerprint, iris scan, photograph, characteristics of the individual's typing pattern), email address, business address, home address, etc. User account data also includes data indicating the extent to which the user is authorized to access, use and/or modify the system, apparatus, or product. In some embodiments, a system, apparatus, or product of the present invention comprises, creates, modifies, or accesses, a database that contains user account data.

### DETAILED DESCRIPTION

Described herein are systems and methods for structured collection and organization of health data. The phrase “structured collection and organization of health data” is intended to indicate that health data are collected and/or organized in a manner that is based on a specification comprising information that identifies one or more types of health data elements to be collected and, for at least some of the types of data elements also identifies a timeframe. The timeframe may be a timeframe for the collection of data elements of that type and/or for the performance of health care events whose occurrence would generate data elements of that type. In general, the health data may be of any of various categories of health data, e.g., symptom data, physiological data, behavioral data, environmental data, or health care event data, to name a few. In some embodiments, a specification for collection and organization of health data relates to a particular condition in that it identifies a particular condition and a set of types of health data elements that are in some way relevant to the status or management of that condition. By way of example, data that may be informative about the degree of severity of the condition in a patient or the efficacy of the patient's treatment, such as the intensity of a particular symptom that may be present in patients with the condition (a relevant symptom) or the value of a particular physiological variable that may be abnormal in patients with the condition (a relevant physiological variable), would typically be considered relevant to that condition; data indicating that a particular medical procedure has been performed, the results of which may be informative about the degree of severity of the condition or the efficacy of the patient's treatment would typically be considered relevant to that condition; data indicating that a patient has taken a particular medication that has been prescribed for treating the condition or has otherwise adhered to the recommendations of the physician who treats the patient for the condition would typically be considered relevant to that condition. Such a specification may be referred to herein as a “health tracking and management plan” or “T-plan”, and the particular condition to which the data specified by the T-plan are relevant and for which the T-plan is intended may be referred to as the “T-plan condition” for that T-plan. A T-plan may be identified at least in part by the name of the particular condition for which it is intended or an abbreviation or identifier thereof. For example, a T-plan for management of chronic obstructive pulmonary disease (COPD) may be referred to as a “COPD T-plan”. A T-plan may have a unique identifier, which may be related in some way to the T-plan condition and may implicitly identify the T-plan condition.

Health data relevant to a condition, which may be specified by a T-plan for that condition, may include any type of health data that a health care provider of ordinary skill in the art would typically obtain if evaluating a patient with the condition and/or would wish to know A symptom that is relevant to a condition may be any symptom that is recognized in the art as being associated with or indicative of the presence of the condition, associated with or indicative of a deterioration (worsening) in a patient's health status as regards the condition, useful in monitoring the status of the condition, useful in assessing the severity or prognosis of a condition, or useful in selecting a therapeutic regimen. In certain embodiments, a relevant type of symptom or type of physiological data is characterized in that its presence, level, intensity, or value is an indicator of the severity of the condition or the likelihood that a patient with the condition will experience an adverse change, e.g., an exacerbation of the condition, or other significant health-related event within a selected time period such as 6, 12, 24, 24, 36, or 48 hours or 1, 2, 3, 4, 5, 6, or 7 days.

A T-plan may be stored on a computer-readable medium and may comprise, identify, be associated with, or be used or referenced by computer-executable instructions that, when executed, obtain, request, receive, or search for data elements of any one or more of the types specified by the T-plan or carry out one or more other tasks as specified in the T-plan. A T-plan may be described herein as performing various tasks, such as obtaining data. However, it should be understood that the T-plan may not actually perform the thing but may merely specify the thing to be performed by, for example, providing information about the thing to be performed, e.g., the types of data elements to be collected and, where applicable, a timeframe. Where the present disclosure refers to a T-plan, or a module or component thereof, doing something, such as obtaining data, it should be understood that the T-plan, component, or module may, for example, comprise, identify, be associated with, or be used or referenced by computer-executable instructions for doing that thing as specified by the content of the T-plan. It should be understood that a T-plan component or T-plan module may or may not correspond precisely to a particular software component or module.

In some embodiments, computer-executable instructions associated with a T-plan may be executed to obtain, request, receive, or search for data elements of any one or more of the types specified by the T-plan in accordance with a timeframe specified in the T-plan for data elements of that type. In some embodiments the computer-executable instructions determine whether or that a data element was obtained in accordance with a timeframe specified for data elements of that type in the T-plan and/or determine whether or that an underlying health care event was performed in accordance with a timeframe specified for data elements of that type in the T-plan. In some embodiments the computer-executable instructions cause one or more actions to take place based on the timeframe.

In some embodiments, a system actively obtains health data specified in a T-plan from patients on an ongoing basis in order, for example, to allow the patient's health status in regard to the condition to be monitored or evaluated. In some embodiments, the system obtains such data by interacting with a patient or caregiver through a user interface of the system or obtains data collected by a personal monitoring device owned or controlled by the patient. In this regard it should be understood that health data “obtained from the patient” may be obtained through interaction with the patient, such as by asking questions to the patient through a suitable user interface and receiving responses, or may be data pertaining to the patient that are acquired by connected monitoring devices without requiring that the data be entered by the patient, or may be obtained without requiring that the patient take any active part in acquiring the data (e.g., the patient may wear a connected monitoring device that automatically or upon request collects data from the patient or a connected monitoring device may be located in the patient's home and collect data from the patient while the patient is in the home such as by detecting motion or body heat in any of a variety of ways).

In some embodiments, the system stores at least some of the health data collected according to a T-plan in association with an identifier of the patient to whom the data pertains (patient identifier). In some embodiments, the system additionally or alternately accesses or receives health data from any of a variety of data sources and stores the health data, data indicating the existence of the health data, and/or information indicating the data source or location where the data is to be found, in association with an identifier of the patient to whom the health data pertains. Each data element or data item received by the system may be assigned a globally unique data identifier (GUID). In some embodiments, at least some types of data elements are stored in association with one or more timestamps. A timestamp may comprise information specifying: the time that the data element was obtained from the patient, the time that the data element was obtained by the system from a source that obtained the data element from the patient, the time that a measurement or procedure that resulted in the particular data element was performed, or other information sufficient to determine a time related to the data element to within the level of absolute or relative accuracy required by a timeframe or specified by the system. In some embodiments, data elements are stored in a way that permits them to be retrieved according to their type. Data elements may, for example, be tagged with metadata that identifies their type, may be stored in a particular location or data structure reserved for data elements of that type, etc. In some embodiments, the particular type of a data element may often be evident by the channel through which it was received (e.g., in an embodiment in which a landline telephone is used exclusively to ask a particular type of question of a patient, a data element obtained over telephone landline could be presumed to be the type of data element which would be provided in response to the questions asked over the phone), or because it was specifically requested, or based on its source (e.g., data elements obtained from a third party weight monitoring application or website could be presumed to correspond to the “body weight” data element type). In some embodiments, a data element may be analyzed to determine its type. In some embodiments, particular data elements of the types specified by a T-plan are selectively retrieved from storage, analyzed, or presented to a user. A T-plan may thus serve as a framework that guides the condition-centric collection of health data, imparts a condition-centric organization to health data, or both.

A T-plan may be assigned to an individual, e.g., a patient, in a variety of ways, as discussed further herein. For example, a health care provider may use the system to prescribe a T-plan to a patient. In some embodiments, an individual may assign a T-plan to himself or herself using the system. In some aspects, the system provides software applications for use by health care providers, patients, or other individuals, wherein such applications comprise user interfaces that permit the configuration and assignment of T-plans. It should be understood that the particular user interfaces, user interface features, and methods of assigning T-plans described herein are exemplary and should not be considered limiting.

After a T-plan has been assigned to a patient, the system may start to obtain and attempt to obtain data elements according to the T-plan. In certain embodiments, at least some of the types of health data collected according to a T-plan are ordinarily obtained from a patient in the course of his or her daily life, between the patient's encounters with health care providers. For example, symptom data, physiological data, behavioral data, and/or environmental data may be obtained from or about patients in their daily lives. The term “daily life” is used to refer to the patient's existence when not hospitalized or receiving care in a medical facility. The data may be obtained while the patient is at home, at work, or wherever else the non-hospitalized patient may spend time.

The health data collected according to a T-plan (i.e., as specified by a T-plan, pursuant to a T-plan) may be referred to as a “T-plan data module”. It constitutes a distinct set of health data relevant to the condition that can be selectively retrieved, analyzed, displayed, or otherwise processed. A T-plan data module may be augmented with additional health data that pertain to the patient, such as demographic data, patient characteristics, and/or past medical history relevant to the T-plan condition.

In some embodiments a T-plan may identify a subset of the types of data elements that it specifies as “patient state data element types”. Such data element types are characterized in that the specific data elements of those types at a given time are considered to constitute a “patient state” with regard to the T-plan condition. In general, such data element types are symptom data or physiological data but might in some cases include behavioral or environmental data. The data element types may be relevant to the T-plan condition or to a significant patient characteristic for the T-plan condition. The collection of valid data elements of those types specified as patient state data elements for a particular condition represents the patient's state at any particular point in time with respect to the particular condition. If a patient has multiple T-plans, the union of patient state data element types for all of the patient's T-plans may be considered to constitute an overall patient state. The data elements that make up a patient state at a particular time may be referred to as “patient state data”. In some embodiments the patient state is updated when or within a specified time period after the system receives a new patient state data element specified by a T-plan that has been assigned to the patient. The overall patient state or patient state with regard to any particular condition or with regard to any one or more types of data elements may be analyzed by the system at any time to determine whether an action should be taken, such as prompting the patient to run a smart symptom tracker (described further below), notifying a caregiver or health care provider, or both. In some embodiments, the patient state is analyzed whenever it is updated. In some embodiments the system may store information identifying a set of patient state data element types for each of a plurality of conditions or without reference to any particular condition.

One or more types of data elements in the patient state may be associated with information specifying how long a data element of that type remains valid. Such information may be as specified in the relevant T-plan (e.g., in the monitoring module that specifies data elements of that type as ones to be obtained).

In some embodiments, a data element may be valid for a specified time period after which it is invalid for all purposes. In some embodiments, a data element may be set to expire once that time has elapsed. In some embodiments, a data element may be valid for a specified time period, after which it is invalid for one or more purposes while remaining valid for one or more other purposes. In some embodiments, expiration of a data element may triggers a query to determine whether a new one to replace it is available.

In some embodiments, each date element type in the patient state data is updated according to a timeframe. If the patient has multiple T-plans, the timeframe may be such as to satisfy the requirements of the monitoring module that specifies the highest frequency for collecting data elements of that type and satisfies any dependencies or constraints that might be specified in any T-plan (e.g., potential need to collect different data element types in a defined sequence or within a defined time interval). In some embodiments, when a new data element is received a time for collecting the next one of that type may be calculated.

In certain embodiments, at least some of the types of health data collected according to a T-plan are health care event data. In some embodiments, health care event data are obtained through input of the data by a patient via a user interface of the system or by a personal monitoring device owned or controlled by the patient. In some embodiments, health care event data are generated as a consequence of a procedure ordered or performed by a health care provider and are not obtained through input of the data by a patient via a user interface of the system or by a connected monitoring device owned or controlled by the patient, but may instead be obtained from any of a variety of data sources, e.g., EMRs under control of a health care organization, reimbursement claims, and the like. In some embodiments, health data may be obtained from diverse data sources that may contain such data.

In addition to collecting and storing health data, a system may provide output of various kinds to a patient or health care provider based on data collected according to a T-plan that has been assigned to the patient. The output may comprise, for example, summaries or analyses of health data collected by the system (sometimes referred to as “health statistics”), notifications or directions to the patient regarding management of the condition, notifications or suggestions to the health care provider regarding management of the condition, or any of a variety of other types of output described herein. At least part of the data, summaries of the data, or analyses of the data may be provided to the patient to assist the patient in, for example, tracking and managing his or her health and/or may be presented to a health care provider, e.g., a health care provider who prescribed the T-plan to the patient. Thus, in some aspects, the invention provides computer-implemented methods by which health care providers can be provided with health data concerning their patients that are obtained while the patients are outside of medical facilities. In some aspects, the invention provides computer-implemented methods of collecting health data from a patient between patient encounters with health care providers or between patient encounters with a particular health care provider. Data pertaining to patients in their daily lives (e.g., symptom data, physiological data, behavioral data) may assist the health care provider in, for example, monitoring the condition between encounters and obtaining an improved understanding of the patient's health as it exists between encounters.

In some embodiments, a health care provider is presented with or able to access health care event data pertaining to procedures performed on the patient by or on the order of a different health care provider or pertaining to medications prescribed to the patient by a different health care provider. Without wishing to be bound by any theory, it is expected that this information will allow physicians to reduce duplicative performing of procedures by requesting (e.g., by email, phone, fax, or other means) a copy of results or reports and/or relevant portions of the patient's medical record at the health care facility where the procedure was performed or ordered. In some embodiments, data resulting from such procedures will be accessible via the system. Avoiding unnecessary repetition of procedures reduces patient risk that may be associated with such procedures as well as reducing expense. Obtaining results of a procedure that has already been performed may provide a physician with the information he or she needs to make a treatment decision more rapidly than would be the case if the physician needed to wait until the procedure could be scheduled and performed.

In certain embodiments a T-plan for a given condition is configured by a system automatically, yet is individualized in that an instance of the T-plan for that condition for a particular patient may vary based on characteristics of that individual patient (patient characteristics). In some aspects, the invention encompasses the insight that, although patients may differ from one another in numerous ways, variation in a relatively limited number of patient characteristics typically accounts for much of the variability in the way a particular physician treats patients with a particular condition in actual practice. In other words, much of the inter-patient variability that may exist typically does not have a material impact on management of patients with any given condition. Thus, at least in the case of many conditions, a relatively limited number of patient characteristics may be identified as being material to the management of the condition. Such patient characteristics may be referred to as “material patient characteristics” for the condition.

As an example, a patient may have two different conditions that could give rise to the same symptoms but which would be appropriately treated using different medications if there is a deterioration in the patient's health caused by one condition versus the other. In order to determine which medication to administer, it would be important to determine which condition is responsible for the deterioration in the patient's health. Thus, each of the two conditions may be a material patient characteristic for the other condition. As another example, a particular set of symptoms and/or physiological data in two different patients with the same condition but one or more different risk factors may have different significance for purposes of selecting the appropriate management. Material patient characteristics for a condition may thus include one or more risk factors, which may be conditions or may be other characteristics such as medications that the patient is taking, or features of the patient's medical history such as the number of times the patient has been hospitalized. As another example, a contraindication to a medication that is a standard of care treatment for a particular condition may be a material patient characteristic for that condition since the presence of the contraindication would mandate against treating the patient with that particular medication as it may be harmful to the patient. As another example, the presence in a patient with a condition of a particular biomarker value that correlates with efficacy of a particular medication in treating the condition, may be a material patient characteristic for the condition since it would mandate in favor of treating the patient with that particular medication.

In addition to material patient characteristics, there may also be a relatively limited number of patient characteristics that, while not necessarily material to management of the condition, may otherwise be of potential interest to health care providers managing the condition because, for example, they may affect the patient's overall health or prognosis or ability to comply with a treatment plan. Collectively the set of patient characteristics that are material to management of a condition or otherwise of potential interest to health care providers managing the condition may be deemed significant in the context of the condition and may be referred to as “significant patient characteristics” for that condition.

In some aspects, the present invention stores information that identifies multiple conditions and multiple patient characteristics. The patient characteristics may include a wide variety of patient characteristics. The database may include at least 5, 10, 20, 30, 40, 50, 60, 70, 80, 90, 100, 150, 200, 250, 300, 350, 400, 450, 500, or more patient characteristics. In some embodiments, a system stores information that identifies, for each of multiple conditions, a set of material patient characteristics. In some embodiments, a system stores information that identifies, for each of multiple conditions, a set of significant patient characteristics. It should be noted that patient characteristics are often described herein as binary (Boolean) characteristics that may be “present” or “absent”. However it will be appreciated that certain patient characteristics may be categorical with two or more levels or may be ordinal. Such characteristics may be handled similarly to the binary characteristics described herein or may be expressed as multiple binary characteristics, of which one would be present in a given patient and the others absent. The material patient characteristics, significant patient characteristics, or both, for a given condition may embody available medical knowledge of health care providers who are specialists in the particular health care discipline whose practitioners would typically treat patients suffering from the condition. The patient characteristics that are deemed significant patient characteristics for a given condition by the system may be defined based on medical knowledge and judgment of those of ordinary skill in the art. The system may utilize the advice of health care providers who are specialists in the treatment of a particular condition to define a set of material patient characteristics or significant patient characteristics for that condition. The set of material or significant patient characteristics for a particular condition may change over time. For example, patient characteristics may be added or removed or the criteria for determining whether a given characteristic is present may be modified. The system can thus readily accommodate changes in the standard of care and advances in medical knowledge, such as recognition of additional patient characteristics that are relevant to treatment or other aspects of management of a condition. In certain embodiments, a system of the invention may be used to facilitate recognizing such characteristics by providing structured, de-identified health data and tools to query and analyze it.

Among other things, the insight that it is possible to identify a set of significant patient characteristics for each of numerous conditions allows the automatic configuration of T-plans for individual patients based on a condition and the particular subset of the set of significant patient characteristics for that condition that the individual patient has. Further, the insight that it is possible to identify a set of material patient characteristics for each of numerous conditions allows the design of algorithms that accurately evaluate a patient's health status and deliver appropriate directions to the patient for managing the condition in an individualized manner. In some aspects, methods described herein assist health care providers in systematically identifying those material patient characteristics that may otherwise be overlooked but that, if unrecognized, may lead to significant complications or inappropriate management decisions. In some aspects, methods described herein assist health care providers in systematically identifying those material patient characteristics that may otherwise be overlooked but that, if recognized, would lead to the use of treatments that may be particularly effective in the subset of patients with the condition that have the particular material characteristic. It should be noted that the set of significant patient characteristics or material patient characteristics for a given condition need not encompass all patient characteristics that a particular health care provider or providers may consider significant or material across the entire scope of patients with that condition. For example, certain patients may have characteristics that are rarely observed in patients with the condition yet are material to its management. In certain embodiments, the significant patient characteristics encompass those patient characteristics that would reasonably be considered significant in the context of the condition as it exists in at least about 70%, 75%, 80%, 85%, 90%, 95%, or more of patients with the condition by health care providers who routinely manage the condition. In certain embodiments, the number of material or significant patient characteristics for a condition is between 0 and 20, e.g., between 0 and 5, between 5 and 10, between 10 and 15, or between 15 and 20. In some embodiments, the number of material or significant patient characteristics for a condition is between 1 and 10 or between 5 and 15.

In certain embodiments, T-plans may be prescribed to patients by their health care providers. Thus a T-plan for a given condition for a particular patient may be individualized in that a T-plan for the given condition is prescribed to that individual patient by a health care provider, indicating that the contents of the T-plan have been approved as being appropriate for that patient by his or her health care provider. In some embodiments, the health care provider may enter information indicating which patient characteristics, among the significant patient characteristics for that condition the health care provider believes that the individual patient has. In some embodiments, the health care provider may be provided with guidance as to how to establish whether or that a patient has a particular patient characteristic as defined by the system. In some embodiments, information identifying at least some of the patient's characteristics is stored in a patient record in a system database. The patient record may comprise a “patient profile” section that identifies the patient's characteristics, as entered by the patient's health care providers.

In certain embodiments, a T-plan for a given condition in a particular patient is customizable in that a T-plan that is automatically configured by a system of the invention may be modified by a health care provider in any of a variety of ways, either before or after it has been prescribed to a patient. In some embodiments, certain elements of a T-plan may require selection by the health care provider before the T-plan can be prescribed. The system can thus accommodate the preferences of individual health care providers as well as the additional degree of individualization, if any, that such health care providers might consider appropriate for a specific patient.

In some embodiments, a T-plan includes data that specifies a health care provider's treatment plan for the particular patient to whom the T-plan is prescribed. The system may provide a default treatment plan for a given condition and subset of the set of significant patient characteristics for that condition, which the health care provider can then modify if desired. The system may do this for any of a wide variety of conditions and, for each condition, the various combinations of significant patient characteristics for each such condition.

A T-plan in its generic, non-customized form may be referred to as a T-plan template. A particular instance of a T-plan template that has been prescribed to a patient by a health care provider may be referred to as an “active T-plan”. Unless specifically indicated, the term “T-plan” should be understood as referring to both T-plan templates and active T-plans unless the context clearly indicates that the term applies only to either T-plan templates or active T-plans.

A T-plan template may be customized prior to being prescribed to the patient or an active T-plan may be customized after being prescribed to the patient. In some embodiments, health care providers can use the system to create and save new T-plan templates or to save modified T-plan templates as new T-plan templates. Such new T-plan templates (whether created de novo or by modification of an existing T-plan template) can subsequently be prescribed to patients by the health care provider as active T-plans, optionally after customization for the individual patient. A new T-plan template that has been saved by a health care provider is stored by the system on a computer-readable medium in association with an identifier of the health care provider. The collection of T-plan templates that have been saved as new T-plan templates by a particular health care provider may be referred to as belonging to that health care provider's “personal T-plan library”. In some embodiments a health care provider may additionally or alternatively, save unmodified, system-provided T-plan templates in his or her personal T-plan library.

For purposes of description it is generally assumed that an active T-plan is prescribed to a patient by a health care provider. However, in some embodiments, a T-plan may be assigned to a person by an individual who is not the person's health care provider. For example, in some embodiments, a person may assign a T-plan to himself or herself. Thus it should be understood that wherever the present disclosure refers to a T-plan being prescribed, the invention includes embodiments in which the T-plan is assigned to a person (who may or may not be a patient) by an individual other than a health care provider. A T-plan assigned to a person by someone who is not the person's health care provider may not include all the features of a T-plan that may typically be included if the T-plan had been prescribed by a health care provider. For example, a T-plan assigned for a condition by a health care provider may include a treatment plan, whereas a T-plan assigned to a person by someone who is not the person's health care provider may not include a smart symptom tracker and/or may not include a treatment plan. Furthermore, a T-plan assigned to a person by someone who is not the person's health care provider may not specify a particular condition for which treatment would be required, but may instead be used for the purpose of helping the person maintain his or her health (e.g., a person in a relatively healthy condition might self assign a T-plan to help maintain that state).

In certain embodiments, an active T-plan for a particular patient is individualized in that the means or medium for communication or passage of information (sometimes referred to herein as “channels” or “interaction modes”) by which data elements of a given type are obtained according to the T-plan and/or by which output is provided to the patient may vary. A channel for obtaining data from a patient may comprise or utilize an application that runs on a mobile device, an application that the patient can access over the Internet using any computer that runs a web browser, an interactive voice response (IVR) system that can be used with a basic phone, or a connected monitoring device that obtain data from the patient or his or her environment. An application that runs on a mobile device or that the patient can access over the Internet may, for example, obtain data through a user interface that presents the patient with one or more questions that request such data and accepts input from the patient. The questions may, for example, be presented on a display screen, and the patient may respond to the questions by selecting among various options presented on the screen or by entering a number or other characters or words. Examples of questions that might be presented to a patient are, “Are you more short of breath today than usual?” or “Please weigh yourself and enter your weight.”

A question may be asked by the system and answered by the patient via computer or phone using any of a variety of output devices, input devices, and user interfaces. Many output devices, input devices, and user interfaces suitable for requesting information from a user and obtaining responses are known to those of ordinary skill in the art, and the invention is not limited in this respect. Questions may be presented on a computer display, asked orally by a computer (which may be a mobile device such as a smartphone) or via a basic phone or via a smartphone in its capacity as a telephone. In some embodiments, text messaging may be used for at least some questions and/or to prompt the patient to run the application. In some embodiments, user input is at least in part via a graphical user interface in which potential responses predetermined by the system are displayed as icons on a touch-sensitive display (touch screen), and the user touches or taps the response he or she wishes to select, as exemplified further below. In some embodiments, user input is at least in part via a graphical user interface comprising check boxes, radio buttons, and/or fields for entry of numbers (e.g., for obtaining certain physiological data elements) or brief natural language answers, etc. The particular user interface may be selected as appropriate for the interaction mode.

In some embodiments, a data collection module comprises or utilizes an interactive voice response (IVR) system. IVR refers to technology that allows a computer to interact with humans through the use of voice and/or dual-tone multi-frequency signaling (DTMF) tones input via keypad. In some embodiments that make use of IVR, questions are asked over fixed phone lines and users' answers are automatically interpreted. In some embodiments, voice input may be accepted. Such systems may include, e.g., voice recognition, transcription, pre-recorded messages or prompts, dynamically selected or generated voice messages, prompts, or responses, etc.

In some embodiments, automatic speech recognition may be used. Any of a variety of techniques and algorithms for speech recognition may be used. Speech recognition software may be part of an IVR system, may be part of computer program product of the present invention, may be an independent software application that interfaces with a computer program product of the invention, or may be part of the operating system that runs on a user's computer. In some embodiments, a user's spoken responses, requests, or commands, may be analyzed and/or stored in the same way as user input received through a touch screen, physical keyboard, keypad, or other input device. In some embodiments, an interaction mode may be selected when the patient initially enrolls with the system, may be changed by the user though a “Settings” option.

A patient may be asked questions in order to obtain physiological data. The patient may be asked, for example, to enter his or her weight, temperature, oxygen saturation, or other physiological data. Alternately or additionally, physiological data may be collected by connected monitoring devices without requiring the patient to enter the data, although the patient may need to be prompted to use or put on the appropriate device. One of ordinary skill in the art readily appreciates that there are a variety of ways by which data collected by a connected monitoring device may be obtained by the system. In some embodiments, a connected monitoring device is or can be synchronized, e.g., wirelessly (e.g., via Bluetooth or Wi-Fi), to a computer or mobile device such as a smartphone and/or uploads data collected by the device to a website (which may be controlled by the maker of the device) or to a patient's personal health record, from where it can be obtained by a system of the invention. The particular way in which physiological data are obtained may depend at least in part on information in a patient record pertaining to which monitoring devices the patient has. Such information may be acquired by the system at the time of patient enrollment or subsequently.

In some embodiments, one or more channel(s) may be selected by the patient or the health care provider, typically based on the patient's preferences and/or the availability of particular monitoring devices to the patient. In some embodiments, the system may use one or more predetermined default channel(s) to collect data elements of a particular type, which channel(s) may, in some embodiments, subsequently be changed by the patient. In some embodiments, the system may request information pertaining to available or preferred channels from the patient or health care provider and use the information to automatically collect data using particular channels based on the information supplied. In some embodiments, a particular language may be selected for communication with the patient, e.g., asking questions, providing directions, providing notifications, providing educational materials. In some embodiments, patient characteristics may include characteristics that may affect the mode by which communications are provided (e.g., whether the patient has vision or hearing problems) or the language or manner in which such communications are provided.

In some embodiments, the system may guide the patient through a process by which particular channels or data sources may be made available to the system. For example, the system may instruct the patient how to authorize or permit the system to obtain data pertaining to the patient that is held by third parties or may instruct the patient how to authorize or permit third parties that hold data pertaining to the patient to provide such data to the system. A “third party” in this context refers to any individual, entity, or system that controls or possesses data pertaining to the patient, to which the system would not otherwise have access without the authorization or permission of the patient. A third party may, for example, be a manufacturer or provider of a connected monitoring device, a payer that provides reimbursement for at least some medical expenses of the patient and therefore possesses claims for such reimbursement, a company that offers genetic testing or other medical testing services directly to the public, or a health care institution or health care organization that holds medical records, e.g., electronic medical records, of the patient.

A patient may have multiple T-plans, each for a particular condition that afflicts the patient. A physician who treats the patient for multiple conditions may prescribe multiple T-plans to the patient, each for managing a different condition. Different physicians who treat the patient for different conditions may each prescribe T-plan(s) to the patient for one or more such conditions that they manage. For example, a patient with diabetes mellitus (DM) and chronic obstructive pulmonary disease (COPD) may be cared for by a primary care physician who treats the patient for diabetes mellitus and prescribes a T-plan for diabetes mellitus to the patient and a pulmonologist who treats the patient for COPD and prescribes a T-plan for COPD to the patient. If the patient develops high blood pressure, referred to as hypertension (HT), which is managed by the primary care physician, the primary care physician may prescribe a T-plan for hypertension to the patient. If the patient develops a new condition and visits a different physician, that physician may prescribe a T-plan for the new condition. A physician who prescribes a T-plan is considered the “managing physician” for that T-plan and has the right to modify it. The system is modular and can readily accommodate addition and removal of T-plans as well as changes in the managing physician. Furthermore, if a patient who has a T-plan for a particular condition switches to a new physician for treatment of that condition, the new physician can be given access to the T-plan for the condition and would then considered the managing physician for that T-plan. In some embodiments, the new managing physician may be asked to confirm any customization of the T-plan that had been performed by the previous managing physician(s). In some embodiments, the patient may be asked by the system. e.g., via the patient application, whether he or she wishes to permit access by or transfer of responsibility for a T-plan to a different physician. In some embodiments, the system may require such permission before granting access or effecting the transfer of responsibility.

In some embodiments, a T-plan or portion thereof is embodied at least in part as or identifies or is associated with a computer program product stored on a computer-readable medium that a patient to whom the T-plan has been prescribed can use through a communication network, as an application on a computer, or both. In some embodiments, the computer program product comprises an application for a mobile device. In general, a T-plan is prescribed to a patient afflicted with a condition and, at least in part through the afore-mentioned computer program product, assists in managing the condition while the patient is not under care in a medical facility. In some embodiments, a T-plan may be prescribed to patient while the patient is an inpatient to assist in managing the condition after discharge. In some embodiments, a T-plan may be prescribed to a patient being treated as an outpatient to assist in managing the condition on an ongoing basis. In some embodiments, computer-implemented systems and methods of the invention assist patients in complying with (adhering to) a treatment plan defined by their health care provider by storing information identifying at least a portion of the treatment plan on a computer-readable medium as part of a T-plan, and providing at least a portion of the treatment plan to a patient via an electronic communication medium or computer. In some embodiments at least a portion of the treatment plan is provided to the patient in the form of a schedule of health care events. In some embodiments at least a portion of the treatment plan is provided to the patient in the form of a health evaluation that may be performed voluntarily by the patient or may be performed when the patient is prompted to do so by the system, which may occur periodically or based on the detection of data that indicative of a deterioration or potential deterioration or notable change in the patient's health. In some embodiments the health evaluation provides directions to the patient for management of the condition that have been approved by the health care provider. In some embodiments a health evaluation may determine whether the patient is experiencing or is at increased risk of experiencing a clinically significant occurrence. If it is determined that the patient is experiencing or is at increased risk of experiencing a clinically significant occurrence, one or more appropriate triage directions, treatment directions, or both, may be provided to the patient. In some embodiments educational materials relating to or embodying at least a portion of the treatment plan are provided to the patient in the form of educational modules.

In some embodiments a T-plan is for a chronic condition such as chronic obstructive pulmonary disease (COPD). A “chronic condition” is a condition that has a course that lasts as least 3 months. Examples of common chronic conditions include arthritis, asthma, cancer, COPD, diabetes, heart disease, hypertension, and HIV/AIDS. Chronic diseases may last for at least 6 months, at least 1, 2, 5, or more years, or indefinitely. COPD is used herein as an example of a chronic condition for purposes of illustrating certain aspects and embodiments of the invention. However, it should be understood that the invention encompasses embodiments pertaining to any condition. Conditions include, e.g., neurodegenerative/neurologic conditions such as Alzheimer's disease, Parkinson's disease, epilepsy, multiple sclerosis; autoimmune diseases such as systemic lupus erythematosus, multiple sclerosis, psoriasis, scleroderma, rheumatoid arthritis, ulcerative colitis, Crohn's disease, celiac disease; ophthalmologic conditions such as age-related macular degeneration, glaucoma, dry eye; cancer; cardiovascular diseases such as cerebrovascular disease, heart failure, ischemic cardiomyopathy, hypertension; inflammatory conditions such as chronic hepatitis and chronic pancreatitis; chronic osteoarticular diseases such as osteoarthritis, rheumatoid arthritis, osteoporosis; chronic kidney disease; chronic respiratory diseases such as asthma, chronic obstructive pulmonary disease, pulmonary fibrosis, pulmonary hypertension; gastrointestinal diseases such as ulcerative colitis, Crohn's disease, celiac disease, gastroesophageal reflux disease; metabolic/endocrinologic conditions such as diabetes mellitus, obesity, hyperthyroidism, hypothyroidism; mental illnesses such as depression, schizophrenia, bipolar disorder; obstetric conditions such as normal pregnancy, high risk pregnancy; post-transplant conditions; post-surgical conditions; conditions requiring wound care; chronic infectious conditions such as HIV infection or AIDS; immunodeficiency states. It should be understood that the afore-mentioned list and classification is non-exhaustive non-limiting. One of ordinary skill in the art will appreciate that there are numerous other conditions, that many conditions may appropriately be classified in two or more categories, and that the absence of a condition from a particular category in the afore-mentioned list is not intended to indicate that it is not a member of that category.

In some embodiments, a T-plan may be for a complication of a particular condition. Diabetes is an example of a disease that may have a variety of complications, at least some of which may be recognized as distinct medical conditions. A patient may have a T-plan for diabetes and may also have T-plan(s) for one or more complications of diabetes that may be recognized as distinct conditions, such as diabetic macular edema. A T-plan for a complication may have its own managing physician who may be a different physician from the physician managing the primary underlying disorder. For example, in the case of a patient with diabetes and diabetic macular edema, a primary care physician who manages the diabetes may refer the patient to an ophthalmologist to check for eye-related complications of diabetes. If the ophthalmologist diagnoses such a complication, e.g., diabetic macular edema, the ophthalmologist may take on responsibility for treating the patient for that condition and may prescribe a T-plan for it. In some embodiments, the ophthalmologist may view the diabetes T-plan prescribed by the primary care physician and health data associated with the diabetes T-plan, and the primary care physician may view the T-plan prescribed by the ophthalmologist and health data associated with the diabetic macular edema T-plan, thus facilitating exchange of information and coordination of care among the patient's physicians.

In some embodiments, a T-plan may be assigned for general health maintenance. Such a T-plan may be prescribed, e.g., to an apparently healthy patient, by a health care provider or may be self-assigned by an individual (who may or may not be a patient) or assigned to an individual by an authorized individual such as a parent or guardian. A health maintenance T-plan may specify monitoring of one or more types that may be designed to detect one or more types of clinically significant occurrences. In some embodiments, such monitoring may detect a warning sign of a clinically significant occurrence, possibly before the patient becomes aware of it. In some embodiments, such a T-plan may be prescribed after a patient has recovered from a condition and is apparently healthy. In some embodiments, such a T-plan may be prescribed to detect a potential recurrence of the condition. In some embodiments, such monitoring may detect a condition that may benefit from appropriate management or a trend that may, if it continues or is not appropriately managed, evolve into such a condition. For example, monitoring of blood pressure may detect the onset of prehypertension or may detect a trend towards prehypertension over a period of several months or more. The patient, one or more of the patient's health care provider(s), or both, may be notified accordingly. Without wishing to be bound by any theory, early detection of clinically significant occurrences or trends in apparently healthy individuals may reduce the likelihood that the patient will develop a more serious condition or may have a better outcome than would otherwise be the case. In some embodiments, monitoring performed according to a health maintenance T-plan may be less frequent than monitoring according to a T-plan for a patient who has a condition.

In some embodiments, a patient obtains care from multiple health care providers, and methods of the invention provide a health care provider who has prescribed a T-plan to the patient with health data that is obtained while the patient is under care of a different health care provider and/or that is obtained through procedures ordered by a different health care provider. In some embodiments, methods of the invention provide a health care provider who has prescribed a T-plan to the patient with information identifying at least some procedures that were performed on the patient by or upon orders of other health care providers. In some embodiments, the information may further identify the health care facility at which a procedure was ordered and/or performed and/or where results of the procedure are available, may identify the health care provider who ordered or performed the procedure, or both. In some embodiments, at least some results of at least some such procedures are made available to the health care provider who prescribed the T-plan.

As will be appreciated by one of ordinary skill in the art, the technology disclosed herein or any one or more aspects thereof may be used to implement, for example, as a system, apparatus, method, or computer program product. Accordingly, one of ordinary skill in the art could implement hardware, software, or embodiments combining software and hardware aspects based on this disclosure. Such implementations, made up of hardware and/or software, may all generally be referred to herein as “systems”, which may comprise one or more “components”. Similary, one of ordinary skill in the art could implement technology based on the present disclosure in the form of a computer program product embodied in any tangible medium (e.g., a non-transitory storage medium) having computer usable program instructions embodied in the medium. Such a computer program product may comprise multiple distinct computer program products that may interface with each other. Such computer program products, sometimes termed “applications”, may provide functions for use by different types of users, e.g., patients, health care providers.

A system of the implementing some or all of the technology disclosed herein may be referred to herein as the “REVON” system or simply “the system” or “a system”. A computer program product or application implementing some or all of the technology disclosed herein may be referred to herein as a T-plan product or REVON application. In some embodiments, a REVON system comprises a REVON application or user interface for use by health care providers and a REVON application or user interface for use by patients. In some embodiments, a health care provider who uses the REVON system in his or her capacity of providing health care to patients may be referred to as a “REVON health care provider”. A patient who uses the REVON system in his or her capacity as a patient may be referred to as a “REVON patient”. The REVON system may comprise a website (“REVON website”), which may provide web portals for health care providers, patients, or both. Web portals may additionally be provided for researchers, sponsors, payers, governmental entities, and/or other user constituencies.

Any combination of one or more computer usable or computer readable medium(s) may be utilized in various embodiments. In certain embodiments, the invention is embodied at least in part as an application for a mobile device for use by patients and a web-based application for use by health care providers. Patients may use a mobile device to, for example, use various patient-directed functionalities associated with a T-plan, such as performing an interactive health evaluation and receiving directions for management of their condition, viewing health data obtained from them through the application or from connected monitoring devices, and/or viewing a schedule of health care events. Health care providers may use the web-based application to, for example, prescribe T-plans to their patients and use various health care provider-directed functionalities associated with a T-plan, such as viewing patient health data obtained according to T-plans that the health care provider has prescribed to his or her patients or that have been prescribed to such patients by other health care providers.

A computer may be, e.g., a personal computer (e.g., a desktop computer) or a portable electronic device. A “portable electronic device” is a computer that is designed for portability, can typically utilize power supplied by a rechargeable battery that can power the device for extensive periods of time, and provides a built-in keyboard plus pointing device. In some embodiments, a portable electronic device is a laptop computer. In some embodiments, a portable electronic device is an electronic device that a user may hold in his or her hand, such as a portable digital assistant (PDA), a smartphone, a tablet computer, etc. Such a portable electronic device is referred to herein as a “mobile electronic device” or “mobile device”. PDAs are small, e.g., hand-held, computers, that can be used for tasks such as maintaining a calendar, list of contacts (e.g., email addresses), and other information. PDAs may contain application programs such as word processing programs, web browsers, PDF viewers, etc. As used herein, a “smartphone” may be any mobile electronic device that combines the functions of a wireless phone and a computer within a single handheld unit and is capable of web browsing and running software applications, known as “apps”. A tablet computer is typically somewhat larger than a mobile phone or personal digital assistant, comprises a flat touch screen, and is primarily operated by touching the screen. It may use an onscreen virtual keyboard. The term “basic mobile phone” refers to a mobile phone that allows a user to make and receive calls but lacks the computing ability of a smartphone. The term “fixed phone” refers to a hard-wired or cordless phone that makes use of a fixed phone line (a phone line that is not a mobile phone line). A “basic phone” refers to a basic mobile phone or a fixed phone.

Often a mobile electronic device may weigh under about 1-2 pounds, e.g., between about 2-3 ounces and about 1.5 pounds. For example, a smartphone or PDA may weigh between about 3 ounces and about 6 ounces and height and width dimensions in the range of less than about 7×5 inches and depth less than about 0.5-1.0 inch, though smaller or larger weight and/or dimensioned devices may be used. Exemplary portable electronic devices include, e.g., a PDA such as an iTouch (Apple, Inc.), a smartphone such as an iPhone (Apple, Inc.) or Galaxy phone (Samsung) or Windows phone (Microsoft), or a tablet computer such as the iPad or iPad mini (Apple, Inc.), Galaxy tablet, Windows Surface, Fire (Amazon), etc. In some embodiments a mobile device may be wearable, e.g., as a wristwatch, armband, computer with a head mounted display (such as Google Glass) optionally attached to or in the form of glasses, etc. In some embodiments, a smartphone or other mobile device comprises a touch screen and is primarily operated by touching the screen, although of course it may also have one or more controls such as buttons, switches, etc., that are distinct from the screen, e.g., on the top, side, bottom, etc., of the device.

A mobile device may include components that may be found in computers, e.g., control circuitry, storage/memory, input/output circuitry, communications circuitry, processing circuitry, etc. In some embodiments, the mobile electronic device may include a proximity sensor, a gyroscope, a power supply such as a battery, a display, a positioning system, a camera, an accelerometer, an ambient light sensor, other sensors, an input mechanism, or multiple instances of one or more such components. In many embodiments, a mobile electronic device may possess wireless connectivity. For example, the device may have Bluetooth, Wi-Fi wireless network connectivity, and/or the ability to connect to wireless Wide Area Networks, such as those provided by cellular telecommunications companies.

It should be understood that the invention may comprise one or more applications that users interact with directly, e.g., on a mobile device or personal computer, and one or more applications or programs that interact with and/or serve to support those applications. They may, for example, be closer to a required resource or have the capability to communicate with a required resource or perform a requested service. It should also be understood that execution of a particular set of computer-executable instructions may be performed by a single processor or divided among multiple processors. Without limiting the foregoing, computer-executable instructions for performing a health evaluation may be executed by a processor which is part of a mobile device, may be executed by one or more processors operating remotely (e.g., on system servers) that communicate with the mobile device over a communication network (e.g., the Internet), or may be executed in part by a processor which is part of a mobile device or personal computer used by an end user and in part by one or more remotely located processors.

Data Collection Modules

In some embodiments, a T-plan may be composed at least in part of one or more modules, referred to herein as “data collection modules” which in turn specify one or more types of data elements to be collected and, for at least some of the types of data elements, also specify a timeframe according to which data elements of that type are to be obtained and/or according to which underlying health care events that generate data elements of that type are to be performed. Information specifying the various data collection modules of which T-plans may be composed may be stored in a system database. Data collection modules may serve as building blocks that can be assembled in any of a wide variety of combinations to create a wide variety of T-plans. Data collection modules may be selected for use in a T-plan in any of a variety of ways. In some embodiments, data collection modules to be included in a T-plan are determined at least in part automatically. For example, in certain embodiments a health care provider identifies a condition and a set of patient characteristics. A set of one or more appropriate data collection modules is selected by the system based on the condition and the set of patient characteristics. A T-plan comprising the set of data collection modules may be assigned to the patient by storing information associating identifiers of those particular data collection modules with an identifier of the patient. In some embodiments, at least some types of data collection modules may also or alternately be assigned as distinct units independent of a T-plan. Such independently assigned data collection modules may use the same devices and interfaces as those assigned as part of a T-plan.

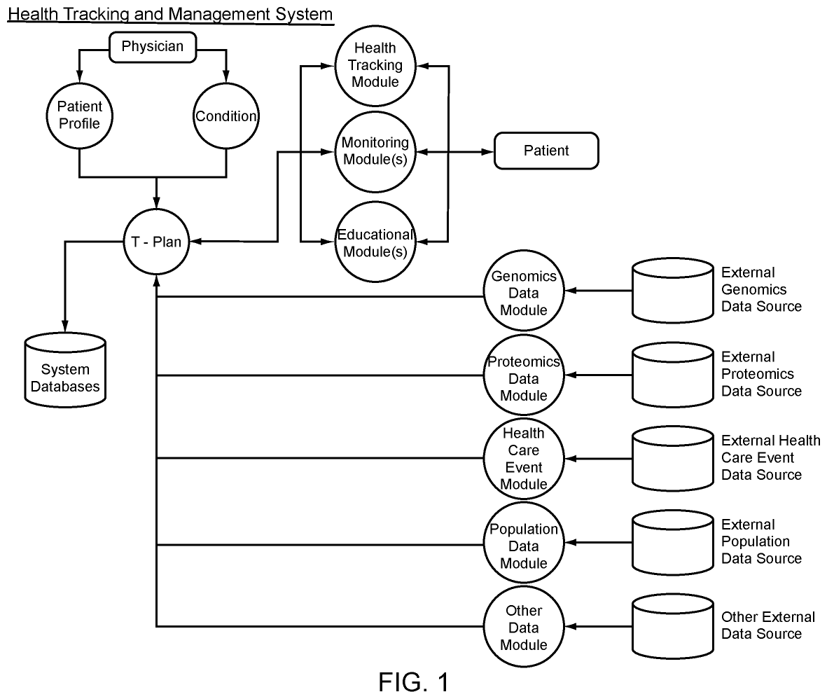

FIG. 1 shows a schematic diagram of a health tracking and management system according to certain embodiments. Referring to FIG. 1, a physician creates a T-plan for a patient by defining a condition and a patient profile for the patient. This results in automatic configuration of a T-plan by the system. The T-plan specifies a set of data collection modules, which include a health tracking module that comprises a smart symptom tracker (described further below), one or more monitoring modules that specify physiological data to be obtained, and one or more educational modules. These modules interact with the patient and gather data which is sent back to the system servers and stored in a system database in association with an identifier of the patient. Additionally, the T-plan specifies data to be obtained from a variety of external data sources via additional modules. Such modules may obtain data from sources such as genomics and proteomics data sources and sources of data pertaining to health care events such as data from reimbursement claims submitted to payers.

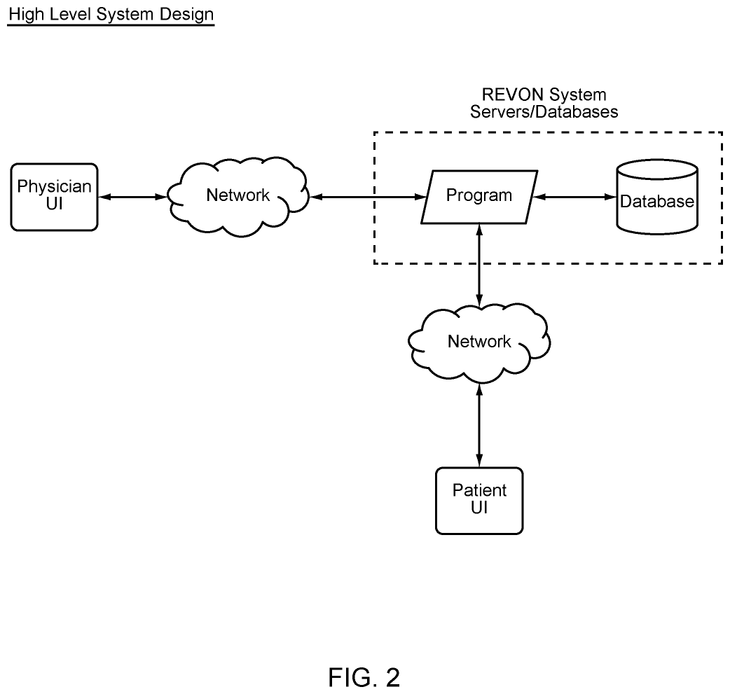

FIG. 2 shows a high level schematic diagram of the system according to certain embodiments. A physician defines a T-plan through his or her user interface (UI) by entering information that identifies a condition and patient characteristics (patient profile) which information is transmitted to the system servers over the network (Internet). System software configures a T-plan based on the information entered by the physician. The patient interacts with the system through the patient user interface, which collects data as specified by the T-plan and provides output to the patient.

In some embodiments, a data collection module may be modified or customized, as described above for T-plans and, once modified or customized by a health care provider, may be saved as a new data collection module in the health care provider's personal T-plan library and used in T-plan templates designed by that health care provider. As described for T-plans, a data collection module in its generic, non-customized form may be referred to as a “data collection module template”. A particular instance of a data collection module template that is part of a T-plan that has been prescribed to a patient by a health care provider as part of an active T-plan may be referred to as an “active data collection module”. Unless specifically indicated, the term “data collection module” should be understood as referring to data collection module templates and active data collection modules unless the context clearly indicates that the term applies only to either data collection module templates or active data collection modules.

A data collection module may specify health data of any of a variety of types, e.g., symptom data, physiological data, behavioral data, environmental data, and/or health care event data, among others. In the case of data collection modules that specify multiple types of data elements, these types may be related to each other in some sort of way. For example, they may be collected using the same channel or from the same data source(s), may be of the same general category, may be useful for one or more of the same purposes, or may be useful together for a particular purpose.

A data collection module may identify a data element type, a timeframe, or both, in any of a variety of ways. Such identification may be explicit or implicit. For example, a data collection module may expressly identify a particular data element type to be obtained, may identify a question that calls for, or would reasonably be understood as calling for, a data element of a particular type as a response, may identify a particular monitoring device that specifically obtains data elements of that type, or may identify a procedure that, when performed, is expected to yield a data element of that type, or may simply identify a monitoring device or procedure, with the data element being whatever result, document, image, report, or thing is produced through performing the procedure. For example, a data collection module that specifies “weight” or that specifies the question: “Please weigh yourself now and enter your weight.” would expressly identify “weight” as a type of data element to be obtained. A data collection module that specifies “pulse oximeter” would implicitly identify “blood oxygen saturation” as a type of data element to be obtained since pulse oximeters obtain this type of data. A data collection module that specifies a “complete blood count” would implicitly identify a set of types of data elements pertaining to blood cells and hemoglobin that would be obtained by performing a complete blood count on a blood sample.

A data collection module may be stored on a computer-readable medium and may comprise, identify, be associated with, be used or referenced by, or be embodied at least in part as computer-executable instructions that, when executed, obtain, request, receive, or search for data elements of any one or more of the types of data elements that it specifies or carry out one or more other tasks as specified in the data collection module. A data collection module that specifies a particular type of data element may, in addition, specify one or more channels that may be used to obtain data elements of that type, may specify a particular channel that is to be used to obtain data elements of that type, or may specify one or more data sources that may be searched for data elements of that type or to which a request for data elements of that type may be transmitted, or from which data elements of a particular type may be received or retrieved.

A data collection module may be described herein as performing various tasks, such as collecting data. However, it should be understood that, as for any module of a T-plan, a data collection module may not actually perform the thing but may merely specify the thing to be performed by, for example, providing information about the thing to be performed, e.g., the types of data elements to be collected and, where applicable, a timeframe. Where the present disclosure refers to a data collection module doing something, such as obtaining data, it should be understood that the data collection module comprises, identifies, is associated with, or is used or referenced by computer-executable instructions for doing that thing as specified by the content of the data collection module.

The REVON system may comprise a database that identifies a plurality of types of data elements and identifies, for each data element type, one or more computer-implemented methods for obtaining data elements of that type or one or more sets of computer-executable instructions for performing such methods. The particular computer-implemented methods or computer-executable instructions used in obtaining the data element types specified by an active T-plan for a particular patient may vary depending on a variety of factors, such as the availability to the patient of mobile devices and/or connected monitoring devices to the patient and/or the availability to the system of data sources that may contain data pertaining to the patient. The system may comprise one or more modules that select one or more appropriate computer-implemented methods or sets of computer-executable instructions for obtaining data elements of a given type for a particular patient. Data identifying the particular methods or sets of computer-executable instructions for obtaining data elements of a particular type may be stored as part of or associated with a T-plan or may be determined at the time the particular data element is scheduled to be obtained according to the timeframe specified for data elements of that type in a data collection module or if required or requested by one or more other modules. When a particular data collection module is included in an active T-plan, specific channel(s) for collecting the data elements specified by that module may be specified based, e.g., on patient preferences and/or availability of connected body monitoring devices. These might subsequently be changed.

In certain embodiments, data collection modules may be classified into any of four main groups, namely health tracking modules, monitoring modules, educational modules, and health care event modules, each of which is described further below. Briefly, a health tracking module specifies health data (typically symptom data, physiological data, behavioral data, environmental data, or a combination thereof) to be obtained from a patient with a condition and, in at least some embodiments, directions to be provided to the patient for managing the condition based on the health data. At least some of the health data may be acquired from the patient by asking the patient questions through a user interface on a mobile device, personal computer, or basic phone, and the directions may be provided through the same channel. A monitoring module specifies physiological data, behavioral data, and/or environmental data to be obtained regarding or relating to the patient. The data may be obtained by asking the patient to enter the data through the user interface on a mobile device, personal computer, or basic phone or may be obtained from one or more connected monitoring devices used by the patient. A monitoring module may be associated with one or more health tracking modules in any of a variety of ways, as described further below, or may be part of a T-plan without necessarily being associated with a health tracking module. An educational module provides educational materials of any of various kinds to the patient through the afore-mentioned user interface and obtains behavioral data relating to use of the educational modules by the patient. A health care event module specifies health care events about which health care event data are to be obtained. A health care event module may specify health care events that are part of a treatment plan for a condition and associated timeframes for at least some of these events, other health care events that are significant in the context of a condition but occur outside the context of a particular treatment plan, or both. A health care event module may collect health care event data from any of a variety of sources, as described further below.

It should be noted that the naming and classification of data collection modules and the types of data elements collected according to any particular data collection module discussed herein should not be considered limiting, nor as requiring or implying any particular architecture or implementation. For example, other names or classification schemes may be used for modules that collect the same types of health data elements and/or other ways of dividing collection of data elements of various types among different modules may be used within the scope of the present invention. It should also be understood that a data collection module may have any of a variety of other roles and tasks in addition to data collection and related to the data collected by the particular module. For example, as mentioned above, a health tracking module may provide directions to the patient for managing the condition in addition to specifying health data to be obtained from the patient.

In some embodiments, a T-plan or one or more modules thereof or features associated therewith may have a specific duration, which may be predetermined. In some embodiments, a duration may be up to 30 days, up to 60 days, up to 90 days, or up to 3, 6, 12, 18, or 24 months. At the end of a predetermined duration, e.g., 30 days, at least some features associated with the T-plan or module may cease. For example, the patient may no longer be provided with the ability to perform a health evaluation. In some embodiments, a T-plan may not have a particular duration but may instead continue indefinitely, at least so long as the patient is under care of the health care provider who prescribed the T-plan or a health care provider who assumes responsibility for managing the condition and becomes the managing physician for T-plan.

In some embodiments, a T-plan may become inactive upon request by the health care provider who prescribed the T-plan to a patient or upon request of the patient. In some embodiments, a T-plan may become inactive if a health care provider or patient indicates to the system that the treatment relationship has ended and no new managing physician has taken over responsibility for the T-plan. In some embodiments, if a T-plan's features have not been used by a patient, prescribing physician, or both, for a predetermined period of time either or both of them may be asked by the system to indicate whether the T-plan should remain active and may switch the status of the T-plan to inactive unless at least one affirmative response is received or, in some embodiments, unless both the physician and patient respond affirmatively. In some embodiments, a T-plan that has become inactive may be re-activated if another physician takes over responsibility for treating the patient, e.g., within a predetermined time period such as within 6 months, or within 1, 2, 3, 4, or 5 years. In some embodiments, a T-plan may remain partially active in the absence of a managing physician. For example, certain features may remain available while other features become unavailable. The patient may continue to be provided with health data collected pursuant to the T-plan. In some embodiments, any customization provided by the prescribing physician may be removed.

We turn now to further description of certain modules that may be included in a T-plan or used in connection with a T-plan.

Health Tracking Modules

In general, a health tracking module is designed at least in part for evaluating the status of a patient's health with respect to a condition at a given point in time and/or for evaluating or following the course over time of a condition that afflicts the patient. In some embodiments, a health tracking module that is part of a T-plan comprises computer-executable instructions for evaluating the patient's health status with respect to the T-plan condition based on data obtained by the system. In some embodiments, a health tracking module for a condition obtains health data relevant to the condition from the patient. The data are analyzed to determine a course of action to manage the condition, and the course of action is suggested to the patient by providing appropriate directions to the patient for implementing the course of action.

In some embodiments, a health tracking module comprises computer-executable instructions for performing a method comprising: (i) obtaining data comprising health data relating to or regarding a patient, wherein the health data are of one or more types that are relevant to a condition or relevant to a material patient characteristic for that condition; and (ii) evaluating the status of a patient's health with respect to the condition based on the data. Performing such a method may be referred to as performing a “health evaluation”. In some embodiments, a health evaluation comprises an interactive health evaluation. In some embodiments, the method further comprises determining directions for management of the condition by a patient or caregiver based on the data. In some embodiments, the method comprises: (i) obtaining health data of one or more types that are relevant to a condition or relevant to a material patient characteristic for that condition; and (ii) determining directions for management of the condition by a patient or caregiver based on the data. In some embodiments, the health data used in evaluating the status of the patient's health and/or in determining directions comprise or consist of symptom data, physiological data, behavioral data, and/or environmental data. In some embodiments, the health data comprises or consists of symptom data and physiological data. In some embodiments, the directions are provided to the patient. In some embodiments, the directions are provided to a caregiver of the patient. In some embodiments, the directions are provided both to the patient and to a caregiver of the patient.

In some embodiments, an evaluation of the patient's health status is implicit in the directions determined by the method. In some embodiments, an explicit evaluation of the patient's health status is performed which might, for example, explicitly describe the patient's health status by way of a severity level or score or other descriptive information in addition to or instead of a set of directions. In some embodiments, information indicating that a health evaluation was performed, the particular data used to perform the evaluation, the results of the evaluation, or any combination thereof, are stored in the patient's record. The information may indicate the time at which the evaluation was performed.

In some embodiments, the method further comprises determining whether or that a notification to a caregiver or health care provider should be issued based on the data. A notification might, for example, inform a caregiver or health care provider of the status of the patient's health as determined by the evaluation. In some embodiments, the method further comprises issuing such a notification to a caregiver or health care provider.

A method comprising performing a health evaluation and providing directions to the patient for management of the condition based on the health data obtained or analyzed in the course of the health evaluation, or a set of computer-executable instructions that, when executed, perform such a method, may be referred to as a “smart symptom tracker” (SST) or “health tracker” herein. It should be noted that an SST may, in some embodiments, not determine directions for management of the condition. Instead, it may perform a health evaluation, results of which may be stored, and/or may determine notifications to be provided to a caregiver or health care provider. Results of the health evaluation may be used for other purposes such as evaluating efficacy of a treatment. A method that comprises obtaining one or more types of symptom data that are relevant to the condition but does not comprise evaluating the status of the patient's health with regard to the condition based on the data or determining directions for management of the condition based on the data may, or a set of computer-executable instructions that, when executed, perform such a method, may be referred to as a “basic symptom tracker”. A health tracking module may comprise one or more smart symptom trackers, basic symptom trackers, or both.

A basic symptom tracker may, for example, be used to track the level of severity of a particular symptom over time. For example, a patient might be asked each day to enter a number or other type of response to indicate the level of severity of the symptom. An example of such a question would be, “How would you rate your pain on a scale of 1 to 10?” or a patient might be presented with the question “How is your cough today?” and a set responses such as “Better than usual”, “About the same as usual”, or “Worse than usual” from which to select a response. Data obtained by a basic symptom tracker may be used, for example, in evaluating the efficacy of a treatment. Particular responses to a basic symptom tracker might trigger the asking of additional questions (follow-up questions). Particular responses to a basic symptom tracker and/or follow-up questions may in some embodiments trigger the running of a smart symptom tracker or may be used in performing a health evaluation.

In some embodiments the REVON system may store information that defines multiple SSTs. Each SST specifies one or more types of data elements to be used to evaluate the status of the patient's health with regard to a condition or to determine directions for management of the condition. A SST may be identified at least in part by the name or identifier of the condition for which it is prescribed, sometimes referred to as the “SST condition”. An SST may be designed at least in part to detect a clinically significant occurrence, such as an exacerbation, that may take place in a patient with a given condition or to detect a deterioration that may develop into such a clinically significant occurrence. The name of the SST may refer to the clinically significant occurrence, sometimes referred to as the “SST occurrence”. For example, an SST designed at least in part to detect a COPD exacerbation or to detect a deterioration that may develop into a COPD exacerbation may be referred to as a “COPD exacerbation SST”. Of course any convenient name may be used. The term “clinically significant occurrence” in regard to health status or physical state refers to any change in a patient's health status or physical state that would be recognized as more significant than typical day to day fluctuations by an ordinary skilled medical practitioner and/or that may, within the judgment of an ordinary skilled medical practitioner, warrant a change in treatment or an evaluation by a health care provider. “Clinically significant occurrence” encompasses “adverse change”, as defined below, as well as changes that may not represent worsening but may warrant medical attention or a change in treatment such as adjusting the dose of a medication. A change that does not represent worsening but may warrant medical attention or a change in treatment (such as adjusting the dose of a medication) may be referred to as a “notable change”. For example, in some embodiments, an increase in blood pressure may be a notable change that leads to a recommendation for salt restriction but may, in some embodiments, not trigger the running of an SST. In some embodiments, an increase in blood pressure may be a notable change that triggers the running of an SST, optionally together with a recommendation for salt restriction. Where the present disclosure refers to an “SST condition” it should be understood that, in certain embodiments, the term “SST condition” should be understood to refer to the clinically significant occurrence that the SST is designed to detect, which may or may not be specific to a specific condition. In certain embodiments, the term “SST condition” may refer both to a particular condition and the particular clinically significant occurrence that the SST is designed to detect that may arise as a consequence of the condition.

The system may provide a set of SSTs for those clinically significant occurrences that are concern to practitioners in any one or more health care disciplines (e.g., specialties) because they occur in a significant number of patients treated within that health care discipline and may result in a need for emergency medical attention or may potentially have severe consequences, particularly if untreated. For example, a set of SSTs for pulmonology may include a COPD exacerbation SST, an asthma exacerbation SST, and a pulmonary embolism SST. A set of SSTs for cardiology may include an arrhythmia onset SST and a congestive heart failure exacerbation SST, among others. In some embodiments, at least some SSTs will be included in T-plans of only a single condition. For example, a COPD exacerbation SST would likely only be prescribed as part of a T-plan for COPD. However, in some embodiments, an SST may be relevant to more than one underlying condition. For example, arrhythmias are a clinically significant occurrence that can occur in patients with any of variety of different cardiac disorders. In some embodiments, an arrhythmia SST may be included in a T-plan for any such disorder. In some embodiments, there may be at most one SST in a T-plan for a particular condition. In some embodiments, there may be at most one SST in any T-plan. In some embodiments, there may be two or more SSTs in a T-plan for a given condition. For example, if the T-plan condition is one in which patients may be prone to two or more distinct types of clinically significant occurrence, such as an acute deterioration or exacerbation that may be detected using different monitors and/or may have different symptoms or different sets of instructions for patient management, the T-plan may include SSTs for more than one such type of acute deterioration or exacerbation or other clinically significant occurrence, e.g., each such type of acute deterioration or exacerbation or other clinically significant occurrence. Thus, an SST may, for example, be a condition-specific SST or an occurrence-specific SST. It should also be understood that a particular SST may be relevant to multiple health care disciplines. It should be noted that SSTs may not be available for certain conditions for which a T-plan may be prescribed. Certain conditions may have a course that is not ordinarily punctuated by exacerbations or other occurrences for which it would be desirable to provide management instructions to a patient. However, it may still be desirable to monitor certain symptoms of the condition. In some embodiments, a T-plan for such a condition may include a basic symptom tracker. Certain conditions may not ordinarily be characterized by symptoms but may still require follow-up. For example, some conditions might merely require periodic evaluation by a health care provider performing an appropriate procedure to make sure that the status of the condition has not changed. For conditions of this type, a T-plan may not include any symptom trackers.

In some embodiments, an SST conducts an interactive health evaluation. The term “interactive health evaluation” refers to an interaction between a user and a system, in which the system presents questions pertaining to symptoms, physiological measurements, or other variables that are of use in evaluating the patient's health status, and the user supplies answers (responses) to the questions presented by the system. It should be understood that a question may take any of a variety of forms. For example, it may be interrogative or imperative. Thus the term “question” should be understood as encompassing any linguistic expression used to make a request for information, or the request made using such an expression. The information requested may be provided in the form of an answer, which could be provided in a variety of different ways. The user is typically a patient or caregiver, although the user may, in some embodiments, be any individual wishing to evaluate the patient's health status. An interactive health evaluation may resemble a conversation between a health care provider and a patient, in which the system presents a question to the user through a suitable user interface and the user responds to the question through the same interface. For example, a patient may be asked “Are you more short of breath than usual?” The response could be provided by entering text, providing a spoken response, selecting from among options presented on a display screen, or any other typical way by which a user can interact with a system. An interactive health evaluation may pertain to a particular condition that the patient has and may evaluate the patient's health status and/or provide directions for management with respect to that condition only or may pertain to multiple conditions and evaluate the patient's health status and/or provide directions for management taking into consideration the fact that the patient has multiple conditions.

In some embodiments, a patient may use a smart symptom tracker voluntarily at any time to evaluate his or her health status with respect to one or more conditions. For example, a patient might voluntarily initiate a smart symptom tracker by selecting a button labeled “Am I OK?”, “Start health evaluation”, or something else indicating a desire to run a health evaluation and receive directions or reassurance. The process of using a smart symptom tracker to perform a health evaluation may be referred to as “running” a smart symptom tracker.

In some embodiments, a patient may additionally or alternatively be prompted to use a smart symptom tracker to evaluate his or her health status upon detection of data (e.g., one or more data elements) indicative of a deterioration or potential deterioration or notable change in the patient's health. Such data may have been obtained by one or more symptom trackers and/or monitoring modules that automatically obtain the data according to a timeframe specified for data elements of that type. In some embodiments, data indicative of a deterioration or potential deterioration or notable change in the patient's health comprises physiological data obtained by one or more monitoring modules.

In some embodiments, a patient may additionally or alternatively be periodically prompted to use a smart symptom tracker to evaluate his or her health status with respect to one or more conditions and obtain management directions. A “prompt” may be any implicit or explicit request or encouragement to a user to do a particular thing. It may consist of or be accompanied by some sort of stimulus that draws the attention of a user such as a sound, vibration, light, string of text, or combination thereof. In some embodiments, a prompt comprises both a sound and a particular string of text such as “Please run your health tracker.” or whatever may be appropriate in the context, optionally accompanied by a sound or vibration at about the same time.

In some embodiments, running a smart symptom tracker comprises analyzing the patient's state with regard to one or more conditions and material patient characteristics (if any) and determining directions (instructions) to the patient based on the patient's state. It should be noted that the data obtained in performing a health evaluation, e.g., by a smart symptom tracker, may have already been obtained by the system and stored in a patient record. In such cases the data may be obtained from wherever it is stored. Thus, obtaining the data may comprise or consist of accessing one or more previously stored data elements. In some embodiments, a determination is made as to whether a previously stored data element remains valid. If the data element has expired, a new data element of that type is obtained, stored, and used. The system may determine, at any time a health tracker is run, which data elements are to be obtained and, where applicable, and how each such data element is to be obtained, e.g., whether it is to be obtained through interaction with a patient or from a connected monitoring device. The questions asked in the course of an interactive health evaluation may be adjusted so as to request only those data elements that need to be obtained through interaction with the patient at the particular time the interactive health evaluation is performed.

In some embodiments, the questions included in an SST may be asked in any order. In some embodiments, the questions included in an SST may be asked in a predetermined order. In some embodiments, questions in an SST may be assigned a priority. In some embodiments, the ordering or priority of questions in an SST may be determined based on patient characteristics. In general, a higher priority question may be asked before a lower priority question. In some embodiments, questions in an SST may be assigned different priorities for use for different purposes. For example, certain questions may have a different priority when asked for purposes of distinguishing between multiple different conditions or clinically significant occurrences (e.g., in the context of a screening session) than when asked for purposes of determining directions to the patient once a specific SST has been selected to be run.

In some embodiments, directions for managing the condition may be of either of two main types, referred to herein as “triage directions” and “treatment directions”. However, in some embodiments, one or more additional types of directions may be issued. Triage directions instruct the patient to seek medical attention (if warranted) or may inform the patient that such attention does not appear to be needed (if appropriate). “Medical attention” in this context refers to medical advice or medical attention through direct interaction with a health care provider. Triage directions may, based on evaluation of factors such as the severity of the patient's condition, indicate the urgency with which medical attention should be sought, may indicate the type of medical advice or attention that should be sought, and/or may instruct the patient how and/or where to seek such medical attention. Triage directions may include instructing the patient to: contact his or her primary care physician or other health care provider to seek medical advice immediately, contact his or her primary care physician or other health care provider to set up an outpatient appointment, seek care at an urgent care clinic or other health care facility, or seek emergency medical attention (such as by calling “911”). Treatment directions may instruct the patient to perform various actions such as taking a medication or performing some other medical intervention to alleviate the condition, reduce the likelihood that the condition will get worse, or serve as prophylaxis to prevent the development of another condition. Depending on the evaluation, triage directions, treatment directions, or both, may be provided to the patient. It should be understood that one or more triage directions and one or more treatment directions may be provided in any format and may be expressed in any way. In some embodiments, one or more triage directions and one or more treatment directions may be provided as part of the same sentence, e.g., “Based on what you have told me, I would like you to take one dose of medication X, and call your physician for an appointment.” or may be provided in two or more separate sentences. In some embodiments, only triage directions are provided. If the evaluation determines that the patient is not in need of medical attention or medical intervention the patient may be informed accordingly and/or may be instructed to continue actions that are part of the patient's ongoing therapy as prescribed by his or her health care providers, such as taking his or her usual medications and the like, thereby providing reassurance to the patient.

In some embodiments, at least some of the directions may be customized. Triage directions may be customized by, for example, specifying the name of a particular health care provider, physician practice, or health care facility; providing contact information such as phone number and/or email address for a particular health care provider, physician practice, health care facility, and the like. Treatment directions may be customized by, for example, specifying a particular medication and/or dose within the context of a generalized direction. For example, a generalized direction might be “double the dose of your diuretic” for the next 24 hours. A customized treatment direction might specify the name of a particular diuretic that has been prescribed to the patient. The particular diuretic may have been specified by the health care provider who prescribed the T-plan to the patient, who may also have prescribed the diuretic. Information required for customizing directions to the patient will typically have been stored in a database as part of the patient's T-plan for that condition.

In some embodiments, a health evaluation may determine whether the patient is experiencing or is at increased risk of experiencing an adverse change in health status. If so, appropriate triage directions, treatment directions, or both, may be provided to the patient. The term “adverse change” refers to any worsening in a patient's health status that would be recognized as more significant than typical day to day fluctuations by an ordinary skilled medical practitioner and/or that may, within the judgment of an ordinary skilled medical practitioner, warrant a change in treatment or may warrant an evaluation by a health care provider. Adverse changes include, e.g., the onset of an infection in an individual who is particularly vulnerable to infections (e.g., an individual who is immunocompromised or suffers from one or more chronic conditions); exacerbations of a chronic condition; acute events such as myocardial infarction, stroke, and embolism; and failure of a patient to adhere to a recommended treatment regimen. The term “exacerbation”, also referred to as a “flare-up” or sometimes a “decompensation” refers to a relatively sudden deterioration of a chronic condition from a previous state, e.g., a patient's usual state of health, e.g., a stable state. Exacerbations, are a common feature of a number of chronic diseases such as congestive heart failure, chronic obstructive pulmonary disease, asthma, lupus, arthritis, inflammatory bowel disease (Crohn's disease, ulcerative colitis), and multiple sclerosis. An exacerbation typically arises over a period of up to an hour, up to a few hours, up to a day, up to a week, or up to two weeks. Exacerbations are typically characterized by a marked increase in one or more symptoms and/or a marked alteration in one or more physiological parameters relative to the patient's usual state and are distinct from the slow, progressive deterioration that is typical of many chronic diseases. Exacerbations may become more frequent and severe over time, may lead to longer term deterioration in the patient's health, and in some conditions may be associated with significant risk of mortality. They are a common cause of emergency room visits and hospitalizations. In some embodiments, directions provided by a health tracking module include medical interventions that may reduce the likelihood that a patient will experience an exacerbation or may reduce the severity of an incipient or ongoing exacerbation. Without wishing to be bound by any theory, it is proposed that early detection and treatment will reduce the likelihood that an exacerbation will escalate to the point where emergency treatment or hospitalization is warranted. In some aspects, systems and methods described herein may reduce the likelihood that a patient will experience an exacerbation or require emergency treatment or hospitalization for a chronic medical condition.

In some embodiments, a SST may instruct a patient to take a medication that is intended to alleviate symptoms, at least partly stabilize the patient's health status, prevent or limit further deterioration in a patient's health status, reduce the likelihood of a further deterioration in a patient's health status, reduce the likelihood of or limit the severity of a secondary condition such as an infection in a vulnerable patient. For purposes of the present disclosure, such medications may be referred to as a “rescue medication”. A rescue medication may, for example, reduce the likelihood that an incipient exacerbation will escalate into a more serious exacerbation, reduce the severity of an exacerbation, reduce the likelihood that one or more symptoms will become serious enough to require emergency treatment or hospitalization, prevent a patient from developing a complication, or the like. A rescue medication may begin to act in a relatively short time period (typically within 24 hours or less) to alleviate symptoms and/or at least partly stabilize the patient's health status. One of ordinary skill in the art will be aware of suitable medications for a variety of conditions and patients with various patient characteristics. For example, in the case of certain conditions that feature inflammation, anti-inflammatory agents such as corticosteroids may be a suitable rescue medication. In the case of respiratory conditions such as COPD and asthma that feature airway flow narrowing or limitation, a patient may be instructed to use a bronchodilator as a rescue medication. In the case of CHF, a patient may be instructed to take a diuretic agent. A rescue medication may be a medication that the patient takes on a regular basis, but is administered at an increased dose or more frequently than usual upon instruction from a health tracking module. In the case of an infection or suspected infection or in a patient who is at increased risk of developing an infection or is at increased risk of developing severe consequences as a result of an infection, a rescue medication may be an antibiotic or antiviral agent. In some embodiments, a rescue medication for an infection or suspected infection may be a broad spectrum antibiotic, which term refers to an antibiotic that is effective against a broad range of bacteria, sometimes including both gram positive and gram negative bacteria. Other rescue measures that a patient may be instructed to take may serve similar purposes as rescue medication but not involve the administration of a medication. For example, such rescue measures may include utilizing a medical device (e.g., a ventilator), physical maneuvers such as elevating or lowering a body part, lying down, and the like. In some embodiments, a health tracking module may instruct a patient to take a rescue medication if available. For example, the instruction may say, “If you have medication X, please take one tablet and make an appointment with your physician immediately.” or the like. In some embodiments, a health tracking module may not include instructions to take a rescue medication.

In some embodiments, management directions may be at least in part conditional or contingent in that, for example, they may vary depending on the availability to the patient of an item needed for a rescue measure and/or may vary depending on the patient's response to a rescue measure. In some embodiments, management directions may include one or more treatment directions and one or more triage directions that may differ based on based on the patient's response to a treatment direction, e.g., whether or not the patient's symptoms or physiological state improves after having followed the treatment direction. For example, a direction may state, “Please take one extra dose of medication X as directed and call your physician for an appointment if your symptoms do not improve within Y hours.” The direction may refer to particular symptom(s) or physiological parameter(s) and/or may indicate the circumstances or timeframe under which the patient should perform particular actions specified by the triage instruction. “Medication X” may be a medication that the patient takes on a regular basis and is specified by one or more of the patient's T-plans. In some embodiments, the patient may be asked to indicate whether he or she has the medication or other item needed to implement the treatment direction available. If the patient responds that he or she does not have the item, one or more alternative directions may be provided.

In some embodiments, management directions may include one or more treatment directions and one or more triage directions that may differ based on the availability of one or more items needed to implement rescue measures (e.g., rescue medications or devices needed to implement rescue measures). For example, in some embodiments, given the same symptom data and physiological data, an outcome may differ based on availability to the patient of certain items required to implement a rescue measure to the patient and, assuming that the item is available, the outcome may further differ based on the patient's response to the rescue measure (which may be ascertained in a follow-up by the system). For example, if a particular item needed for a rescue measure is not available to the patient, the outcome may be a first triage instruction, whereas if the item is available the patient may be directed to implement the rescue measure, wait for a period of time, and then if the patient's state has not improved follow the first triage instruction else follow the second treatment instruction if the condition has improved. For example, if a particular item is not available to the patient, the outcome may be a triage instruction that directs the patient to visit an urgent care clinic, whereas if the item is available the patient may be directed to implement the rescue measure, wait for a period of time, and then visit the urgent care clinic if the patient's state has not improved else make an appointment to see the patient's physician if the condition has improved.

In some embodiments, directions provided by a health tracker may change adaptively over time, based on health data obtained according to the T-plan and/or based on data gathered from a population of patients. In some embodiments, directions take the time of year into consideration. For example, patients at risk of severe influenza virus infection may be instructed to take an anti-influenza virus agent such as Tamiflu during times of the year in which influenza commonly occurs. In some embodiments, directions take population-based data into consideration. For example, patients at risk of severe influenza virus infection may be instructed to take an anti-influenza virus agent during parts of the year when the prevalence or incidence of influenza is above a certain threshold in the geographic region where the patient lives.

The rescue medications/measures may be preselected taking into account patient characteristics, health care provider or physician practice preferences, protocols used at the particular health care institution at which the SST is prescribed, formulary of medications available at a particular health care institution and/or that are approved to be prescribed under a particular insurance policy or program (e.g., Medicare, Medicaid, or any health care insurance policy that a patient has), and other factors as appropriate. Rescue medications and other rescue measures suggested by an SST may be those that a health care provider may ordinarily suggest during a direct interaction with the patient, such as by phone. The rescue medications/measures may in some embodiments be as set forth in discharge instructions provided to the patient at the time of hospital discharge. As described herein, in some embodiments an SST is at least in part customizable, and, in some embodiments, rescue measures/medications are among the at least partly customizable features. For example, a health care provider, health care institution may be able to select from a panel of different pharmaceutical agents that are generally accepted as substitutable for use in a particular context.

In some embodiments a patient may access and use health trackers and various other patient-directed features associated with T-plans through a computer program product of the present invention. As mentioned above, in some embodiments, a patient may do so using a mobile device or personal computer. In certain embodiments of particular interest, the invention provides an application for use on a mobile phone, comprising a user interface (patient interface) through which patients can run health trackers, receive directions, and receive health statistics based on health data pertaining to them collected pursuant to the T-plans that have been prescribed to them.

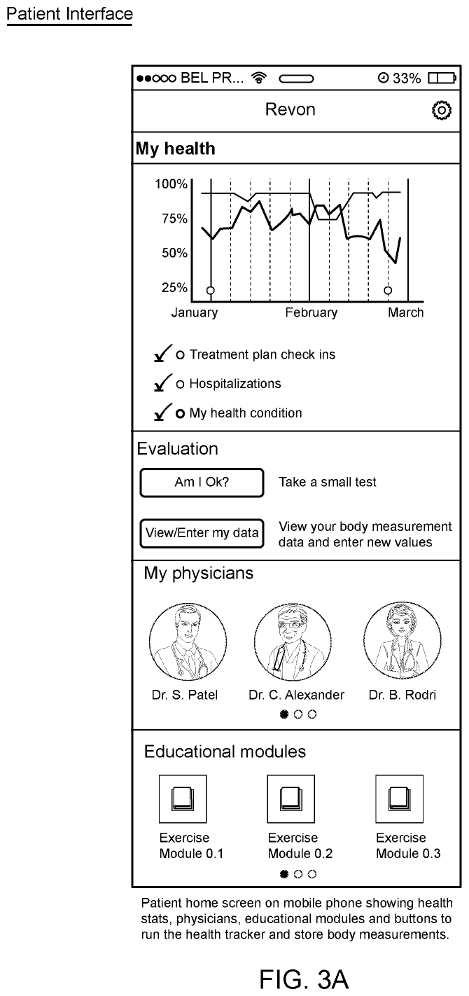

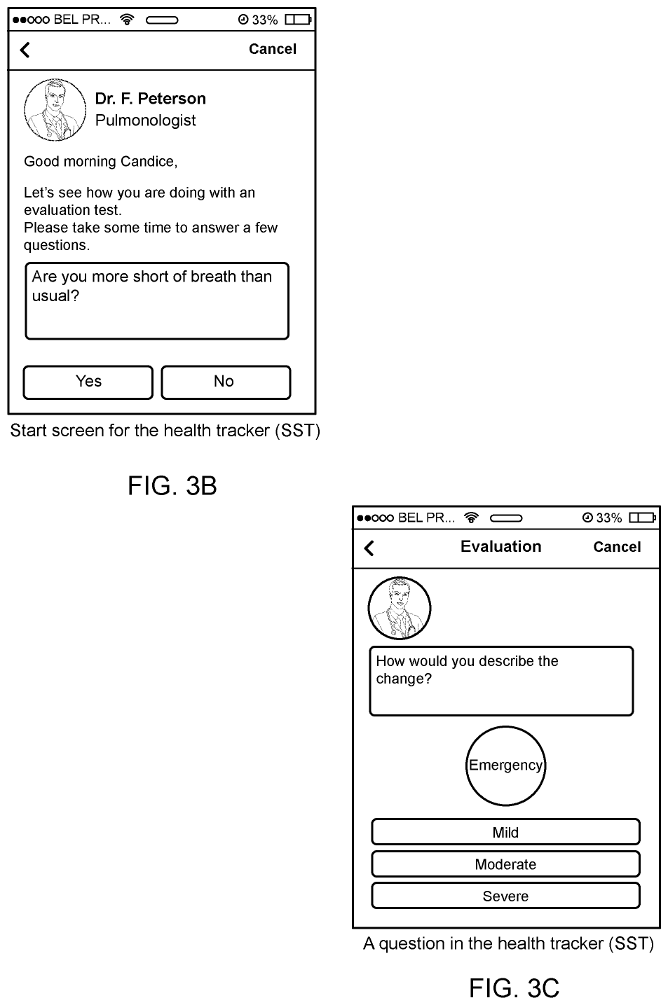

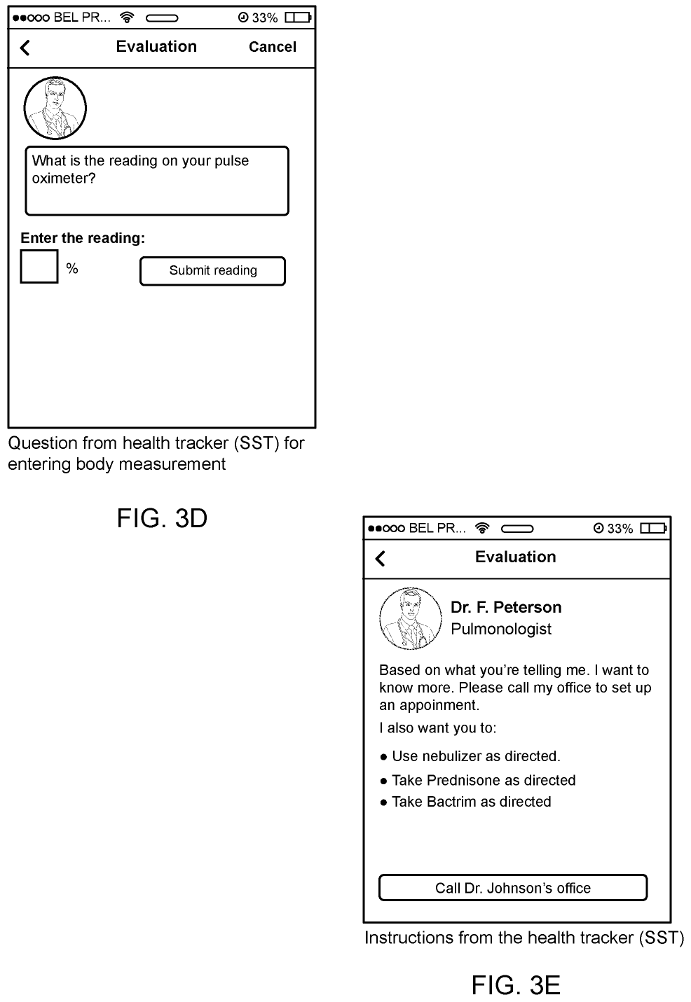

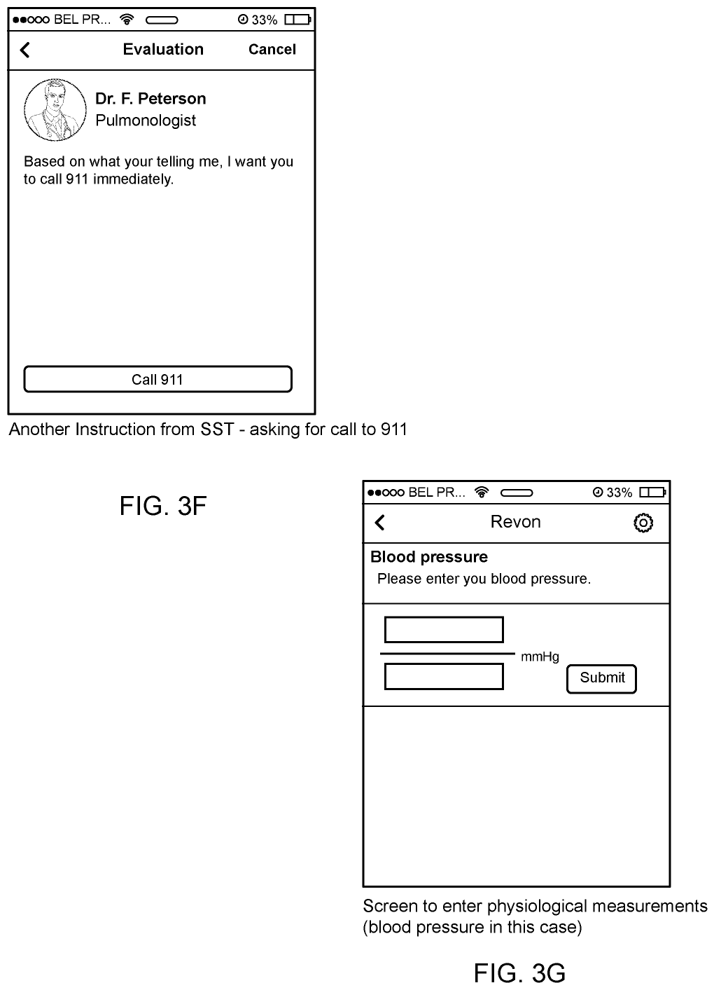

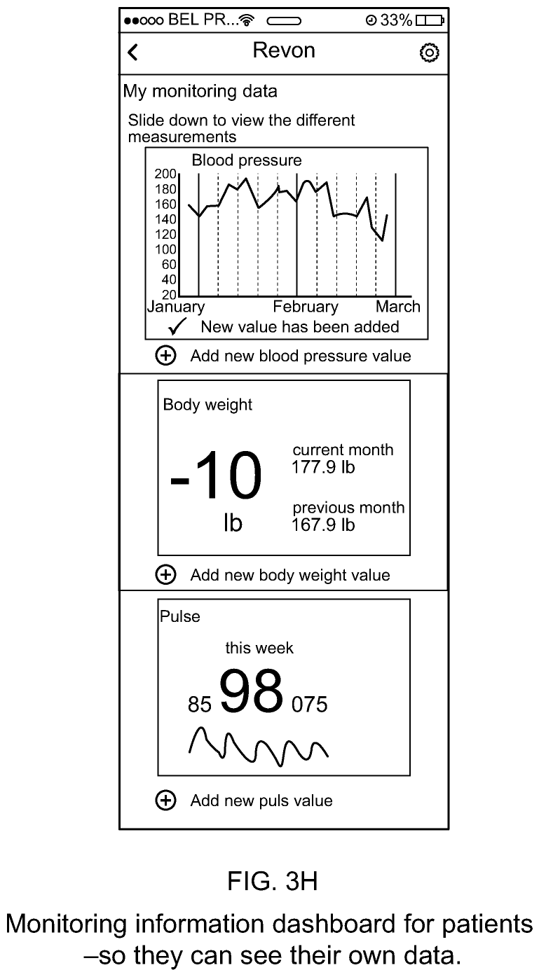

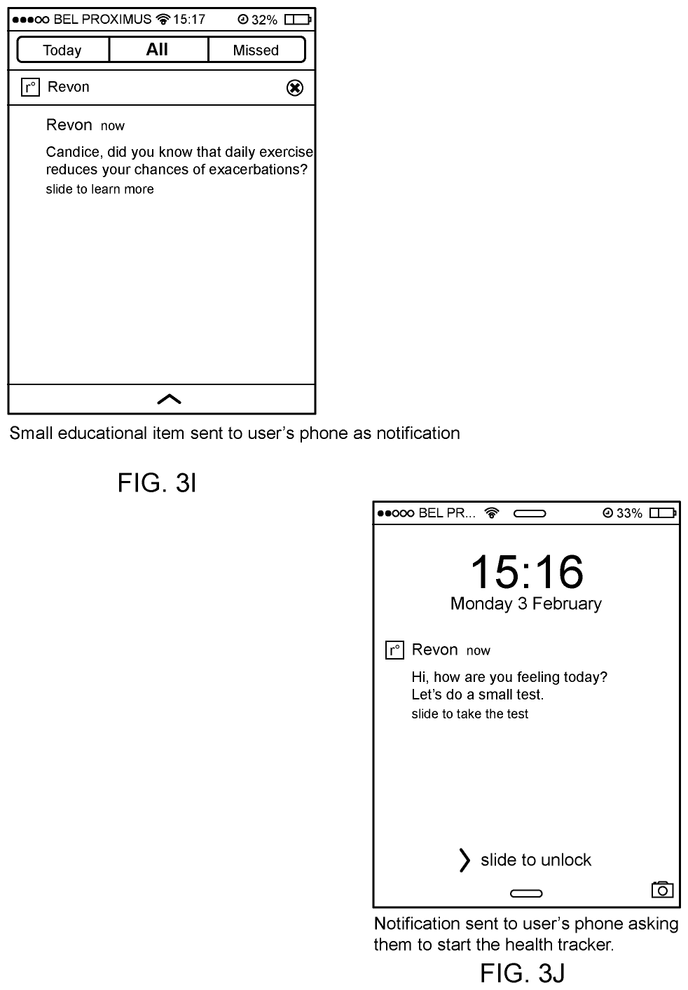

By way of example, FIG. 3A shows a home screen of an embodiment of a patient application on a mobile phone showing health statistics, physicians that have prescribed T-plans to the patient, educational modules prescribed as part of T-plans (discussed further below), and buttons to run a health tracker and store physiological measurements. Tapping the button labeled “Am I OK?” starts the health tracker, which may proceed to ask the patient a series of questions. If the patient has a single T-plan with a single SST the questions may typically focus on symptoms and physiological parameters relevant to the SST condition. In some embodiments, if the patient has multiple conditions, questions relating to multiple conditions and/or clinically significant occurrences may be integrated. In some embodiments, the “Am I OK” button asks open-ended questions and/or allows a patient to select one or more symptoms or physiological parameters of concern. In some embodiments, based on the patient's responses, the system determines which SST(s) to run (if any) and runs them.

FIG. 3B shows a start screen for an embodiment of a smart symptom tracker for COPD. As exemplified on this figure, the name and photo (if available) of the physician who prescribed the T-plan for COPD may appear on screens that relate to the COPD T-plan. The first question asks about shortness of breath (a common symptom in patients with COPD) to which the patient may select “Yes” or “No”. If the patient selects “Yes”, additional questions relating to shortness of breath may be asked. For example, the patient may be asked to describe the change (FIG. 3C). Additional questions may be asked pertaining to symptoms and/or physiological data useful to evaluate the patient's health status and determine a course of action. For example, FIG. 3D shows a screen that asks the patient to enter a reading from a pulse oximeter, which indicates the oxygen saturation level in the patient's blood (an important physiological indicator in patients with COPD). Once sufficient health data have been obtained to evaluate the patient's health status, directions are provided to the patient based on the data. FIG. 3E shows a screen that instructs the patient to take certain medication and call the physician's office. FIG. 3F) shows a screen instructing the patient to call 911.

Returning to the home screen of the patient application (FIG. 3A), in some embodiments the patient can access condition-specific screens by selecting the physician who treats the patient for that condition (FIG. 3A), can swipe across the screen to bring up condition-specific screens, and/or can access condition-specific screens by tapping buttons listing the various conditions for which the subject has T-plans (not shown on FIG. 3A). The home screen may show a summary of health statistics for the patient based on data obtained by the patient's various health tracking modules.

In some embodiments, each condition for which the patient has been prescribed a T-plan may have a main screen. For example, in the case of a patient with T-plans for COPD, diabetes, and hypertension (HT), there may be a main screen for a COPD T-plan, from which modules of the COPD T-plan can be accessed; a main screen for a diabetes T-plan from which modules of the diabetes T-plan can be accessed; and a main screen for HT from which modules of the HT T-plan can be accessed. In some embodiments, the application may provide screens that show consolidated information. For example, there may be a screen that shows a list of symptom trackers (e.g., SSTs) consolidated across all of the patient's T-plans, a screen that shows consolidated health data across all conditions for which the patient has T-plans, a screen that shows consolidated monitoring data across all conditions for which the patient has T-plans, a screen that shows consolidated medications across all conditions for which the patient has T-plans, a screen that shows a list of educational modules consolidated across all conditions for which the patient has T-plans, a screen that shows a consolidated schedule across all conditions for which the patient has T-plans, etc. For example, FIG. 3H shows a screen showing a dashboard that presents consolidated monitoring data for a patient according to certain embodiments. It will be understood that buttons or icons or other representations may be used instead of lists. The patient can select a particular symptom tracker, medication, educational module, etc., from the consolidated screen.

In some embodiments, e.g., if the patient has two or more conditions, a smart symptom tracker may begin with one or more starter questions, or a screening session may be performed to determine which among various condition-specific or occurrence-specific smart symptom trackers should be run and/or which other action(s) the system should take. A “screening session” refers to a method that comprises obtaining health data that pertains at least in part to a patient's current state, wherein at least some of the health data is obtained interactively from the patient, and determining, based on the data, whether the patient has experienced, is experiencing, or is at risk of experiencing a clinically significant occurrence. Typically the method further comprises, if the patient is determined to have experienced, be experiencing, or be at risk of experiencing, a clinically significant occurrence, determining the identity of the clinically significant occurrence, determining the identity of a condition associated with the clinically significant occurrence, or both. A screening session may result in a determination of which actions should be taken, if any, may result in prioritizing further data to be obtained (e.g., further questions to be asked to a patient), or both. For example, a screening session may determine which, if any, SSTs should be run.

A screening session may be performed in any of a variety of ways. For example, a patient may be presented with a list of symptoms and/or physiological parameters and asked to select those that currently concern the patient and/or may be presented with one or more starter questions. Based on the data entered by the patient (e.g., which symptoms and/or physiological parameters are selected or the content of responses provided in response to the starter question(s)) the health tracking module may determine which condition-specific or occurrence-specific SST(s) to run and/or which other action(s) to take. In some embodiments, the starter question(s) comprise one or more high priority questions from one or more of the SSTs included in the patient's T-plans. For example, a screening session may ask one or more high priority questions from each SST. In some embodiments, the screening session determines, based on responses to previous questions asked during the screening session, which further questions to ask, until a definitive determination of which SSTs to run and/or which other actions to take can be made.

In some embodiments, a patient has one or more T-plans that collectively comprise two or more SSTs, and a screening session comprises sufficient questions to permit the system to determine which of the different clinically significant occurrences that the patient's various SSTs are designed to detect is at least reasonably likely or most likely to be responsible for one or more symptoms or physiological data of concern to the patient. In some embodiments, a patient has one or more T-plans that collectively comprise two or more SSTs, and a screening session comprises sufficient questions to permit the system to prioritize the patient's various SSTs according to the likelihood that the clinically significant occurrence that an SST is designed to detect is responsible for one or more symptoms or physiological data of concern to the patient. In some embodiments, a screening session may gather sufficient data to differentiate between the different clinically significant occurrences that a patient's various SSTs are designed to detect. For example, if a patient enters “cough” and “shortness of breath” as symptoms of concern, and the patient's T-plans include SSTs for COPD exacerbation and CHF exacerbation, the screening session may ask one or more questions designed to gather data sufficient to determine whether to run a COPD exacerbation SST or a CHF exacerbation SST and may make such a determination based on the data.

In some embodiments, a screening session is performed if the patient selects a button or other GUI widget labeled “Am I OK?” or the like. For example, if a patient's T-plans include SSTs for COPD exacerbation and CHF exacerbation and the patient selects “Am I OK?” the screening session may ask one or more questions that allow the system to determine whether to run a COPD exacerbation SST or a CHF exacerbation SST and may make such a determination based on the data.

In some embodiments, a screening session may determine, instead of or in addition to determining which SSTs to run, which other action(s), if any, the system should take. For example, in some embodiments, a screening session may determine that the patient is in need of emergency medical attention and may direct the patient to obtain it regardless of which clinically significant occurrence or condition is responsible for the emergency situation. In some embodiments, a screening session may determine that the patient's caregiver or health care provider should be notified regardless of which clinically significant occurrence or condition is responsible for the emergency situation or may determine which health care provider should be notified. The determination of which health care provider to notify may be based on which clinically significant occurrence the patient is determined to have experienced, be experiencing, or be at risk of experiencing, determining the identity of a condition associated with the clinically significant occurrence, or both. For example, if a patient is determined to be at risk of experiencing a particular clinically significant occurrence associated with a particular, the health care provider responsible for prescribing a T-plan for the condition to the patient may be notified.

A health tracking module may use patient state data, patient profile data, or other data regarding the patient as well as or instead of data obtained through a screening session to determine which SST(s) to run, their order, and/or the order in which questions within an SST or among multiple SSTs are to be asked and/or to determine which other actions, if any, the system should take. In some embodiments, a screening session may provide the patient with the option to select one or more condition-specific or occurrence-specific SSTs to be run. In some embodiments, if the patient has multiple SSTs, such SSTs may be run consecutively or questions pertaining to different conditions or clinically significant occurrences may be interspersed.

A patient may be prompted to perform a health evaluation, e.g., to run a smart symptom tracker, at various times. In some embodiments, a patient may be prompted to run a smart symptom tracker based on physiological data collected by a monitoring module indicative of a deterioration or potential deterioration or other clinically significant occurrence in the patient's health. The particular criteria used to determine whether data are indicative a deterioration or potential deterioration or other clinically significant occurrence in the patient's health will vary depending on the condition and the types of data elements being considered. The system may store information identifying normal ranges, limits below which a value is considered to indicate a deterioration or potential deterioration in the patient's health, or limits above which a value is considered to indicate a deterioration or potential deterioration in the patient's health. Sometimes any value markedly outside the normal range may indicate a deterioration or potential deterioration in the patient's health. Sometimes a value markedly below the normal range or below a particular value may indicate a deterioration or potential deterioration in the patient's health. Sometimes a value markedly above the normal range or above a particular value may indicate a deterioration or potential deterioration or other clinically significant occurrence in the patient's health. For some variables, a change from a previously measured value may be indicative of a deterioration or potential deterioration or other clinically significant occurrence in the patient's health. For example, data indicating a marked increase from one day to the next in the weight of a patient with CHF may indicate that the patient is experiencing or is at risk of experiencing a CHF exacerbation. The patient may be automatically prompted to run an SST for CHF exacerbation asked further questions. Responses to such additional question(s) may be used to either determine whether the patient is likely experiencing a CHF exacerbation or is at risk of experiencing a CHF exacerbation, to determine directions to the patient, to determine whether other action should be taken such as notifying a caregiver or health care provider, or to determine whether additional data should be obtained in order to definitively make any of the afore-mentioned determinations. In some embodiments, the health tracker iteratively selects and makes one or more additional data requests based on data received in response to previous data requests until directions for management of the condition by a patient or caregiver are definitively determined. In some embodiments, a patient has multiple SSTs, and a particular SST may be run based on detecting particular physiological data suggestive of a deterioration or potential deterioration in the SST condition or suggestive of the onset or increased risk of onset of the SST occurrence for that SST. FIG. 3J shows a screen in which the patient is being prompted to run a health tracker. In some embodiments, the patient may be informed that the system detected an abnormal value and may be informed which value was abnormal. This may motivate the patient to run the SST. For example, the patient may receive a message such as “Your weight has increased by 3 pounds since yesterday. Please run your CHF exacerbation SST.” or “Your weight has increased by 3 pounds since yesterday. I would like to ask you some a few questions about how you are feeling.”

A trigger for prompting a patient to perform a health evaluation, e.g., to run a smart symptom tracker, may be based on data elements of any one or more types. In some embodiments, data elements of different types may be assigned weights according to their diagnostic value in terms of determining whether the patient is at sufficient risk of experiencing a deterioration to warrant performing a health evaluation. The diagnostic value of a particular type of data element may vary depending on the particular condition(s) and the patient characteristics that the patient has. A combination of the values of the data elements and their weights may be used to determine a score, which may be used to determine whether to perform a health evaluation, whether to run one or more SSTs and, in some embodiments, the order in which to run them. The values of the data elements may be absolute values, deviation from normal value (which may be adjusted for demographic characteristics such as age and sex), change from patient's baseline, rate of change from patient's baseline, or a combination thereof

In some embodiments, a patient may be prompted to run a smart symptom tracker on a periodic basis. The frequency of such prompts may vary depending on the SST condition, the clinically significant occurrences that may be associated with the SST condition, and/or data previously collected from the patient. For example, a patient might be asked weekly about their symptoms at that particular time or over the previous week.

In some embodiments, health data collected by connected monitoring devices may be used in performing a health evaluation in addition to or instead of data collected by asking questions to the patient. In some embodiments, data that has been previously collected by monitoring modules is checked to determine whether the most recently collected value for a data element of a particular is valid or whether it has expired. If the value is valid, it may be used in performing a health evaluation. If it has expired, then a new value for the particular data element type is obtained. This may be done, for example, either by requesting the most recent data obtained by a connected monitoring device, by requesting a connected monitoring device to obtain new data, or by asking the patient to enter the data. The particular way the new data are obtained will vary depending on the available channels for obtaining that data for the particular patient.

In some embodiments, one or more follow-up questions may be asked or further directions (collectively “follow-up”) may be provided to the patient after a health evaluation is performed. Follow-up may be performed if particular health data are obtained or particular directions are provided to the patient. For example, if patient is instructed to take a medication or make an appointment with a health care provider, the patient may subsequently be asked whether he or she took the medication or made the appointment as directed. In some embodiments, a follow-up question may, for example, be designed to ascertain whether the patient's health status improved, e.g., whether a symptom has become less severe or a physiological variable is within or closer to a normal range after taking a rescue medication. In some embodiments, further directions might direct the patient to take an additional dose of a rescue medication or refrain from taking an additional dose of a rescue medication. In some embodiments, if the patient reports worsening symptoms despite having indicated that he or she took the rescue medication, the patient may be directed to seek urgent medical attention. In some embodiments, a notification may be issued to a caregiver or health care provider based on responses to follow-up questions, or lack thereof. For example, if the patient reported worsening symptoms despite having taken the rescue medication, a health care provider may be notified.

In general, a health evaluation or an SST may utilize any of a variety of different algorithms to determine a patient's health status with respect to a condition and/or to determine directions for management of the condition. It should be understood that the invention is in no way limited in this respect. Algorithms may rely entirely on embedded medical knowledge that maps each particular combination of inputs to an SST to a particular output, may employ any of the various tools of artificial intelligence. In some embodiments, an algorithm may be embodied as a decision tree. In some embodiments, an algorithm may be embodied as a set of rules and an inference engine. In some embodiments, an algorithm may make use of fuzzy logic, probabilistic methods (e.g., Bayesian networks, Hidden Markov models) neural networks, or other approaches.

In some embodiments, an SST determines health status and/or directions according to an algorithm that considers both (1) the SST condition (i.e., the particular condition for which the SST or T-plan containing that SST is prescribed and/or the particular clinically significant occurrence that the SST is designed to detect) and (2) a set of patient characteristics. In some embodiments, the operation of the algorithm is completely determined by the condition and the set of patient characteristics. In some embodiments, the algorithm is not specified and fixed for a particular condition but instead provides an evaluation and/or directions that may vary depending on patient characteristics. In some embodiments, the algorithm is not specified for a fixed combination of a condition and acomorbidity but instead provides an evaluation and/or directions that may vary depending on patient characteristics. The particular set of patient characteristics used by the algorithm for a given condition are those patient characteristics deemed material to management of the condition (the “material patient characteristics”). In some embodiments, the algorithm applies a set of rules that map from (1) a condition (e.g., a particular disease) or clinically significant occurrence and a subset of a set of material patient characteristics for that condition to (2) a subset of a set of directions relevant to managing that condition or clinically significant occurrence. The rules operate on input comprising health data collected by the system. For purposes of convenience, it will be assumed that the output of the algorithm is a set of management directions, but the same principles could apply if the output were an explicit description of the patient's health status. The same principles may also be applied to determine notifications and follow-up questions.

Certain algorithms and examples of SST logic are provided herein, but it should be understood that these are only representative and should not be considered limiting.

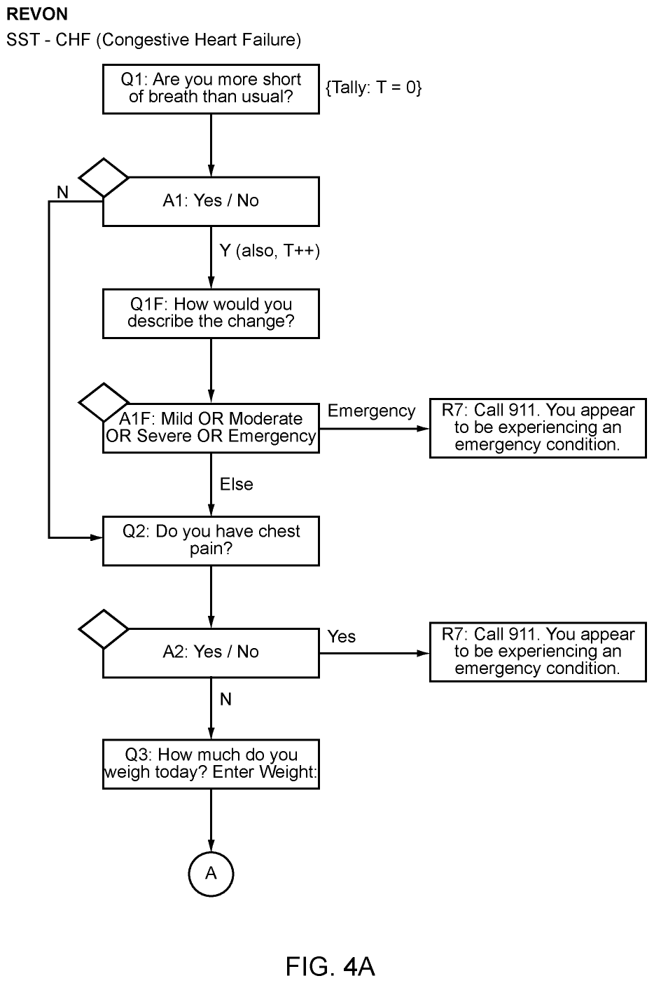

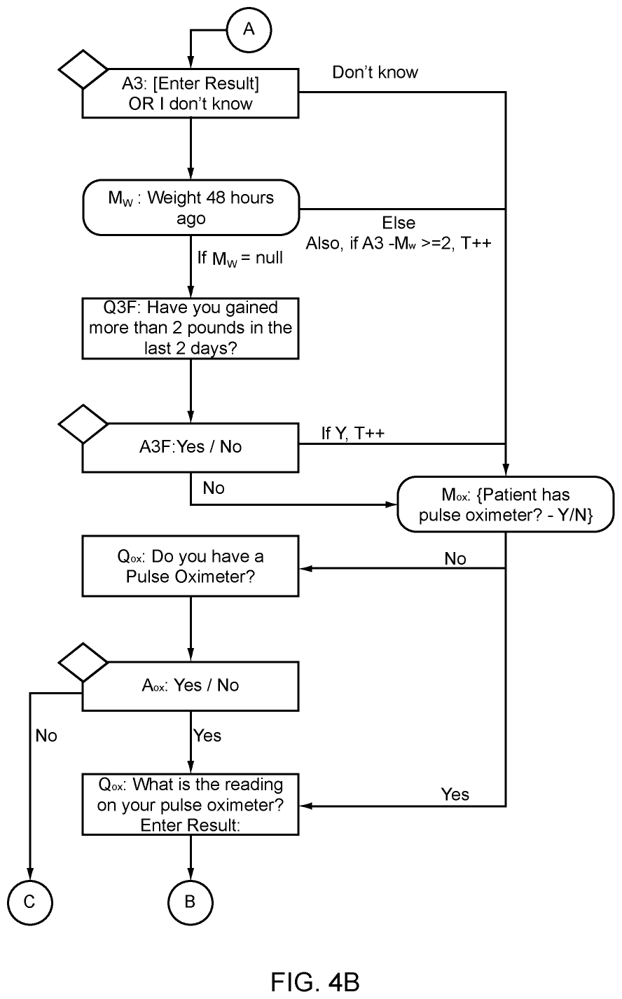

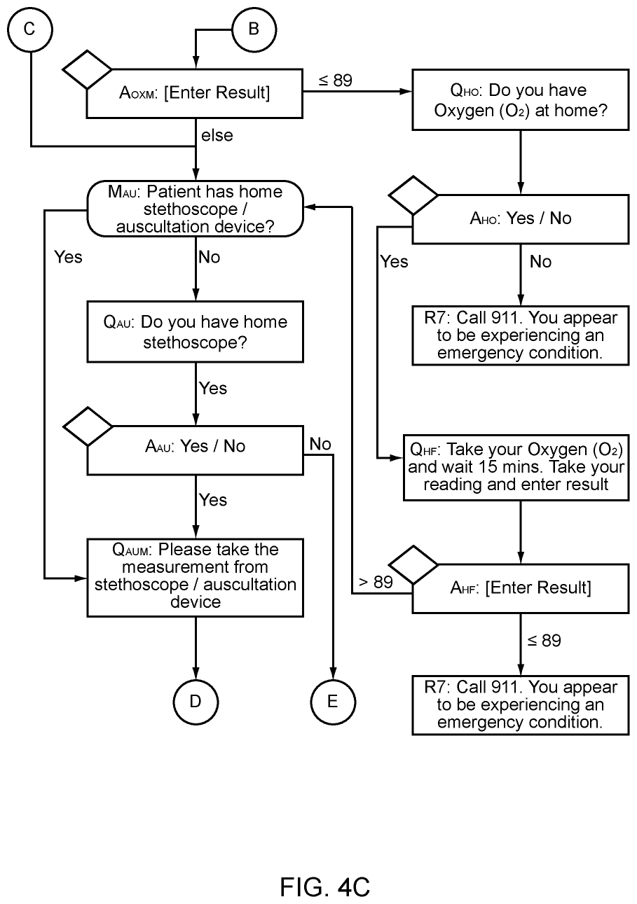

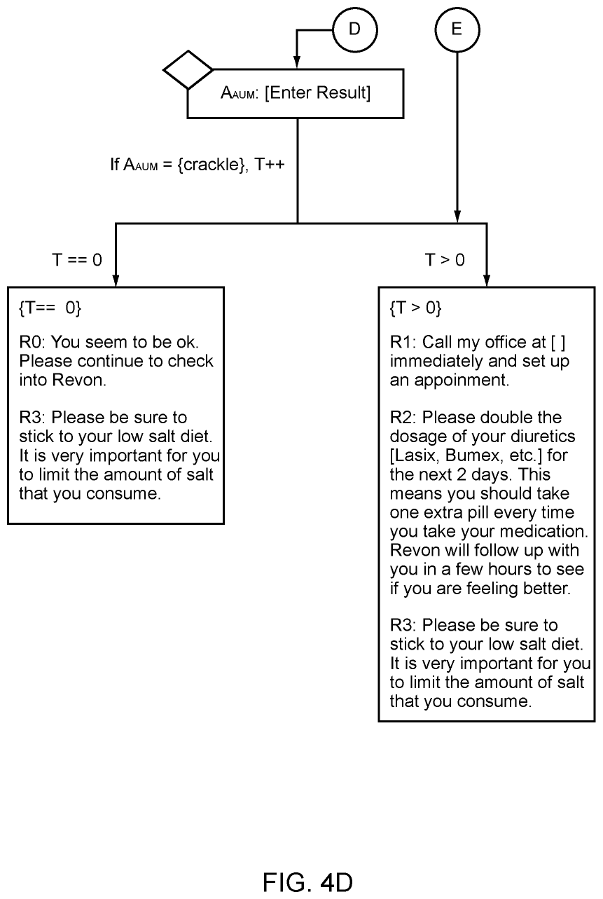

In some embodiments an SST may be represented in a decision tree format. FIGS. 4A-4D present a schematic diagram of an embodiment of an SST for congestive heart failure (CHF) in a decision tree format using flowchart type symbols in which gray rectangles indicate questions asked to the patient, blue rectangles with diamonds at left corner represent responses from the patient to the questions, pink ovals indicate data elements for which data are obtained or requested from a monitoring module, and green rectangles indicate management instructions issued to the patient upon reaching an end node. Yellow circles labeled A, B, C, D, and E are not part of the decision tree but instead are used to show how portions of the decision tree that span two sheets of the figure align (e.g., the A near the bottom of FIG. 4A aligns with the A near the top of FIG. 4B. The algorithm exemplified by FIGS. 4A-4D makes use of embedded medical knowledge to arrive at appropriate management directions. For example, one of ordinary skill in the art appreciates that rapid weight gain by a patient with CHF often indicates fluid retention, which is frequently associated with a CHF exacerbation. Thus, a weight gain of, for example, at least 2 pounds over 2 days may indicate that the patient is experiencing or is at increased risk of experiencing a CHF exacerbation.

It should be understood that the underlying algorithms executed by an SST may vary. In some embodiments, an algorithm may make use of a decision tree. In some embodiments, the algorithm may not make use of a decision tree but may be represented as a decision tree and/or may generate the same results as would a decision tree. Any of a wide variety of algorithms for analyzing the data and generating a course of action may be used and the invention is not limited in this respect. For example, data elements collected by the SST may be assigned various weights, which may be added to provide a score, with a particular score resulting in particular directions. This approach is also illustrated in FIGS. 4A-4D by way of a tally indicated by “T”, which is incremented when a data element is entered that is consistent with a possible incipient exacerbation. On FIG. 4D one sees that a final tally of zero results in a determination that the subject is OK and instructions consistent with that determination, while a tally of greater than zero results in instructing the patient to make an appointment with the health care provider and take a particular medication, implying that the patient may, be at increased risk of a CHF exacerbation or may be in the early stages of a CHF exacerbation.

In some embodiments the system comprises a knowledge base represented as a collection of decision lists. An example of a simple decision list representing knowledge that may be employed in an SST for COPD exacerbation is as follows;

```
If “body temperature”≥ 104 or “cough” = severe then
    call 911
Elseif “cough” = moderate and “shortness of breath” = moderate then
    call doctor now
Elseif “cough” = mild or “shortness of breath” =mild or “body
        temperature” ≥ 99 and < 104 then
    call doctor for appointment tomorrow
Else
  No recommendation at this time. Continue with usual treatment.
```

Conceptually, the knowledge base may be organized by outcomes, where there may be two types of outcomes: triage instruction and treatment instruction (directions). In some embodiments, there may be one outcome or more than two outcomes. (Note that “outcome” as used here refers to potential outputs of the algorithm, not outcomes of the condition.) Each triage instruction and each treatment instruction may have a predetermined priority level, which may reflect the importance of the instruction or the urgency with which it should be carried out. Within each type of outcome, the priority level indicates the priority with which that instruction is to be given, with 1 indicating the highest priority. In general, a higher priority correlates with a more serious patient state for the given condition. For example, an instruction to “call 911” would have higher priority than an instruction to make an appointment with a physician since it would be important to detect a patient state that warrants emergency medical attention rapidly and it would be important to provide the patient with the instruction(s) to seek emergency medical attention in preference to alternative triage instructions. The system may store information that assigns each potential triage instruction a priority level. Similarly, the system may store information that assigns each potential treatment instruction a priority level. These priority levels might be specified for each condition or in at least some cases might be global across multiple or all conditions (particularly in the case of triage instructions). For example, “call 911” may always be assigned the highest priority. Generally, the priority levels would embody medical knowledge. Treatment directions for a given condition may all be assigned the same priority or may be assigned different priorities. In general, it may be appropriate to give a single treatment direction or multiple treatment directions.

Entries in the knowledge base specify which questions imply certain outcomes. (In this description it is assumed that any input is a response to a “question” though it could be, for example, data obtained from a connected monitoring device (e.g., previously stored valid data, data obtained by a request to a data source for such data at the time the SST is run).

An example of an embodiment of a COPD exacerbation SST is provided below. There are two tables—one each for triage instructions and treatment instructions. The SST would be run by asking the patient a series of question regarding symptoms such as “Are you more short of breath than usual? If so, describe the change as mild, moderate, severe, or emergency.” and similarly for cough, wheezing, and chest pain. Questions could also be asked to obtain objective data such as body temperature or oxygen saturation.

COPD SST Triage Instructions

| Short- | | | Body | | | | |
|---|---|---|---|---|---|---|---|
| ness of | | Wheez- | temp- | Chest | | Triage | |
| Breath | Cough | ing | erature | Pain | O2sat | Outcome | Priority |
| None | None | None | None | None | | No | 4 |
| | | | | | | recom- | |
| | | | | | | mendation | |
| Mild | Mild | Mild | | | | Call office | 3 |
| | | | | | | if no im- | |
| | | | | | | provement | |
| Mod/ | Mod/ | Mod/ | <104 | | | Call for | 2 |
| Sev | Sev | Sev | | | | appt | |
| Em | Em | Em | >=104 | Yes | <82 | Call 911 | 1 |

COPD SST Triage Instructions

| Short- | | | Body | | | Instruc- | |
|---|---|---|---|---|---|---|---|
| ness of | | Wheez- | temp- | Chest | | tion | |
| Breath | Cough | ing | erature | Pain | O2sat | Outcome | Priority |
| M/M/S | M/M/S | M/M/S | 99-104 | | | Nebulizer | 5 |
| Mod/ | Mod/ | Mod/ | <104 | | | Prednisone | 5 |
| Sev | Sev | Sev | | | | | |
| Mod/ | Mod/ | Mod/ | <104 | | | Bactrim | 5 |
| Sev | Sev | Sev | | | | | |
| | | | >=99 | | | Tamiflu | 5 |
| | | | <104 | | | | |

M/M/S = mild OR moderate OR severe

For example, the algorithm outcome “call 911” is the result of either “cough=emergency” or “wheezing=emergency” etc. The rows are disjunctive—any one answer within a given row implies the outcome in that row. The above table can readily be modified to account for conjunctions by creating one row for each conjunction that implies an outcome. Each outcome would then appear in several rows. The above model is used simply for ease of exposition. The SST triage instruction table corresponds to one decision list. The SST treatment instruction table is interpreted similarly. The algorithm would run as follows:

```
Within the Triage Instruction SST
  For each priority P=1, 2, 3, 4
    Ask every question in the table which could result in priority
    P, and which has not already been asked before
  Output the outcome which matches first
Within the Treatment Instruction SST
  For priority P=5 (in this case, only one priority appears)
    For every instruction with priority P
    If the instruction applies to the patient (i.e. patient has medication
    available), ask every question which could result in the instruction,
    and which has not already been asked before
  Output instruction if the answer to the question matches
Output “no recommendation” if no other output
```

This algorithm is believed to be correct (assuming that the knowledge base is consistent) because it only provides outcomes which appear as matches in the knowledge base. The algorithm is believed to be complete because it assumes (in the triage case) that every possible input is covered within the knowledge base, and because the “no recommendation” outcome is the default for the instruction SST. If a patient has two or more SSTs that need to be run, they may be combined by asking all the priority 1 questions of each, then priority 2, etc., through the last. The algorithm is correct and complete (again assuming consistency of the knowledge base). However, it may be medically the case that a patient with multiple conditions would have escalated or different reasons that merit particular triage directions, e.g., calling 911, that a patient with only one condition would not, or different treatment directions may be appropriate in the presence of two conditions than in the presence of a particular condition in the absence of the other. To specify this in the knowledge base override rows may be defined. An override row considers all of the questions applicable to any of two or more SSTs and provides an outcome that replaces the outcome in either SST. An SST algorithm with overrides may be represented be as follows:

1. Output the “overrides” triage instructions

2. If none, output combined triage instructions for each condition

3. Output overrides treatment instructions

4. If none, output combined treatment instructions for each condition

In certain embodiments, step 4 of the algorithm may output the combined treatment instructions for each condition regardless of whether any treatment instructions were output in step 3, other than those already output in step 3. In other words, step 4 may be 4. Output combined treatment instructions for each condition

In certain embodiments, an SST algorithm in which the outcomes are specified based on condition and material patient characteristics may operate as follows, wherein for purposes of description, the data elements that an SST may use in determining directions will be referred to as “variables”. The set of all data elements that may be obtained by the collection of SSTs in the REVON system may be referred to as the “SST variables”. Each SST variable may assume one or more different values. These may be, for example, “yes” or “no” or a numerical value. Certain values may be converted by the system into other values or may be considered equivalent. For example, all blood pressure values within a given range may be converted to a particular value or may be considered equivalent or not distinct for purposes of analysis by an SST algorithm. In some embodiments, the REVON system comprises a knowledge base that enumerates for a condition, for every possible combination of material patient characteristics for that condition, for each instruction that may be provided for management of the condition, every possible combination of values for the SST variables that could lead to that particular instruction. As above, each triage instruction and each treatment instruction may have a priority level. Each such entry in the knowledge base may be represented as a row, e.g., as follows, wherein it is assumed that A, B, C, and D represent variables (each of which may have the value 1, 2, 3, or 4), asterisks represent irrelevant variables in the context of the particular SST (i.e., variables that are not used in determining the management directions), and a particular combination of variables, e.g., A=1 and B=2 would result in a particular instruction or combination of management instructions (the management instructions are not shown, but it should be assumed that each row would have an associated set of one or more management instructions). Note that an asterisk means that the value of the variable could be anything so that, for example, the first row actually enumerates multiple different patient states, i.e., those patient states in which A=1, B=2 and both C and D could independently be 1, 2, 3, or 4.

Initial State: Knowledge Base

| A | B | C | D |
|---|---|---|---|
| 1 | 2 | * | * |
| * | 3 | 1 | * |
| * | * | 3 | 2 |
| * | 4 | * | 2 |

When invoked (i.e., when a particular SST is run for a given patient), the algorithm first selects, from the knowledge base, those entries that represent combinations of SST condition and material patient characteristics for the SST condition that the given patient has. In the above representation it is assumed that this filtering has already been performed.

The algorithm determines which row matches the patient's current state with regard to variables A, B, C, and D. At iteration 0 (before the first iteration of the algorithm) the knowledge base and patient state may be represented as follows, where the values of all variables are unknown:

Initial State:

| Knowledge Base | | | | Patient State | | | |
|---|---|---|---|---|---|---|---|
| A | B | C | D | A | B | C | D |
| 1 | 2 | * | * | ? | ? | ? | ? |
| * | 3 | 1 | * | | | | |
| * | * | 3 | 2 | | | | |
| * | 4 | * | 2 | | | | |

In the patient state a question mark indicates an unknown (or perhaps an expired) value. In each iteration of the algorithm, a “working set” may be defined, which represents all entries (rows) from the knowledge base that match the patient state during that iteration with respect to A, B, C, and D. For example, if it is determined that A=2, then the first row in the representation above would be removed from the working set since it is inconsistent with A=2. The other three rows would remain. At each iteration, the working set contains the subset of the knowledge base that matches the patient state during that iteration. A question is selected from the remaining unknown, relevant variables, and the patient state is updated with the value received in response to the question. An example of running the algorithm through multiple iterations to obtaining a unique match is presented below. The variables that the algorithm may want to ask about are called “current variables” (Current Vars). Assume the current working set all have the same outcome “call 911”.

Initial State:

| Knowledge Base | | | | Patient State | | | |
|---|---|---|---|---|---|---|---|
| A | B | C | D | A | B | C | D |
| 1 | 2 | * | * | ? | ? | ? | ? |
| * | 3 | 1 | * | | | | |
| * | * | 3 | 2 | | | | |
| * | 4 | * | 2 | | | | |

Begin SST algorithm (911 triage instruction)

| Working Set | | | | Patient State | | | |
|---|---|---|---|---|---|---|---|
| A | B | C | D | A | B | C | D |
| 1 | 2 | * | * | ? | ? | ? | ? |
| * | 3 | 1 | * | | | | |
| * | * | 3 | 2 | | | | |
| * | 4 | * | 2 | | | | |

Current Vars: ABCD
Select A and ask the patient. Patient answers A = 3

| Working Set | | | | Patient State | | | |
|---|---|---|---|---|---|---|---|
| A | B | C | D | A | B | C | D |
| * | 3 | 1 | * | 3 | ? | ? | ? |
| * | * | 3 | 2 | | | | |
| * | 4 | * | 2 | | | | |

Current Vars: BCD
Select C and ask the patient. Patient answers C = 1

| Working Set | | | | Patient State | | | |
|---|---|---|---|---|---|---|---|
| A | B | C | D | A | B | C | D |
| * | 3 | 1 | * | 3 | ? | 2 | ? |
| * | 4 | * | 2 | | | | |

Current Vars: BD
Select B and ask the patient. Patient answers B = 4

| Working Set | | | | Patient State | | | |
|---|---|---|---|---|---|---|---|
| A | B | C | D | A | B | C | D |
| * | 4 | * | 2 | 3 | 4 | 1 | ? |

Current Vars: D
Select D and ask the patient. Patient answers D = 2

| Working Set | | | | Patient State | | | |
|---|---|---|---|---|---|---|---|
| A | B | C | D | A | B | C | D |
| * | 4 | * | 2 | 3 | 4 | 1 | 2 |

Exit with triage instruction output

The operation of the algorithm may be described in further detail as follows:

1. Select an initial working set from the knowledge base by finding rows that match a patient's condition and material patient characteristics

2. For each triage priority level, in order (i.e., starting with the highest priority triage instruction), do the following
a. Select from the working set only the rows with instructions corresponding to the current triage level
b. Repeat the following until the working set is one row (a match) or no match can be found
   i. Remove any rows which do not match the patient state
   ii. Select one relevant variable and query the patient (or other data sources as applicable)
c. Output the current triage level output if there is a match

3. Repeat step 2 with treatment instruction priority levels

In step 2.b.i, “match” is defined as: every known patient state variable appears in the working set either with exactly the same value or with NULL (asterisk, meaning “irrelevant”). In regard to defining the selection of rows in step 1, it will be understood that some rows will correspond to one condition only. Others will correspond specifically to condition+one or more comorbidities. All the matching rows from each category are selected.

In 2.b.ii, questions to be asked may be selected in a predetermined order or, in some embodiments, in a random order. In some embodiments, an ordering based on splitting the working set near evenly to minimize the expected number of questions, or using data collected to correlate variables and order question based this knowledge, may be used.

If desired, the knowledge base may be checked for inconsistencies by, e.g., comparing all rows pairwise and determining whether one is a subset of the other, and any such consistencies may be corrected.

A pseudocode version of the algorithm is presented below.

```
Given:
Condition X (T-Plan X), assume this is the same for all below
Characteristics: char[1],...,char[N] as boolean variables
Variables: var[1]:,...,var[M]as discrete variables (multiple choice
responses, may be “NULL” to signify immaterial or unknown)
Datatypes:
Patient = {
    personal_info,
    condition,
    characteristics as chars,
    variables as vars}
row = {
    condition,
    characteristics as chars,
    variables as vars,
    advice}
Define Knowledge_base = an array of rows
Define function Match(patient, row):
/* returns TRUE iff a patient state matches a row,
where a match is based on currently known patient data and non-null
entries in the row.
A match means all currently known patient data matches all non-null
variables, and all characteristics match exactly */
  If {
    patient.chars[i]==row.chars[i] for all i
    and
    (patient.vars[i]==row.vars[i]
      or row.vars[i]==NULL
      or patient.vars[i]==NULL or expired) for all i
    }
return True
else
return False
Define function perfectMatch(patient, row):
/* return true only if a patient unambiguously matches a row - all
known vars match all diagnostic vars */
  If
    (Patient.var[i]==row.var[i] OR /* they match OR */
    Row.var[i] ==NULL) for all i /* we don't care anyway
  Then return True
  Else return False
define SST-algorithm(patient, knowledge_base):
/* triage 911 */
Let WS = all rows, i, in knowledge_base where
row[i].outcome=”Call 911”
repeat {
    /* current variables is anything that still matters, but
      we don't know yet about the patient */
    Let CV = all vars, j, in knowledge_base where
        WS.row[i].var[j] != NULL AND
        patient.var[j]]== NULL or expired
    /* get patient input */
    Select a variable, k, from CV and ask patient
    Let patient.vars[k]=answer from patient
    /* the working set is all rows that match a patient */
    let WS = all rows, i, in knowledge_base where
        Match(patient,row[i])==true
}
until (size(WS)==0 or
        (size(WS==1) and perfectMatch(WS, patient)))
If size(WS)==1 AND then output “call 911”
If size(WS)==0 then exit
/* now do next triage level */
/* etc */
```

In some aspects, an algorithm such as those described herein based on a T-plan condition and significant patient characteristics provides for generating directions that can be personalized to a particular patient without the need to construct different algorithms for each potential combination of significant patient characteristics. Furthermore, such algorithm(s) may be readily modified to allow for addition or removal of significant patient characteristics and/or adding, removing, or changing the directions by, for example, adding or modifying the appropriate entries in the knowledge base. For example, as medical knowledge grows and/or as new therapies become available, it may become evident that additional patient characteristics (e.g., newly identified biomarkers) are material characteristics for a particular condition. By adding appropriate such patient characteristics to the set of significant patient characteristics for that condition, and adding or modifying one or more entries in the knowledge base, the algorithm(s) can evolve. It should be noted that a single underlying algorithm may thus be used to provide an evaluation and/or directions for a condition given any of a wide variety of patient characteristics or combinations of patient characteristics. The same algorithm may be applied to different conditions, provided a suitable knowledge base exists for each condition.

In some embodiments, an SST algorithm may use a variety of data in addition to health data obtained from the patient. Such data may include, for example, outdoor environment data, population-based data, geographic data, or any combination of the foregoing. For example, a particular set of symptom data and physiological data in combination with certain environmental conditions may result in recommending a different course of action than would be the case if the environmental conditions were more favorable. In some embodiments, for example, a patient may be instructed to avoid or limit outdoor activity during periods of extreme heat or cold or high levels of pollen or pollutants such as ozone or particulates or may be instructed to increase or decrease the indoor temperature or go to a cooler or warmer place. In some embodiments, population-based data may be considered. For example, a particular set of symptom data and physiological data in combination with certain population-based data may result in recommending a different course of action than would be the case if the population-based data were different. In some embodiments, for example, if there is a high level of influenza virus infection in a given region, patients in that region with symptoms consistent with influenza may be instructed to take an appropriate anti-influenza virus agent such as Tamiflu.

As noted above, in some embodiments, an outcome may include both a treatment instruction and a triage instruction. In some embodiments, an outcome may include one or more instructions that may be conditional or contingent in that, for example, they may vary depending on the availability of an item needed for a rescue measure and/or may vary depending on the patient's response to a rescue measure. The system may handle such outcomes, and other types of outcomes that combine various types of instructions and/or instructions that are conditional and/or contingent on the availability of certain items to the patient and/or the conditional and/or contingent on patient's responses to rescue measures by, for example, appropriate modifications of the algorithms described above. In some embodiments, the availability of an item needed to implement a rescue measure may be considered a variable. In some embodiments, the patient may be asked whether he or she has the item prior to executing the algorithm.

In some embodiments, the same type of algorithm described above for generating instructions in the context of an SST may be used to configure T-plans given a suitable knowledge base. The inputs to such an algorithm would be the significant patient characteristics. The outputs would be a set of data collection modules. The same type of approach may be used to configure portions of a data collection module, such as a treatment plan, given a suitable knowledge base. For example, a knowledge base may enumerate, for a condition, for every possible combination of material patient characteristics for that condition, one or more medications and/or procedures along with the appropriate timeframes.

In some aspects, an SST may be considered to be composed of one or more types of diagnostic units. In this regard “diagnostic unit” refers to any question or other data request along with a data element responsive to the data request, that may be used to evaluate the status of the patient's condition (i.e., is diagnostic with regard to the status of the condition). Diagnosis in this context is not intended to refer to an initial diagnosis of the condition (although the same types of diagnostic units may also be used for this purpose). A diagnostic unit may relate to a particular symptom, such as “cough” or “shortness of breath” or to a particular physiological parameter such as “blood glucose” or “oxygen saturation”. By way of example, in the case of a COPD SST, diagnostic units may comprise questions about diagnostically significant symptoms such as shortness of breath and cough such as “Are you feeling more short of breath than usual?” and “Is your cough worse than usual?”, and answers thereto, or may be questions that ask the patient to measure and enter the value of a physiological variable that is diagnostically significant in the context of COPD and the data provided in response. Asking the question or making the data request and receiving the data or otherwise obtaining the data called for by a diagnostic unit, and operating on the data, may be referred to as executing the diagnostic unit. In some embodiments, a diagnostic unit requests two or more data items or data elements that are closely related, directed towards the same symptom, physiological measurement, or diagnostic end, and/or would be considered as a diagnostically cohesive unit by one of ordinary skill in the art. For example, a first question may ask whether a patient is experiencing a symptom, and a second question may ask about its intensity or change relative to the patient's usual state. A diagnostic unit that comprises multiple questions need not be fully executed. For example, if a patient indicates that he or she is not experiencing a particular symptom, additional questions that pertain to symptom intensity need not be asked.

The diagnostic units in an SST may determine at least in part which monitoring modules are included in a T-plan that includes the SST. For example, if an SST comprises a diagnostic unit that calls for a particular type of physiological data element, a monitoring module that collects data elements of that type may be included in the T-plan. For example, an SST for COPD may contain a diagnostic element that operates on an oxygen saturation measurement. Therefore, the system may automatically assign a monitoring module for obtaining oxygen saturation measurements as part of a COPD T-plan.

In some embodiments, an SST may comprise one or more “starter diagnostic units”. In some embodiments, a “starter diagnostic unit” is a diagnostic unit that can be used to assess a patient's health status in regard to the SST condition and determine whether the patient's health status is stable in regard to the SST condition or whether the patient's health status in regard to the SST condition may be deteriorating or may have deteriorated. If the patient's health status is determined to be stable based on the starter diagnostic unit(s), at least some of the remaining questions in the SST may not be asked or may be deprioritized as compared with questions in other SSTs. If the patient's health status is determined to be possibly deteriorating or to have possibly deteriorated based on the starter diagnostic unit(s), remaining questions in the SST may be asked and the complete SST algorithm may be run. By way of example, in the case of a COPD SST, starter diagnostic unit(s) may comprise questions about diagnostically significant symptoms such as shortness of breath and cough such as “Are you feeling more short of breath than usual?” and “Is your cough worse than usual?” If the patient answers “No” to both questions, then the system may determine that the patient's health status is stable with regard to COPD, and the remainder of the SST for COPD is not executed. In some embodiments, physiological data collected automatically from connected monitoring devices may be used in place of questions as starter diagnostic unit(s)

A diagnostic unit may be present in multiple different SSTs for which it provides relevant diagnostic information. For example, a diagnostic unit that pertains to “cough” or “shortness of breath” may be included in an SST for COPD and in an SST for CHF, since cough and shortness of breath carry diagnostic significance in both conditions. Diagnostic units may have weights assigned to them, wherein the weight reflects the diagnostic value of the particular diagnostic element. A diagnostic unit may have different weights in different SSTs, depending, e.g., on the diagnostic value of that particular element in the context of the condition and/or the other diagnostic units in the SST.

In some aspects, diagnostic units are modular and can be assembled in any order or combination to construct or modify an SST. Diagnostic units may be added to an existing SST, removed from an existing SST, re-ordered (changing the sequence in which data for the diagnostic elements is collected and/or analyzed), replaced by different diagnostic units, and the like. Construction of an SST from diagnostic units may be performed by the system or by a user. In some embodiments, a user interface that provides means for constructing an SST from diagnostic units is provided. Diagnostic units may be represented as icons that can be “dragged” on a computer display and joined by various connectors to assemble an SST, which may be in the form of a decision tree such as that depicted in FIGS. 4A-4D. The connectors may indicate an order in which data elements called for by the particular diagnostic units are collected. In some embodiments, clicking on an icon representing a particular diagnostic unit causes a description of the diagnostic unit to be displayed. The system may permit the user to assign weights to the diagnostic units. Diagnostic units may also or alternately be created de novo by a user in certain embodiments.

It is noted that an SST represented as a set of diagnostic units may also be represented as a set of entries in a knowledge base. In SST embodiments that employ “starter diagnostic units”, these would correspond to the first questions asked by the SST algorithm. For example, as noted above questions may be asked in a predetermined order. In some embodiments, the system may convert an SST from one representation, e.g., a decision tree format, into a different representation, e.g., a set of entries in a knowledge base. A decision tree format may provide an intuitive approach for construction of SSTs by health care providers, while converting such decision trees into a knowledge base representation described herein may allow these HCP-designed SSTs to be executed using the same algorithm that is used to execute SSTs provided by the system.

Monitoring, Monitoring Modules and Monitoring Devices

As discussed above, a T-plan may comprise one or more monitoring modules that specify physiological data, behavioral data, and/or environmental data to be obtained regarding or relating to the patient. Typically, the types of data specified by a monitoring module are objective, such as measurements obtained from a monitoring device. Subjective data, e.g., data pertaining to symptoms, would not typically be obtained by a monitoring module but may be obtained instead by a symptom tracker, as described above. In some embodiments a health tracking module may use objective data obtained by one or more monitoring modules that collect health data, e.g., physiological data, to be used in a health evaluation in evaluating the patient's health status with regard to the condition and/or in determining directions to the patient. In some embodiments, data obtained by a monitoring module may trigger performance of a health evaluation if, for example, the data are indicative of a deterioration or potential deterioration or notable change in the patient's health or may be used in a health evaluation. In some embodiments, the monitoring modules included in a T-plan, e.g., a T-plan that is configured automatically, would include at least those monitoring modules that obtain data elements of a type that is used in a health evaluation with regard to the T-plan condition or that may trigger a health evaluation. In some embodiments, a monitoring module may be included in a T-plan regardless of whether or not data obtained by that module would be used by a health tracking module or would trigger a health evaluation. Data obtained by such monitoring modules may be useful to the patient or health care provider for other purposes, such as prognosis, evaluating the efficacy or side effects of treatment, and/or evaluating the patient's compliance with treatment recommendations. Certain patient characteristics may result in particular monitoring modules being included in a T-plan that is configured for a patient who has those characteristics. For example, a T-plan for a COPD patient who has “smoker” as a patient characteristic may include a monitoring module for smoking. A monitoring module for smoking might, for example, ask the patient each day how many cigarettes he or she smoked, monitor use of a nicotine patch or nicotine gum in a patient who is trying to quit smoking, or monitor behaviors associated with smoking.

A system may collect data obtained by or using any of a wide variety of monitoring devices. Examples of monitoring devices include body weight scales (e.g., wireless scales such as Withings Wireless Scale and Wi-Fi Body Scale), blood pressure cuffs, blood glucose meters, activity tracking devices (such as Fitbit products (http://www.fitbit.com/), UP or UP24 products (Jawbone; https://jawbone.com/), and BodyMedia® FIT Armband (www.bodymedia.com)), exercise machines (e.g., treadmills, bicycles, or other equipment useful for exercise purposes, which may be equipped with ergometers), medication dispensers that detect opening, removal, or use of a medication, sleep monitors, pulse oximeters, or any type of device that gathers physiological data, behavioral data, or environmental data. In some embodiments, a monitoring device may be a wearable monitoring device, skin patch, implanted monitoring device, swallowed monitoring device, or indwelling monitoring device. In some embodiments, a monitoring device is a medical device that is used for therapeutic purposes such as a machine that delivers positive airway pressure (e.g., continuous positive airway pressure (CPAP) machine) to patients with conditions that may benefit from such treatment. In some embodiments, it is envisioned that data concerning the indoor environment of a patient may be detected using a dedicated device or a mobile device equipped with an appropriate sensor, e.g., a connected device in the patient's home such as connected home automation devices (e.g., Nest programmable thermostat).

A monitoring module may obtain data in any of a variety of ways. The particular way in which a monitoring module obtains data and, in some cases or embodiments, the type of data obtained and/or the timeframe according to which the data are obtained, may depend on factors such as the availability to the patient of particular monitoring devices. In some embodiments, patients may manually or verbally enter data either voluntarily or in response to requests presented via a user interface as described above in regard to health tracking modules. The user interface may provide means by which patients can voluntarily enter data whenever convenient. Patients may, for example, be asked to obtain a measurement of a physiological parameter using an appropriate monitoring device or may be asked questions about behaviors. In some embodiments, the system integrates or interfaces with one or more connected monitoring devices so that data gathered by such devices can be obtained automatically without requiring patient input.

A variety of ways of acquiring data from connected monitoring devices are contemplated. Such data may, for example, be gathered through wires or wirelessly by direct transmission to the patient's computer, by interfacing with different apps on the patient's computer that acquire the data from the devices, by interfacing with a personal health record software product such as HealthVault (Microsoft) or Dossia that lets patients upload data from health and fitness devices and share health information or the 2net™ Platform (Qualcomm Life). The system may obtain the data from websites operated by third parties, e.g., the makers or providers of the connected monitoring device in question. Such data may be made available through appropriate APIs, e.g., open APIs. In some embodiments, a patient may have or be invited to download one or more monitoring application(s), e.g., apps. Such monitoring application(s) may be third party apps that may interface directly with a REVON application on a patient's mobile device or send data to a website from which it is later acquired by the REVON system. Such application(s) may be third party apps that receive data from connected monitoring devices and interface directly with a REVON application on a patient's mobile device or send data to a website from which it is later acquired by the REVON system.

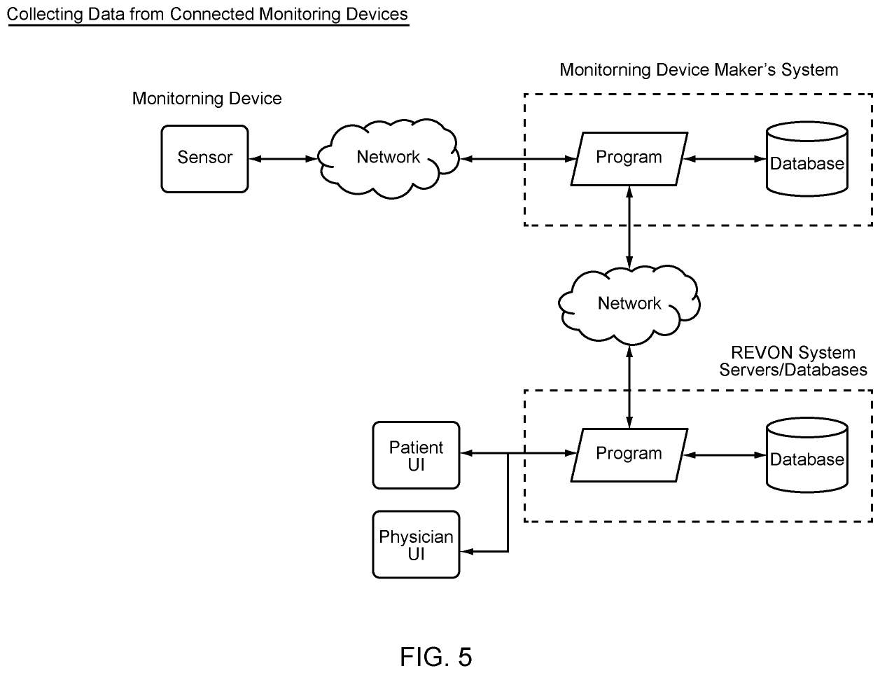

FIG. 5 depicts an architecture which can be used in obtaining health data collected by a connected monitoring device according to certain embodiments. Monitoring devices may include a variety of different sensors that acquire various types of data from the patient or the patient's environment. The data are sent over a network to their manufacturer's systems (servers). The REVON system collects data from these third party servers as specified by monitoring modules of patients' T-plans, stores it in association with an identifier of the patient to whom it pertains, and makes it available to patients and physicians. The data may be obtained in various ways, which may differ depending on the particular connected monitoring device. For example, it may be requested by REVON or may be provided automatically by the third party server. It may be provided periodically on a fixed schedule or may be provided as it becomes available on the third party server.

The REVON system may comprise one or more modules that acquire the data and may process it as required for purposes of storing it in the system's existing database(s). For example, data gathered from different monitoring devices (e.g., different brands or models of the same type of device) may represent the same types of data differently or use different units. The system may convert data obtained from diverse devices to a uniform representation and units. The system may check data for plausibility and may request confirmation, repeat of the measurement, or repeated transmission of the data if the data appear likely or potentially to be incorrect (e.g., incompatible with life or markedly different from previous readings).

In some embodiments, when health data specified by a monitoring module in a T-plan assigned to a patient are received, the patient's state is updated accordingly. In some embodiments, when a particular data element expires, a monitoring module that specifies data elements of that type causes execution of the appropriate computer-executable instructions to obtain a new data element of that type. It should be noted that the way in which the monitoring modules actually obtain data need not be specified by the prescriber of a T-plan in which such monitoring modules are included. These details may be managed appropriately by the system depending at least in part on the availability of various monitoring devices. For example, the default may be to ask the patient to enter the required data manually. However, if the patient has a connected monitoring device and provides the system with access to data collected by such device, a monitoring module may obtain such data instead of asking the patient to enter the data.

In some embodiments, a patient may receive directions on how to make it possible for the system to obtain data collected by various connected monitoring devices so that monitoring modules specified by T-plans prescribed to the patient can obtain such data without requiring it to be entered by the patient. In some embodiments, such directions are provided to the patient when the patient obtains an account with the REVON system. In some embodiments, the system may provide information, e.g., on a website that indicates those connected monitoring devices from which the system is equipped to obtain data. In some embodiments, the system provides means for the patient to order at least some such devices online, e.g., through such a website. In some embodiments, there may be certain connected monitoring devices that may be available only to patients who are enrolled with the REVON system or for whom such device is part of a treatment plan as specified in a T-plan. In some embodiments, the system may provide information, e.g., on a website, that guides the patient through the process of specifying connected body monitoring devices to be used and/or granting the system access to data collected by such connected monitoring devices. In some embodiments, the system may provide information, e.g., on a website, that indicates to a patient whether or to what extent the cost of particular monitoring device(s) is reimbursed by the patient's health insurance provider or other payer. The system may use information regarding the patient's insurance policy or other health care coverage to provide such information. In some embodiments, the system may provide the patient with a list of those monitoring devices that are at least partly covered by the patient's insurance policy or other health care coverage. In some embodiments, the system may automatically submit a claim for reimbursement if the patient purchases an eligible device. In some embodiments, the system may inform the patient if a monitoring device requires prior authorization for reimbursement. In some embodiments, the patient may view a list or other representation of their T-plans on the website and the types of monitoring devices specified by such T-plans. The system may suggest, based on the patient's insurance policy or other health care coverage, which device(s) the patient may wish to acquire.

In some embodiments, a monitoring module may operate in “regular monitoring” mode, “as-needed monitoring” mode, or both. In “regular monitoring” mode, a monitoring module obtains one or more data elements on a recurrent basis, regardless of whether or not a health tracker that may use such data is run. The data may be obtained with a selected frequency, e.g., daily, weekly, or with whatever frequency is deemed appropriate. The appropriate frequency for a given data element or monitoring device may be different for different patients based, for example, on T-plan condition, one or more patient characteristics, and/or the availability of connected monitoring devices. For example, it may be appropriate to obtain a blood pressure reading daily from certain patients, weekly from others, or monthly from others. A monitoring module that obtains data on a recurrent basis regardless of whether or not a health tracker that may use such data is run may be referred to as a “regular monitoring module”. It should be noted that the frequency with which a regular monitoring module obtains data may vary over time and/or may be responsive to changes in the patient's state (e.g., the frequency with which a particular data element is obtained may increase if values indicate a trend suggestive of a potential deterioration in the patient's state is detected, and may decrease if the trend does not continue or the value returns to baseline). Furthermore, in addition to a frequency, there may be a time window within which the data may be obtained. For example, a frequency such as “daily” does not necessarily require that the data be obtained at the same time or approximate time each day, although it may be. Thus the use of the term “regular” in the context of “regular monitoring” or “regular monitoring module” is not intended to imply that the data must always be obtained with a constant or definite pattern, e.g., with the same time interval or approximate time interval between individual instances of data collection, although it may be.

In addition to performing regular monitoring, a regular monitoring module may (at least in the case of certain monitoring modules that obtain data elements that may be used by a health tracker) also operate in an “as-needed” mode, in which it obtains data as needed for the running of a health tracker. For example, if a health tracker is run and requires a data element of a particular type for its execution, the system may check whether the most recently obtained value of that particular data element is valid or has expired. If the data element has expired, a new data element of that type may be obtained by the appropriate regular monitoring module, outside of the regular monitoring performed by that monitoring module. Obtaining a data element in response to the need for such data element for purposes of running a health tracker may be referred to as “as needed monitoring”. In some embodiments, a monitoring module may function on an “as-needed” basis when prescribed as part of certain T-plans that, for example, include particular health trackers that use data elements obtained by that monitoring module. For example, in the context of certain T-plans a monitoring module for body temperature may obtain data only on an as-needed basis when a health tracker that uses body temperature in its execution is run. It may not obtain data on a recurrent basis outside the context of running the health tracker. However, in the context of certain T-plans it may be appropriate to obtain body temperature on a regular basis regardless of the running of a health tracker. Thus, a monitoring module may in some embodiments be capable of performing regular monitoring, as-needed monitoring, or both. The particular monitoring mode(s) in which a monitoring module runs may vary depending, for example, on the T-plan condition, patient characteristics, and/or availability to the patient of connected monitoring devices. In some embodiments a monitoring module may obtain data only on an as needed basis.

In some embodiments, a monitoring module may have two or more modes of operation. The mode(s) of operation of a given monitoring module may, in some embodiments, be set by a health care provider who prescribes the monitoring module to a patient or may, in some embodiments, be set automatically by the system when a T-plan containing the module is configured. In some embodiments, a health care provider may change the setting or the setting may be changed automatically by the system in response to various occurrences. In some embodiments, a monitoring module may operate in two or more of the modes simultaneously, i.e., they are not mutually exclusive.

In some embodiments, a monitoring module may be able to operate in any of the additional four modes, which may be referred to as: (1) Basic Mode; (2) Risk Detection Mode; (3) Health Tracker Mode; and (4) Heightened Sensitivity Mode. A monitoring module may be capable of operating in any one, two, three, or four of these modes. Thus, certain monitoring modules may only be capable of operating in one, two, or three of the modes. In some embodiments, a monitoring module may operate in two or more of the modes simultaneously, i.e., they are not mutually exclusive. For example, at any time, a monitoring module may be operating in Risk Detection Mode and Health Tracker Mode or may be operating in Risk Detection Mode, Health Tracker Mode, and Heightened Sensitivity Mode. In some aspects, Risk Detection Mode and Health Tracker Mode represent two ways in which monitoring modules may function within the activities of a T-plan, while Heightened Sensitivity Mode represents the monitoring modules operating in either or both of such modes with increased frequency and/or with a lower threshold for triggering the running of an SST, potentially conferring greater likelihood of detecting a deterioration early in its course. Heightened Sensitivity Mode may be particularly appropriate for use at times when patients are particularly susceptible to experiencing a deterioration, such as shortly after discharge from hospital or in the near term after a patient has experienced a deterioration. It should be noted that when running in Risk Detection Mode and/or Heightened Sensitivity Mode, a monitoring module will typically be operating in regular monitoring mode.

In Basic Mode a monitoring module is not linked to an SST in the sense of collecting data that may be used by an SST or in the sense that data obtained by the monitoring module may trigger running an SST. Instead, an SST may merely provide a way for patients to monitor the particular physiological variable(s) specified by the monitoring module. A patient may assign a monitoring module in Basic Mode to himself or herself using a patient app or on the system website and may voluntarily or when prompted enter the relevant measurement or utilize a connected monitoring device to have the data automatically collected. A health care provider may assign a monitoring module in Basic Mode to a patient if, for example, the patient or health care provider is interested in monitoring the particular physiological parameter(s) specified by the monitoring module. Tools for viewing and analyzing health data provided by the system may be applied to the data collected by in Basic Mode. For example, the data may be displayed on a patient or physician dashboard along with other health data.

In Risk Detection Mode the monitoring module checks the data, e.g., physiological data, specified by the module as the data is acquired and determines whether it is indicative of a deterioration or potential deterioration or notable change in the patient's health status, e.g., an adverse change such as an exacerbation. If so, further action, such as running an SST may be taken. Thus, a monitoring module in Risk Detection Mode may be linked to an SST in the sense that the running of an SST may be triggered by data collected by the monitoring module. In some embodiments, an abnormal value may be rechecked one or more times to confirm its accuracy, and the additional action(s), or one or more of them, is taken only if the value is confirmed as abnormal. The system may employ any of a variety of criteria to determine whether a value is confirmed as abnormal. For example, in some embodiments two consecutive abnormal readings, or two abnormal reading outs of three readings constitute confirmation that a value is abnormal. The particular action to be taken, e.g., the particular SST to be run, may be selected based on the type of physiological data that was determined to be abnormal. Typically, an abnormal value will clearly implicate one or a small number of conditions as potentially being responsible for the abnormal value, and SSTs designed to detect a deterioration or other notable change in those one or more conditions may be run. Sometimes, the SST will be part of the same T-plan as is the monitoring module that triggers the running of the SST. For example, an abnormally low blood oxygen saturation measurement may be detected by a monitoring module that obtains blood oxygen saturation measurements, which may be part of a T-plan for COPD. The COPD T-plan would also include a COPD exacerbation SST, and the running of this SST may be triggered by the abnormally low blood oxygen saturation measurement. If the abnormal value might be caused by more than one condition for which the patient has SSTs, each SST may be run, or additional data, e.g., physiological data or symptom data, may be obtained to determine which SST(s) to run. A monitoring module may store information specifying which SST(s) are to be run and/or which other actions are to be taken, if any, in the event of detecting an abnormal value.

In at least some embodiments, not all monitoring modules may trigger running an SST when an abnormal value is detected. In some embodiments, detection of an abnormal value may, instead of or in addition to triggering the running of an SST, trigger a different action such as (i) notifying the patient of the abnormal value and, optionally, providing appropriate management direction(s); (ii) notifying a caregiver of the patient of the abnormal value and, optionally, providing appropriate management direction(s); (iii) notifying a health care provider of the patient of the abnormal value; or (iv) any combination of (i), (ii), and (iii). In some embodiments, the system performs a danger stratification if an abnormal value is detected. A danger stratification may assess the level of danger that is posed or potentially posed by the abnormal value. For example, a body temperature of 105 degrees Fahrenheit typically represents a dangerous physical state for which urgent medical attention may be needed, whereas a body temperature of 100 degrees Fahrenheit in a patient who is not immunocompromised or otherwise vulnerable to infection may represent a physical state for which treatment or continued monitoring may be appropriate but typically does not represent a dangerous physical state for which urgent medical attention may be needed. A result of a danger stratification may be expressed, for example, as a number indicating the level of danger, which may be on a relative or absolute scale. A danger stratification may serve to indicate the urgency with which the patient should obtain medical attention or treatment. In some embodiments, danger stratification may determine that the patient is in need of emergency medical attention and may instruct the patient to obtain it, e.g., by calling 911. It should be understood that the danger stratification, notification and triggering functions may utilize data from any of the patient's monitoring modules, data obtained interactively from the patient, and/or other available data regarding the patient (e.g., patient characteristics) in performing danger stratification and/or in determining which action(s) to trigger. In some embodiments, if a first abnormal value is detected by a first monitoring module, the system may utilize data obtained by any of the patient's other monitoring modules, data obtained previously by the same monitoring module, data obtained interactively from the patient, data in the patient's patient profile, or any combination thereof in performing danger stratification and/or in determining which action(s) to trigger. In some embodiments, different patient characteristics or other patient data may result in different danger stratification results and/or in triggering different action(s). For example, the presence of one or more risk factors may trigger issuing a direction to the patient to obtain urgent medical attention, whereas in a patient who lacks the risk factor, such a direction may not be issued.

In some embodiments, a monitoring module in Risk Detection Mode may detect that a patient has not been following one or more recommendations of a treatment plan prescribed as part of a T-plan by the patient's health care provider. For example, a monitoring module may detect that the patient has not been taking one or more prescribed medications. Such data may be used in a danger stratification or may trigger a notification, as described above for detection of an abnormal value. In some embodiments, data indicating that a patient has not been following one or more recommendations of a treatment plan may be used, together with an abnormal value, in a danger stratification or to determine which action(s) should be taken, e.g., which SSTs to run.

A monitoring module may be in Risk Detection Mode by default when prescribed by a health care provider as part of a T-plan and may have default settings for detection of abnormal values. However, the mode may be turned off or its settings adjusted for a particular patient. It should be noted that not all monitoring modules will have a Risk Detection Mode, and even if a particular monitoring module has a Risk Detection Mode, it may not be turned on when the monitoring module is prescribed as part of a particular T-plan. In particular, a monitoring module that operates only in an “as-needed” mode may, in at least some embodiments, not have a Risk Detection Mode.

In Health Tracker Mode the monitoring module obtains physiological data specified by the module and stores it. The stored data may be used whenever needed for running an SST. In some embodiments, when an SST is run, the monitoring module may automatically obtain new data or may obtain new data if requested to do so, in order to supply valid data for use by the SST. In Health Tracker Mode a monitoring module may or may not detect whether data that it obtains is abnormal. However, it does not trigger the running of an SST.

In Heightened Sensitivity Mode, the timeframe according to which the monitoring module obtains data is adjusted such that the data is obtained more frequently than would otherwise be the case and/or the threshold for triggering the running of an SST may be lower (meaning that, for example, a smaller deviation from normal or baseline may trigger the running of the SST than when the monitoring module is not in Heightened Sensitivity Mode). The frequency may be increased by at least a factor of 1.5, 2, 3, or more. For example, a patient may be asked to enter his or her blood pressure daily instead of every 3 days. A monitoring module may be switched into or out of Heightened Sensitivity Mode by a health care provider, patient, or automatically by the system. In some embodiments, Heightened Sensitivity Mode is automatically switched on in one, more than one, or all of the monitoring modules in a patient's T-plans that have the capability of operating in such mode if the system detects that the patient is hospitalized or likely hospitalized. In some embodiments, one or more of the monitoring modules may continue operating in Heightened Sensitivity Mode for a predetermined time period (e.g., 30 days, 60 days) or indefinitely until switched to a different mode. In some embodiments, patients can switch a monitoring module on or off or adjust its mode if desired. In some embodiments, a health care provider may switch a monitoring module to Heightened Sensitivity Mode if, for example, a patient experiences an exacerbation or is hospitalized or recently hospitalized.

Educational Modules

Educational modules may contain any type of educational material relevant to a condition or patient characteristic. In general, an educational module may be composed of one or more educational items. Educational items may be provided in any of a variety of media such as text, images, audio, video, or combinations thereof. They may include podcasts, webcasts, chat rooms, online question-and-answer sessions, individual facts that may be presented as “tips” for disease management, quizzes, etc. For example, FIG. 31 shows a brief educational item informing the patient that daily exercise can reduce the chance of exacerbations. The patient may be provided an opportunity to learn more by taking an action such as tapping the screen and would then be presented with more detailed educational information on the topic.

Educational items may comprise information about the causes, symptoms, diagnostic approaches, treatment approaches, prognosis, epidemiology, risk factors (e.g., for exacerbations, complications, or other morbidities), appropriate ways to administer medication, care for wounds, or any other type of information. In some embodiments an educational item comprises information regarding a behavior that is relevant to the condition. An educational item may be similar, substantially identical, or identical in content to educational materials that a health care provider may give to the patient in forms such as pamphlets, brochures, and the like, during an in-person encounter, at the time of discharge from a hospital, or any other time at which a health care provider would have occasion to provide educational materials to a patient. In some embodiments, educational material includes periodic updates/information on the T-plan condition. In certain embodiments, the system provides means by which health care providers, health care institutions, or health care organizations to provide their own educational items for use in T-plans prescribed to their patients. For example, a health care provider may make a video that could be provided as an educational item to his or her patients or may provide his or her preferred educational brochures. In some embodiments, an educational module may guide the patient in an exercise program, physical therapy program, or rehabilitation program such as pulmonary rehabilitation or cardiac rehabilitation. In some embodiments, an educational module may guide the patient in a smoking cessation program.

Educational modules may collect a variety of kinds of data. For example, they may collect data indicating the extent to which or way in which the patient used the educational materials. Such information may include, for example, which educational items the patient viewed or listened to or interacted with, how long the patient spent on various educational items, acknowledgement from the patient that he or she read and understood the educational material, patients' responses to quizzes or questions about the educational material, and the like.

Data regarding patients' use of the educational materials may have a variety of uses. Such data may be used, for example, by the system to evaluate the effectiveness of the material or by a health care provider in counseling the patient. Data obtained by educational modules may be used together with data obtained by monitoring modules for such purposes. Educational modules may be changed over time. Data regarding patients' use of the educational materials may be used to select or refine an overall educational program for a patient.

Health Care Event Modules

As mentioned above, a health care event module specifies health care events about which health care event data are to be obtained. A health care event module may specify health care events that are part of a treatment plan for a condition (“health management events”) and associated timeframes for at least some of the health management events. A health care event module may alternatively or additionally specify other health care events that are significant in the context of a condition but occur outside the context of a particular treatment plan (also referred to as “significant health care events”). In some embodiments the system comprises a database specifying a wide variety of health management events, significant health care events, or both. Such events may include patient events, physician events, or both, of any of a wide variety of types.

Health management events encompass any physician event or patient event that is part of a health care provider's treatment plan for managing a condition. A treatment plan may include, for example, medications to be administered, medical devices to be used, follow-up visits to be conducted, procedures to be performed, and/or recommended behaviors such as diets to be followed, exercises to be performed, smoking cessation, and the like. A treatment plan for a particular condition in a patient is typically determined after the condition has been diagnosed. A treatment plan for a condition may include a set of procedures, treatments, and other diagnostic and therapeutic interventions that a health care provider recommends or prescribes to the patient or performs for purposes of managing the condition in the patient. The procedures, treatments, and other diagnostic and therapeutic interventions of a treatment plan are typically to be performed according to a predetermined schedule, which is part of the treatment plan.

A health care event module may specify those health management events that are part of a health care provider's treatment plan for the particular patient to whom the T-plan is prescribed. The system may provide a default treatment plan for a given condition and subset of the set of significant patient characteristics for that condition, which the health care provider can then modify if desired. The system may do this for any of a wide variety of conditions and, for each condition, the various combinations of significant patient characteristics for each such condition. Such treatment plans may be encoded in a knowledge base as described above for SSTs and configured by an algorithm as described above for SSTs.

In some embodiments, based on condition and significant patient characteristics for that condition, a treatment plan that includes patient events and physician events may be automatically configured by the system. Such a treatment plan may include, for example, (a) medication (if appropriate), (b) diet and/or exercise (if appropriate); (c) schedule for physician visits and procedures (if appropriate). In some embodiments, a treatment plan is configured by applying a set of rules that map from (1) a condition (e.g., a particular disease) and a subset of a set of significant patient characteristics for that condition to (2) a subset of a set of patient events and physician events relevant to managing that condition. The treatment plan may specify, for example, medications to be used for management of the condition, a schedule of physician visits for follow-up and particular procedures to be performed at such visits, diet and/or exercise regimens, and the like. Thus, a treatment plan that encompasses the various items that make up a treatment plan (e.g., for a chronic condition) may be generated automatically according to certain embodiments of the invention. In some embodiments, the system may comprise such a set of rules for any of a wide variety of conditions.

In some embodiments, a treatment plan that encompasses health tracking (e.g., tracking of at least symptom data and physiological data, and periodic evaluations thereof) and issuance of appropriate directions to the patient to address potential adverse changes between patient visits to the health care provider based on collection and analysis of relevant health data from the patient, as well the various items that make up a treatment plan for a chronic condition may be generated automatically according to certain embodiments of the invention. As an example, a treatment plan for asthma might specify “bronchodilator” as a medication or might specify a particular bronchodilator and dose. A physician prescribing a T-plan for asthma to a particular patient or generating a personal T-plan template for asthma could select or change the particular bronchodilator or dose if desired or remove the bronchodilator entirely. As another example, a treatment plan for COPD might specify “chest X-ray” as a physician event and “yearly” as the frequency. A physician prescribing a T-plan for COPD to a particular patient or generating a personal T-plan template for COPD could adjust the frequency if desired or remove the event entirely.

A health care event module for a condition obtains health care event occurrence data and/or health care event result data pertaining to the particular health management events specified in the treatment plan for that condition. Such data may be obtained in various different ways, as appropriate for the particular type of data elements. As described further below, such data may be obtained through medication modules, monitoring modules, physician event modules, or other modules. At least some of the data are stored by the system in association with an identifier of the patient to whom the data pertains. In some embodiments, the data includes data that indicates that a specified health care event has occurred. In some embodiments, data indicating the occurrence of event may be entered by a patient. In some embodiments, data indicating the occurrence of a physician event may be entered by a health care provider. In some embodiments, data indicating the occurrence of a event may be obtained from any of a variety of data sources, such as an EMR or a claim for reimbursement of health care services that include the physician event.

In addition to, or instead of, obtaining data pertaining to health management events, a health care event module may obtain data pertaining to significant health care events. A significant health care event may be any physician event that a health care provider may consider to be of interest in regard to his or her management of the condition in the patient, e.g., any physician event of which the HCP may wish to be made aware. For example, certain events, such as hospitalizations or visits to an emergency room, may be of interest to any HCP who treats the patient for a condition because, for example, they may indicate a significant deterioration in the patient's health status in regard to the condition managed by that HCP, may indicate a significant deterioration in the patient's health status that may affect the patient's ability to manage the condition managed by that HCP or may merit a change in the treatment plan for that condition, and/or may result in changes in the patient's medications that may affect the future evolution or appropriate treatment of the condition. In some embodiments, a notification may be issued to a caregiver or a health care provider of the patient if a possible or likely or confirmed hospitalization event or a possible or likely or confirmed emergency room (ER) visit event is detected. For example, a health care provider who cares for the patient on an ongoing basis may be notified of the event. In some embodiments, a notification includes the location and/or name of the hospital or other health care facility where an event occurred. In some embodiments, the health care provider may contact the hospital or health care facility. In some embodiments, the health care provider may participate in the patient's care and/or may request records of the hospitalization in order, for example, to facilitate the patient's follow-up care after discharge. In some embodiments, the occurrence or a possible or likely or confirmed hospitalization event or ER visit event is presented to a health care provider as part of the patient summary data. The health care provider may ask the patient about the event during the patient's next appointment.

Certain procedures may be of interest to an HCP who treats the patient for a condition because the procedure may yield diagnostic information relevant to the condition and/or because the fact that the procedure was performed may indicate that another health care provider suspected or detected a significant deterioration in the condition. For example, a physician who manages a patient for COPD may consider procedures that generate information about the respiratory system, such as chest X-rays, chest CT scans, and pulmonary function tests, to be of interest, and such procedures may be significant health care events for COPD. In some embodiments, physicians will be able to rapidly know whether procedures relevant to the disease they are treating were performed on a patient, when (or approximately when), and will be provided with sufficient information to permit determining which HCP performed or ordered the procedure and/or where the procedure was performed or where results were reported. It is expected that this information will allow physicians to reduce duplication by requesting (e.g., by email, phone, fax, or other means) a copy of results or reports and/or relevant portions of the patient's medical record at the HCO where the procedure was performed or ordered. In some embodiments, data resulting from such procedures will be accessible via the system. Avoiding unnecessary repetition of procedures reduces patient risk that may be associated with such procedures as well as reducing expense. Obtaining results of a procedure that has already been performed may provide a physician with the information he or she needs to make a treatment decision more rapidly than would be the case if the physician needed to wait until the procedure could be scheduled and performed.

A health care event module may comprise one or more modules that relate to different types of health care events or different aspects of health care events. Each such module may specify health management events, significant health care events, or both, within the particular domain of the module. For example, a health care event module may contain one or more of the following modules: a medication module, a physician events module, and a schedule module, each of which may specify health management events, significant health events, or both, within the domain of the module. Any of these modules may be automatically configured based on condition and material patient characteristics using an algorithm such as described above. Thus the system may contain an appropriate knowledge base for this purpose.

A medication module may fulfill any of a variety of purposes relating to medications. For example, a medication module in a T-plan template for a given condition may store information specifying one or more default medications, which may represent a typical, standard-of-care treatment approach for a patient with the condition and a particular subset of the set of significant patient characteristics for that condition; a medication module in a health care provider's personal T-plan template library may store information specifying a particular health care provider's preferred medication(s) for treating patients with the condition and a particular subset of the set of significant patient characteristics for that condition. An active T-plan may store information specifying medications that the health care provider has prescribed for that patient for treatment of the T-plan condition. The information relating to a medication may specify the name of the medication and may also specify other relevant information such as the dose, dosing timeframe (e.g., dosing interval or frequency), route of administration, and/or any other information that may be found in a prescription. A medication module may obtain additional data or perform one or more other activities pertaining to medications. For example, it may obtain data specifying medication allergies, may check prescribed medications for potential medication allergies or contraindications.

In some embodiments, a medication module obtains data indicating the administration of a medication to the patient (e.g., self-administration by the patient or administration by a caregiver) and/or provides reminders to the patient or caregiver to administer the medication(s) at appropriate times according to the specified timeframe. The system may determine one or more time windows relating to administration of a medication. This may be done based on stored information specifying a time window for each of a plurality of medications and/or may be calculated based on factors such as the dosing interval. The system may for example, determine at what time, relative to a scheduled dose, a reminder or other notification should be issued or at what time the scheduled dose should be considered to be “missed”. In some embodiments, a medication module comprises or obtains data through a monitoring module. For example, a monitoring module may ask the patient one or more questions about his or her medication usage or may obtain data from a connected monitoring device that tracks medication usage. Data indicating the administration of medications may be stored as part of the patient record. In some embodiments, such data may be used in determining a compliance score for a patient.

A physician events module of a T-plan comprises information specifying physician events to be performed according to a treatment plan for a condition. A physician events module in a T-plan template for a given condition may store information specifying the procedures and associated timeframes of a default treatment plan for the condition, which may represent a typical, standard-of-care treatment approach for a patient who has the condition and has a particular subset of the set of significant patient characteristics for that condition. The system may provide a set of default treatment plans for a condition given a particular set of the material patient characteristics for that condition. A physician events module in a health care provider's personal T-plan template library may store information specifying the procedures and associated timeframes of a particular health care provider's preferred treatment plan for treating all patients who have condition or those patients with the condition who have any particular subset of the set of significant patient characteristics for that condition. An active T-plan may store information specifying procedures that the health care provider has ordered for that patient for treatment of the T-plan condition, and associated timeframes for performing at least some of the procedures. The procedure(s) and timeframe(s) specified in an active T-plan for a patient with a condition may be identical to the procedures specified in a default treatment plan or in a personal T-plan template for the condition or may have been customized specifically for the patient by the health care provider.

In some embodiments, a physician events module obtains data indicating the performance of a procedure on or directed to the patient (e.g., performance of a diagnostic procedure by a health care provider). In some embodiments, reminders are issued to the patient to have the procedure performed at appropriate times according to the specified timeframe. For example, suppose that a physician event module in a T-plan for COPD specifies a timeframe of one per year for chest X-rays. The health care event module searches for health care event data indicating the performance of a chest X-ray in appropriate available data sources and/or REVON system databases that have acquired data from such sources. If no such data indicating the performance of a chest X-ray within the preset timeframe of one year is found, the patient is reminded to get one. If such data is found, the patient is not reminded to get a chest X-ray. The system may determine one or more time windows relating to a physician event. This may be done, for example, based on stored information specifying a time window for each of a plurality of physician events. The system may for example, determine at what time, relative to a scheduled physician event, a reminder or other notification should be issued to the patient.

A schedule module may provide a schedule for a patient that may include health management events (patient events, physician events, or both) that have been definitely scheduled and/or that are to take place in the future but for which a specific date and, where applicable, time of day have not yet been arranged. In such instances, a physician event as presented on a schedule may comprise a reminder to arrange a specific time and day for the associated actual physician event to take place, e.g., a reminder to make an appointment for a procedure. If a specific date and time have been arranged, a schedule may reflect that fact. A schedule may have any of a variety of formats in which events are associated with times. For example, a schedule may comprise a timeline in which the passage of time is shown from left to right and events that are to occur or that have occurred are indicated at various positions along the timeline, e.g., below it. A schedule may be in the form of a two-dimensional grid with time along the x-axis, and events of different types distributed along the y-axis (or vice versa). A symbol such as an X may indicate the time or approximate time at which an event is to occur or occurred (different symbols or colors may be used to distinguish events that occurred or are to occur or did not occur as scheduled). A schedule may be displayed as a calendar with events indicated in different boxes representing different days. Various display formats may be used. A schedule may have one or more capabilities, data, and/or functions associated with it. For example, events may have associated data, which may, in some embodiments, be accessed from the schedule (e.g., by clicking on the event). The data used to populate a schedule may be stored in one or more databases and used for any of a variety of purposes, e.g., as described herein.

In some embodiments, a patient may be able to confirm having performed a particular action associated with a patient event such as taking a medication or making or attending an appointment. In some embodiments, a patient may do so by, for example, tapping on a button or icon representing the action in the schedule. In some embodiments, a patient may be asked by the system whether he or she has performed certain actions associated with a patient event. In some embodiments, a patient may be asked by the system whether an event that is a significant patient event for one or more T-plans, such as a hospitalization or emergency room visit, has occurred. In some embodiments, it is envisioned that certain patient events will populate in a schedule as having occurred by means of data obtained by the REVON system from connected monitoring devices, such as connected medication dispensers, connected glucometers, etc. In some embodiments, a schedule may distinguish between events that have occurred and those that have not occurred, e.g., by using different colors. A patient who forgets whether he or she has taken a scheduled dose of medication may check the schedule in order to determine whether or not the event has occurred.

In some embodiments, a schedule comprises or consists of an appointment schedule that may provide the patient with a schedule of upcoming health management events (e.g., appointments for procedures and/or follow-up) that are to be performed as part of a health care provider's treatment plan for managing the condition. For those health management events that have not been definitively scheduled, an appointment schedule may indicate a time window within which the health management event should occur according to the timeframe specified for that event in the health care event module. An event is considered “definitively scheduled” if a specific date and, in some embodiments, a specific time on that date, on which the event is to occur have been established. Generally, an appointment with a health care provider is considered definitively scheduled once the patient and health care provider or health care facility have agreed on the specific date and, typically, specific time, which are typically recorded by the health care provider and/or health care facility. For those health management events that have been definitively scheduled, an appointment schedule may indicate the specific date and time that the event is to take place. In some embodiments, reminders are issued to the patient to definitively schedule a health management event, e.g., an appointment, in accordance with a timeframe specified for that event in the health care event module. In some embodiments, reminders are issued to remind the patient of the day and time of upcoming definitively scheduled appointments. In some embodiments, reminders are issued to the patient to definitively schedule a health management event, e.g., an appointment, if health care event data collected by the system indicates that such event has not taken place in accordance with a timeframe specified in a health care event module. The determination of when to issue reminders, and the issuance of reminders, may be performed by the schedule module or by a different module.

In some embodiments, a health care provider may be able to confirm the occurrence of a physician event such as a procedure or follow-up visit of a patient within a physician application, as described further below. In some embodiments, the occurrence of physician events is confirmed by the system obtaining data indicating that the event occurred. Such data may come, for example, from the patient's EMR or from a claim for reimbursement for the physician event.

In some embodiments, if a patient has multiple T-plans, each with a physician events module, the health management events that are to be performed as part of the treatment plans for those conditions may be consolidated into a single schedule. In some embodiments, patients may be able to view condition-specific schedules and a consolidated schedule.

T-Plans and T-Plan Configuration

In some embodiments, the system may provide the capacity to configure and prescribe T-plans in any one or more health care disciplines. In some embodiments such T-plans may collectively encompass those conditions that afflict at least about 70%, 80%, 90% or more of the patients that a typical practitioner in the health care discipline would treat. In some embodiments such T-plans may encompass at least the 3 to 5 conditions most commonly treated by practitioners (e.g., typical practitioners) in that health care discipline, i.e., at least the top 3 to 5 conditions in terms of number of patients with the condition that receive treatment by practitioners in the health care discipline. For example, in some embodiments, T-plans for pulmonology may include at least a COPD T-plan, an asthma T-plan, a pulmonary embolism T-plan, a sarcoid T-plan, and a pneumonia T-plan. In some embodiments, T-plans for cardiology may include at least a coronary artery disease T-plan and a heart failure (or congestive heart failure) T-plan, among others. It should be noted that a particular T-plan may be relevant to multiple health care disciplines whose practitioners may treat patients with the T-plan condition.

In some embodiments, a T-plan may be specified by information that identifies a condition and information that identifies the set of data collection modules included in the T-plan. In some embodiments, identifying the set of data collection modules of included in the T-plan is sufficient to implicitly specify the condition. In some embodiments, a T-plan is further specified by information identifying any changes or additions that have been made in a T-plan template to modify the T-plan template as a new T-plan template or to customize the T-plan template as an active T-plan. The set of data collection modules in a particular T-plan may be determined at least in part by the system. In some embodiments, T-plans and/or data collection modules, may sometimes be designed at least in part by individual health care providers, members of health care teams, health care institutions, health care organizations, or the system. Sometimes the designer of a T-plan or data collection module may be a health care provider who prescribes the T-plan to a patient. Sometimes a health care provider uses a T-plan template made available by the system, which may be designed in collaboration with or approved by a particular physician practice, health care institution, or health care organization through which the patient receives care.

In some aspects, the invention provides a database that stores a library of data collection modules that can be assembled in a wide variety of combinations to produce multiple distinct T-plans. The modular nature of T-plans allows for the creation of personalized T-plans in a rapid and at least partly automated fashion, based on input that identifies a condition and a set of patient characteristics. Certain embodiments of the invention allow a user to create T-plans in a flexible manner by specifying a condition and a set of patient characteristics, which results in automatic selection of a set of one or more appropriate data collection modules by the REVON system. In each case, the selected data collection modules define a T-plan that can be stored and/or prescribed to a patient. In certain embodiments, a user can modify a T-plan by, for example, adding or removing modules and/or customizing certain elements of certain modules as described further below. It should be noted that other modules, which may facilitate operation of one or more data collection modules or carry out other T-plan-associated functions described herein, may also be included as part of a T-plan specification.

In some embodiments, the REVON system automatically selects a set of one or more modules for a T-plan using an algorithm whose inputs identify (1) a condition and (2) a subset of a set of significant patient characteristics for that condition. The subset of a set of significant patient characteristics would be those of a particular patient to whom the T-plan is to be prescribed. Typically, the T-plan comprises at least a health tracking module that provides a health tracker for the specified condition and one or more monitoring modules that obtain physiological data relevant to the condition, e.g., physiological data that may be used by the health tracker in conducting a health evaluation.

In some aspects the invention provides a database that associates, with each of a plurality of conditions, one or more monitoring modules that obtain physiological data, behavioral data, or environmental data relevant to the condition. These monitoring modules or a subset thereof may be included in a T-plan. For example, the system may automatically select such modules as part of the T-plan configuration process. Data obtained by such modules may be relevant in that, for example, it may be used in a health evaluation in evaluating the patient's health status with regard to the condition and/or in determining directions to the patient, may trigger performance of a health evaluation if, for example, the data are indicative of a deterioration or potential deterioration or notable change in the patient's health, and/or may be useful to the patient or health care provider for purposes such as determining prognosis, monitoring progression, monitoring improvement, evaluating the efficacy, side effects, or risk/benefit ratio of treatment, and/or evaluating the patient's compliance with treatment recommendations.

In some embodiments the database also associates, with each of a plurality of conditions, one or more educational modules comprising educational materials pertaining to the condition. These educational modules or a subset thereof may be included in a T-plan. For example, the system may automatically select such modules as part of the T-plan configuration process.

In some embodiments, a T-plan comprises (a) a health tracking module for the T-plan condition and (b) one or more monitoring modules that obtain physiological data to be used by the health tracking module to evaluate the patient's health status with regard to the T-plan condition and/or one or more educational modules that provide educational material relating to the T-plan condition. In some embodiments, the health tracking module comprises at least one symptom tracker for the T-plan condition. In some embodiments, the T-plan comprises an SST for the T-plan condition. When a T-plan for a particular condition is prescribed, the system may automatically select, as part of the T-plan a health tracking module comprising such an SST, and monitoring modules that obtain physiological data, behavioral data, or both, that may be used by the SST or may trigger running of the SST. For example, if the T-plan condition is COPD, the T-plan will often comprise a health tracking module that includes an SST for COPD (e.g., COPD exacerbation), which may utilize oxygen saturation measurements in evaluating the patient's health status. The system may automatically select a monitoring module for obtaining oxygen saturation measurements as part of the COPD T-plan. If the T-plan condition is one in which patients may be prone to two or more distinct types of clinically significant occurrence, the T-plan configuration module may select an SSTs for each such type of acute deterioration or exacerbation or other clinically significant occurrence, and appropriate monitoring modules for obtaining data that may be used by or may trigger such SSTs. The system may additionally select an educational module containing educational materials about COPD as part of the COPD T-plan.

In some embodiments, the T-plan configuration module may additionally select a health care event module that specifies health care events relevant to the T-plan condition. As described above, the heath care event module may comprise a medication module, a physician events module, or both. In some embodiments, a physician events module provided as part of an automatically configured T-plan may comprise information specifying physician events to be performed according to a default treatment plan for the condition. In some embodiments, if a health care provider prescribing the T-plan has saved a treatment plan for the condition in his or her personal T-plan template library, the T-plan configuration module may utilize that treatment plan. Thus in some embodiments, an automatically configured T-plan may comprise a physician events module that stores information specifying the procedures and associated timeframes of a particular health care provider's preferred treatment plan for treating the condition. In some embodiments the health care event module further comprises a schedule module. The T-plan configuration module may generate a schedule module based on the treatment plan.

As noted above, in some embodiments, the set of modules in a T-plan may be determined at least in part based on significant patient characteristics in addition to the T-plan condition. In general, significant patient characteristics for a condition may affect the choice of modules to be included in a T-plan for that condition and/or may affect the content of one or more modules. For example, certain modules may be included or not depending on whether or not the particular patient characteristic is present. In some embodiments, one or more monitoring modules, one or more educational modules, or both, is included if a particular patient characteristic is present or absent. The content of one or more modules may be affected by significant patient characteristics in various ways. For example, an appropriate default treatment plan for the condition to be provided as part of a health care event module vary depending on whether or not a particular material patient characteristic is present, or appropriate directions for management of the condition to be provided by an SST may vary depending on whether or not a particular material patient characteristic is present. Material patient characteristics for a given condition may include one or more additional conditions whose co-existence in the patient along with a T-plan condition or SST condition may be material to the management of the condition in that it (a) has the potential to affect the proper interpretation of health data relevant to the T-plan condition or SST condition, (b) merits an alteration in the process of obtaining health data (e.g., collection of additional types of health data, more frequent collection) as compared to in the absence of the additional condition, and/or (c) has the potential to affect the appropriate directions to be provided by an SST based on a given set of health data. Significant patient characteristics for a given condition may include conditions that occur reasonably commonly in patients with the given condition and have the potential to materially affect the patient's health or prognosis regardless of whether they fulfill any of the afore-mentioned criteria, behaviors that unfavorably affect the health or prognosis of patients with the condition and/or increase the likelihood that a patient with the condition will experience an adverse change within a predetermined time period, and complications of the condition.

Certain patient characteristics may themselves be conditions. Such co-existing conditions are sometimes referred to as “comorbidities”. As used herein, “comorbidity” refers to a condition that exists simultaneously with another condition, regardless of their causal relationship. Comorbid conditions may exist independently or may be connected in that, for example, a first condition in a patient causes, is caused by, complicates, affects the treatment of, or is otherwise related to, another condition in the same patient. In some embodiments, a T-plan configuration method selects those monitoring modules, educational modules, or both, as are specified for a comorbidity that is a material patient characteristic for the T-plan condition and includes them in T-plans for the T-plan condition for those patients for whom the comorbidity is indicated as a patient characteristic. In some embodiments, a T-plan configuration method selects at least those monitoring modules that specify collection of physiological data, behavioral data, or both, as are specified for a comorbidity that is a material patient characteristic for the T-plan condition and includes them in T-plans for the T-plan condition for those patients for whom the comorbidity is indicated as a patient characteristic. In some embodiments, a T-plan configuration method selects those monitoring modules, educational modules, or both, as are specified for a comorbidity that is a significant patient characteristic for the T-plan condition and includes them in T-plans for the T-plan condition for those patients for whom the comorbidity is indicated as a patient characteristic. In some embodiments, a T-plan configuration method selects at least those monitoring modules that specify collection of physiological data, behavioral data, or both, as are specified for a comorbidity that is a significant patient characteristic for the T-plan condition and includes them in T-plans for the T-plan condition for those patients for whom the comorbidity is indicated as a patient characteristic. In some embodiments, a set of monitoring modules that specify collection of physiological data, behavioral data, or both, for a comorbidity that is a significant patient characteristic for the T-plan condition may be the same as the set of monitoring modules that would be included in a T-plan for which that comorbidity was the T-plan condition. In some embodiments, a set of monitoring modules that specify collection of physiological data, behavioral data, or both, for a comorbidity that is a significant patient characteristic for the T-plan condition may differ from the set of monitoring modules that would be included in a T-plan for which that comorbidity was the T-plan condition. For example, in some embodiments a different monitoring regimen, e.g., more extensive or less extensive monitoring, may be performed pursuant to a T-plan when the condition is present as a comorbidity of the T-plan condition as compared to if the comorbidity was the subject of its own T-plan. In some embodiments the monitoring for a comorbidity may be tailored appropriately in light of the fact that it is being monitored as a comorbidity.

In some embodiments, a T-plan configuration method selects at least those monitoring modules that specify collection of physiological data, behavioral data, or both, that is needed by the system to distinguish between a clinically significant occurrence arising from the T-plan condition from a clinically significant occurrence arising from the comorbidity. In some embodiments, a T-plan configuration method selects at least those monitoring modules that specify collection of physiological data, behavioral data, or both, that is needed by the system to accurately determine management directions for the T-plan condition taking into account the fact that the patient has the comorbidity. In some embodiments, this is performed for one, more than one, or each such comorbidity that is specified as a material patient characteristic for the T-plan condition. In some embodiments, this is performed for one, more than one, or each such comorbidity that is specified as a significant patient characteristic for the T-plan condition. In some embodiments, such T-plans may not include a health tracking module specifically for the comorbidity. In some embodiments, such T-plans may include a health tracking module for the comorbidity. In some embodiments, a health tracking module for the comorbidity does not comprise a smart symptom tracker for the comorbidity. In some embodiments, a health tracking module for the comorbidity comprises a smart symptom tracker for the comorbidity. In some embodiments, the SST for the comorbidity may include triage directions and treatment directions. In some embodiments, the SST for the comorbidity may include triage directions but not treatment directions. For example, it may be desirable in certain embodiments and/or in the case of certain T-plan conditions and/or comorbidities to avoid a scenario in which a physician responsible for managing the T-plan condition becomes responsible for prescribing management directions for the comorbidity in addition to management directions for the T-plan condition or it may be desirable in certain embodiments to avoid a scenario in which a physician responsible for managing the T-plan condition becomes responsible for prescribing treatment directions to the patient for the comorbidity. In general, a T-plan for a condition would often not typically include a health care management module for the comorbidity. However, in some embodiments, it may do so, at least in the case of certain comorbidities. In some embodiments, a health care provider who prescribes a T-plan for a condition may be asked whether he or she wishes to also prescribe a T-plan for the comorbidity, e.g., if the patient does not already have a T-plan for the comorbidity. In some embodiments, the patient may be asked whether he or she has a health care provider for a comorbidity for which no T-plan has been prescribed to the patient.

In some embodiments, the system stores (i) information identifying combinations of two or more conditions or combinations of at least one condition and at least one characteristic that, if present, may warrant an alteration in the monitoring as compared with the monitoring that would be performed by a combination of the monitoring modules for each of the conditions and/or characteristics; and (ii) information specifying the alteration. Such alteration may, for example, comprise monitoring one or more types of physiological data elements, behavioral data elements, or environmental data elements, that would not be relevant in the absence of one or more of the conditions or characteristics, or monitoring in a different monitoring mode, or using a different monitoring device. The T-plan configuration module may detect the presence of such combinations of conditions or combinations of at least one condition and at least one characteristic when automatically configuring a T-plan for a patient and make the appropriate alteration(s) in the monitoring module(s) of the T-plan. For example, one or more additional monitoring modules may be added to the T-plan.

In some embodiments, the system stores (i) information identifying combinations of two or more conditions or combinations of at least one condition and at least one characteristic that, if present, may warrant an alteration in the educational modules as compared with a combination of the educational modules for each of the conditions and/or characteristics and (ii) information specifying the alteration. Such alteration may, for example, comprise providing one or more educational items, that would not be relevant in the absence of one or more of the conditions or characteristics, or modifying or removing one or more educational items. The T-plan configuration module may detect the presence of such combinations of conditions or combinations of at least one condition and at least one characteristic when automatically configuring a T-plan for a patient and make the appropriate alteration(s) in the educational module(s) of the T-plan.

In some embodiments, due to the automatic configuration of T-plans based on condition and significant patient characteristics, at least some components of different automatically configured T-plans may largely or completely overlap, at least in regards to SSTs, monitoring modules, and educational modules. For example, when a T-plan is prescribed to a patient who already has a patient profile, those significant patient characteristics that are already specified in the patient profile may be pulled in to the automatic T-plan configuration process (unless, in some embodiments, switched off by the health care provider prescribing the T-plan). However, in some embodiments when a T-plan is prescribed for a condition that appears as a comorbidity in the context of a different T-plan, one or more components of the T-plan prescribed for the condition may at least in some respects take precedence over the corresponding components when prescribed under a different T-plan in the comorbidity context. In some embodiments, the more stringent requirements for monitoring may take precedence, e.g., a higher frequency would take precedence over a lower frequency.

In some embodiments, there may be multiple different instances of a T-plan configuration method, which may employ different sets of significant patient characteristics deemed to be significant in the context of a given condition. For example, different health care institutions or health care organizations may have different policies or preferred protocols for treating patients with particular conditions or may serve patient populations in which different comorbidities are common, making it appropriate to deem different patient characteristics significant in the context of a given condition. In some embodiments, a health care provider, physician practice, health care institution, or health care organization may define or contribute to defining a set of patient characteristics to be deemed significant in the context of a given condition, which patient characteristics are then used to configure T-plans for that condition which are prescribed by that health care provider, physician practice, health care institution, or health care organization.

In some aspects, a system of the invention provides a user interface through which health care providers can configure T-plans and prescribe them to their patients. When a health care provider wishes to configure a T-plan the health care provider may select a particular T-plan condition and is then presented with a “menu” of the significant patient characteristics for that T-plan condition, from which the health care provider can select those patient characteristics present in that particular patient. The health care provider may be prompted to select the patient characteristics present in the particular patient and may be required to do so in order to proceed with prescribing the T-plan. The selected patient characteristics may be stored in a patient profile for that patient. In some cases a patient profile may already exist in a system database. For example, a patient profile may have been created by a different health care provider. If a patient profile already exists, the health care provider may be asked to confirm or update those significant patient characteristics for the T-plan being prescribed. In some embodiments, the health care provider may be able to add additional patient characteristics beyond those offered as options in a menu. It should be noted that, in some embodiments, a patient profile of a particular patient may identify one or more characteristics that are determined by the system to be patient characteristics of that patient based on data that may be acquired from a variety of sources in addition to or instead of those selected by a health care provider during use of the REVON system. For example, the system may determine, based on data from external data source(s) that a patient has a particular condition, is taking a particular medication, or has been hospitalized a certain number of times within a certain time period. Such characteristics may be included in a patient profile, may be significant patient characteristics in the context of one or more conditions, may be material patient characteristics in the context of one or more conditions, or any combination of the foregoing. In some embodiments, a health care provider may be informed of the system-determined patient characteristics, e.g., when he or she prescribes a T-plan to the patient. In some embodiments, a health care provider may not be asked to confirm or update those material or significant patient characteristics that are system-determined.

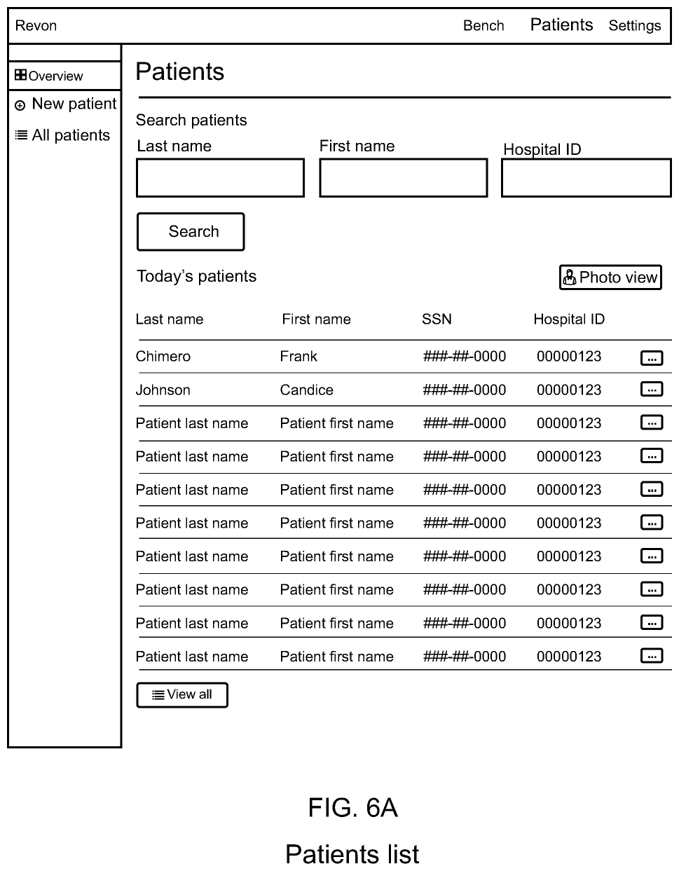

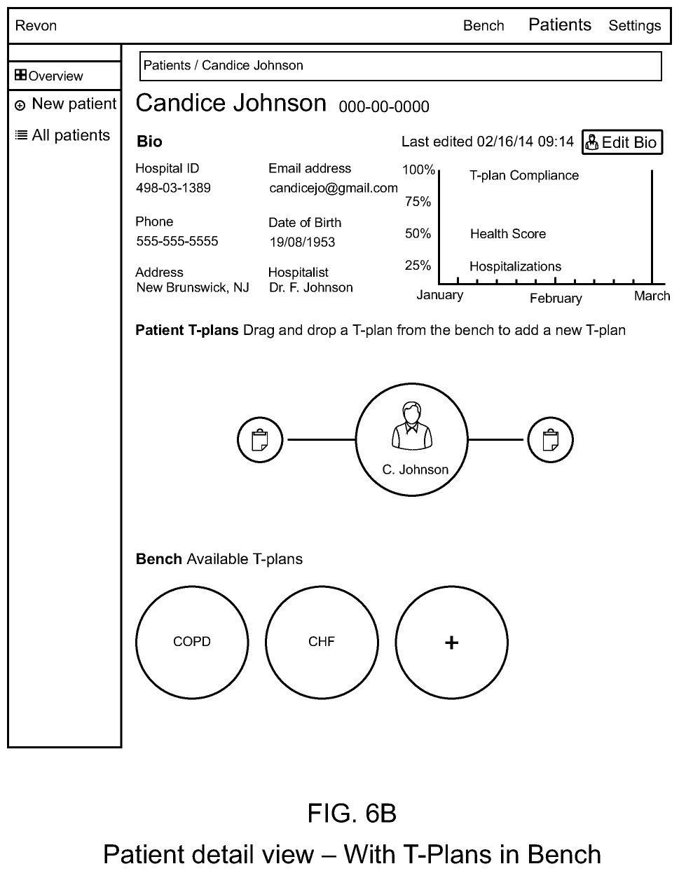

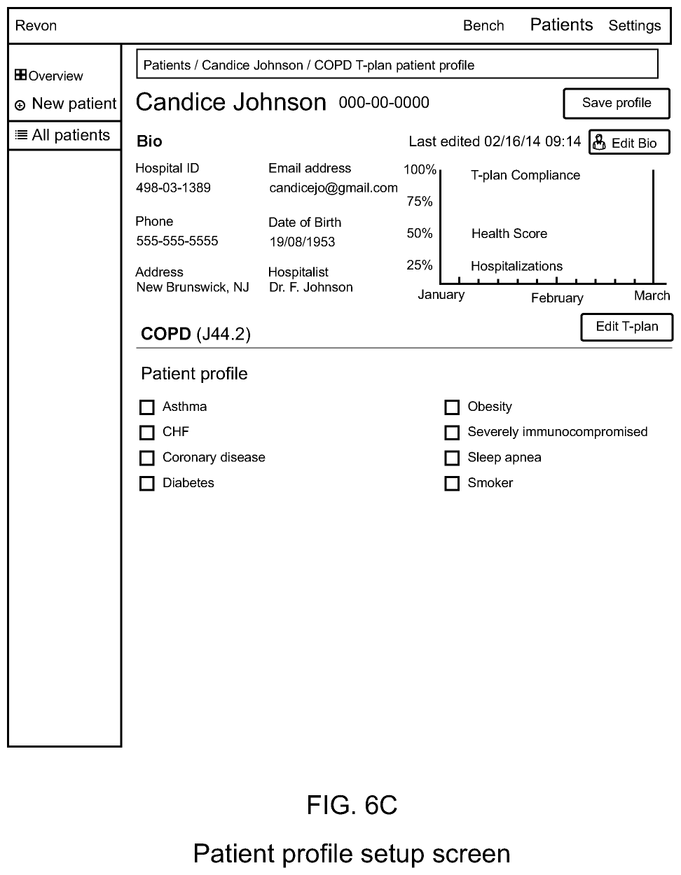

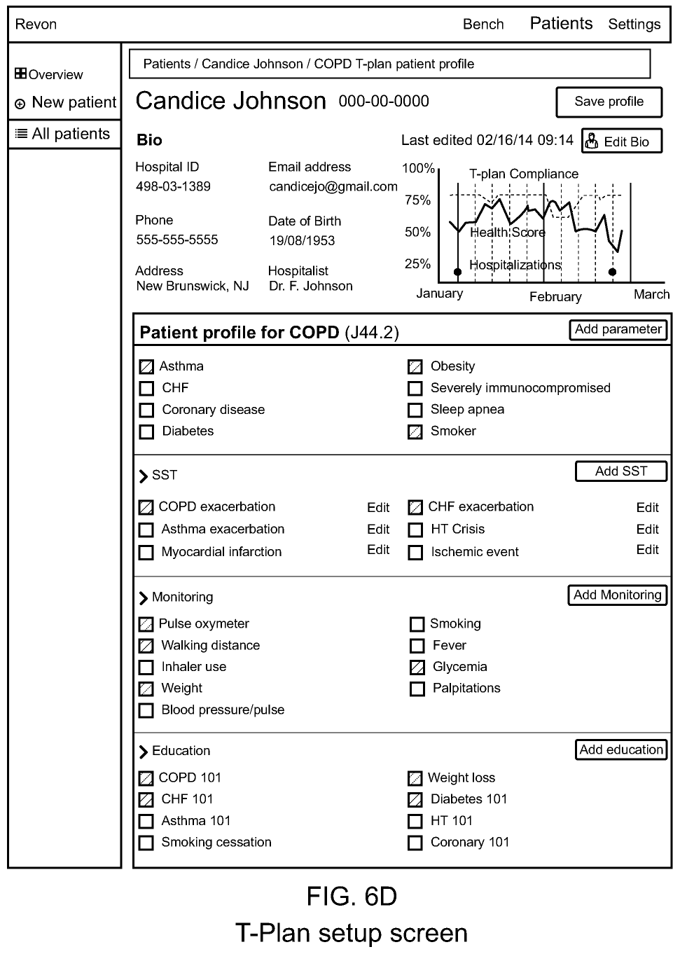

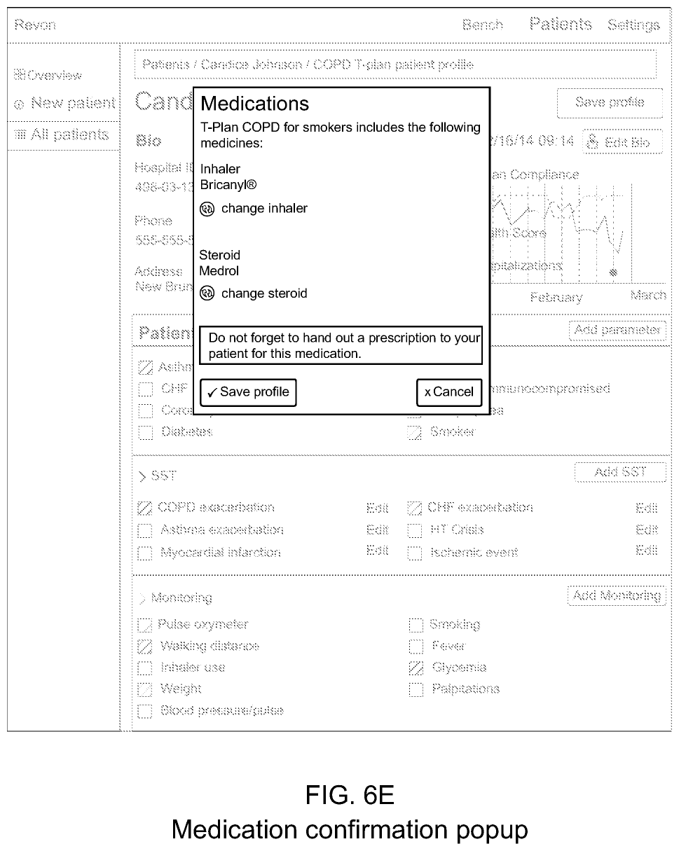

FIG. 6A shows a screen of an embodiment of a physician application of the present invention in which a health care provider is able to select a patient from a list, e.g., for purposes of prescribing a T-plan or viewing a T-plan or T-plan data. FIG. 6B shows a screen in which a health care provider is able to select a T-plan for either COPD or CHF to prescribe to a selected patient. FIG. 6C shows a screen in which a health care provider prescribing a T-plan for COPD is able to select particular patient characteristics from a set of significant patient characteristics for COPD, thus establishing a patient profile. Selecting particular patient characteristics may result in automatic inclusion in the T-plan of certain modules relating to the selected patient characteristic(s). In some embodiments, the selected characteristics are stored in the patient record as part of a patient profile. In some embodiments, if the patient is already enrolled with the system and has a patient profile, the existing patient characteristics in that profile would be indicated in the patient profile selection menu displayed to the health care provider. The health care provider may confirm or update the patient profile. In some embodiments, changing the patient profile may cause the system to inform other health care providers who are at that time managing any of the patient's T-plans of the change that has been made to the patient profile.

Based on the condition and significant patient characteristics the system may automatically determine which modules are to be included in the T-plan for the patient and/or may automatically determine certain content of certain of the modules. For example, if a patient has a particular material patient characteristic, the system may automatically include monitoring module(s) appropriate to obtain the physiological data relevant to that characteristic that may be required by the SST algorithm for the condition given that particular material patient characteristic. For example, if the T-plan condition is COPD and the patient has CHF as a patient characteristic, the system may automatically assign a monitoring module for obtaining body weight as part of the COPD T-plan because body weight measurements may be useful in distinguishing a COPD exacerbation from a CHF exacerbation. As another example, if the T-plan condition is COPD and the patient has hypertension as a patient characteristic, the system may automatically include a blood pressure monitoring module in the T-plan. The blood pressure monitoring module would specify “blood pressure” and a timeframe, and the system would obtain the patient's blood pressure according to the timeframe specified through an appropriate channel (e.g., by asking the patient to enter it or from a connected monitoring device if available.

FIG. 6D shows a screen showing an example of a T-plan for COPD for a patient with a particular set of patient characteristics. It should be noted that FIG. 6D is presented by way of example to illustrate various types of data collection modules that may be included in a T-plan in various embodiments and to illustrate aspects of a physician interface that allows a physician to select a subset of a set of significant patient characteristics for COPD and to select particular health tracking modules or portions thereof (e.g., SSTs), monitoring modules, educational modules to be included in or removed from a T-plan. It is not intended to represent the way a particular T-plan for COPD would actually be configured based on the condition and the patient characteristics indicated. The health care provider may, if desired, add or delete modules or portions thereof, e.g., SSTs. However, if the health care provider attempts to delete a monitoring module that obtains data element types that are required by an SST, the system may inform the health care provider accordingly and may remove the SST unless the monitoring module is retained or the data element type is already being obtained by a monitoring module that is assigned to the patient, e.g., as part of a different T-plan. If the health care provider adds an SST, the system may automatically add the appropriate monitoring modules to obtain the data element types required by the SST.

The system may automatically assign one or more educational modules for the T-plan condition and one or more educational modules for any one or more significant patient characteristics for the T-plan condition. For example, if a patient with conditions X and Y is to be prescribed a T-plan for condition X, and condition Y is a significant patient characteristic for condition X, the system may assign an educational module containing educational material about condition Y as part of the T-plan for condition X. Of course, if the patient already has a T-plan for condition Y or already has a T-plan for a condition for which condition Y is a significant patient characteristic, the patient may already have been prescribed an educational module for condition Y, in which case the system may not assign additional educational modules pertaining to condition Y. However, this is but one of the possibilities that may be encoded by an algorithm to be applied by a T-plan configuration module in configuring a T-plan for condition X for a patient that has condition Y. For example, if the co-existence of conditions X and Y in the patient renders aspects of the educational modules for condition X or condition Y incomplete in the context of a patient with both conditions, one or more distinct educational modules pertaining specifically to the combination of conditions may be assigned. In some embodiments, the presence of particular patient characteristic may cause a particular module to always be included as a default, regardless of condition and patient characteristics. For example, if a patient has “smoker” as a patient characteristic he or she may always receive a monitoring module for smoking and an educational module for smoking cessation. In general, a T-plan configuration method may utilize any of a variety of different algorithms to automatically configure a T-plan. It should be understood that the invention is in no way limited in this respect.

In some aspects, a T-plan configuration module comprises or accesses a knowledge base that specifies, for each of a plurality of conditions, a set of monitoring modules to be included in a T-plan for managing that condition in a patient who has that condition. In some embodiments the knowledge base further specifies, for each of a plurality of conditions, a set of monitoring modules to be included in a T-plan for a different condition, where the condition is a comorbidity. In some embodiments the knowledge base further specifies, for each of a plurality of conditions, one or more educational modules to be included in a T-plan for managing that condition in a patient who has that condition. In some embodiments the knowledge base further specifies, for each of a plurality of conditions, a set of educational modules to be included in a T-plan for a different condition, where the condition is a comorbidity. In some aspects, the knowledge base further specifies, for each of a plurality of patient characteristics (other than conditions), a set of monitoring modules to be included in a T-plan for a patient who has that particular patient characteristic as a significant patient characteristic. In some embodiments the knowledge base further specifies, for each of a plurality of patient characteristics (other than conditions), a set of educational modules to be included in a T-plan for a patient who has that particular patient characteristic as a significant patient characteristic.

In some aspects, a T-plan configuration module may automatically configure a T-plan for a condition and a subset of significant patient characteristics for the condition by performing a method comprising: (i) selecting a health tracking module for the condition; (ii) selecting those monitoring modules, if any, that are relevant to the T-plan condition, e.g., as specified in the knowledge base; (iii) for each material patient characteristic among the subset of significant patient characteristics, selecting those monitoring modules, if any, that are relevant to the characteristic, e.g., as specified in the knowledge base. For those material patient characteristics that are conditions, the appropriate monitoring modules may be those that are specified for the condition in the knowledge base (i.e., the same set of monitoring modules as would be included if the condition was the subject of its own T-plan) or may be those specified in the knowledge base for use when the condition is a comorbidity. In some embodiments the method may further comprise: (iv) selecting additional monitoring modules for one or more significant characteristics that are not material, e.g., as specified in the knowledge base. In some embodiments the method may further comprise (v) determining whether alteration of the monitoring modules is warranted and, if so, making appropriate alteration(s). In some embodiments the method may alternately or additionally further comprise; (vi) selecting one or more educational modules that pertain to the condition, e.g., as specified in the knowledge base (i.e., the same set of educational modules as would be included if the condition was the subject of its own T-plan) or may be those specified in the knowledge base for use when the condition is a comorbidity; (vii) for one or more material patient characteristics, selecting one or more educational modules that pertain to the characteristic, e.g., as specified in the knowledge base; (viii) for one or more significant patient characteristics, selecting one or more educational modules that pertain to the characteristic, e.g., as specified in the knowledge base; and/or (ix) determining whether alteration of the educational modules is warranted and, if so, making appropriate alteration(s). In some embodiments the T-plan configuration method further comprises selecting a health care event module for the T-plan condition, e.g., as described above.

In some aspects, a T-plan configuration module comprises or accesses a knowledge base that specifies, for a condition and each possible subset of the significant patient characteristics for that condition, a set of data collection modules to be included in a T-plan for managing that condition in a patient who has a particular subset of the significant patient characteristics. For example, if condition X has 5 significant patient characteristics (PC1, PC2, PC3, PC4, and PC5), the knowledge base would specify those data collection modules to be included in a T-plan for condition X for a patient who has any one or more of PC1, PC2, PC3, PC4, and PC5, e.g., PC1 only, PC1 and PC2, PC2 only, PC1 and PC3, PC3 and PC5, etc. The knowledge base comprises such information for each of a plurality of conditions.

The set of modules for a given condition is all modules that may be assigned by the system for the condition taking into consideration every potential combination of significant patient characteristics. The particular significant patient characteristics specified for a given patient with that condition map to a subset of these modules, and it is this subset that is assigned to the patient as part of the T-plan for the condition. The particular mapping may be stored in a knowledge base. The table below provides an example of how such information might in certain embodiments be represented for a particular condition, e.g., COPD, for three different combinations (P1, P2, and P3) of five significant patient characteristics, PC1, PC2, PC3, PC4, and PC5. P1, P2, and P3 might represent patient profiles of three different patients with COPD, or at least those patient characteristics of the patient profile that are significant for COPD.

TABLE 1
Schematic Representation of a Portion of a T-plan
Configuration Knowledge Base

| | PC1 | PC2 | PC3 | PC4 | PC5 | HTM | MM | EM | HCEM |
|---|---|---|---|---|---|---|---|---|---|
| P1 | | × | | × | | 1 | 1, 2, 3, | 1, 3, 4 | 1 |
| | | | | | | | 4, 6 | | |
| P2 | × | × | × | | | 1 | 1, 2, 4, | 1, 3, 8, | 1 |
| | | | | | | | 7 | 9 | |
| P3 | | | | | × | 1 | 1,2 | 1 | 1 |

The table above indicates which health tracking modules (HTM), monitoring modules (MM), educational modules (EM) and health care event modules (HCEM) would be included in an automatically configured COPD T-plan for a given patient.

HTM1 might be a health tracking module for COPD, which may be included in any T-plan for COPD. The prescribing physician may have modified HTM1 and saved it in his or her personal library or may have customized it for a particular patient, e.g., by specifying a particular rescue medication for a COPD exacerbation.

MM1 and MM2 might be monitoring modules for physiological variables that may be relevant to any patient with COPD, e.g., pulse oximeter and peak flow meter measurement. MM3 might be a monitoring module for body weight, which may be relevant to a patient with COPD who has CHF as a patient characteristic (because a rapid increase in body weight may indicate an exacerbation of CHF, which may need to be distinguished from an exacerbation of COPD). MM3 might also be relevant to a COPD patient who has obesity as a significant patient characteristic regardless of whether the patient also has CHF but might not be relevant to a COPD patient of normal body weight who does not have CHF. PC4 in the table above might represent CHF; thus a T-plan for a patient with patient profile P1 (which includes PC4) would include MM3, as indicated. MM4, MM6, and MM7 might be monitoring modules for physiological variables or behaviors that are relevant to patients with COPD having particular patient characteristics or particular combination(s) of patient characteristics but that are not relevant in the absence of those characteristics or combinations of patient characteristics. For example, PC2 might be “smoker”, in which case a monitoring module pertaining to smoking behavior might be included in a T-plan for COPD. This might be represented as MM4, for example, which is included in a T-plan for COPD if PC2 is specified as a patient characteristic (as in the case of P1 and P2) but not otherwise.

Likewise, EM1 might be an educational module pertaining to COPD, which would be relevant to any patient with COPD. EM3, EM4, EMB, and EM9 might be educational modules containing educational materials that are relevant to patients with COPD having particular patient characteristics or particular combination(s) of patient characteristics but not relevant in the absence of those characteristics or combinations of patient characteristics. For example, if PC2 (“smoker”) is a characteristic of a patient with COPD, an educational module pertaining to smoking cessation might be included in a T-plan for COPD for that patient. This might be represented as EM3, for example, which is included in a T-plan for COPD if PC2 is specified as a patient characteristic but not otherwise.

HCEM1 represents a health care event module for COPD which might, for example, specify “chest X-ray”, “chest CT scan”, “pulmonary function test”, “emergency room visit”, and “hospitalization” as significant health care events and/or might include a treatment plan specifying patient events and physician events for management of COPD. HCEM1 might be a system-provided default health care event module for COPD or might be a version of a system-provided default health care event module for COPD that has modified by the prescribing physician and saved in that physicians' personal workbench. HCEM1 might have been modified for a particular patient by the health care provider. Modification by the physician may include adding or removing significant health care events, adding or removing physician events from a default treatment plan or altering their timeframes, or changing or removing default medications.

In some embodiments, the content and/or operation of at least some of the data collection modules may not be the same for all patients to whom such modules are assigned but rather may depend in part, for example, on which significant patient characteristics the particular patient has. For example, HTM1 includes an SST for COPD, and, as described above, the particular questions asked to a patient with patient profile P1, which does not include CHF as a patient characteristic, may differ from those asked to a patient with patient profile P2, which includes CHF as a patient characteristic.

In some embodiments, the system may configure T-plans using an algorithm similar to that described above in regard to SSTs, considering the significant patient characteristics as inputs. In some embodiments, the same type of algorithm may be used to configure portions of the modules, such as portions of the health care event module, e.g., treatment plan.

In some embodiments, a health care provider who prescribes an SST that includes rescue measures, e.g., rescue medication, as a possible course of action is asked to confirm that the particular rescue measure, e.g., rescue medication, is appropriate for the patient within the judgment of the health care provider. The prescriber may be asked to check a box or provide some other affirmative indication. In some embodiments, health care provider is reminded or requested to prescribe the rescue medication or is asked to confirm that the patient has received a prescription for the rescue medication. For example, FIG. 6E shows a medication confirmation pop-up that may be presented to a health care provider who has prescribed a T-plan for COPD to a patient who has “smoker” as a patient characteristic. The patient may receive a prescription for a rescue medication or may receive the rescue medication itself at the time of discharge.

After a T-plan is configured, a health care provider may be presented with a list or graphic representation of the modules to be included in the T-plan and the particular course of action and/or directions to the patient that would result from running an SST with particular health data or combinations of particular health data. In some embodiments, a prescriber may select any particular module and obtain details about its contents. For example, the various health data or combinations of health data that would result in an SST directing the patient to take a rescue medication, make an appointment with the health care provider, or seek emergency treatment may be displayed. The prescriber may review the information before approving the T-plan. In some embodiments, the various courses of action and/or directions that may be issued by an SST included in a T-plan are represented by displaying each course of action and/or corresponding directions along with the particular health data that would result in determining that course of action to be appropriate and providing the corresponding directions to the patient.

In the case of certain conditions, T-plans, T-plan modules, or their operation (e.g., the output of an SST) may vary depending on the grade, stage, class, or level of severity of the condition, which may be assessed using various classification schemes that are well known in the art. In some embodiments, a level of severity may be expressed as a grade, stage, class, or the like. For example, COPD may be classified into four stages according to the Global Initiative for Chronic Obstructive Lung Disease (GOLD) classification scheme. In some embodiments, different levels of severity of a condition may be considered to be distinct conditions. In some embodiments, the grade, stage, class, subtype, or level of severity of a condition is considered a significant patient characteristic for that condition.

In some embodiments, different grades, stages, classes, subtypes, forms, underlying etiologies, or levels of severity of a condition may be included in a T-plan category. For example, there may be T-plans for COPD GOLD1, COPD GOLD2, COPD GOLD3, and COPD GOLD4, which may all be included within a T-plan category entitled “COPD”. In some embodiments, a T-plan category may include only one member. In some embodiments, two or more conditions that are related in some way and/or share one or more features (e.g., clinical manifestation, etiology) in common may be included in a T-plan category. A T-plan category may correspond to a category or group of conditions that is recognized in clinical medicine. For example, Crohn's disease and ulcerative colitis may be included in a T-plan category entitled “inflammatory bowel disease”. It should be understood that T-plan categorization, if used, may differ in different health care disciplines. For example, “lung cancer” may be considered a T-plan category containing a single T-plan (lung cancer) in the context of pulmonary medicine but may be a category or supercategory that contains multiple distinct T-plans or T-plan categories in the context of oncology.

In some embodiments, T-plans, T-plan modules, or their operation (e.g., the output of an SST) may vary depending on the scenario in which the T-plan is to be used, e.g., whether for post-discharge use or chronic care use. In some embodiments, the scenario in which a T-plan for a condition is to be used may be considered a significant patient characteristic or may be handled in the same way as a significant patient characteristic in preparing a knowledge base. In some embodiments, post-discharge use for a given condition and chronic care use for that condition may be considered two distinct conditions. In some embodiments, a physician user interface allows a health care provider to select whether the T-plan will be used in the post-discharge or chronic care scenario. The particular scenario can be toggled from one to another through a simple check box or other GUI element. Switching from one scenario to the other may add or remove monitoring modules or alter their timeframe, may alter the operation of an SST or have one or more other effects.

In some embodiments, the REVON system provides basic T-plan templates for a variety of conditions, which may comprise a fixed set of basic data collection modules. In some embodiments, the system may provide a basic set of T-plan templates in any one or more health care disciplines, which may encompass at least the top 3 to 5 conditions most commonly treated by practitioners in that health care discipline. The basic T-plan templates may include a basic set of data collection modules that would be appropriate for any patient with the condition. In some embodiments, such basic T-plan templates may not include an SST. In some embodiments, such basic T-plan templates may include an SST only if the SST would produce acceptable evaluation and/or instructions for any patient without taking material patient characteristics into account. Basic T-plans of this type might be prescribed to a patient with a particular condition if the health care provider does not know which significant or material patient characteristics for that condition the particular patient has or if the particular condition does not have any significant or material patient characteristics or if the health care provider simply wants to rapidly prescribe a T-plan to the patient. A T-plan created using such a T-plan template may subsequently be changed to account for patient characteristics.

The system may rationalize the T-plan(s) of a particular patient across multiple data collection modules and, if the patient has multiple T-plans, across the multiple T-plans. Such rationalization may include methods for, for example, reducing redundant data collection, ensuring that data is collected in an efficient way, and integrating new T-plans into the existing framework for the patient. For example, when a new T-plan is prescribed to the patient, the system may update the “Am I OK” feature of the T-plan product for the patient to include questions about additional symptoms for which the newly prescribed T-plan includes a relevant SST.

If a patient has monitoring modules for the same type of data element in multiple T-plans, the data element may be obtained according to a timeframe that satisfies the requirements of the monitoring module that specifies the highest frequency for collecting the data element. This would typically constitute obtaining the data according to the timeframe for each monitoring module. For example, a patient with CHF might have a body weight monitor that specifies obtaining body weight daily, for purposes of facilitating detection of an incipient CHF exacerbation. The same patient might also have diabetes and might have a diabetes T-plan that specifies obtaining body weight weekly. The patient's body weight would be obtained daily, and the patient would not be asked twice to enter his or her weight on one day of the week. The system may maintain a global schedule for obtaining the various types of data elements specified by the patient's collective T-plans. The global schedule may specify the particular types of data elements to be obtained and a frequency for obtaining each one. The frequency would be the highest frequency specified for that type of data element across all of the patient's T-plans. When a new T-plan is prescribed or an existing T-plan is updated, the global schedule for the patient is modified to include any types of data element types specified in the newly prescribed T-plan that were not already being obtained, and adjust the frequency of data element types already in the global schedule if necessary to meet the requirements of the newly prescribed T-plan. If a T-plan is terminated, the global schedule may be updated to remove any data element types that were specified by that T-plan and not by any other T-plans prescribed to the patient or to adjust the frequency downwards if permitted by the remaining T-plans. In some embodiments, a T-plan may specify instances in which two or more data element types are linked such that they need to be obtained with a defined temporal relationship or must both be obtained within a defined time period (e.g., for use together). The global schedule may take such requirements into account. As an example, a global schedule for a particular patient may be represented as follows (where “q” stands for “each” and “d” stands for “day”): {Pt ID; blood pressure: q7d; body temperature: q1d; blood oxygen sat: q1d; body weight q7d}. If the patient is prescribed a T-plan that specifies that body weight is to be obtained daily, the frequency would be adjusted for that data element type. Similar global schedules may be maintained for medications and other patient events, procedures and other physician events.

Pulmonology T-Plan Collection

Without limiting the invention in any way, an exemplary set of T-plans for conditions that are commonly treated by pulmonologists, and certain components thereof (SSTs and monitoring modules), is described in Tables 2-6 below. These T-plans and components may be included in a workbench for pulmonologists. It should be understood that the contents of Tables 2-6 are merely exemplary. The particular sets of pulmonology T-plans, T-plan components (e.g., SSTs, monitors), significant patient characteristics, etc., in any given implementation may vary. It should be understood that a T-plan may contain additional modules described herein, e.g., educational modules, health care event module.

Table 2 presents a list of T-plans for 17 conditions that are commonly treated by pulmonology practitioners.

Table 3 presents a list of 10 types of physiological data element types, behavioral data element types, and/or monitoring devices that collect one or more such data element types, which are of use in the context of one or more pulmonology T-plans. Each such data element type or monitoring device (and/or the data element types collected by such a monitoring device) may correspond to a particular monitoring module. However, the way in which the monitoring module collects the relevant data element type(s) may vary depending, for example, on the monitoring devices available to the patient. It should be understood that at least some of these monitors may additionally be of use in T-plans for other conditions, which conditions may sometimes be treated by practitioners in different health care disciplines, such as cardiology or primary care. Furthermore, whether or not a given monitor is included in a particular pulmonology T-plan may depend on the particular significant patient characteristics of the patient to whom the T-plan is prescribed, as described herein.

Table 4 presents a list of 7 smart symptom trackers of use in the context of one or more of the pulmonology T-plans. It should be understood that at least some of these SSTs may additionally be of use in T-plans for other conditions, at least some of which may sometimes be treated by practitioners in different health care disciplines, such as cardiology or primary care. Certain monitors listed in Table 3 may trigger running of an SST upon detection of an abnormal value. For example, a COPD Exacerbation SST may be triggered by an abnormal value from monitor 1 (pulse oximeter).

Table 5 lists SSTs and monitoring modules that would be included in an automatically configured T-plan for each of the 17 listed T-plans. It should be noted that additional monitoring modules may be included, which may obtain data as needed by an SST. For example, a Body Temperature monitoring module may obtain data to be used as needed in running an SST for pneumonia.

Table 6 lists significant patient characteristics for COPD. It should be understood that at least some of these patient characteristics may additionally be significant patient characteristics in the context of one or more other conditions, at least some of which may sometimes be treated by practitioners in different health care disciplines, such as cardiology or primary care. Table 5 also shows, for each patient characteristic, the SSTs and monitoring modules for regular monitoring that would be included in an automatically configured COPD T-plan for a patient who had that particular patient characteristic. A health care provider could add or remove one or more SSTs or monitoring modules. As described above for Table 5, it should be noted that additional monitoring modules may be included, which may obtain data as needed by an SST.

TABLE 2
Pulmonology T-plan List

| T-Plan Category | T-Plan |
|---|---|
| COPD | GOLD 1 |
| | GOLD 2 |
| | GOLD 3 |
| | GOLD 4 |
| Asthma | Mild Intermittent |
| | Mild Persistent |
| | Moderate Persistent |
| | Severe Persistent |
| Pulmonary Embolus | Pulmonary Embolism |
| Lung Cancer | Lung Cancer |
| Pulmonary Fibrosis | Pulmonary Fibrosis |
| Interstitial Lung Disease | Interstitial Lung Disease |
| Sarcoidosis | Sarcoidosis |
| Pneumonia | Tuberculosis |
| | Atypical Mycobacterial |
| | Fungal |
| Obstructive Sleep Apnea | Obstructive Sleep Apnea |

TABLE 3
Monitoring Modules

| # | Body Monitor |
|---|---|
| 1 | Oxygen Saturation/Heart |
| | Rate (Pulse Oximeter) |
| 2 | Blood Pressure |
| 3 | Body Weight |
| 4 | Peak Flow (Peakflow Meter) |
| 5 | Distance Walked |
| 6 | Electronic Stethoscope |
| 7 | Positive Airway Pressure |
| 8 | Smoking Cessation |
| 9 | Salt Intake |
| 1o | Body Temperature |

TABLE 4
Smart Symptom Trackers

| # | SST |
|---|---|
| 1 | COPD Exacerbation |
| 2 | Asthma Exacerbation |
| 3 | CHF Exacerbation |
| 4 | Pulmonary Embolism |
| 5 | Pneumonia |
| 6 | Pulmonary Fibrosis Flare-up |
| 7 | Interstitial Lung Disease Exacerbation |

TABLE 5
T-plan Components

| | | | | | | | | | Smart Symptom | | | | | | |
|---|---|---|---|---|---|---|---|---|---|---|---|---|---|---|---|
| T-Plan | | Monitors | | | | | | | Trackers | | | | | | |
| Category | T-Plan | 1 | 2 | 3 | 4 | 5 | 6 | 7 | 1 | 2 | 3 | 4 | 5 | 6 | 7 |
| COPD | GOLD 1 | × | | | | × | | | × | | | | | | |
| | GOLD 2 | × | | | | × | | | × | | | | | | |
| | GOLD 3 | × | | | | × | | | × | | | | | | |
| | GOLD 4 | × | | | | × | | | × | | | | | | |
| Asthma | Mild | × | | | × | | | | | × | | | | | |
| | intermittent | | | | | | | | | | | | | | |
| | Mild persistent | × | | | × | | | | | × | | | | | |
| | Moderate | × | | | × | | | | | × | | | | | |
| | persistent | | | | | | | | | | | | | | |
| | Severe | × | | | × | | | | | × | | | | | |
| | Persistent | | | | | | | | | | | | | | |
| Pulmonary | Pulmonary | × | × | | | | | | | | | × | | | |
| Embolus | Embolism | | | | | | | | | | | | | | |
| Lung Cancer | Lung Cancer | × | | × | | | | | | | | | × | | |
| Pulmonary | Pulmonary | × | | × | | × | | | | | | | | × | |
| Fibrosis | Fibrosis | | | | | | | | | | | | | | |
| Interstitial | Interstitial Lung | × | | | | × | | | | | | | | | × |
| Lung Disease | Disease | | | | | | | | | | | | | | |
| Sarcoidosis | Sarcoidosis | × | | | | | | | | | | | × | | |
| Pneumonia | TB | × | | × | | | | | | | | | × | | |
| | Atypical | × | | × | | | | | | | | | × | | |
| | Mycobacterial | | | | | | | | | | | | | | |
| | Fungal | × | | × | | | | | | | | | × | | |
| Obstructive | Obstructive | | | × | | | | × | | | | | | | |
| Sleep Apnea | Sleep Apnea | | | | | | | | | | | | | | |

TABLE 6
Significant Patient Characteristics for COPD (Comorbidities)

| | | | Monitoring Modules (for |
|---|---|---|---|
| # | Patient Characteristic | SST | scheduled monitoring) |
| 1 | Asthma | 2 | 1, 4 |
| 2 | CHF | 3 | 1, 2, 3, 5, 9 |
| 3 | Coronary Disease | (may include one or more | (may include one or more |
| | | from cardiology) | from cardiology) |
| 4 | Diabetes | (may include one or more | (may include one or more |
| | | from endocrinology/metabolism | from endocrinology/metabolism |
| 5 | Obesity | N/A | 3 |
| 6 | Severely Immunocompromised | 5 | 3 |
| 7 | Sleep Apnea | N/A | 3, 7 |
| 8 | Smoker | N/A | 8 |

Physician Application

In some aspects, a computer program product of the present invention provides an application that allows a health care provider to design a T-plan for a condition, customize a T-plan as appropriate for a particular patient, prescribe a T-plan to a patient, modify a T-plan that has been previously designed and/or prescribed to a patient, view health data acquired according to a T-plan between a patient's visits to the health care provider, view analyses of such health data, and, in some embodiments, view T-plans that have been prescribed to the patient by other health care provider of the patient. The application may be a web-based application accessed via a web browser and may be used on personal computers, mobile devices, or both. The application provides a user interface through which a health care provider can interact with the application, perform any of a variety of activities, and use any of a variety of features relating to T-plans. In some embodiments, when a health care provider opens the REVON application, the health care provider may be presented with options such as accessing existing patient records of his or her patients (from which the health care provider can view health data collected according to the T-plans prescribed to the patient by the health care provider), configuring a new T-plan to be prescribed to a patient, or any of a variety of other tasks described herein.

In some embodiments, the REVON system allows health care providers to view a patient list, along with, in some embodiments, biographical information, graphical data regarding compliance, condition, patient photo (if available) and, in some embodiments in which the patient is an inpatient, an admission date and/or admission diagnoses. It should be understood that not all such data may be displayed on a single screen or for all patients. For example, a new patient may not have any available compliance data. In some embodiments a health care provider can search for or select a patient from a list (FIG. 6A) and view information about the patient such as a graphical representation of the patient's existing T-plans, health score, T-plan utilization score, compliance score, can prescribe one or more T-plans to the patient, e.g., by a “drag-and-drop” functionality (FIG. 6B). The health care provider can assign, change, or confirm patient characteristics by toggling on/off buttons for conditions relevant to the underlying condition for which he or she is prescribing or has prescribed a T-plan (FIG. 6C), view, add, or remove symptom trackers, monitoring modules, educational modules, and/or other modules of a T-plan (FIG. 6D). The health care provider can customize the T-plan for the particular patient by, for example, entering or selecting particular medications as rescue medications, where applicable. For example, if the T-plan includes administering a course of oral corticosteroids or an antibiotic as a rescue medication, the health care provider may enter a particular corticosteroid or antibiotic and specify the dose, dosing instructions, and the like. In some embodiments, the health care provider can customize treatment plan details by adding evaluation questions to an SST, adding monitoring modules for particular physiological data, and/or adding educational modules.

As described above, in some embodiments, the system provides one or more “default” T-plans. A health care provider may elect to use such T-plans if desired or modify them, subject to applicable policies of the practice, health care institution, or health care organization at which the health care provider works. A modified T-plan may be saved as a new T-plan template and prescribed to patients.

In some embodiments, a health care provider may have a collection of T-plans, which may be stored as files associated with the health care provider's user account. The health care provider may store any number of T-plans, optionally up to a system-imposed limit. The health care provider may name the T-plans as desired and may customize and prescribe them to his or her patients as desired. The set of T-plans and individual modules (e.g., SSTs) available to a physician may be referred to as the physician's “bench” or “workbench”. The physician can customize the bench by adding T-plans or modules (e.g., by modifying existing ones and saving them as new ones), or removing T-plans or modules. The bench may be displayed in a portion of a display screen. For example, it may be displayed in the lower portion of the screen during the process of prescribing a T-plan. The physician may drag icons representing a selected T-plan to a representation of the patient to initiate prescription of the T-plan to a patient.

In some embodiments a user interface of a physician application includes at least the following four screens:

A first screen (screen 1) provides a list of patients (see, e.g., FIG. 6A for an example). In some embodiments, e.g., for a health care provider treating outpatients, the listed patients on a given day will be those patients who are scheduled for an appointment that day. In some embodiments, e.g., for a health care provider treating inpatients, the listed patients will be those under the physician's care in a given health care facility. In some embodiments the health care provider may select a patient from the list, may search the list (e.g., by patient name, patient ID, etc.), and/or may search all patients. In some embodiments, the first screen will be the initial screen presented when the health care provider logs on or opens the physician application. In some embodiments, in addition to a patient list, the first screen displays at least part of a T-plan workbench for the particular health care discipline in which the health care provider practices (not shown on FIG. 6A). For example, T-plans of a pulmonology T-plan workbench would be displayed to a pulmonologist. The T-plan workbench may include system-provided T-plans, personal T-plans of that health care provider, or both. (Other parts of a complete T-plan workbench, e.g., individual modules available for use, may not be displayed on this first screen in at least some embodiments.) A health care provider may customize the bench by, for example, adding or removing T-plans. In some embodiments, on the first screen, the health care provider can prescribe an automatically configured T-plan simply by dragging an icon representing a particular T-plan to the name of the patient. In some embodiments, a health care provider can enroll a patient in the REVON system from screen 1 by entering the patient's email address in a box displayed on the first screen. Doing so will cause the system to send an email to the patient for completion of the registration process.

A second screen (screen 2) is a patient-specific screen (see, e.g., FIG. 6B for an example). In some embodiments, the first screen automatically changes to a second, patient-specific screen, when that particular patient checks in automatically for an appointment or when the health care provider selects that particular patient from the list on screen 1. Screen 2 displays basic biographical data about the patient and a dashboard showing health data gathered by the system pursuant to T-plans prescribed by that particular health care provider. (It will be understood that the dashboard may be blank until some data has been collected.) The patient biographical information and dashboard may be in an upper section of the screen. Elsewhere on screen 2, e.g., in the lower half, the T-plan workbench for that particular health care provider is displayed. Elsewhere on the screen 2, an icon or photo representing the patient may be displayed, e.g., in the middle third of the screen between the patient biographical information at the top and the T-plan workbench at the bottom. In some embodiments, the health care provider can prescribe a T-plan by dragging a representation of the T-plan to the representation of the patient. In some embodiments, the various T-plans assigned to a patient populate in a flower-like arrangement, wherein each T-plan is a “petal” of the flower. In some embodiments, clicking on a T-plan brings up a screen that displays components of that particular T-plan. In some embodiments, a health care provider can view the name and, in some embodiments, contact information, of a different health care provider who prescribed the T-plan. In some embodiments, the health care provider can contact the different health care provider by email directly within that screen. In some embodiments, the flower-like arrangement of T-plans may additionally be presented to a patient by the patient application. In some embodiments, the flower can populate with suggested T-plans based on known comorbidities of the patient or data input from peripheral data sources such as third party EMR-input. In some embodiments, such suggested T-plans are visually distinguishable from the patient's prescribed T-plans. For example, they may be labelled as “suggested” or the like, may have their name “grayed out”, may be lighter in color, or shaded, etc. In some embodiments, when a user (e.g., a health care provider or patient), clicks on a suggested T-plan, a list of health care providers who practice in the particular health care discipline that would typically treat patients afflicted by the T-plan condition of the suggested T-plan may be displayed. In some embodiments, the list is limited to those health care providers whose practice is located within a specified distance from the patient's home, is limited to REVON physicians, or both. In some embodiments, additional information, such as contact information of the health care provider or his or her health care facility (e.g., phone, email address), rating of the health care provider or his or her health care facility, whether the health care provider is accepting new patients, wait time for next appointment, or other relevant information may be displayed or accessible from the suggested T-plan. In some embodiments, REVON physicians may opt in or out of being included in such a physician list.

The third screen (screen 3) allows the health care provider, once having selected a T-plan to prescribe to the patient on screen 2, to select patient characteristics for automatic T-plan configuration. In some embodiments, screen 2 automatically changes to a screen 3 after the health care provider selects a T-plan on screen 2. Screen 3 displays the significant patient characteristics for that T-plan. In some embodiments, if the patient already has a patient profile in the system (e.g., because he or she already has a T-plan, possibly prescribed by a different health care provider), the significant patient characteristics that are already stored in the patient profile are indicated (e.g., as checked or colored boxes, as depicted on FIG. 6D). For example, if the patient profile indicates that the patient has diabetes, this may be indicated on screen 3. The health care provider can select the significant patient characteristics that he or she believes the patient to have. In some embodiments, once the health care provider has selected the patient characteristics, he or she may save the profile. Doing so may automatically bring up screen 4 or may bring up a question asking the health care provider whether he or she wishes to review the automatically configured T-plan. In some embodiments, there may be a button or other GUI element that the health care provider can select to start the automatic T-plan configuration process. The health care provider may opt to move to screen 4 or may be asked whether he or she wishes to view the T-plan.

The fourth screen (screen 4) displays a listing of components of the automatically configured T-plan (see FIG. 6D for an example). For example, it may display one or more SSTs, one or more monitoring modules, one or more educational modules, and/or one or more health care event modules (the latter not shown on FIG. 6D). In some embodiments, the health care provider can customize an automatically configured T-plan from screen 4 by, for example, adding or removing individual T-plan modules or T-plan components, which may be displayed or accessible from screen 4. In some embodiments, screen 4 may request particular information needed for an SST that is part of the T-plan, such as the name of a particular rescue medication. In some embodiments, screen 4 also provides access to a health care event module template for a health care provider to configure or modify a health care event module specifying the health care provider's treatment plan for the patient.

In some embodiments, health care providers can post T-plans or modules thereof, e.g., SSTs, in an online marketplace where they can be viewed by other health care providers, who may, in some embodiments, be able to prescribe such T-plans or modules and, in some embodiments, copy, download, and/or modify them. In some embodiments, a fee may be required for using (e.g., prescribing, copying, downloading, and/or modifying) a T-plan or module posted by another user. The individual or entity who posts a T-plan or module may receive part of the revenue derived from such T-plan.

In some embodiments, a system, e.g., through a T-plan, allows a health care provider to follow, in a condition-specific manner, at least the particular health data that are relevant to conditions that the health care provider manages in his or her patients. In some embodiments, a system allows a health care provider, through a T-plan, to be informed of the occurrence of condition-relevant events by accessing a T-plan and/or by receiving notifications via means such as email or text message. Such information may permit the health care provider to, e.g., assess patient compliance with treatment recommendations and become aware of condition-relevant events that occurred between patient visits. Health care providers may use the T-plan to review health data and significant health care events in advance of a patient visit, which may permit more effective use of time spent with the patient. In some embodiments, the system provides health care providers with access to health data pertaining to conditions managed by other health care providers who have prescribed T-plans to the patient. In some embodiments, a patient's health care providers may be able to contact each other (or the practice or health care institution at which another health care provider of the patient works) through the T-plan product. The T-plan product may thus serve as a communication platform to connect a patient's health care providers.

In some embodiments, a physician T-plan product displays a representation of a patient's T-plans as icons positioned around an icon or photo representing the patient, in a flower-like arrangement (see FIG. 6B in which 2 T-plans are shown—one on either side of the patient icon). By clicking on a particular T-plan, details of its contents and/or data collected pursuant to it may be viewed. A new T-plan may be assigned by dragging an icon representing the T-plan to an icon or photo representing the patient.

In some embodiments, the REVON system provides one or more dashboards for use by health care providers to review patient health data obtained pursuant to T-plans. For purposes of the present disclosure, a dashboard is a user interface that shows an at least partly graphical or schematic presentation of the current status and/or trends of one or more parameters or indicators. An indicator may, for example, be an indicator of the health or compliance of a particular patient or group of patients, an indicator of use of a system of the present invention or a component thereof by a particular user or group of users, symptom data, physiological data, or any information or statistic derived therefrom. A dashboard may be presented on a single screen, which may be the first screen that the health care provider is presented with upon selecting a particular patient from the patient list.

A dashboard may provide a summary of data pertaining to a patient, a group of patients (e.g., those that have a particular diagnosis or have been assigned a particular T-plan) and/or data pertaining to the health care provider's use of the REVON system. In some embodiments a dashboard may present a summary or average of aggregated data acquired over a period ranging from 1 week up to 1 year, or more, with respect to one or more data elements. In some aspects, it is envisioned that health care provider dashboards pertaining to an individual patient may be particularly useful to health care providers who treat that patient as an outpatient, e.g., “chronic care use” scenarios described further below. Data elements may include compliance data, symptom data, physiological data, or any other types of data described herein. The data to be displayed for a particular patient depends at least in part on the content of the patient's T-plans, e.g., the particular monitoring modules prescribed. For example, if the T-plan is for COPD, the patient's average or daily oxygen saturation level may be displayed. In some embodiments, data regarding significant health care events such as hospitalizations for the T-plan condition and/or other conditions may be presented.

A dashboard may facilitate rapid assimilation of data, recognition of new, recurring, or potential health problems, recognition of changes in a patient's health status, rapid making of informed decisions, and/or reduce the likelihood that important health data will be overlooked. In some embodiments, a dashboard may inform a health care provider in seconds how well a patient has been doing with respect to a condition over a time period of interest, such as the time period since the patient's last appointment. Patients may have dashboards available with which to view their health data.

A dashboard may represent data in a variety of ways. Any of a variety of information graphics, i.e., graphic visual representations of data or knowledge may be used. For example, images that resemble the meters and indicators typically found on a dashboard or instrument panel in a vehicle, such as a speedometer, fuel gauge, etc., may be used. Plots, charts (e.g., line charts, pie charts, bar charts), may be used. The particular type of graphic used for a given data element may be selected as appropriate for that item. It will be understood that certain data elements may be appropriately represented in a variety of ways. In some embodiments, a dashboard provides one or more links that allow a user to obtain more detailed information regarding the data presented in a particular graphic on the dashboard, i.e., to “drill down” into one or more of the graphics. In certain embodiments, a graphic may represent progress towards (or away from) a particular goal. For example, a graphic pertaining to patient who has been advised to lose weight may show a target weight and a timeline for reaching the target weight. In some embodiments, the graphical display for at least a portion of a dashboard may utilize D3.js (sometimes called D3 for Data-Driven Documents), a JavaScript library of use to drive the creation and control of dynamic and interactive graphical forms that run in web browsers from digital data.

In some embodiments, the system may support electronic prescription generation by health care providers for medications, medical devices, or other treatments that an SST may instruct a patient to use or that may be included in a treatment plan specified by an SST. In some embodiments, such prescriptions may be generated automatically, without additional physician input beyond prescribing the T-plan. In some embodiments, such prescriptions may be generated automatically but require additional physician input (e.g., confirmation), in order for them to become effective and/or be electronically transmitted to a pharmacy. In some embodiments, an electronic prescription feature includes determining whether a particular medication, medical device, monitoring device, or other item that a health care provider wishes to prescribe (e.g., as a rescue medication/rescue measure in an SST) is covered by a patient's health insurance policy or other policy or plan that provides for reimbursement for health care services and health care products. In some embodiments, if the particular medication, medical device, monitoring device, or other item is not covered, the system may suggest an alternative. In some embodiments, if the particular medication, medical device, monitoring device, or other item requires prior authorization, the system may inform the health care provider, may automatically submit the required documentation for prior authorization, or both.

Patient Engagement

In some aspects, a patient engagement program may be provided as part of a T-plan product. In some embodiments, patient engagement includes the input of data by the patient and/or other use of the patient application by the patient. In some embodiments, a patient engagement program generally includes elements that are intended to increase the level of a patient's cognitive, emotional, and behavioral investment in interactions with the T-plan. A patient engagement program may stimulate repeated interactions between the patient and the T-plan that strengthen the psychological or physical investment a patient has in the T-plan. In general, it may strive to create positive experiences with the T-plan that strengthen that investment and motivate the patient to become further engaged. A patient engagement program may, for example, aim to have the patient maintain healthy habits or improve habits based on feedback from monitoring modules and/or based on information in educational modules, read or listen to educational modules, respond to educational modules, apply information in educational modules, or perform activities as directed by educational modules. A patient engagement program may promote patient satisfaction, loyalty, and/or desire to tell others about their positive experiences with the T-plan product. Elements of a patient engagement program may provide patients with the ability to create and/or participate in online communities, provide patients with ways to communicate, create, and/or share content relating to health with each other online, provide patients with incentives to do so, provide patients with means to submit suggestions about the T-plan product, provide a blog, chat room, online forum, or other means by which users can communicate with each other and/or with developers of the T-plan product. Thus, in some embodiments, patients may contribute to refinements or new releases of the T-plan product. Online communities may comprise or consist of patients that have the same condition, patients that have one or more patient characteristics in common, patients that live in a particular geographic region, or other categories. A patient engagement program may include games, quizzes, contests, and other means by which patients may interact with the system and/or with each other. A patient engagement program may include segments or episodes that change daily and, in some embodiments, tell a compelling story to keep patients interested and motivated to keep returning. Content provided by a patient engagement program may be related to health care, unrelated to health care, or both.

In some embodiments, the patient engagement program for a particular patient is individualized based at least in part on one or more patient characteristics and/or based on analyzing patient interactions with the system. The system may analyze a patient's characteristics and select a patient engagement program from a library of patient engagement programs or may generate a patient engagement program from a library of patient engagement elements. In some embodiments, the system collects data on patient engagement metrics and uses the information to improve the patient engagement program. Patient engagement metrics can include any measurement that reflects patient use of a T-plan or portion thereof, such as measurements of the number of times the patient uses the T-plan or a portion thereof, average length of time spent viewing an educational item, or the like.

In certain embodiments, the system provides means by which health care providers, health care institutions, or health care organizations can provide their own patient engagement elements or programs for use in T-plans prescribed to their patients. In certain embodiments, a patient engagement program may include one or more elements that promote patient engagement with a health care provider or health care institution or organization in addition to one or more elements that promote patient engagement with the T-plan.

Health Score, Compliance Score, Utilization Score

In some embodiments, one or more scores may be generated based on data obtained according to a T-plan assigned to a patient. In general, a score provides a quantitative assessment of something, such as the patient's health, compliance with one or more elements of a treatment plan, or utilization of one or more patient-directed features associated with a T-plan. Scores may be calculated based on data specified by a single T-plan or based on data specified by multiple T-plans. For example, individual health scores may be generated for each condition for which a patient has an active T-plan; individual compliance scores may be generated for treatment plans in each T-plan; individual utilization scores may be generated for each T-plan. Each individual score would be based on data specified by a single T-plan. Composite health scores, compliance scores, and/or utilization scores may alternately or additionally be generated based on data collectively specified by two or more of a patient's active T-plans, e.g., all of the patient's active T-plans.

A score may be generated using any of a variety of methods. Different implementations may use different methods to compute a score, and it should be understood that the invention is not limited in this respect. The system may provide one or more predetermined methods to compute a health score, utilization score, and/or compliance score.

A health score may be based on symptom data, physiological data, or a combination thereof. A health score may reflect health status at a particular point in time or may reflect health status over a period of time, such as a week, a month, or any time interval of interest. In general, multiple types of data elements may be considered in generating a health score. The particular data elements used to generate a health score will typically vary depending on the condition. Different types of data elements may be assigned different weights. In general, a method that generates health score that correlates in some reasonable way to the health status of the patient as would be judged by a person of ordinary skill in the art may be used. In some embodiments a health score may be adjusted for demographic parameters such as age, sex, or the like. A health score may be relative to normal subjects (optionally adjusted for demographic parameters), subjects with the same condition, or the subject's own previous health status. In some embodiments a health score may provide an indication of the patient's improvement or decline in health status over time.

A utilization score may indicate the extent to which the patient ran a health tracker voluntarily or when prompted, the patient's use of educational modules voluntarily or when prompted, etc. A utilization score typically reflects utilization over a period of time, such as a week, a month, or any time interval of interest. A utilization score might indicate, for example, the average number of minutes per week that the patient spent using educational modules. In general, a method that generates a utilization score that correlates in some reasonable way to the utilization by the patient of one or more patient-directed features associated with a T-plan may be used.

A compliance score may indicate the extent to which the patient performed one or more actions specified by patient events in a treatment plan, such as taking medications according to the specified timeframe for taking such medications, making and keeping appointments for follow-up visits according to the specified timeframe for such visits, etc. There may be individual compliance scores for different elements of a treatment plan, e.g., medications, follow-up appointments. In general, a method that generates a compliance score that correlates in some reasonable way to the compliance by the patient with one or more elements of a treatment plan associated with a T-plan may be used.

A score may comprise a numerical score, percentage, percentile, letter score, graphical representation, or the like. For example, a percentage score might be “Medication compliance: 95%”, indicating that the patient took the appropriate medication within the appropriate timeframe 95% of the time. A numerical score might be normalized, e.g., to range between 0 and 100.

One or more scores may be presented to a patient, health care provider(s) of the patient, or other users of the system. A score may be presented in any of a variety of ways, e.g., numerically or graphically. In some embodiments, a score is presented in a graphical format showing changes over time. Occurrence of hospitalizations or other significant health care events may be indicated on a graphical representation of a score.

In some embodiments, patients may be compared to their fellow patients (e.g., all patients in the network or patients in the network who have the same disease) and ranked for health, compliance, or utilization with their physicians' recommendations, e.g., using a percentile system. In some embodiments, patients may be stratified, e.g., according to the severity of their condition and/or the complexity of their physician's recommendations so that, for example, patients who have a complicated medication regimen are not compared with patients who have a much simpler medication regimen. Patients may be informed of their ranking on an overall or condition-specific basis.

Scores may serve any of a variety of purposes, of which several non-limiting examples are now described. A health score may provide a health care provider with a rapid means of ascertaining which of his or her patients are at significant risk of deteriorating, have deteriorated over a time period of interest (e.g., since their previous appointment), and/or are in need of additional or urgent attention. In some embodiments, health care providers may be able to view a list of patients ranked according to health score. A health care provider may view the list when desired, e.g., daily, weekly, and may, if desired, contact patients whose scores are low or have fallen significantly over a given time period. Compliance scores may be useful for counseling patients and/or assessing efficacy of treatment. For example, a patient with a high compliance score but a poor response to treatment may benefit from a change in medication. A patient with a low compliance score may benefit from the health care provider identifying and attempting to address the reasons underlying the poor compliance. Patients may view their scores to get an overall idea of their health status with regard to one or more conditions and/or how they compare with other patients with the same condition(s). Knowing that a compliance score is being provided to a patient's physician may encourage the patient to maintain a high compliance score. In some embodiments a patient may be provided with suggestions as to how they may improve one or more of their score(s). A compliance score may be useful to individuals and entities that perform or sponsor clinical trials. For example, such individuals and entities may wish to enroll patients with a high compliance score in order to be able to more reliably determine the safety and/or efficacy of an experimental therapy. A utilization score may be useful for purposes of assessing and improving the effectiveness of patient-directed features of a T-plan product.

Caregiver Features

In some embodiments, a caregiver plan may be assigned to a caregiver of a patient in conjunction with a T-plan prescribed to the patient. Such plan may be provided by a caregiver module. A caregiver plan may, in some embodiments, be assigned only with appropriate authorization by the patient or the patient's guardian or legal representative. Caregiver plans may differ depending on the particular relationship between the caregiver and the patient. For example, a caregiver plan for a caregiver for whom caregiving is a job may differ from a caregiver plan for a family member. A caregiver plan may have modules that are counterparts of any of the data collection modules described above, e.g., health tracking module, monitoring module, education module, health care event module, and a caregiver user interface. Features associated with a caregiver plan may be used through various channels through which a patient may use features associated with a T-plan. For example, a caregiver may use such features through a user interface on a mobile device or personal computer. A caregiver may download an application for his or her mobile device that provides the features associated with a caregiver plan.

In some embodiments, a caregiver counterpart of a module of a T-plan differs from the corresponding patient module so as to make the caregiver counterpart more suitable for use by a caregiver. For example, a caregiver counterpart of a health tracking module may include one or more symptom trackers, e.g., one or more SSTs, with questions about patient symptoms, but instead of directing the questions to the patient, the questions would be directed to the caregiver and expressed as questions about the patient. For example, a caregiver may be asked “Does your patient seem more short of breath than usual?” or may be instructed to “Please ask your patient if he or she is more short of breath than usual”. A caregiver counterpart of a monitoring module may instruct a caregiver to obtain a particular physiological data element, e.g., “Please enter your patient's blood pressure”. Such questions or instructions may be customized to include the patient's name or relationship to the caregiver, e.g., “Please enter Ms. Jones' blood pressure.” or “Please enter your mother's blood pressure.” Data obtained by such modules would be stored in the patient record of the particular patient. In some embodiments, the caregiver may be presented with directions for management of the condition in the patient by the caregiver. For example, the caregiver may be instructed to administer a rescue medication or seek medical attention for the patient. Any of the triage directions or management directions provided by an SST, as discussed above, may be provided to a caregiver instead of or in addition to a patient. Thus, wherever the present disclosure refers to providing directions to a patient (or similar expressions), it should be understood that such directions or a version thereof may be provided to a caregiver instead of, or in addition to, being provided to the patient.

A caregiver educational module may provide educational materials that teach the caregiver about the condition, teach various skills such as changing dressings, or teach the caregiver various warning signs that the caregiver should watch for that might warrant seeking medical attention for the patient. Data regarding the caregiver's use of the educational module may be collected.

A caregiver health care event module may provide a schedule of patient events and/or physician events for use by a caregiver who may, for example, administer medications to the patient, arrange appointments, provide transportation, or otherwise facilitate a patient's compliance with a treatment plan. A caregiver may be asked to confirm the occurrence of certain patient events such as administration of medications. Such data may be stored in the patient record.

Caregivers may receive notifications, e.g., reminders. In general, caregivers may receive any of the various types of notifications described herein as being issued to a patient. The content of the notifications would generally be expressed as notifications about the health status of the patient or notifications about things that the caregiver should do for or regarding the patient. For example, a caregiver may be reminded to perform an action associated with a patient event, such as administering a medication. Notifications may be issued based on data obtained by data collection modules of the patient's T-plan, the caregiver plan, or both.

A caregiver engagement program may have any of the features of a patient engagement program described above. For example, a caregiver engagement program may provide for online communities of caregivers. Such communities may comprise or consist of caregivers of patients that have the same condition, caregivers of patients that have one or more patient characteristics in common, caregivers of patients that live in a particular geographic region, caregivers who are family members of patients, caregivers who are spouses of patients, caregivers who are children of patients, caregivers that are employed as caregivers, or may be segmented into any other types of categories.

Remote Caregiver Features

In some embodiments, a T-plan is associated with features relating to remote caregivers. Such features may be provided by a remote caregiver module. A remote caregiver module may, in some embodiments, be assigned only with appropriate authorization by the patient or the patient's guardian or legal representative who may, for example, provide the remote caregiver's email address, mobile phone number, or other information that allows for communication with the remote caregiver. A remote caregiver module may provide remote caregivers with at least partial access to any one or more features associated with a patient's T-plan, such as a patient dashboard. In some embodiments, a remote caregiver module may provide updates to a remote caregiver on a regular basis, e.g., daily or weekly, or upon request by the remote caregiver. The updates may summarize the patient's health status and/or upcoming health management events, or provide any of a variety of other types of information. In some embodiments, a remote caregiver module may provide notifications of various types to the remote caregiver based on data specified in the patient's T-plan or collected in association with a patient's T-plan. For example, a remote caregiver may be notified if a patient experiences an adverse change, receives directions from a health tracking module to seek medical attention (e.g., emergency medical attention), visits a hospital, is hospitalized. A remote caregiver module may integrate information across multiple T-plans or may be limited to information pertaining to a single T-plan. An individual patient or T-plan may have one or more remote caregiver modules associated therewith. For example, there may be a remote caregiver module for each remote caregiver. A remote caregiver may access the remote caregiver features on a mobile device or through a personal computer.

Communication Features

In some embodiments, a T-plan product provides features by which patients may communicate with their health care providers and/or by which health care providers can communicate with patients in addition to the questions asked automatically by a health tracker. Such communication may include making appointments online or sending messages by email, by text message, by phone, and the like.

In some embodiments, a T-plan product provides means by which caregivers may communicate with each other, with the patient, and/or with a patient's health care providers. Such communication may be used for any of a variety of purposes, such as scheduling of caregiver visits to the patient, coordination among different caregivers, scheduling appointments with health care providers, or the like.

In some embodiments, a T-plan product provides means by which a remote caregiver who has been assigned a remote caregiver module can communicate with the patient. For example, the remote caregiver may be able to send messages that would be displayed to the patient. Such messages may be presented on a screen within a patient T-plan app on a mobile device. The patient may be able to enter responses that are transmitted to the remote caregiver. In some embodiments, a remote caregiver can set automatic messages to be transmitted to the patient.

In some embodiments, a T-plan product provides means by which remote caregivers may communicate with each other, with caregivers who provide physical assistance to the patient, and/or with a patient's health care providers.

User Accounts

In some embodiments, a user may have a user account on a system of the present invention. A user account typically allows a user to authenticate to a system and be granted authorization to access one or more services or functions provided by the system. To log into an account, a user is typically required to authenticate himself or herself with a password, biometric identifier, or other credentials for, e.g., accounting, security, logging, and/or resource management purposes. In some embodiments, two-factor authentication is used. A user account may have a home directory, which may store files pertaining specifically to that user's activities (e.g., files created through the user's interactions with the system), which is protected from access by other users (though certain users or categories of users such as system administrators may have or be able to gain access). The process of obtaining a user account may be referred to as “registration” or “enrollment”. A user may obtain an account in any of a variety of ways. It will be understood that these processes and implementations may vary. For example, a user may download an application for a mobile device or may access an application via a web browser that allows the user to enter data required to obtain an account. A user may be sent an email inviting the user to provide at least the required data, which may be entered within the email (e.g., in boxes or fields) or may be entered in a form that may be accessed from a link in the email. In some embodiments, a health care provider or associated personnel may enter a patient's email address through a physician user interface, and the system then sends an email to the patient. The email may contain a link that the patient may use to download or access a patient application or receive directions on how to download or access a patient application or may contain directions on how to download or access a patient application. The email may ask the patient to enter appropriate information to obtain an account. A user may visit a website that provides a form to be completed to obtain an account. A link to the website may be provided in the afore-mentioned email or the patient may be informed of or become aware of the website by other means.

Different categories of users may have different types of account. Different account types may provide different levels of access and different privileges to use various features of the system. For example, health care provider accounts may permit a health care provider to use at least those features and functions pertaining to the prescription of treatment plans as described herein and such other activities as health care providers may perform with regard to treatment plans as described herein. The particular access rights may vary depending at least in part on the type of user. In some embodiments, a health care provider account allows a health care provider to access records in the system, or portions thereof, that contain health data from his or her current patients and associate treatment plans with those patient's records. In some embodiments, patient authorization may be required prior to a health care provider being given access to at least some of the health data pertaining to the patient. In some embodiments, a health care provider may have access to only certain portions of the health data pertaining to at least some of their patients. For example, a first health care provider may only be able to access treatment plans prescribed by that provider and health data obtained by those treatment plans. Access by the first health care provider to treatment plans prescribed by other health care providers and health data obtained by such treatment plans (to the extent not also acquired by treatment plans prescribed by the first health care provider) may require patient authorization. Patient accounts may permit a patient to use at least those features and functions pertaining to the use of treatment plans that have been prescribed to them as described herein and such other activities as patients may perform with regard to treatment plans as described herein.

In general, a user typically supplies various data elements to obtain an account. The required and/or requested data may vary. Different types of users may be required or requested to provide different data elements to obtain an account. For example, a patient may be required to provide at least his or her name. Other data elements that may be requested or required from a patient user may include at least a portion of the patient's social security number, date of birth, name(s) of at least one of the patient's current health care provider(s), and/or email address. An individual attempting to obtain an account as a physician account may be required to provide the physician's name and a phone number. In some embodiments, the system may access a list of licensed physicians and their National Provider Identifier (NPI) or equivalent sufficient to verify that the name provided by the individual is that of a licensed health care provider. NPI numbers may be obtained, e.g., from the NPI Registry (https://nppes.cms.hhs.gov/NPPES/NPIRegistryHome.do). The individual may be phoned at the number provided in the account request to confirm the individual's identity, i.e., that the individual who requested the account is indeed the physician. Other types of users, e.g., individuals who wish to use the system for research, clinical trial recruitment, or various other purposes may receive accounts differently.

In some embodiments, health care providers, e.g., physicians, may be required to provide their license number, DEA number or other prescriber number, employer name or hospital affiliation (if applicable), mobile phone number, business address, and/or email address. In some embodiments health care provider names and/or other identifying information may be provided by health care institutions or organizations that employ or use the services of such health care providers.

Scenarios for Physician and Patient Acquisition and Use of T-Plan Product

As described above, in some aspects, the REVON system allows health care providers, e.g., physicians, nurses, and other health care providers, to prescribe T-plans to their patients. Health care providers will typically have established accounts with the REVON system, which may entail providing information such as medical license or registration number, prescriber number, or other relevant information, selecting user name and password, and other standard account setup information. This may be done in a standard manner, e.g., through a secure website which may provide a web form with appropriate fields to be completed. In some embodiments, a health care institution, organization, or physician practice that decides to use the REVON system manages at least part of the account setup process on behalf of health care providers and/or associated personnel who may use the system in the course of their work. Without limiting the invention in any way, it is envisioned that health care providers may prescribe T-plans for use in two main scenarios, referred to herein as “post-discharge use” and “outpatient use”. The latter may also be referred to as “chronic care use”.

In some embodiments, a physician practice, health care institution, or health care organization may approve one or more T-plans for use as a standard measure for its patients who are afflicted with the conditions addressed by those T-plans. For example, if a health care institution, health care organization, or physician practice decides to use the REVON system, the appropriate personnel of the institution, organization, or practice may review the preconfigured T-plans provided by the REVON system for one or more conditions of interest and may propose changes to the diagnostic units, inputs to an SST, instructions, default medications, or other items as may be desired, e.g., to conform the T-plan with applicable health care provider protocols, workflows, or preferences. Appropriate changes may be made to the instance of the T-plan configuration algorithm and/or to one or more modules of a T-plan to be used by the health care institution, organization, or practice. In some embodiments, at least partial integration of the REVON system with the EMR system used by health care institution, organization, or practice may be performed, e.g., so as to enable the EMR system to populate certain patient information in the REVON system automatically and/or to identify patients with those conditions for which the institution, organization, or practice uses T-plans for enrollment in the REVON system.

In the post-discharge use scenario, according to some embodiments, a health care provider prescribes a T-plan to an inpatient or to a patient who has recently been discharged. The T-plan may be for use during the early post-discharge time period, e.g., during the first 30 days after discharge, or up to about 60 to 90 days after discharge. Such T-plans may be designed to assist patients in following discharge instructions that may also be given to the patient orally or in writing at or around the time of discharge, may allow patients to evaluate their symptoms periodically when prompted by the system or whenever desired by the patient, and provide appropriate directions to the patient based on the evaluations, as described above. In some embodiments, a post-discharge T-plan may assist with a patient's transition to outpatient care by, for example, reminding the patient to make or keep an appointment with his or her outpatient health care provider, asking the patient whether he or she needs transportation to the appointment, or reminding a caregiver of a patient appointment. These functions may be handled by a schedule module as described above.

In the case of a T-plan for post-discharge use, the patient will typically be prescribed a T-plan for a condition that is present at discharge, typically one that is indicated in a discharge note or discharge summary. Often the condition is one that resulted in the admission, although it may be a different condition for which the patient is under treatment while admitted. In some embodiments, patients who have a condition for which a T-plan is available are identified at or after admission. This may occur automatically through an EMR system, e.g., when an admission diagnosis is entered or based on patient history, or may be entered by personnel working at the health care facility, e.g., personnel such as the admitting physician who are involved in admitting the patient. Patient biographical information may be entered into the REVON system at that time by personnel or by the EMR system or updated as appropriate if the patient is already enrolled with the REVON system.

A T-plan for post-discharge use may be prescribed at any time during the patient's hospital stay or shortly thereafter (e.g., within up to about 1 to 10 days after discharge). For example, the admission process may include a T-plan prescription or a health care provider who is authorized according to the health care institution's policies to prescribe a T-plan for the patient may do so. For example, a T-plan may be prescribed by a hospitalist or other physician who has overall responsibility for the patient's care. In some embodiments, a patient may already have a T-plan for the condition for chronic care use. The T-plan may be modified by replacing one or more modules with a module appropriate for post-discharge use.

In some embodiments, a patient may be introduced to the T-plan product as part of preparations for discharge, e.g., 1-3 days prior to a planned discharge date or on the day of discharge, by a member of the patient's health care team or any personnel tasked with doing so. The patient may be assisted in downloading the app or establishing an account via the web. The patient's preferred interaction mode and other settings may be entered during this time, and the patient may experiment with use of the product. A video demonstrating use of the program may be provided for the patient to view on the television or on a mobile device in his or her room. The discharge nurse or other personnel involved in the discharge process may check the T-plan to make sure that the patient's characteristics and medications (e.g., as indicated in the discharge summary or patient's EMR) are reflected properly in the T-plan and may make any changes indicated by the physician to the patient's T-plan. The discharge personnel may demonstrate the use of the T-plan product and associated monitoring devices to the patient and any caregivers.

After discharge, the patient uses the T-plan product as described herein, e.g., to run SSTs. If the patient's outpatient health care provider responsible for managing the T-plan condition after discharge of the patient uses the REVON system as part of his or her practice, that health care provider may be given access to patient health care data and/or may be issued notifications upon occurrence of certain events such as detection of an exacerbation, issuance of a management instruction to call 911, or readmission of the patient.

In some embodiments, upon discharge from the hospital or upon enrollment in the chronic care context, a patient receives: (1) (A) a download of an application that provides features for patients as described herein onto his/her smartphone or other mobile device; or (B) a smartphone or other mobile device with the patient application already installed; or (C) instructions for using one or more features over a basic phone, optionally together with (A) or (B); and (2) either: (A) prescriptions for his or her rescue medications as specified by health tracking modules prescribed to the patient; or (B) rescue medications filled by the hospital pharmacy or provided at the physician's office; (C) in some embodiments, a pamphlet or other material with instructions regarding use of the REVON system; and (3) in some embodiments, one or more monitoring devices, which may be enabled for wireless synchronization, may be pre-synced with the mobile device, and/or may be approved by the FDA or other applicable regulatory agency responsible for regulating devices of that type. In some embodiments, at least partial integration of a system of the invention with an EMR system used at the hospital or practice may be performed, and data in the EMR system is extracted and used to automatically populate at least some items of a patient record in the REVON system and/or used by the REVON system to identify patients admitted for or diagnosed with selected conditions.

In some embodiments, a T-plan for post-discharge use, or a T-plan module or portion thereof for post-discharge use, may be prescribed by a health care provider who manages the patient's chronic care for the condition and may have already prescribed a T-plan for chronic care use to the patient. In some embodiments, the T-plan may have multiple modes, such as a post-discharge mode and a chronic care mode. The post-discharge mode may include switching monitoring modules to a Heightened Sensitivity Mode as described above.

In some embodiments, the REVON system may detect that the patient may be hospitalized by, for example, detecting the location of the patient's mobile device, detecting a sudden change in the patient's pattern of use of the T-plan product, asking the patient via the patient's mobile device, or other means and may notify the health care provider. The system may also detect when the patient has been discharged and notify the health care provider. The health care provider may, optionally after confirmed that the patient was hospitalized for the T-plan condition (e.g., by contacting the patient), prescribe a T-plan for post-discharge use or a T-plan module or component thereof for post-discharge use, for that condition to the patient. Thus a T-plan for post-discharge use may, in some embodiments, be prescribed without involvement of the health care providers who are responsible for the patient's care only while the patient is hospitalized. In some embodiments, the system automatically changes the T-plan to a post-discharge mode if a hospitalization or likely hospitalization is detected by the system. In some embodiments, the post-discharge mode comprises or consists of switching all monitoring modules capable of operating in Heightened Sensitivity Mode into this mode.

In the chronic care use scenario, it is envisioned that a patient who visits a health care provider who uses the REVON system in an outpatient setting such as a physician practice or clinic may in some embodiments first access the REVON system and/or download a REVON app while in a waiting area prior to an appointment. The patient may be one who is visiting that health care provider for the first time or a patient who is already a patient of the health care provider. The patient may download the REVON app on his or her mobile device or may access the REVON system from a device already located in the waiting room and establish an account. Waiting rooms at physician practices or clinics may be supplied with such devices, on which patients can register with the REVON system. Nurses or associated personnel at the physician practice or clinic may instruct the patient about the REVON system and may assist the patient with downloading the REVON app and/or establishing accounts.

In some embodiments, a patient who visits a health care provider who uses the REVON system in an outpatient setting such as a physician practice or clinic or a hospitalized patient may be asked (e.g., by a health care provider or associated personnel) whether he or she would like to use the REVON system. If the patient responds affirmatively, the health care provider or associated personnel may enter the patient's email address into an appropriate field in the physician user interface. The patient may be sent an email inviting him or her to provide at least the required data, which may be entered within the email (e.g., in boxes or fields) or may be entered in a form that may be accessed from a link in the email. The REVON system requests entry of appropriate data for patient registration, which will typically include at least the patient's name and may include the name or other identifying information of the physician. The patient may upload a photo of himself or herself. In some embodiments, the patient may be guided through entering a medical history, such as that which patients are frequently asked to fill out when visiting a health care provider for the first time. The REVON application may permit the patient to print, email, or otherwise produce or provide a hard copy or electronic copy of the medical history to another individual, e.g., a health care provider.

When the patient sees the physician, the physician may prescribe a T-plan to the patient either during the visit or shortly thereafter (e.g., within up to about 1 to 10 days after discharge) or at any subsequent time. The T-plan functionality for patients (e.g., ability to run SSTs and view health data) is made available to the patient via computer, as an application on the patient's mobile device, or through other means as described herein.

Check-In Feature

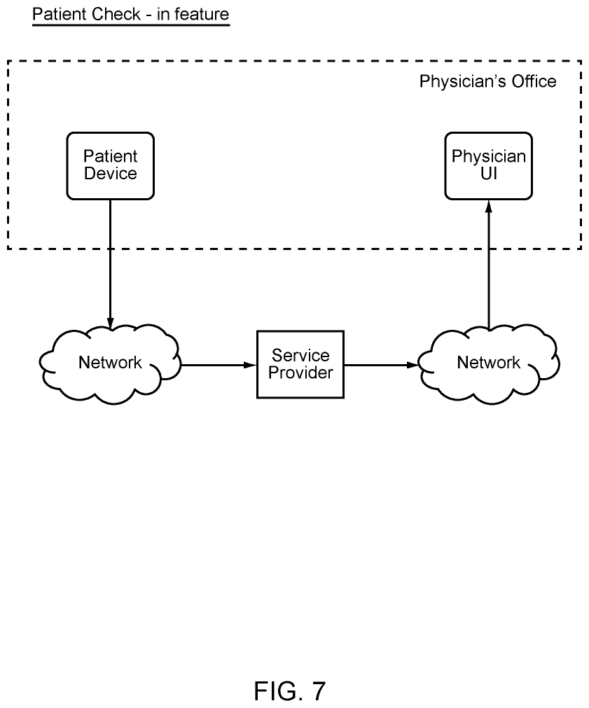

In some aspects, the system provides a computer-implemented check-in process that allows patients to automatically notify physicians of their arrival in the physician's office. FIG. 7 is a schematic depiction of a check-in process according to certain embodiments. As depicted in FIG. 7, when a patient enters a physician office with his or her mobile device, the patient's device sends a signal over the network to the REVON system that it has entered the physician office. The REVON system then sends a notification to the physician that the patient is in the office, which notification becomes visible on the physician's user interface. In some embodiments, on arrival at the physician's office, the patient will access the REVON application on his or her device or on a device that is located in a waiting area, and with a few clicks (or, in some embodiments, without requiring any action on the part of the patient other than, in some embodiments, accessing the REVON application on his or her mobile device), check in at the physician's office. In turn, a notification will appear on a computer screen within the REVON application for the selected physician, notifying him/her that the patient has arrived. In some embodiments, if the patient is already registered with the system, the check-in may ask the patient to select the current appointment or physician (e.g., from a list of the patient's appointments for that day and/or from a list of physicians who have assigned T-plans to the patient), and then select “check in”. In some embodiments, the system uses location awareness based on the location of the patient's mobile device (e.g., using any of a variety of mobile device tracking methods and/or apps) and automatically brings up the current appointment or physician for the patient to check in (rather than requiring the patient to select an appointment or physician) or automatically checks the patient in without action by the patient. The location of the patient's mobile device may be compared with the known locations of the office(s) of the patient's physicians in order to determine that the patient is located at a particular physician's office and which physician the patient is there to see. “Physician's office” is used in a broad sense to refer to a physician's workplace, which may be anywhere that a physician sees patients for purposes of providing health care. The patient may check in any time upon arrival at the physician's workplace. In some embodiments, associated personnel at the physician's office are notified of the patient's arrival through the check-in process in addition to or instead of the physician.

Physician Viewing of Patient Data on Mobile Device

In some aspects, the system provides a computer-implemented method whereby when a patient phones a physician or sends an electronic communication such as an email to the physician (e.g., from the patient's mobile device), an application on the physician's mobile device automatically detects which patient is calling, accesses data from the patient's T-plan and/or from the patient's EMR and displays it on the screen, or provides a button on the screen that the physician can tap to access the data. Thus, the physician can see the patient's health data immediately, without having to search for it. The data may be automatically displayed for the physician on the physician's mobile device when the physician answers the phone call, or may be made available for immediate access when the physician opens the electronic communication. In some embodiments, an external service (e.g., a mobile carrier) provides incoming call information to the app. In some embodiments, the app has access to incoming call data directly from the device. In some embodiments, the device provides incoming call information to the app. The app uses the incoming call information to determine which patient is calling based on the patient's phone number having been provided by the patient (e.g., during registration) and stored by the system. In the case where the electronic communication is an email, the patient's email address is used to determine which patient the email is from, based on the patient's email address having been provided by the patient (e.g., during registration).

Data Sources and Data Integration

Data sources used by aspects of the present invention to obtain data may include patients, health care providers, and connected monitoring devices. For example, symptom data may be obtained from the patient by asking questions through a user interface; physiological data, behavioral data, and/or environmental data may also be obtained in this manner or via a connected monitoring device in various embodiments. As noted above, monitoring modules may obtain data from websites operated by third parties, e.g., the makers or providers of a connected monitoring device.

Data sources from which the system may obtain health event data may include patients and health care providers who perform or engage in a health care event. In some embodiments, health care providers may provide data through a user interface of the present invention. Alternatively or additionally, sources of health event data may include any variety of data sources that may hold such data, e.g., EMRs under control of a health care organization that has provided health care services to the patient, pharmacy records, electronic prescriptions, reimbursement claims to payers who are obligated to cover at least part of the costs of such health care services, and the like. In some embodiments, the system may provide information, e.g., on a website, that guides the patient through the process of specifying payers or other entities to be used as data sources and/or granting the system access to data held by such entities or requesting that such entities provide the system with access to such data. For example, the system may request the name of a payer (e.g., a health insurer), type of benefit plan, policy holder, identification number, policy number, or whatever else may be appropriate.

In some embodiments, physician events are reported to the REVON system when they are performed and billed for (or are otherwise referred to in a financial or administrative transaction, which may be an electronic transaction), e.g., by the treating physician or by other physicians, HCOs, or other entities that provide the relevant service (e.g., clinical laboratories). After a procedure has been performed, a provider (e.g., a health care provider, health care facility, or other individual or entity that provides the services) may submit a bill to a payer or the patient, indicating the procedure(s) performed, for which the payer or patient is requested to pay the provider (sometimes referred to as reimbursing the provider). A procedure for which payment is sought is typically identified by one or more codes (e.g., procedure codes). In some embodiments, when a provider, e.g., a physician, bills a payer or patient electronically or engages in another transaction in which the procedure is referenced (e.g., by procedure code) the REVON system is informed (e.g., the REVON system receives at least sufficient information to determine the procedure and patient on whom the procedure was performed). The REVON system may be informed directly by the provider, by an intermediary between the provider and the payer, or by the actual payer. This may be facilitated by use of a payer code assigned to the REVON system or to an entity that at least in part owns, controls, or manages the REVON system. An intermediary between the provider and the payer may be, e.g., a claims clearinghouse or other entity that engages in generating, processing, transmitting, and/or analyzing bills or claims. Information sufficient to identify a patient may comprise, e.g., a patient's name, social security number, patient ID, policy number, etc. In general, such information will have been provided to the REVON system, e.g., by the patient, during registration, as described herein. In some embodiments, software means are provided that permit a provider or a bill or claim submitter to enter two payer codes for a bill or claim or to supply an electronic copy of a bill or claim to the REVON system. In some embodiments, billing/claims information may be entered and/or submitted electronically to a payer and/or to a system of the present invention. In some embodiments, billing/claims information may be provided in hard copy form. In some embodiments, billing/claims information may be provided to the REVON system by a payer. For example, following receipt of billing/claims information from a provider, a payer may provide at least some such information to the REVON system. Typically the information is provided electronically. In some embodiments, practice management software, hospital management software, or claims processing software may be modified or provided with a plug-in that electronically contacts the REVON system or searches a database comprising a list of patients enrolled in the REVON system to determine whether a particular bill, claim, or transaction pertains to a patient who is enrolled. If so, the software submits to the REVON system at least sufficient information to identify the patient, the procedure, and the provider who is presenting the bill or claim pertaining to the procedure. In some embodiments, a patient may request their physician to notify the REVON system and/or a payer may request or require that the REVON system is notified as a condition for claim eligibility for patients that are registered with the REVON system. In some embodiments, payers may provide patients with cards that list a patient ID and/or policy number and, if applicable, indicate that the patient is registered with the REVON system. In some embodiments, such software may automatically verify the patient's eligibility for receiving benefits with a payer (e.g., an insurance company) using a standard electronic data interchange connection when a patient makes an appointment or checks in or registers at a physician practice, hospital, or other HCO and may, in parallel or in a similar manner, verify that the patient is registered with the REVON system. If so, the software may tag the patient's record so that the REVON system is automatically notified with the physician event-relevant information when the software is used to generate or process a bill or claim pertaining to that patient. In some embodiments, the amount of reimbursement or payment requested or paid or other information not necessary to identify the patient, procedure, and provider may be omitted from the information provided to the REVON system. In some embodiments, health data pertaining to physician events may be obtained, e.g., from EMRs or payers, in a retrospective fashion, e.g., health data pertaining to events that have already occurred at the time the patient enrolls in the REVON system may be obtained.

A data collection module may also or alternately collect health data from any of a wide variety of other data sources external to the system. For example, epidemiologic data such as data regarding the prevalence of incidence of infectious diseases, or data regarding the outdoor environment, e.g., in particular cities, zip codes, or other relevant geographic regions that define where a patient lives or works, may be collected from various government agencies, businesses, or other entities that collect such data. Data sources might also include companies that offer certain services that generate health data and that patients may obtain outside the context of a treatment relationship with a health care provider, such as companies that offer genotyping and, potentially, genome sequencing services, or other bioanalytical or imaging services that patients may choose to obtain outside the context of a treatment relationship with a health care provider. It should be understood that the data sources and particular types of health data described herein are not to be considered limiting. Any data source that may contain health data regarding or relating to the patient may be searched or may provide data to the system.

A patient's mobile device may serve as a data source in any of a variety of ways. It may comprise one or more sensors that directly collect health data of interest. In some embodiments, the location of a patient's mobile device or a pattern of locations, may be used to determine whether or that particular health care events may have occurred, when they occurred, where they occurred (e.g., at which health care facility), or any combination thereof. Location data may be used in combination with other data in this regard. In some embodiments, a possible hospitalization event (hospitalization of the patient) is detected by a method comprising (a) detecting or receiving information indicating the location of a patient's mobile device; and (b) determining that the patient's mobile device is located at a known hospital location, thereby detecting a possible hospitalization event. In some embodiments, the location is determined multiple times over a period of time, and the location data so obtained are analyzed to determine the likelihood that the patient is or was hospitalized. In some embodiments, a possible hospitalization event may be classified according to likelihood of actually being a hospitalization event as opposed, for example, to a situation in which the patient is at the hospital for a different reason, such as visiting someone who is hospitalized or attending an outpatient appointment at a hospital or visiting an emergency room. In some embodiments, a hospitalization event may be distinguished from an emergency room visit or other short-term visit based on the length of time over which the device is detected at the hospital without being detected away from the hospital. For example, the time period over which the mobile device is detected at the hospital without being detected away from the hospital may be determined. In some embodiments, a possible hospitalization event may be classified as a likely hospitalization event if there is at least a reasonable likelihood that the patient has been admitted to hospital. In some embodiments, if the device is detected at a hospital for at least 12 hours, at least 16 hours, at least 20 hours, or at least 24 hours, without being detected away from the hospital during this time period, the possible hospitalization event may be classified as a likely hospitalization event. On the other hand, in some embodiments, if the device is detected at a hospital during one hour and then detected away from the hospital during the next hour, the event may be classified as unlikely to be a hospitalization event. In some embodiments, if a possible or likely hospitalization event is detected the patient may be presented with a request for data indicating whether the patient is hospitalized. Such a request may include a question such as, “Based on your location, you seem to be in a hospital. Please tell me if you have been admitted to hospital.” The response, if any, may confirm or deny that the patient was hospitalized. A patient who has been admitted to hospital is considered to have experienced a hospitalization event whether the hospitalization event is ongoing or completed.

The system may employ appropriate methods to analyze the pattern of location data obtained over a period of time to correctly classify a possible hospitalization event. The method described here is provided merely by way of example. Other types of events may be detected based on location, such as the patient arriving at or being at a physician's office for a scheduled appointment, as mentioned above. The system may maintain information identifying the location of various health care facilities, e.g., hospitals, physician practices.

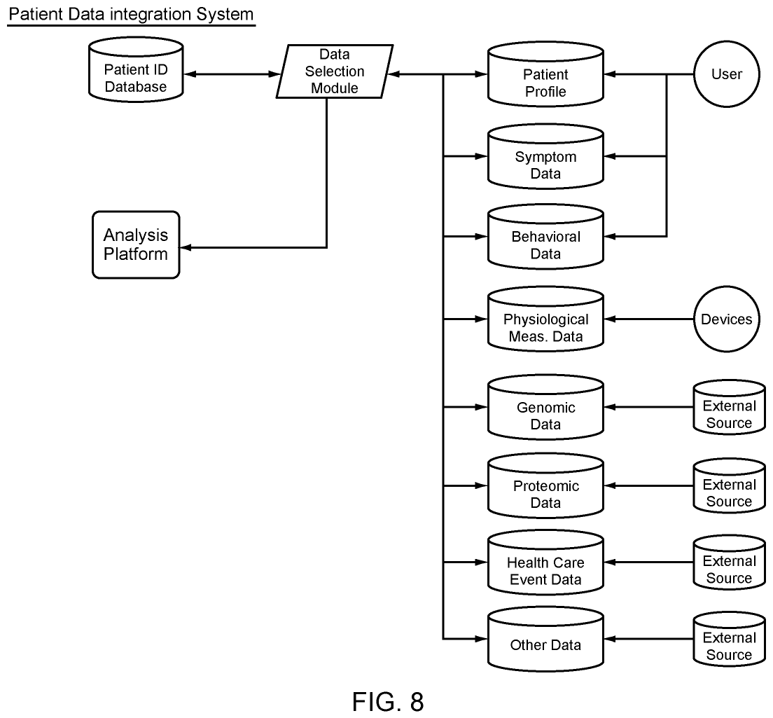

FIG. 8 shows a schematic diagram of a patient data integration system according to some embodiments. The REVON system collects various kinds of data about the patient from many different sources. Each type of data is stored in its own database, where it is linked to the patient using the patient ID. The data selection module accesses or retrieves any relevant patient information by first finding the patient ID from the Patient ID database and then finding the relevant information for that patient ID from all the databases that hold health data. The health data retrieved from the health data databases for the given patient ID may then be made available to an analysis platform for analysis. The system may collect large volumes of patient data from various sources (e.g., EMRs, claims databases) periodically and store it according to its type and tagged with patient ID. When needed for purposes of a given T-plan condition (e.g., in order to assemble a T-plan data module or in order to provide data pertaining to a particular patient to a physician who prescribed a T-plan for the condition to the patient) the data for the patient may be searched for in the relevant database or according to the relevant type.

Database and Subscriptions

In some aspects, a computer-implemented process of assembling a database comprising health data is presented. In some embodiments, the data are organized around specific conditions. In some embodiments, the data are de-identifiable or de-identified. In some embodiments, the health data comprise health data obtained as specified by a T-plan as described herein and stored in a patient record. In some embodiments, the data may be organized as data modules containing data that pertain to a particular patient with a particular condition (T-plan data modules). The type and/or format of the data included in the database and/or the extent to which an individual or organization is permitted to access or use the database or particular data in the database may vary. The data may be de-identified or de-identifiable, by removing or lacking personally identifying information. In some embodiments, one or more copies of data that comprises personally identifiable information and one or more copies of the data without personally identifiable information are maintained.

A database comprising health data may be used by any of a variety of individuals and/or entities. HCPs who use a system of the invention in their practice may use the database in the ordinary course of providing care for their patients, to view patient health data while in or away from the office, to analyze their patient population, or explore their own hypotheses or research questions. In some embodiments, patient users may use the database to review their health information. In some embodiments, individuals or entities may be permitted to access the database, e.g., in exchange for a fee. Such individuals or entities (sometimes referred to as “subscribers” herein) may use the database, for example, to perform medically related research or for any of a variety of other purposes.

Some uses of the database may include, for example, identifying risk factors for diseases or adverse drug reactions, identifying unnecessary or counterproductive utilization of medical resources, identifying instances of failure to implement appropriate treatment or preventive measures, identifying or tracking outbreaks of infectious diseases or foodborne illnesses, initiating and tracking recalls of medications or medical devices, tracking treatment outcomes (e.g., comparing outcomes achieved through different therapeutic approaches, comparing the efficacy of different medications or surgical procedures, determining whether certain elective surgeries result in clinical benefit), identifying patients who may be eligible for clinical trials or otherwise eligible to receive experimental therapies, comparing treatment outcomes achieved by different health care providers or health care facilities, analyzing the effect of particular follow-up or discharge procedures on readmission rates, identifying patient characteristics that are associated with an increased likelihood of readmission, identifying patients who are poorly compliant with treatment recommendations, to name a few.

In some embodiments, the database may be used to identify undiagnosed, untreated, or undertreated conditions among patients within a given population, e.g., within a payer's patient population or within a health care facility's patient population or a given geographic region. Such conditions may be identified, for example, through analysis of data obtained by monitoring modules, health care event data, significant patient characteristics entered by health care providers when prescribing T-plans, or a combination thereof. In some embodiments such analysis may be performed by a payer, health care facility, or government entity. In some embodiments, such analysis may be performed automatically by the system. Results may be provided to the relevant payer, health care facility, or government entity and/or to the patients so identified.

In some embodiments, computer-based tools (which may be embodied in hardware, software, or a combination thereof) for querying the database, retrieving data, etc., may be provided. For example, a component may provide users with a GUI that facilitates query creation. In some embodiments, the system may support the creation and performance of multi-dimensional queries. In some embodiments, the system may support the creation and performance of natural language queries

In some embodiments, a database may be structured in a way that facilitates the ability to perform medically related research, such as to collect information regarding outcomes or side effects associated with various treatments, medications, or combinations thereof or any of various types of analysis (which may be referred to as “meta-analysis”).

In some embodiments, certain types of data may be analyzed with online analytical processing (OLAP) tools. The data may be stored in multi-dimensional array storage (e.g., for analysis using multidimensional online analytical processing (MOLAP) tools), or a relational database (e.g., for analysis using relational online analytical processing (ROLAP)) or combinations thereof (e.g., HOLAP (hybrid online analytical processing)) such as where part of the data is stored in a MOLAP store and another part of the data in a ROLAP store. It should be noted that such tools may be used in analysis of data for presentation in physician dashboards, patient dashboards, or both, in addition to or instead of to analyze data for other purposes. In some embodiments, one or more tools may be provided that permit a user to extract data into standard available data analysis or statistical software programs, packages or environments such as SAS®, SPSS, Systat®, Minitab®, R, etc. or to analyze data using tools such as those provided in such software.

In certain embodiments, a system or database described herein may provide makers or providers of medications, medical devices (e.g., diagnostic devices, therapeutic devices), diagnostic tests, and/or services (e.g., diagnostic testing services) with access to data relating to such medications, devices, tests, and/or services. Such data may be used, e.g., to better understand how such medications, devices, tests, and/or services are used in clinical practice or to identify opportunities or needs for improved or new medications, devices, tests, and/or services.

In certain embodiments, a system or database described herein may provide the medical research community with access to data that will permit the development of new insights into diseases and patient populations.

In certain embodiments, a system or database described herein may provide health care organizations (e.g., hospitals, physician practices) with any one or more of the following benefits: improved quality and/or improved patient outcomes (e.g., reduced readmissions), which may lead to higher reimbursement under performance-based payment schemes (sometimes termed “pay-for-performance” or “outcomes-based” payment schemes); increased validity of performance-based reimbursement; improved ability to comply with accreditation standards, laws, and/or regulatory requirements, guidelines, or mandates; a database that can be mined to gather information useful, e.g., in making institutional decisions or policies.

In certain embodiments, a system or database described herein may provide payers with any one or more of the following benefits: increased feasibility and validity of performance-based reimbursement; improved monitoring and encouraging compliance with therapy; improved monitoring of symptoms or changes in a patient's condition that may permit timely intervention; actively directing the patient to follow particular directions for managing the condition to, e.g., reduce the likelihood of readmission or other deterioration in the patient's health requiring medical attention.

Subscribers may be, for example, medical researchers, organizations such as pharmaceutical companies or insurance companies, government entities (e.g., Federal, state, and/or local government entities), or simply individuals interested in the content of the database. In some embodiments, the arrangement under which access is granted or the set of access rights provided may be referred to as a “subscription”. There may be multiple classes of subscriptions that, for example, allow subscribers different access rights. For example, access rights may differ in terms of number of access sessions or queries permitted, the type of information that may be accessed, the type of analysis that may be performed, etc. In some embodiments, the different classes of subscriptions may have different fees and/or fee structures, which may depend at least in part on the extent and nature of the associated access rights. For example, a subscription may provide a single access session to the database in exchange for a one-time fee. In some embodiments, a subscription allows the subscriber to access the database multiple times over a defined period of time, such as 1 month, 3 months, 1 year, etc. The number of access sessions and/or queries permitted within the defined time period may be limited or unlimited in various embodiments. In the case of organizations or other entities, a site license may be provided that allows multiple users to have access to the database. Each subscriber and/or user may select or may be assigned a user ID and, in at least some embodiments, a password. Some types of subscription may permit a user to print and/or download information while other types may only permit viewing. In some embodiments, a system may comprise one or more components that at least in part manages database subscriptions. For example, in some embodiments, the component may keep track of access rights, use of the database by subscribers, payments due and received, etc. Such a component may be, e.g., part of a user account manager component.

In some embodiments, terms and/or conditions under which a subscription is provided are embodied at least in part in a subscription agreement. In some embodiments, a subscription agreement may be accepted electronically by an entity or individual wishing to become a subscriber. In some embodiments, terms of a subscription may include a requirement for payment (e.g., royalties) on any new products or new uses of existing products that are discovered or identified at least in part through use of the database. In some embodiments, terms of a subscription may include a requirement for payment (e.g., royalties) on any new products or new uses of existing products that approved for marketing at least in part through use of the database. In some embodiments, at least some such payments may be distributed to individuals or entities that contributed data used in the identification, discovery, or approval of a new product or new use for an existing product. In some embodiments, a subscription agreement comprises a license agreement that secures payments (e.g., royalties) on any new products or new uses of existing products that are discovered or identified or approved for marketing at least in part through use of the database.

Subscribers may, in some embodiments, download T-plans, T-plan data modules, or portions thereof onto their own computer systems for analysis independently of the system (e.g., using their own proprietary tools) or may perform analysis using computer-based tools provided by the system or may use a combination of such approaches.

In some embodiments, the system may provide one or more computer-based tools that include facilities for data analysis, calculation, graphical display, and/or statistical analysis of T-plans and/or T-plan data modules.

In some embodiments, a database may be used to identify patients of interest. Patients of interest may be patients with a particular condition, particular patient characteristic(s), particular health data, particular compliance data, particular monitoring devices, or any combination of the foregoing. In some embodiments, patients of interest are patients who may be eligible to receive an experimental therapy, e.g., in a clinical trial, expanded access program, or other program. “Experimental therapy” refers to a therapy, e.g., a medication or medical device, that either has not been approved for marketing or for treatment of a particular condition in the United States by the US Food & Drug Administration (FDA) in the case or patients who reside or receive care in the US or has not been approved for marketing or for treatment of a particular condition by the relevant regulatory authority in the country or region where a patient resides or receive care. In some embodiments, a database may be used to identify patients who may be eligible to participate in clinical trials sponsored by pharmaceutical companies. Patients who are potentially eligible typically have the condition for which the experimental therapy is being studied and may meet certain criteria, which may include, for example, being within a certain age range, absence of certain other conditions or complications, and/or others. Patients of interest may be identified without disclosing patient identifying information by searching de-identified patient records that can be linked back to the patient through use of a key or other data to which the user does not have access. Patients of interest may be contacted through the REVON system or separately via e-mail, phone, text message, or other communication modes and provided with information about the therapy and/or the program or trial through which the therapy may be available, and information about how to participate in the program or trial (e.g., contact information for clinical trial sites or programs). Such recruitment activities may also be conducted without disclosing patient identifying information. In some embodiments, patients may be able to opt in or opt out of use of their patient records or any portion of their health data for such purposes.

In some embodiments, a database may be used to identify health care providers that have one or more patients of interest. Such health care providers may then be contacted through the REVON system or separately via e-mail, phone, text message, or other communication modes, informed about the trial or program, and/or invited to serve as investigators in the trial or program.

Experimental Therapies and Risk Management Programs

In some embodiments, the REVON system may be used in trials of experimental therapies. As discussed above, a REVON system database may be used to identify and, in some embodiments, recruit patients who may be eligible for trials. In some embodiments, additionally or alternately, the REVON system may be used in the conduct of trials of experimental therapies. For example, a T-plan or SST appropriate for or specifically designed or modified for obtaining health data relevant to the trial may be prescribed to a patient who participates in a trial. In some embodiments, a health care event module of such a T-plan provides a treatment plan that constitutes a protocol for a trial of an experimental therapy, e.g., a clinical trial sponsored by a pharmaceutical company.

In some embodiments, a T-plan includes an SST, basic symptom tracker, or monitoring module that is designed at least in part to monitor symptoms and/or physiological measurements that are indicative of safety or potential efficacy of an experimental therapy. In some embodiments, a T-plan that includes an SST, basic symptom tracker, or monitoring module that is designed at least in part to detect the occurrence of potential adverse events, e.g., undesired reactions to or consequences of a therapy, may be prescribed to a patient to whom such a therapy has been prescribed or recommended or administered in a trial. In some embodiments, the SST instructs the patient to seek medical attention or take other appropriate action in the event such a potential adverse event is detected. In some embodiments, the therapy is a pharmaceutical agent. In some embodiments, the therapy is an experimental therapy. In some embodiments, the T-plan notifies the investigator or sponsor of a trial in which such therapy is being tested if data indicative of a potential adverse event are detected. In some embodiments, the therapy is a marketed therapy. In some embodiments, prescription of a T-plan that includes an SST, basic symptom tracker, or monitoring module may be part of a post-market surveillance (Phase IV trial) or risk management program required or recommended by a regulatory authority such as the FDA or required or recommended or conducted by a pharmaceutical company that markets the therapy. In some embodiments, the T-plan notifies the marketer of such therapy of the occurrence of potential adverse events.

Recommendation Engine and Marketplace

In some embodiments, a REVON application or T-plan may provide or serve as a recommendation engine for product(s), individual(s), service provider(s) and/or facilit(ies) suitable for managing a T-plan condition and/or may act or provide access to a marketplace through which a patient can acquire a product or make an appointment with an individual, service provider, or facility suitable for managing a T-plan condition. Recommendations or reviews may originate from the patient's HCP, from other patient users, from physician users, etc. A product may be, for example, a monitoring device. An individual may be, for example, a health care provider, counselor, educator, or fitness instructor. A service provider may, for example, provide rehabilitation services, smoking cessation services, counseling services, education services, or fitness training services. A facility may be a health care facility, counseling facility, educational facility, or fitness facility. A recommendation may comprise data that identifies a specific product model and/or brand, a specific individual, a specific service provider, or a specific facility, optionally wherein the product integrates with the patient's T-plan (e.g., so that the device may transmit data for entry into a T-plan) or the individual, service provider, or facility has a record or account in a database accessible by the computer program product so that, for example, the individual or employees of the service provider or facility may (with appropriate permission from the patient) access the patient's T-plan and/or prescribe a new T-plan for the patient (e.g., if the individual is a physician to whom the patient is being referred via this mechanism). In some embodiments, within a T-plan or elsewhere in the REVON application, a patient may view recommendations or reviews, may access links to websites that allow purchase of a recommended product or service, or directly purchase a recommended product or service, contact or make an appointment with a recommended service provider or individual, etc. In some embodiments, within a T-plan or elsewhere in the REVON application, a patient may post recommendations or reviews, which may be viewed by other patients. In some embodiments, the website is accessible from within the REVON patient application user interface, e.g., on a mobile device. In some embodiments, after a T-plan has been assigned, the system may provide the individual to whom the T-plan has been assigned, the individual who assigned the T-plan, or both, with a list of recommended connected monitoring devices for use to collect data specified by the T-plan. In some embodiments, the list specifies whether or to what extent the cost of particular device(s) is covered by the health insurance or other health care coverage of the individual to whom the T-plan was assigned. A user may log in to the website using the same credentials (e.g, userID and password) as he or she uses to access the patient application.

In some embodiments, a patient or other user on behalf of the patient may use the website to determine whether or to what extent the cost of a particular monitoring device (e.g., a monitoring device that obtains data elements specified by a T-plan assigned to the patient) or other product or service is reimbursed by the patient's health insurance provider or other payer. The system may use information regarding the patient's insurance policy or other health care coverage to provide such information. In some embodiments, the system may provide the patient (or other user) with a list of those monitoring devices that are at least partly covered by the patient's insurance policy or other health care coverage. In some embodiments, the system may automatically submit a claim for reimbursement if the patient purchases an eligible device. In some embodiments, the patient (or other user) may view a list or other representation of their T-plans and the types of monitoring devices that obtain data elements of the types specified by the T-plans. In some embodiments, the system indicates which devices are connected monitoring devices that the system can use to automatically obtain data. In some embodiments, if a device or other product or service requires prior authorization to be covered by a patient's health insurance or other health care coverage the system may inform the user.

In some aspects a system thus provides a marketplace in which users, e.g., patients, may access any of a variety of types of products, services, and/or information, relating to health. In some embodiments, use of the marketplace, e.g., transactions, may be tracked.

In some embodiments, advertisements for products and/or services relevant to a T-plan condition may be displayed or sent to patients who have been prescribed a T-plan for that condition. In some embodiments, patients who have been prescribed a T-plan for a condition may be asked or provided an opportunity to rate and/or review products and/or services relevant to the condition and/or providers of such products and/or services. Products relevant to the condition may include, e.g., monitoring devices, prosthetic devices, mobility aids, certain foods (e.g., foods that are free of one or more allergens), over-the-counter or prescription medications, etc. In some embodiments, questionnaires, rating scales, or instructions are provided to help enhance the usefulness and objectivity of the ratings or reviews. In some embodiments, products and/or services may be relevant to wellness/wellbeing in addition to or instead of being relevant to particular condition(s). Such products and/or services may include, e.g., exercise/fitness equipment, gym memberships, exercise/fitness programs or classes, wellness coaches, personal trainers, etc.

Interfacing and Integrating with Various Systems and Software

In some aspects, the invention provides a computer-implemented process of augmenting an electronic medical record system. An EMR system that does not use T-plans may be augmented by integrating a computer program product comprising tools for use by health care providers in connection with T-plans as described herein. Such integration may, for example, allow a health care provider to prescribe a T-plan as described herein from within such an EMR, may allow health data collected by the REVON system to be viewed from within an EMR.

In some embodiments, an EMR system that does not use T-plans may be equipped with functionality that makes it possible to use T-plans within, or in connection with, such EMR system. In some embodiments, systems and methods of equipping an EMR system, with functionality that allows users of such EMR system to design, view, modify, prescribe, and/or otherwise use T-plans are described herein. For example, it is envisioned that health care providers may design and/or prescribe T-plans from within an EMR system, optionally within a patient record in an EMR system, and/or may view physician dashboards or otherwise view health data acquired by the REVON system from within an EMR system. In some embodiments, access to the REVON system or at least some functions of the REVON system may be provided through a tab, link, or icon displayed on a screen by an EMR system, e.g., within a patient record of the EMR system. In some embodiments, access to such functions is provided via a component, e.g., a software component, which component may be referred to as a “T-plan EMR component”. In some aspects, a non-transitory computer-readable medium comprising a T-plan EMR component is disclosed herein. A T-plan EMR component may be provided in any suitable way in various embodiments. For example, in some embodiments, a plug-in that comprises or consists of a T-plan EMR component may be downloaded from a website or provided on a tangible computer-readable medium. As will be appreciated, the term “plug-in” (sometimes termed “add-on” or “extension”) refers to a software component or set of software components that adds specific functionality (abilities) to another software application. In some embodiments, a T-plan component is designed to function specifically with a particular EMR system or with any of multiple different EMR systems. In some embodiments, a T-plan EMR component may be provided as part of an upgrade of an EMR system. In some embodiments, a T-plan EMR component may be an option that may be furnished together with or after adoption of an EMR system. An EMR system that utilizes T-plans may be referred to as a “T-plan equipped EMR system”. In some embodiments, a T-plan equipped EMR system displays a link within EMRs of at least some patients of an HCP who uses the system. Such link(s), when active, may allow a user to access and use T-plans and related functions for design and prescription of T-plans as described herein.

In some embodiments, the REVON application may interface with one or more additional health-related applications for an electronic device, e.g., a mobile electronic device. Such applications may be, for example, a medication tracking application, an exercise tracking application (e.g., a pedometer application), a weight tracking application, a calorie counting application, a heart or blood pressure monitoring application, an application that is specific to one or more conditions, etc. In some embodiments, such applications may be made available by third parties. The applications may interface with and operate together with the REVON application. In some embodiments, the REVON system interfaces with a plurality of applications made or controlled by third parties, each of which may provide functionality for managing or tracking one or more variables or conditions. The patient may activate one or more of these applications for use with the REVON system. In some aspects, the REVON system may act as a platform through which multiple third party applications can be used by patients having particular conditions for which such applications or components may be useful. In some embodiments, the REVON system may supply or recommend such an application, optionally based at least in part on patient characteristics. For example, if a patient has diabetes as a patient characteristic, REVON may supply or recommend a third party application useful for or intended for management of diabetes.

In some aspects, a system, application, or module (e.g., any system, application, or module described herein) interfaces with or is capable of interfacing with any of a variety of external or internal systems or modules. Such systems or modules may include, e.g., EMR/HER or electronic data capture (EDC) systems of various providers, external authentication tools, web services APIs such as Google maps, device APIs for device data such as geolocation, to name a few.

In some embodiments, a system interfaces with practice management software, hospital management software, and/or scheduling software that may already be used in a health care facility to make patient appointments. In some embodiments, a T-plan facilitates making appointments with health care providers who may not be affiliated with or employed by particular health care facility or health care organization. In some embodiments, once a physician has assigned a T-plan to a patient, the T-plan can be accessed by other personnel in the health care facility, e.g., other health care providers or associated personnel in the physician's office, who may then assist the patient in regard to one or more of the health care events specified by the T-plan. Such personnel may assist the patient in any of a variety of ways. For example, they may make an appointment for the patient, may assist the patient regarding a future encounter (e.g., by providing contact information or address of a health care provider or facility to whom the patient was referred, etc.), may provide explanations of medication administration or other self-care, etc.

Implementation

This section includes discussion of certain aspects relating to implementation of the present invention. It should be understood that various aspects pertaining to implementation are discussed elsewhere herein. In general, the invention may be implemented with any suitable combination of hardware and software in various embodiments. If implemented as a computer-implemented apparatus, the present invention is implemented using means for performing those steps and functions described herein that have been selected for the particular embodiment of the invention being implemented.

The present invention may be included in an article of manufacture (e.g., one or more computer program products) comprising, for instance, “computer useable media” (which term is used interchangeably with “computer readable media”). The media has embodied therein, for instance, computer readable program code means for providing and facilitating the mechanisms of the present invention. The article of manufacture may be included as part of a computer system or sold separately.

The computer-usable or computer-readable medium may be, for example but not limited to, an electronic, magnetic, optical, electromagnetic, infrared, or semiconductor system, apparatus, device. Examples of a computer-readable medium include the following: a hard disk, a random access memory (RAM), a read-only memory (ROM), an erasable programmable read-only memory (e.g., EPROM or EEPROM), flash memory, a portable compact disc read-only memory (CDROM), a floppy disk, an optical storage device, or a magnetic storage device. A computer-usable or computer-readable medium may, in some embodiments, be paper or another suitable medium on which the program is printed or embodied, as the program may be electronically captured, for instance, via optical scanning of the paper or other medium (optionally employing optical character recognition), then compiled, interpreted, or otherwise processed in a suitable manner, if necessary, and then stored in a computer memory and/or executed by a computer processor. In the context of this document, a computer-usable or computer-readable medium may be any medium that may contain, store, communicate, propagate, or transport the program for use by or in connection with the instruction execution system, apparatus, or device. The computer-usable medium may include a propagated data signal with the computer-usable program code embodied therein. The computer usable program code may be transmitted using any appropriate medium, including but not limited to wireless, physical wires, wireline, optical fiber cable, etc.

In some embodiments, one or more aspects, components, or features of a computer program product described herein may be delivered or made available to users as an online service, which may be a cloud-based service. In some embodiments, users may interface with the service using web browsers on personal computers (and/or in some embodiments web browsers that run on mobile devices) and/or using applications on mobile devices such as smartphones and tablets. Web browsers that may be used in certain embodiments may include, e.g., Internet Explorer, Safari, Firefox, Chrome. A software application that runs on a mobile device may be referred to as an “app”, as is common in the art. In some embodiments, a computer program product of the present invention is delivered or made available to users over the Internet. A computer program product may include interfaces for personal computers (such as PC and Mac, desktops, laptops, netbooks, notebooks, and/or ultrabooks) and/or for mobile devices (such as Android and iOS based smartphones and tablets). In some embodiments, a system or product may be utilized without requiring the downloading or installing of product software on any user device. In certain embodiments, the only downloadable or installed software is a mobile application that runs on a user's mobile device. In some embodiments, a mobile device may be provided for use by patients, caregivers, and/or health care providers. A computer program product of the present invention may be pre-installed or downloaded on the device.

In some embodiments, a system or computer program product may comprise multiple different user interfaces or portals, e.g., web portals. Different user interfaces or portals may have different features, which may be based at least in part on the purposes or needs of different user categories and/or limitations on the type of data to which users in those categories may be granted access. For example, users of a pharmaceutical industry portal may have access only to de-identified data. In some embodiments, a system or product is modular to different user interfaces, in that additional user interfaces, portals, or portal types can be readily added without modifying other components or modules of the product.

In some embodiments, a system or computer program product may interact with users (e.g., via a standard graphical user interface (GUI), analyze health data, and/or assemble health data into one or more modules. As noted above, a system may comprise multiple components. For example, one or more components may receive and/or transmit health data and/or other information to or from users; one or more components may analyze health data and/or other information; one or more components may add information to a database; one or more components may generate output to be transmitted or presented to users; one or more components may extract information from a database in response to a request or query. One or more components may analyze extracted data and/or convert the data into an appropriate format for transmission or display. One or more components may receive and/or transmit information between other component(s) of the system or external to the system. It should also be understood that components may operate in parallel or sequentially at various times for various purposes and may interact with each other in a variety of ways.

As will be evident, certain aspects described herein involve transmission of data. Data may be transmitted or received via any type of electronic transmission in various embodiments. An electronic transmission may occur over a communication network, e.g., a computer network such as the Internet, or a phone network. Where reference is made to “entering” or “entry” of data, it should be understood that such data may be transmitted unless otherwise indicated. Entry and/or transmission may occur in response to a request or action initiated by a user or in response to a request initiated by a system. For example, at least some health data entered by a user may remain stored on a user's computer for a period of time prior to being transmitted. Such transmission may occur in response to a request initiated by a system, which may occur automatically, e.g., at predetermined time intervals or at times that may depend at least in part on system usage level, priority of the health data, or other parameters. In some embodiments, at least some health data may remain stored on a computer or data storage system owned or controlled at least in part by a user or outside the control of a system of the present invention but is made available to a system of the present invention for analysis and/or retrieval. In some embodiments, health data may be entered at least in part orally. A system may include, make use of, or accept input from appropriate voice recognition software and/or speech recognition software. In some embodiments, a system may ask questions to a patient by presenting the questions on a computer screen, orally, or combinations thereof. The system may receive responses, process the responses, and/or add the responses or processed responses to a database. Various electronic data entry systems and input devices may be supported such as touch screens, light pens, keyboard, digital pen, stylus, scanners, cameras, telephone, etc. In some embodiments, handwriting recognition software and/or voice recognition software may be employed. In some embodiments, at least some data may be timestamped (with date and with the time within the 24 hours of the day) and stored in a database in association with an identifier of the patient, optionally including information identifying the type or source of the data. A timestamp may indicate when a measurement was made (e.g., which may be taken to be the time that the measurement was entered into an application on a patient's mobile device) or when it was received by a “backend” server of the REVON system to which it is transmitted from the mobile device. There may be multiple timestamps for a given data element. In some embodiments, a basic check may be performed to determine whether a measurement was likely to be a valid measurement or response. For example physiological data incompatible with life would be considered invalid. In some embodiments, a module may determine what to do in regard to invalid data (e.g., ask patient to re-enter, etc.). This module may be part of an app and run on a mobile device or may be part of the backend and run on a REVON system server.

In certain aspects, data entry and submission, and/or use of database content may be facilitated by use of standard GUI elements (sometimes referred to as GUI widgets), such as buttons (e.g., boxes to check or fill in, radio buttons), lists (e.g., scroll-down or drop-down lists from which to select among various options), menus, etc. For example, in some embodiments, entry of health data may be facilitated by providing on a computer one or more document(s) (forms) that contain a structured format in various input fields are provided. At least some of the input fields may be presented as buttons or lists of options from which to select. Typical webform elements may be used. In some embodiments, a form may contain at least some fields that are to be completed by (a) making a selection from a set of predetermined options (whether presented visually, orally, or otherwise); (b) entering a numerical value; (c) answering questions to which there may be a limited number of permitted responses (such as yes/no questions).

In general, a user may enter and/or retrieve health data in any of a variety of ways in various embodiments. It is envisioned that a system may interact with a user via one or more computer-based documents (e.g., web pages, e.g., dynamic web pages). In some embodiments, Ajax technology may be used. Ajax represents a broad group of web technologies that can be used to implement a web application that communicates with a server in the background, without interfering with the current state of the page. A user may navigate between different pages, screens, or portions thereof by clicking on “links” (which may be indicated using text of a different color or font), arrows, and other navigation methods typical of web page or app navigation. For example, users may log on to a system using a computer and be presented with a computer-based document (displayed on a screen) from which various options may be selected. The particular options available to the user may depend on the type of user (health care provider, patient, subscriber, etc.).

In some aspects, one or more identifiers, definitions, descriptions, code sets, and/or codes, e.g., identifiers, definitions, descriptions, code sets and/or codes for procedures, medications, providers, terminology, health care providers, and/or payers may be used, e.g., to represent, analyze, retrieve, process, or store one or more data elements. An identifier, definition, description, or code may be from any of a number of sources or code sets. For example, a code or portion thereof used in ICD-10 or ICD-10-CM may be used. Other code sets include code sets in Systematized Nomenclature of Medicine Clinical Terms (SNOMED-CT) (http://www.ihtsdo.org/snomed-ct), Logical Observation Identifiers Names and Codes (LOINC) (http://loinc.org/). Identifiers of health care providers and/or health plans as assigned by the US National Plan and Provider Enumeration System (NPPES) may be used, which are available from the National Provider Identifier (NPI) Registry). One or more conversion tables, e.g., mapping of codes from one code set to another, may be used if appropriate. A medication code may be, e.g., a National Drug Code (NDC). A code set typically includes codes and descriptors of the data elements that are represented by the codes. Code sets may be revised or updated periodically. Aspects and embodiments described herein may use, recognize, or include revisions or updates to code sets and/or may use the same descriptors for any of such data elements as used in any code set but may assign distinct codes. In some embodiments, two or more descriptors may be combined into a single descriptor or divided into two or more descriptors.

In certain embodiments, a system may include or may access (e.g., over a communication network) any of a variety of collections of information. For example, such information may include information pertaining to, e.g., diseases, diagnoses, diagnostics, procedures, laboratory tests, medications (e.g., National Drug Data File Plus drug database), identifiers for health care providers and/or health plans, medical terms (e.g., glossaries, translations), code sets, medical coding systems, means for converting between different coding or terminology systems, etc. Such compilations may be in the form of data files, tables or files in a database, or other records. In some embodiments, a system may use such information in analyzing health data and/or performing other functions. Aspects and embodiments described herein may be equipped or modified to use, recognize, or include revisions or updates to code sets or other collections of information.

It will be understood that details of computer-implemented processes and computer program products described herein may be customized for any particular electronic device. In some embodiments, an application running on a first device, e.g., a mobile electronic device, may synchronize with corresponding application(s) running on other electronic devices that may be owned or used by the same user so that a seamless and integrated user experience is provided. It should thus be understood that an application may operate across multiple computers. For example, a patient or physician may access and use the system from a mobile device, desktop computer, notebook computer, or any appropriate computer. Information may be synchronized across devices so that, for example, data entered into a first device is available essentially immediately across the various devices on which the application is installed or accessible. It should also be understood that where reference is made to “installing” or “downloading” (e.g., downloading or installing a component such as an app, update, etc.), such terms may be used interchangeably and may refer to both downloading and installing, activating a pre-installed component such as an application or update, or taking other action that makes a component available for use with a particular device. Embodiments in which one or more applications are provided on a computer-readable medium such as a USB flash drive (sometimes termed a “memory stick” or “thumb drive”, CD or DVD are provided. It should also be understood that, in some embodiments, at least some computer-executable instructions may be executed remotely, e.g., on one or more remote servers (e.g., in the cloud), rather than on the device that is being physically used by a user. It should also be understood that data entered into a device, e.g., via an app, may be stored remotely, on a user's device, or at least in part on both.

In certain embodiments, at least some of the same functions and information as are available in an application on a mobile device, organized in the same general way, may be available to a user by visiting a website and entering the user's userID and password (or other appropriate login credentials). The screens are displayed in a browser, and the user can view screens, navigate using similar navigation tools, enter data, and perform other activities. The particular functions available when an account is accessed using a browser or certain electronic devices may be limited based, e.g., on the capabilities of the device being used and/or the limitations of the browser. Functions that require use of capabilities that are not available on the device being used may not be available. For example, a desktop computer may not have geolocation or phone call capabilities. Alternate means of communication or navigation may be provided. For example, in some embodiments, navigation or data entry on a mobile device that has a touchscreen may be performed at least in part by swiping or tapping or pinching, while if the device does not have touchscreen capability, information may be presented in a list format and navigated or selected using tabs, checkboxes, and the like. In some embodiments, if the device has voice recognition capability, the user may provide instructions to the application at least in part orally instead of or in addition to by input via the screen. As used herein, “swipe”, “tap”, or “pinch” encompasses actions in which the user's finger or other body part is in physical contact with the screen or other input device during at least part of the action. In the case of devices equipped with appropriate gesture recognition software, a gesture equivalent to a swipe, tap, or pinch may be performed without physical contact with the screen. For example, a gesture such as waving a hand or finger in the air above a screen or eye movement may be used

In some embodiments, an application for a mobile device may be available through the same channels by which apps are ordinarily available for the particular device. For example, apps for Apple devices such as iPhone or iPad may be available from the iTunes App Store (http://www.apple.com/itunes/); apps for devices running an Android operating system may be available from Google Play (http://www.android.com/apps/#). In some embodiments, an application may be available through a channel specific for such apps in addition to, or instead of, a general app store. A website may be provided that offers the apps for downloading. In some embodiments, an application is free. In some embodiments, a fee may be charged for an application or for certain functions of an application. For example, there may be different versions, such as a standard version (which may be free) and a premium version (which may require a fee and may include extra features that are not available in the standard version).

It will be understood that a system may include a variety of components that carry out various tasks described herein. For example, a system may comprise components that (1) provide for creation, modification, management of T-plans and associated databases; (2) manage user enrollment and accounts; (3) process input from health care providers and patients; (4) provide output to health care providers and patients; (5) provide scheduling functions, e.g., for data collection; (6) provide data analysis tools; (7) provide for networking functions, among others. The system may comprise interfaces between various components, databases, and/or external systems, as appropriate.

The system may comprise one or more databases that store various items of data used by the system. In general, a database (which term may be used interchangeably with data store) is an organized collection of data. The data may be organized in a way that supports processes that use the data (information) in the database. A database may be implemented using a database management system (DBMS). In some embodiments a relational database management system (RDBMS) is used. Various RDBMS software packages are available, e.g., from Microsoft (e.g., Microsoft SQL Server), Oracle (e.g., MySQL), Informix, and IBM (e.g., DB2). Non-SQL based DBMSs, e.g., object database management systems, may be used in various embodiments of the invention. It should be understood that the data may be stored in multiple distinct databases, which may be stored in different locations. Thus reference to a “database” should be understood as referring to one or more databases. In some embodiments, data elements of a given type are stored in a particular database or area of data storage, which may be reserved for data elements of that type and may explicitly or implicitly identify the data elements stored therein as being of a particular type. In general, data element types may be structured data that conforms to a predefined data model or may be unstructured data, which may, in some embodiments, be tagged with an identifier of its type. In some embodiments, data are stored in encrypted form in one or more data bases or storage locations and in de-identified, non-encrypted form in one or more other databases or storage locations. Data stored in de-identified form lack personally identifiable information but may have an identifier that can be used to determine the patient to whom the data pertain. However, the key or code by which the de-identified data could be re-identified may be stored separately from the data itself. Data may be stored and retrieved using standard approaches. It will be understood that data may be stored in a database in any suitable manner. The database may contain references, e.g., pointers, to the data itself, which data may be stored within the system or externally.

The invention or aspects thereof may be implemented using one or more computer systems, which may each comprise one or more computers. A computer system of use in the present invention may be a general-purpose computer system that is programmable using a high-level computer programming language. A computer system may be implemented at least in part using specially programmed, special purpose hardware. In general, a computer system includes a processor, which may be a commercially available processor in various embodiments. Such a processor usually executes an operating system which may be, for example, a Windows operating system (Microsoft), MAC OS (Apple), Linux available from various sources, UNIX available from various sources, etc. Many other operating systems may be used. It will be understood that portable electronic devices may use different operating systems from those running on larger devices, e.g., iOS (Apple), Android (Open Handset Alliance), etc. A processor and operating system together provide a computer platform for which application programs in high-level programming languages are written. It should be understood that the invention is not limited to a particular computer system platform, processor, operating system, or communication network. It would be apparent to those skilled in the art that the present invention could be implemented using any of a wide variety of programming languages or computer systems. It should be appreciated that the invention is not limited to any particular architecture, communication network, or communication protocol. Various embodiments of the invention may be implemented as programmed or non-programmed elements, or any combination thereof. Various embodiments of the present invention may be programmed using an object-oriented programming language, such as Python, Java, or C++. Other object-oriented programming languages may also be used. Functional, scripting, and/or logical programming languages may be used. One or more elements of the invention or aspects thereof may include one or more application programming interfaces (APIs). Such APIs may, for example, facilitate communication between existing electronic medical record systems and a system of the present invention. One or more elements of the invention or aspects thereof may be implemented as or using a “web service” (which term refers to a software system designed to support interoperable machine-to-machine interaction over a communication network, e.g., the Internet). Web services of the present invention may employ a variety of architectures and architectural styles. In some embodiments aspects of the present invention may comprise RESTful web services. It will be understood that a computer system may include various standard components such as one or more peripheral devices, e.g., one or more input devices (e.g., keyboard, mouse, etc.), one or more output devices (e.g., a display), data storage/memory component(s) (e.g., random access memory, read only memory), communications circuitry, etc. It will be understood that different users may employ computer systems having any of a wide variety of different components or configurations.

One or more components of an inventive system may be distributed across one or more computer systems, one or more of which may be coupled to a communication network. For example, various embodiments of the invention or components thereof may be distributed among one or more computer systems configured to provide a service (e.g., servers) to one or more client computers, or to perform a task as part of a distributed system. For example, various embodiments of the invention or components thereof may be performed on a client-server system that includes components distributed among one or more server systems that perform various functions according to various embodiments of the invention. These components may communicate over one or more communication networks using a communication protocol. A “communication network”, refers to a computer network or group of interconnected computer networks (e.g., the Internet), phone network, or any other collection of terminal nodes, links and any intermediate nodes which are connected so as to enable communication between the terminals through electrical signals or electromagnetic waves. Unless otherwise indicated or specified, a communication network may include one or more intranets or the Internet. It will be understood that a connection may be wired or wireless.

A system may comprise a website, which may provide web portals for physicians and patients. A web portal may be a page or section of a website that is dedicated to a particular constituency, such as physicians or patients, and may serve as an entry point to portions of the website that serve that constituency, e.g., by providing access to particular web pages, e.g., web pages through which users interact with a system or computer program product. Web portals may be provided for researchers, sponsors, payers, regulators, or other user constituencies.

In some embodiments, computer-executable instructions for performing any of the processes and/or methods described herein, and/or databases generated as described herein, may be stored or executed remotely from locations at which patient data are generated or entered. In some embodiments, such computer-executable instructions and/or databases may be at least in part cloud-based, wherein access to such computer-executable instructions and/or databases, is provided (e.g., as a service) over a communication network, e.g., the Internet or, e.g., a virtual private network. A cloud may be a public cloud, wherein cloud services are provided by, e.g., public cloud service providers that make such services available to the general public, such Amazon (Amazon Web Services), Microsoft (Microsoft Azure), or Google (Google Compute Engine), or may be a cloud that is not generally or broadly available to the public.

A cloud computing environment may include one or more resource providers which may provide computing resources. In some implementations, computing resources may include any hardware and/or software used to process data. For example, computing resources may include hardware and/or software capable of executing algorithms, computer programs, and/or computer applications. In some implementations, exemplary computing resources may include application servers and/or databases with storage and retrieval capabilities. Each resource provider may be connected to any other resource provider in the cloud computing environment. In some implementations, the resource providers may be connected over a computer network. Each resource provider may be connected to one or more computing devices, over a computer network.

The cloud computing environment may include a resource manager. The resource manager may be connected to the resource providers and the computing devices over the computer network. In some implementations, the resource manager may facilitate the provision of computing resources by one or more resource providers to one or more computing devices. The resource manager may receive a request for a computing resource from a computing device. The resource manager may identify one or more resource providers capable of providing the computing resource requested by the computing device. The resource manager may select a resource provider to provide the computing resource. The resource manager may facilitate a connection between the resource provider and the computing device. In some implementations, the resource manager may establish a connection between a resource provider and a computing device. In some implementations, the resource manager may redirect a computing device to a resource provider with the requested computing resource.

In some embodiments, a computer program product of the present invention or one or more features thereof is delivered or made available to users over the Internet. The product may include interfaces for personal computers (such as PC and Mac, desktops, laptops, netbooks, notebooks, and/or ultrabooks) and/or for mobile devices (such as Android and iOS based smartphones and tablets). In some embodiments, a system or product may be utilized without requiring the downloading or installation of product software on any user device. In certain embodiments, the only downloadable or installed software is a mobile application that runs on a user's mobile device.

Security, Data Integrity, and Legal Compliance

In some aspects, the system comprises security features that guard against the fraudulent or unauthorized submission of health data or other information, help protect the integrity of the health data and other information, and help limit the rights to access, contribute, or change information to those persons with credentials that entitle them to do so. Security and data integrity may be addressed in any of a variety of ways. It is contemplated that any methods known in the art for identity and access management may be used or adapted for use in one or more aspects or functions of the present invention. In some embodiments, one or more passwords may be selected by or assigned to a user and may be used for access control. Passwords may be required to be “strong” passwords and/or may be required to be changed on a regular or irregular basis. Access from a particular computer, device, or Internet address may be automatically disabled after a predetermined number of incorrect password entries. Alternatively or additionally, smartcards (which may contain an embedded computer chip or magnetically stored identifying information), digital certificates (optionally encrypted in a smart card), and/or biometric identification may be used to control access. In some embodiments, an application, database or at least a portion thereof, is accessible via a virtual private network (VPN). In some embodiments a VPN is a mobile VPN.

In some embodiments, a system maintains a list of an HCP's patients, which may be referred to as an HCP list or roster. An HCP may be permitted to access T-plans of patients on his or her roster but may not be permitted to access those of patients not on his or her roster (except to the extent such access may be permitted to the HCP as a subscriber, e.g., in de-identified form). An HCP may be permitted to modify (e.g., change or add information to) one or more T-plans of patients on his or her roster but may not be permitted to modify T-plans of patients not on his or her roster. In some embodiments, a list of the HCPs who are authorized to access and/or modify a patient's T-plan(s) or portion thereof may also be maintained and used for similar purposes (i.e., verification that an HCP is authorized to modify a T-plan for a particular patient).

In some embodiments, a location-based identification system may be used in certain aspects of the present disclosure. In some embodiments, for example, a T-plan may only be accessed from a particular computer if an electronic device belonging to an individual authorized to do so is located within a specified distance of the computer. In some embodiments, a location-based identification system may be used to facilitate patient check-in at a health care facility, e.g., for an appointment with a HCP.

An electronic device may include a suitable positioning system. The positioning system may include any suitable system such as, for example, a global positioning system (“GPS”) receiver for, e.g., accessing a GPS application function call that returns the geographic coordinates (i.e., the geographic location) of the electronic device. In some embodiments the positioning system may utilize any suitable trilateration or triangulation technique to determine the geographic coordinates of the electronic device. In some embodiments localization may occur either via multilateration (e.g., trilateration) of radio signals between radio towers and the phone. In some embodiments, the positioning system may determine various measurements (e.g., signal-to-noise ratio or signal strength measurements) of a network signal (e.g., a cellular telephone network signal, a wireless network access point or “hot spot”, or any other suitable network signal) associated with the electronic device to determine its location. While it is envisioned that mobile phones may often be used for localization, it should be understood that other personal, localizable electronic devices could be used for the same purpose.

In some embodiments, patient consent may be required in order for health care provider to prescribe a T-plan to a patient or for a health care provider or other user to gain access to the patient's T-plan(s) prescribed by other users. In some embodiments, patient consent could be verified, for example, using a password-based approach, wherein both the health care provider (or other user) and the patient (or the patient's agent) need to enter a password into the same electronic device, and/or using a location-based approach, wherein the patient (or the patient's agent) and the user need to be co-localized (as determined, for example, by mobile phone tracking). In some embodiments, a patient (or their agent) could authorize different levels of access. For example, a patient may want a particular HCP to be able to update that patient's T-plan with new information and/or add new T-plans. In some embodiments, a feature is provided that may, e.g., if selected by the patient, permit overriding the requirement for patient consent. For example, patient consent may be overridden in case of an emergency, such as the patient being admitted unconscious to a hospital after an accident. In some embodiments, a signature capture pad may be used in various aspects herein, e.g., to document informed consent for diagnostic tests, treatments, etc. and/or for capturing HCP signature.

In many embodiments, at least some data, e.g., at least some health data, may be encrypted, e.g., at least while stored and/or while being transmitted over a network. Encryption may conform to any applicable standards for health data storage or transmission. In some embodiments public-key cryptography, also known as asymmetric cryptography, which generally refers to a cryptographic algorithm which requires two separate keys one of which is secret (or private) and one of which is public, may be used. In some embodiments, an encryption standard that has been adopted by the U.S. Federal government or mandated by U.S. federal law (e.g., under the Health Information Technology for Economic and Clinical Health (HITECH) Act (part of the 2009 American Recovery and Reinvestment Act) as it may be amended or updated from time to time may be used. For example, the encryption standard known as Advanced Encryption Standard (AES) or its successors may be used.

In some embodiments, voice recognition technology may be used to facilitate security and/or integrity of the health information. For example, in some embodiments, only a user whose voice may be recognized by the system as belonging to an individual authorized to access specific data or perform a specific action may be able to do so.

In some embodiments, a system may comply with and/or facilitate HCP and/or health care institution or organization compliance with legal requirements such as those of the HITECH Act and/or HIPAA. In some embodiments, a system may qualify as an EMR system whose adoption and demonstration of one or more elements of meaningful use may entitle HCPs and/or health care institutions (e.g., eligible professionals (EPs), eligible hospitals, and critical access hospitals) to incentive payments available under U.S. federal laws such as the HITECH Act and regulations issued pursuant thereto (see 42 CFR Parts 412, 413, 422 et al. Medicare and Medicaid Programs; Electronic Health Record Incentive Program; Final Rule, published in the Federal Register on July 28, 2010, Vol. 75, No. 144; http://edocketaccess.gpo.gov/2010/pdf/2010-17207.pdf), as they may be amended from time to time. In some embodiments, a system may provide compliance with one or more elements of meaningful use when used together with one or more other systems. In some embodiments, a system may satisfy standards, implementation specifications, and/or certification criteria set forth in 45 CFR Part 170; Health Information Technology: Initial Set of Standards, Implementation Specifications, and Certification Criteria for Electronic Health Record Technology; Final Rule, published in the Federal Register on July 28, 2010, Vol. 75, No. 144; http://edocket.access.gpo.gov/2010/pdf/2010-17210.pdf), and/or any subsequent standards issued pursuant to the HITECH Act. In some embodiments, a system or at least some components or functions thereof, comply with applicable HL7 standards version 2.x or version 3, if any, and/or are adapted to interface with other systems that comply with such standards. In some embodiments, a system is certified by an organization recognized by the Office of the National Coordinator for Health Information Technology (ONC) as an Authorized Testing and Certification Body (ONC-ATCB). In some embodiments, a system is certified by the Certification Commission for Health Information Technology (CCHIT®) http://cchit.org and/or another certification body.

It is expressly contemplated that each of the various aspects, embodiments, and features thereof described herein may be freely combined with any or all other aspects, embodiments, and features. The resulting aspects and embodiments (e.g., products and methods) are within the scope of the invention. It should be understood that headings herein are provided for purposes of convenience and do not imply any limitation on content included below such heading or the use of such content in combination with content included below other headings.

All articles, books, patent applications, patents, other publications, websites, and databases mentioned in this application are incorporated herein by reference. In the event of a conflict between the specification and any of the incorporated references the specification (including any amendments thereto) shall control. Unless otherwise indicated, art-accepted meanings of terms and abbreviations are used herein.

Those skilled in the art will recognize, or be able to ascertain using no more than routine experimentation, many equivalents to the specific embodiments of the invention described herein. The scope of the present invention is not intended to be limited to the above Description, but rather is as set forth in the appended claims. In the claims articles such as “a”, “an” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. It is to be understood that the invention encompasses all variations, combinations, and permutations in which one or more limitations, elements, clauses, descriptive terms, etc., from one or more of the listed claims is introduced into another claim. For example, any claim that is dependent on another claim may be modified to include one or more elements, limitations, clauses, or descriptive terms, found in any other claim that is dependent on the same base claim. Furthermore, where the claims recite a product (e.g., an apparatus or device or computer-readable medium), it is to be understood that methods of using the product according to any of the methods disclosed herein, and methods of making the product, are included within the scope of the invention, unless otherwise indicated or unless it would be evident to one of ordinary skill in the art that a contradiction or inconsistency would arise. For example, methods comprising executing computer-readable instructions to perform one or more acts or steps relating to a T-plan, EMR, or database, such as accessing, retrieving, or analyzing one or more data elements therein, are provided. Any method may comprise a step of receiving a transmission, which transmission may comprise a query. Any method may comprise a step of analyzing a transmission, which transmission may comprise a query. Any method may comprise a step of transmitting (e.g., following receipt of a query), which transmission may comprise a response to a query. An apparatus may comprise one or more computer-readable media (e.g., memory). A memory may comprise one or more non-transitory computer-readable media. In some embodiments, a memory may comprise at least a first medium and a second medium, wherein the first medium comprises a database and the second medium comprises the instructions. A database, or instructions, or both, may be stored on or divided among any number of computer-readable media, in various embodiments. An apparatus may comprise one or more processors. An apparatus may comprise one or more computer-readable media and one or more processors. A system may comprise an apparatus, which may itself comprise one or more systems or apparatuses. A claim expressed at least in part in terms a system may be expressed at least in part in terms of an apparatus (or apparatuses), or vice versa. Where a user or an act performed by a user are described, such user may, in at least some embodiments, be a designee of the user, and/or such act may be performed by a designee of the user, e.g., under direction of the user. Where elements are presented as lists, it is to be understood that each subgroup of the elements is also disclosed, and any element(s) may be removed from the group. The invention provides all such embodiments.

The terms “approximately” or “about” in reference to a number generally include numbers that fall within ±10%, in some embodiments ±5%, in some embodiments ±1%, in some embodiments ±0.5% of the number unless otherwise stated or otherwise evident from the context (except where such number would impermissibly exceed 100% of a possible value). Where ranges are given, endpoints are included. Furthermore, it is to be understood that, unless otherwise indicated or otherwise evident from the context and understanding of one of ordinary skill in the art, values that are expressed as ranges may assume any specific value or subrange within the stated ranges in different embodiments of the invention, to the tenth of the unit of the lower limit of the range, unless the context clearly dictates otherwise. Any one or more embodiment(s), element(s), feature(s), aspect(s), component(s) etc., of the present invention may be explicitly excluded from any one or more of the claims.

1.-135. (canceled)

136. A computer program product comprising a non-transitory computer-readable medium having computer-executable instructions stored thereon for performing a method for configuring a health tracking and management plan (T-plan) for a condition, the method comprising: (a) receiving information that identifies a condition; (b) receiving information that identifies a set of patient characteristics; (c) selecting one or more data collection modules based on the condition identified by step (a) and the patient characteristics identified by step (b); and
(d) storing information that identifies the set of data collection modules selected in step (c) in association with an identifier for the T-plan, and an identifier of the condition, thereby configuring a T-plan for the condition.

137. The computer program product of claim 136, wherein step (c) comprises: accessing a database that stores, in association with identifiers of each of a plurality of conditions and each of a plurality of patient characteristics, identifiers of one or more data collection modules; selecting identifiers of those data collection modules having identifiers stored in association with the identifiers of the condition identified in step (a); and selecting identifiers of those data collection modules having identifiers stored in association with the identifiers of the patient characteristics identified in step (b), if any.

138. The computer program product of claim 137, wherein the method comprises receiving information that identifies a patient; and storing information that identifies the set of data collection modules selected in step (c) in association with an identifier of the patient, thereby assigning the T-plan to the patient.

139. The computer program product of claim 138, wherein the method further comprises providing access to at least some of the data collection modules data and, optionally, a schedule of health care events, to the patient through an application on a mobile device or through an electronic communication network.

140. The computer program product of claim 138, wherein at least some of the data collection modules obtain data over a course of time regarding or relating to a patient to whom the T-plan has been assigned, and the computer-executable instructions further perform a method that comprises: receiving data obtained over a course of time regarding or relating to the patient by at least one of said modules; and storing at least some of said data in association with an identifier of the patient and, optionally, in association with an identifier of the source of the data.

141. The computer program product of claim 140 wherein at least some of the data received regarding or relating to the patient are received while the patient is not an inpatient.

142. The computer program product of claim 136, wherein at least one of the data collection modules comprises a health tracking module that obtains symptom data and/or physiological data of one or more types that are relevant to the condition identified in step (a) or to a patient characteristic identified in step (b), a monitoring module that obtains physiological data of one or more types that are relevant to the condition identified in step (a) or to a patient characteristic identified in step (b), or an educational module that provides educational material relevant to the condition identified in step (a) or to a patient characteristic identified in step (b) and obtains behavioral data concerning use of the educational module by a patient to whom the T-plan has been assigned.

143. The computer program product of claim 142, wherein the data collection modules, themselves or together with one or more associated modules, collectively comprise computer-executable instructions for performing a method comprising: (i) obtaining data comprising symptom data, physiological data, behavioral data, and/or environmental data of one or more types that are relevant to the condition identified in step (a) or to a patient characteristic identified in step (b); and (ii) determining directions for management of the condition by a patient or caregiver based on the data and, optionally, determining whether a notification to a caregiver or health care provider should be issued based on the data.

144. The computer program product of claim 143, wherein (I) the method is performed at any time in response to a request from the patient; (II) the method is performed automatically on a recurring basis, and, optionally, the frequency or time at which the method is performed is automatically adjusted based on data regarding or relating to the patient that was previously obtained by the computer program product; (III) the method is performed automatically based on detection of physiological data indicative of a deterioration or potential deterioration or notable change in the patient's health; or (IV) any combination of (I), (II), and (III).

145. The computer program product of claim 143, wherein the method comprises conducting an interactive health evaluation regarding the condition.

146. The computer program product of claim 136, wherein at least one of the data collection modules comprises a health tracking module that obtains symptom data, physiological data, or both, by presenting one or more questions to a patient or caregiver through an application on a mobile device or through an electronic communication network and receiving responses thereto.

147. A computer program product comprising a non-transitory computer-readable medium having stored thereon computer-executable instructions for performing a method comprising: providing a user interface on a computer display that permits a user to select or configure a computer program product according to claim 143 and assign it to a patient.

148. The computer program product of claim 147, wherein the data collection modules, themselves or through one or more associated modules, determine directions for management of the condition by a patient or caregiver based on data received by the data collection modules and provide the directions to a patient or caregiver, and wherein the user interface permits the user to (i) view a representation of a process by which directions for management of the condition by a patient or caregiver are determined based on a given set of data, optionally wherein the process is represented by displaying the various combinations of data that result in particular directions, (ii) remove a feature that provides directions for management of the condition by a patient or caregiver, (iii) customize one or more elements of the directions, or (iv) any combination of (i)-(iii).

149. A computer program product comprising computer-executable instructions stored on a non-transitory computer-readable medium for performing a method comprising: conducting an interactive health evaluation regarding a condition and presenting directions for management of the condition to the patient, wherein the directions are determined based on data obtained through the interactive health evaluation, wherein the interactive health evaluation (i) is conducted automatically upon detection of physiological data indicative of a deterioration or potential deterioration or notable change in the patient's health and/or (ii) is conducted automatically on a recurring basis, optionally wherein the interactive health evaluation is, in addition to (i) and/or (ii), also conducted at any time in response to a request received from a patient who suffers from the condition.

150. The computer program product of claim 149, wherein the interactive health evaluation is, in addition to (i) and/or (ii), also conducted at any time in response to a request received from a patient who suffers from the condition.

151. The computer program product of claim 149, wherein the interactive health evaluation is conducted by a health tracking module that is configured to be assignable to a patient by a health care provider or has been assigned to a patient by a health care provider.

152. The computer program product of claim 149, wherein the method further comprises transmitting data obtained through an interactive health evaluation to a computer that stores it in a database in association with an identifier of the patient and accessible for analysis or presentation to a health care provider of the patient.

153. The computer program product of claim 149, comprising an application that provides a user interface that permits a patient or caregiver to (1) evaluate the patient's condition at any time by requesting and participating in an interactive health evaluation, (2) be prompted to participate in and participate in an interactive health evaluation that is conducted automatically on a recurring basis and/or is conducted automatically upon detection of physiological data indicative of a deterioration or potential deterioration or notable change in the patient's health, and (3) receive directions for management of the condition, wherein the directions are determined based on data obtained through the interactive health evaluation, optionally wherein the application is for a mobile device or is a web-based application.

154. A computer program product comprising a non-transitory computer-readable medium having computer-executable instructions stored thereon, the computer-executable instructions for performing a method comprising: (a) receiving from a user information that identifies a patient and a condition; (b) presenting to the user a user interface configured to permit the user to select a subset of patient characteristics that apply to the patient from a predetermined set of patient characteristics that are deemed significant in the context of the condition; (c) receiving, from the user, information selecting a subset of the set of patient characteristics; (d) selecting a set of one or more data collection modules based on the condition and the patient characteristics selected by the information received from the user, wherein at least one of the data collection modules obtains symptom data, physiological data, or both, regarding the patient; (e) providing access to at least some of the data collection modules selected in step (d) to the patient or a caregiver through an application on a mobile device or through an electronic communication network; (f) receiving data entered by the patient or a caregiver or collected by a connected monitoring device over a course of time; and (g) storing at least some of the data received in association with an identifier of the patient.

155. The computer program product of claim 154, wherein the method further comprises determining directions for management of the condition by a patient or caregiver based on the data; and providing the directions to the patient or a caregiver through the application on a mobile device or through the electronic communication network.
